SEQUENCE LISTING (325 sequences)

SEQ ID NO: 1 - protein, 7 aa, Homo sapiens
SQAIATK

SEQ ID NO: 2 - protein, 7 aa, Homo sapiens
GVPIPNK

SEQ ID NO: 3 - protein, 7 aa, Homo sapiens
GVPIPNK

SEQ ID NO: 4 - protein, 7 aa, Homo sapiens
INSGAHK

SEQ ID NO: 5 - protein, 7 aa, Homo sapiens
LAIPEGK

SEQ ID NO: 6 - protein, 7 aa, Homo sapiens
VSQEGLK

SEQ ID NO: 7 - protein, 7 aa, Homo sapiens
QSELSAK

SEQ ID NO: 8 - protein, 7 aa, Homo sapiens
NFATSNK

SEQ ID NO: 9 - protein, 7 aa, Homo sapiens
DADDLQK

SEQ ID NO: 10 - protein, 7 aa, Homo sapiens
AYSDLSR

SEQ ID NO: 11 - protein, 8 aa, Homo sapiens
GSLHANGR

SEQ ID NO: 12 - protein, 7 aa, Homo sapiens
AQAWGER

SEQ ID NO: 13 - protein, 7 aa, Homo sapiens
QSSEITR

SEQ ID NO: 14 - protein, 7 aa, Homo sapiens
EQVAEVR

SEQ ID NO: 15 - protein, 8 aa, Homo sapiens
GNTEGLQK

SEQ ID NO: 16 - protein, 8 aa, Homo sapiens
ELQAAQAR

SEQ ID NO: 17 - protein, 8 aa, Homo sapiens
HDAHLNGK

SEQ ID NO: 18 - protein, 8 aa, Homo sapiens
GNYDAAQR

SEQ ID NO: 19 - protein, 8 aa, Homo sapiens
LNGESNLR

SEQ ID NO: 20 - protein, 8 aa, Homo sapiens
QELNGNTK

SEQ ID NO: 21 - protein, 9 aa, Homo sapiens
SFNPNSPGK

SEQ ID NO: 22 - protein, 8 aa, Homo sapiens
QLTPYAQR

SEQ ID NO: 23 - protein, 9 aa, Homo sapiens
VNDESTEGK

SEQ ID NO: 24 - protein, 9 aa, Homo sapiens
TDASDVKPC

SEQ ID NO: 25 - protein, 9 aa, Homo sapiens
IKQSELSAK

SEQ ID NO: 26 - protein, 9 aa, Homo sapiens
GAYQNNEIK

SEQ ID NO: 27 - protein, 9 aa, Homo sapiens
ESDEETQIK

SEQ ID NO: 28 - protein, 9 aa, Homo sapiens
HSHESQDLR

SEQ ID NO: 29 - protein, 11 aa, Homo sapiens
VKSPELQAEAK

SEQ ID NO: 30 - protein, 11 aa, Homo sapiens
EGTHGSHVYTK

SEQ ID NO: 31 - protein, 10 aa, Homo sapiens
EDEDFQESNK

SEQ ID NO: 32 - protein, 10 aa, Homo sapiens
QQTEWQSGQR

SEQ ID NO: 33 - protein, 10 aa, Homo sapiens
DNEDFQESNR

SEQ ID NO: 34 - protein, 12 aa, Homo sapiens
HQDGSYSTFGER

SEQ ID NO: 35 - protein, 17 aa, Homo sapiens
VQAAVGTSAAPVPSDNH

SEQ ID NO: 36 - protein, 14 aa, Homo sapiens
HSHNNNSSDLHPHK

SEQ ID NO: 37 - protein, 9 aa, Homo sapiens
SPELQAEAK

SEQ ID NO: 38 - protein, 12 aa, Homo sapiens
SLAPYAQDTQEK

SEQ ID NO: 39 - protein, 8 aa, Homo sapiens
QYTDSTFR

SEQ ID NO: 40 - protein, 20 aa, Homo sapiens
YTYNYEAESSSGVPGTADSR

SEQ ID NO: 41 - protein, 7 aa, Homo sapiens
ETLEDTR

SEQ ID NO: 42 - protein, 7 aa, Homo sapiens
QIGSVYR

SEQ ID NO: 43 - protein, 13 aa, Homo sapiens
AASGTTGTYQEWK

SEQ ID NO: 44 - protein, 8 aa, Homo sapiens
IADAHLDR

SEQ ID NO: 45 - protein, 9 aa, Homo sapiens
ATVVYQGER

SEQ ID NO: 46 - protein, 7 aa, Homo sapiens
AHVDALR

SEQ ID NO: 47 - protein, 7 aa, Homo sapiens
LPSNVVK

SEQ ID NO: 48 - protein, 7 aa, Homo sapiens
FFGEGTK

SEQ ID NO: 49 - protein, 8 aa, Homo sapiens
FRPDGLPK

SEQ ID NO: 50 - protein, 9 aa, Homo sapiens
LEEQAQQIR

SEQ ID NO: 51 - protein, 7 aa, Homo sapiens
QAEAVLK

SEQ ID NO: 52 - protein, 7 aa, Homo sapiens
YYELEEK

SEQ ID NO: 53 - protein, 9 aa, Homo sapiens
LAVYQAGAR

SEQ ID NO: 54 - protein, 8 aa, Homo sapiens
EAVEHLQK

SEQ ID NO: 55 - protein, 9 aa, Homo sapiens
TAAQNLYEK

SEQ ID NO: 56 - protein, 11 aa, Homo sapiens
LLPHANEVSQK

SEQ ID NO: 57 - protein, 11 aa, Homo sapiens
DFSAEYEEDGK

SEQ ID NO: 58 - protein, 11 aa, Homo sapiens
TQVSTQAEQLR

SEQ ID NO: 59 - protein, 8 aa, Homo sapiens
YGAATFTR

SEQ ID NO: 60 - protein, 9 aa, Homo sapiens
IPTTFENGR

SEQ ID NO: 61 - protein, 11 aa, Homo sapiens
SNTVASLHTEK

SEQ ID NO: 62 - protein, 10 aa, Homo sapiens
VEPYGENFNK

SEQ ID NO: 63 - protein, 7 aa, Homo sapiens
LRDLYSK

SEQ ID NO: 64 - protein, 13 aa, Homo sapiens
IVLGQEQDSYGGK

SEQ ID NO: 65 - protein, 9 aa, Homo sapiens
LDNIYSSDK

SEQ ID NO: 66 - protein, 9 aa, Homo sapiens
VAWHYDEEK

SEQ ID NO: 67 - protein, 7 aa, Homo sapiens
VSSFYAK

SEQ ID NO: 68 - protein, 11 aa, Homo sapiens
LPPNVVEESAR

SEQ ID NO: 69 - protein, 13 aa, Homo sapiens
VNKDDEEFIESNK

SEQ ID NO: 70 - protein, 7 aa, Homo sapiens
EAQEVFK

SEQ ID NO: 71 - protein, 15 aa, Homo sapiens
VGAHAGEYGAEALER

SEQ ID NO: 72 - protein, 9 aa, Homo sapiens
LQAEAFQAR

SEQ ID NO: 73 - protein, 7 aa, Homo sapiens
VLNQELR

SEQ ID NO: 74 - protein, 11 aa, Homo sapiens
AGHIAWTSSGK

SEQ ID NO: 75 - protein, 7 aa, Homo sapiens
AAIQALR

SEQ ID NO: 76 - protein, 7 aa, Homo sapiens
VDPVNFK

SEQ ID NO: 77 - protein, 9 aa, Homo sapiens
GPGGVWAAK

SEQ ID NO: 78 - protein, 14 aa, Homo sapiens
CPNPPVQENFDVNK

SEQ ID NO: 79 - protein, 8 aa, Homo sapiens
QIDDIDVR

SEQ ID NO: 80 - protein, 11 aa, Homo sapiens
RPSEIVIGQCK

SEQ ID NO: 81 - protein, 9 aa, Homo sapiens
IDQNVEELK

SEQ ID NO: 82 - protein, 11 aa, Homo sapiens
ATEHLSTLSEK

SEQ ID NO: 83 - protein, 7 aa, Homo sapiens
YNILPEK

SEQ ID NO: 84 - protein, 7 aa, Homo sapiens
YNILPEK

SEQ ID NO: 85 - protein, 9 aa, Homo sapiens
SLDEHYHIR

SEQ ID NO: 86 - protein, 9 aa, Homo sapiens
LHVDPENFR

SEQ ID NO: 87 - protein, 12 aa, Homo sapiens
EEGTDLEVTANR

SEQ ID NO: 88 - protein, 9 aa, Homo sapiens
LGPLVEQGR

SEQ ID NO: 89 - protein, 10 aa, Homo sapiens
QGYFVEAQPK

SEQ ID NO: 90 - protein, 9 aa, Homo sapiens
EFGNTLEDK

SEQ ID NO: 91 - protein, 9 aa, Homo sapiens
AKPALEDLR

SEQ ID NO: 92 - protein, 16 aa, Homo sapiens
DALSSVQESQVAQQAR

SEQ ID NO: 93 - protein, 9 aa, Homo sapiens
LDVTTSIGR

SEQ ID NO: 94 - protein, 15 aa, Homo sapiens
AAQVTIQSSGTFSSK

SEQ ID NO: 95 - protein, 7 aa, Homo sapiens
VHELIER

SEQ ID NO: 96 - protein, 7 aa, Homo sapiens
FLDSNIK

SEQ ID NO: 97 - protein, 7 aa, Homo sapiens
GVISIPR

SEQ ID NO: 98 - protein, 11 aa, Homo sapiens
EALQGVGDMGR

SEQ ID NO: 99 - protein, 13 aa, Homo sapiens
VNVDEVGGEALGR

SEQ ID NO: 100 - protein, 7 aa, Homo sapiens
FIIPSPK

SEQ ID NO: 101 - protein, 9 aa, Homo sapiens
TYLPAVDEK

SEQ ID NO: 102 - protein, 10 aa, Homo sapiens
LTISEQNIQR

SEQ ID NO: 103 - protein, 7 aa, Homo sapiens
ALVDTLK

SEQ ID NO: 104 - protein, 12 aa, Homo sapiens
INNQLTLDSNTK

SEQ ID NO: 105 - protein, 9 aa, Homo sapiens
LEPYADQLR

SEQ ID NO: 106 - protein, 12 aa, Homo sapiens
VVAGVANALAHK

SEQ ID NO: 107 - protein, 14 aa, Homo sapiens
EVGPTNADPVCLAK

SEQ ID NO: 108 - protein, 9 aa, Homo sapiens
QHIEAIDVR

SEQ ID NO: 109 - protein, 7 aa, Homo sapiens
DYWSTVK

SEQ ID NO: 110 - protein, 18 aa, Homo sapiens
IHSGSFQSQVELSNDQEK

SEQ ID NO: 111 - protein, 9 aa, Homo sapiens
LVELAHQYK

SEQ ID NO: 112 - protein, 9 aa, Homo sapiens
LAPLAEDVR

SEQ ID NO: 113 - protein, 7 aa, Homo sapiens
TPALHFK

SEQ ID NO: 114 - protein, 15 aa, Homo sapiens
FVEGSHNSTVSLTTK

SEQ ID NO: 115 - protein, 8 aa, Homo sapiens
TFIEDVNK

SEQ ID NO: 116 - protein, 11 aa, Homo sapiens
AIGYLNTGYQR

SEQ ID NO: 117 - protein, 9 aa, Homo sapiens
LTPYADEFK

SEQ ID NO: 118 - protein, 10 aa, Homo sapiens
IQNILTEEPK

SEQ ID NO: 119 - protein, 12 aa, Homo sapiens
EFTPPVQAAYQK

SEQ ID NO: 120 - protein, 8 aa, Homo sapiens
ALDLINKR

SEQ ID NO: 121 - protein, 8 aa, Homo sapiens
QSFDLSVK

SEQ ID NO: 122 - protein, 10 aa, Homo sapiens
DDEEFIESNK

SEQ ID NO: 123 - protein, 9 aa, Homo sapiens
LSNVLQQVK

SEQ ID NO: 124 - protein, 7 aa, Homo sapiens
GEAFTLK

SEQ ID NO: 125 - protein, 7 aa, Homo sapiens
LFLEETK

SEQ ID NO: 126 - protein, 10 aa, Homo sapiens
YEGLQEWEGK

SEQ ID NO: 127 - protein, 9 aa, Homo sapiens
INDVLEHVK

SEQ ID NO: 128 - protein, 10 aa, Homo sapiens
IISDYHQQFR

SEQ ID NO: 129 - protein, 13 aa, Homo sapiens
LGEVNTYAGDLQK

SEQ ID NO: 130 - protein, 9 aa, Homo sapiens
LRVDPVNFK

SEQ ID NO: 131 - protein, 8 aa, Homo sapiens
DLKPAIVK

SEQ ID NO: 132 - protein, 15 aa, Homo sapiens
AATVGSLAGQPLQER

SEQ ID NO: 133 - protein, 7 aa, Homo sapiens
ALDLINK

SEQ ID NO: 134 - protein, 9 aa, Homo sapiens
FEVQVTVPK

SEQ ID NO: 135 - protein, 15 aa, Homo sapiens
SGSSTASWIQNVDTK

SEQ ID NO: 136 - protein, 11 aa, Homo sapiens
LKEFGNTLEDK

SEQ ID NO: 137 - protein, 10 aa, Homo sapiens
QGFFPDSVNK

SEQ ID NO: 138 - protein, 12 aa, Homo sapiens
QSEDSTFYLGER

SEQ ID NO: 139 - protein, 7 aa, Homo sapiens
INPLALK

SEQ ID NO: 140 - protein, 7 aa, Homo sapiens
LNELSFK

SEQ ID NO: 141 - protein, 18 aa, Homo sapiens
TQFNNNEYSQDLDAYNTK

SEQ ID NO: 142 - protein, 7 aa, Homo sapiens
LSLPDFK

SEQ ID NO: 143 - protein, 9 aa, Homo sapiens
VSTAFVYTK

SEQ ID NO: 144 - protein, 13 aa, Homo sapiens
SVSLPSLDPASAK

SEQ ID NO: 145 - protein, 8 aa, Homo sapiens
EDSPFALK

SEQ ID NO: 146 - protein, 9 aa, Homo sapiens
DSYDLHDLK

SEQ ID NO: 147 - protein, 6 aa, Homo sapiens
WYEIEK

SEQ ID NO: 148 - protein, 12 aa, Homo sapiens
AYSLFSYNTQGR

SEQ ID NO: 149 - protein, 10 aa, Homo sapiens
EENDDFASFR

SEQ ID NO: 150 - protein, 7 aa, Homo sapiens
AQIPILR

SEQ ID NO: 151 - protein, 16 aa, Homo sapiens
EHEGAIYPDNTTDFQR

SEQ ID NO: 152 - protein, 15 aa, Homo sapiens
FNSSYLQGTNQITGR

SEQ ID NO: 153 - protein, 8 aa, Homo sapiens
EILNSLDK

SEQ ID NO: 154 - protein, 13 aa, Homo sapiens
NPNLPPETVDSLK

SEQ ID NO: 155 - protein, 11 aa, Homo sapiens
THLAPYSDELR

SEQ ID NO: 156 - protein, 8 aa, Homo sapiens
QWAGLVEK

SEQ ID NO: 157 - protein, 7 aa, Homo sapiens
STVELFK

SEQ ID NO: 158 - protein, 10 aa, Homo sapiens
ALVQQMEQLR

SEQ ID NO: 159 - protein, 14 aa, Homo sapiens
SFDYHQFVDETNDK

SEQ ID NO: 160 - protein, 8 aa, Homo sapiens
QVFLYPEK

SEQ ID NO: 161 - protein, 11 aa, Homo sapiens
ESLSSYWESAK

SEQ ID NO: 162 - protein, 7 aa, Homo sapiens
GEVFTLK

SEQ ID NO: 163 - protein, 11 aa, Homo sapiens
VGFYESDVMGR

SEQ ID NO: 164 - protein, 9 aa, Homo sapiens
YENYELTLK

SEQ ID NO: 165 - protein, 11 aa, Homo sapiens
SSGSLLNNAIK

SEQ ID NO: 166 - protein, 11 aa, Homo sapiens
SKEQLTPLIKK

SEQ ID NO: 167 - protein, 10 aa, Homo sapiens
QTLSWTVTPK

SEQ ID NO: 168 - protein, 9 aa, Homo sapiens
IPSVQINFK

SEQ ID NO: 169 - protein, 10 aa, Homo sapiens
IYSLWEHSTK

SEQ ID NO: 170 - protein, 11 aa, Homo sapiens
NILTSNNIDVK

SEQ ID NO: 171 - protein, 21 aa, Homo sapiens
ATFGCHDGYSLDGPEEIECTK

SEQ ID NO: 172 - protein, 9 aa, Homo sapiens
FPEVDVLTK

SEQ ID NO: 173 - protein, 12 aa, Homo sapiens
EVGTVLSQVYSK

SEQ ID NO: 174 - protein, 12 aa, Homo sapiens
YGMVAQVTQTLK

SEQ ID NO: 175 - protein, 9 aa, Homo sapiens
ATVLNYLPK

SEQ ID NO: 176 - protein, 22 aa, Homo sapiens
ADGTVNQIEGEATPVNLTEPAK

SEQ ID NO: 177 - protein, 10 aa, Homo sapiens
VGEYSLYIGR

SEQ ID NO: 178 - protein, 10 aa, Homo sapiens
SKEQLTPLIK

SEQ ID NO: 179 - protein, 15 aa, Homo sapiens
SELEEQLTPVAEETR

SEQ ID NO: 180 - protein, 7 aa, Homo sapiens
FIIPGLK

SEQ ID NO: 181 - protein, 8 aa, Homo sapiens
EQLTPLIK

SEQ ID NO: 182 - protein, 13 aa, Homo sapiens
HIQNIDIQHLAGK

SEQ ID NO: 183 - protein, 13 aa, Homo sapiens
WNFYYSPQSSPDK

SEQ ID NO: 184 - protein, 15 aa, Homo sapiens
ATLYALSHAVNNYHK

SEQ ID NO: 185 - protein, 14 aa, Homo sapiens
LQDFSDQLSDYYEK

SEQ ID NO: 186 - protein, 14 aa, Homo sapiens
IEFEWNTGTNVDTK

SEQ ID NO: 187 - protein, 7 aa, Homo sapiens
DFSLWEK

SEQ ID NO: 188 - protein, 8 aa, Homo sapiens
DNELLVYK

SEQ ID NO: 189 - protein, 11 aa, Homo sapiens
LVPFATELHER

SEQ ID NO: 190 - protein, 7 aa, Homo sapiens
NLLVALK

SEQ ID NO: 191 - protein, 12 aa, Homo sapiens
NEDSLVFVQTDK

SEQ ID NO: 192 - protein, 6 aa, Homo sapiens
FWDYLR

SEQ ID NO: 193 - protein, 10 aa, Homo sapiens
HANWTLTPLK

SEQ ID NO: 194 - protein, 9 aa, Homo sapiens
MFLSFPTTK

SEQ ID NO: 195 - protein, 9 aa, Homo sapiens
DNVFDGLVR

SEQ ID NO: 196 - protein, 13 aa, Homo sapiens
LNGEIQALELPQK

SEQ ID NO: 197 - protein, 20 aa, Homo sapiens
LEIQSQVDSQHVGHSVLTAK

SEQ ID NO: 198 - protein, 20 aa, Homo sapiens
TFYEPGEEITYSCKPGYVSR

SEQ ID NO: 199 - protein, 9 aa, Homo sapiens
VNSFFSTFK

SEQ ID NO: 200 - protein, 9 aa, Homo sapiens
EHSSLAFWK

SEQ ID NO: 201 - protein, 14 aa, Homo sapiens
EVQPVELPNCNLVK

SEQ ID NO: 202 - protein, 10 aa, Homo sapiens
LVGFIDDAVK

SEQ ID NO: 203 - protein, 7 aa, Homo sapiens
WELALGR

SEQ ID NO: 204 - protein, 15 aa, Homo sapiens
ALYWVNGQVPDGVSK

SEQ ID NO: 205 - protein, 9 aa, Homo sapiens
ATVVYQGER

SEQ ID NO: 206 - protein, 15 aa, Homo sapiens
HIYAISSAALSASYK

SEQ ID NO: 207 - protein, 17 aa, Homo sapiens
AHTSFQISLSVSYTGSR

SEQ ID NO: 208 - protein, 9 aa, Homo sapiens
NIILPVYDK

SEQ ID NO: 209 - protein, 10 aa, Homo sapiens
IEIPLPFGGK

SEQ ID NO: 210 - protein, 11 aa, Homo sapiens
MREWFSETFQK

SEQ ID NO: 211 - protein, 14 aa, Homo sapiens
ATVAVYLESLQDTK

SEQ ID NO: 212 - protein, 23 aa, Homo sapiens
QQNAQGGFSSTQDTVVALHALSR

SEQ ID NO: 213 - protein, 9 aa, Homo sapiens
LLIGTVFHK

SEQ ID NO: 214 - protein, 15 aa, Homo sapiens
VSALLTPAEQTGTWK

SEQ ID NO: 215 - protein, 9 aa, Homo sapiens
EWFSETFQK

SEQ ID NO: 216 - protein, 16 aa, Homo sapiens
VNQNLVYESGSLNFSK

SEQ ID NO: 217 - protein, 14 aa, Homo sapiens
GGEGTGYFVDFSVR

SEQ ID NO: 218 - protein, 10 aa, Homo sapiens
DEPTYILNIK

SEQ ID NO: 219 - protein, 11 aa, Homo sapiens
TEVIPPLIENR

SEQ ID NO: 220 - protein, 11 aa, Homo sapiens
EALQGVGDMGR

SEQ ID NO: 221 - protein, 16 aa, Homo sapiens
TYFPHFDLSHGSAQVK

SEQ ID NO: 222 - protein, 12 aa, Homo sapiens
ALYLQYTDETFR

SEQ ID NO: 223 - protein, 14 aa, Homo sapiens
VCPFAGILENGAVR

SEQ ID NO: 224 - protein, 17 aa, Homo sapiens
LGPHAGDVEGHLSFLEK

SEQ ID NO: 225 - protein, 23 aa, Homo sapiens
ADSVVDLLSYNVQGSGETTYDHK

SEQ ID NO: 226 - protein, 11 aa, Homo sapiens
GWVTDGFSSLK

SEQ ID NO: 227 - protein, 11 aa, Homo sapiens
EFQVPTFTIPK

SEQ ID NO: 228 - protein, 15 aa, Homo sapiens
NIQEYLSILTDPDGK

SEQ ID NO: 229 - protein, 13 aa, Homo sapiens
GAYPLSIEPIGVR

SEQ ID NO: 230 - protein, 11 aa, Homo sapiens
DKVNSFFSTFK

SEQ ID NO: 231 - protein, 14 aa, Homo sapiens
LLDNWDSVTSTFSK

SEQ ID NO: 232 - protein, 16 aa, Homo sapiens
LPQQANDYLNSFNWER

SEQ ID NO: 233 - protein, 11 aa, Homo sapiens
LKEFGNTLEDK

SEQ ID NO: 234 - protein, 12 aa, Homo sapiens
NTLELSNGVIVK

SEQ ID NO: 235 - protein, 19 aa, Homo sapiens
ASGSLPYTQTLQDHLNSLK

SEQ ID NO: 236 - protein, 20 aa, Homo sapiens
ENLTAPGSDSAVFFEQGTTR

SEQ ID NO: 237 - protein, 22 aa, Homo sapiens
VTQVYAENGTVLQGSTVASVYK

SEQ ID NO: 238 - protein, 11 aa, Homo sapiens
ESLSSYWESAK

SEQ ID NO: 239 - protein, 20 aa, Homo sapiens
AEEEHLGILGPQLHADVGDK

SEQ ID NO: 240 - protein, 13 aa, Homo sapiens
MVSGFIPLKPTVK

SEQ ID NO: 241 - protein, 11 aa, Homo sapiens
YVYIAELLAHK

SEQ ID NO: 242 - protein, 10 aa, Homo sapiens
LLVVYPWTQR

SEQ ID NO: 243 - protein, 11 aa, Homo sapiens
NILTSNNIDVK

SEQ ID NO: 244 - protein, 17 aa, Homo sapiens
ELHHLQEQNVSNAFLDK

SEQ ID NO: 245 - protein, 12 aa, Homo sapiens
LPYTIITTPPLK

SEQ ID NO: 246 - protein, 10 aa, Homo sapiens
TGISPLALIK

SEQ ID NO: 247 - protein, 9 aa, Homo sapiens
ATVLNYLPK

SEQ ID NO: 248 - protein, 10 aa, Homo sapiens
VGEYSLYIGR

SEQ ID NO: 249 - protein, 10 aa, Homo sapiens
SKEQLTPLIK

SEQ ID NO: 250 - protein, 19 aa, Homo sapiens
VVAEGFDFANGINISPDGK

SEQ ID NO: 251 - protein, 14 aa, Homo sapiens
IVQILPWEQNEQVK

SEQ ID NO: 252 - protein, 17 aa, Homo sapiens
SLAELGGHLDQQVEEFR

SEQ ID NO: 253 - protein, 6 aa, Homo sapiens
FWDYLR

SEQ ID NO: 254 - protein, 10 aa, Homo sapiens
GHFSISIPVK

SEQ ID NO: 255 - protein, 12 aa, Homo sapiens
QTIIVVLENVQR

SEQ ID NO: 256 - protein, 13 aa, Homo sapiens
VKDLATVYVDVLK

SEQ ID NO: 257 - protein, 12 aa, Homo sapiens
GFEPTLEALFGK

SEQ ID NO: 258 - protein, 16 aa, Homo sapiens
IFFYDSENPPASEVLR

SEQ ID NO: 259 - protein, 16 aa, Homo sapiens
NLTDFAEQYSIQDWAK

SEQ ID NO: 260 - protein, 17 aa, Homo sapiens
FEVDSPVYNATWSASLK

SEQ ID NO: 261 - protein, 16 aa, Homo sapiens
GIISALLVPPETEEAK

SEQ ID NO: 262 - protein, 23 aa, Homo sapiens
SVPPSASHVAPTETFTYEWTVPK

SEQ ID NO: 263 - protein, 16 aa, Homo sapiens
VNWEEEAASGLLTSLK

SEQ ID NO: 264 - protein, 16 aa, Homo sapiens
TYFPHFDLSHGSAQVK

SEQ ID NO: 265 - protein, 17 aa, Homo sapiens
LGPHAGDVEGHLSFLEK

SEQ ID NO: 266 - protein, 11 aa, Homo sapiens
GWVTDGFSSLK

SEQ ID NO: 267 - protein, 13 aa, Homo sapiens
GAYPLSIEPIGVR

SEQ ID NO: 268 - protein, 14 aa, Homo sapiens
LLDNWDSVTSTFSK

SEQ ID NO: 269 - protein, 17 aa, Homo sapiens
TYYIAAVEVEWDYSPQR

SEQ ID NO: 270 - protein, 10 aa, Homo sapiens
LAPGELTIIL

SEQ ID NO: 271 - protein, 12 aa, Homo sapiens
SWFEPLVEDMQR

SEQ ID NO: 272 - protein, 10 aa, Homo sapiens
LLVVYPWTQR

SEQ ID NO: 273 - protein, 17 aa, Homo sapiens
LSLESLTSYFSIESSTK

SEQ ID NO: 274 - protein, 10 aa, Homo sapiens
TGISPLALIK

SEQ ID NO: 275 - protein, 20 aa, Homo sapiens
YEDGTLSLTSTSDLQSGIIK

SEQ ID NO: 276 - protein, 18 aa, Homo sapiens
DQEVLLQTFLDDASPGDK

SEQ ID NO: 277 - protein, 18 aa, Homo sapiens
NHLQLEGLFFTNGEHTSK

SEQ ID NO: 278 - protein, 9 aa, Homo sapiens
DGYLFQLLR

SEQ ID NO: 279 - protein, 11 aa, Homo sapiens
EQHLFLPFSYK

SEQ ID NO: 280 - protein, 16 aa, Homo sapiens
VLGAFSDGLAHLDNLK

SEQ ID NO: 281 - protein, 15 aa, Homo sapiens
ISASSEVAFLSIQIK

SEQ ID NO: 282 - protein, 16 aa, Homo sapiens
IFFYDSENPPASEVLR

SEQ ID NO: 283 - protein, 13 aa, Homo sapiens
IAIANIIDEIIEK

SEQ ID NO: 284 - protein, 14 aa, Homo sapiens
VLLDQLGTTISFER

SEQ ID NO: 285 - protein, 21 aa, Homo sapiens
AQNLYQELLTQEGQASFQGLK

SEQ ID NO: 286 - protein, 14 aa, Homo sapiens
EEYFDPSIVGWTVK

SEQ ID NO: 287 - protein, 13 aa, Homo sapiens
TLADLTLLDSPIK

SEQ ID NO: 288 - protein, 15 aa, Homo sapiens
TSSFALNLPTLPEVK

SEQ ID NO: 289 - protein, 18 aa, Homo sapiens
LEVLNFDFQANAQLSNPK

SEQ ID NO: 290 - protein, 9 aa, Homo sapiens
DGYLFQLLR

SEQ ID NO: 291 - protein, 14 aa, Homo sapiens
DSPVLIDFFEDTER

SEQ ID NO: 292 - protein, 16 aa, Homo sapiens
ADLFYDVEALDLESPK

SEQ ID NO: 293 - protein, 19 aa, Homo sapiens
FDHTNSLNIAGLSLDFSSK

SEQ ID NO: 294 - protein, 12 aa, Homo sapiens
ALLAYAFSLLGK

SEQ ID NO: 295 - protein, 22 aa, Homo sapiens
LEAGINQLSFPLSSEPIQGSYR

SEQ ID NO: 296 - protein, 13 aa, Homo sapiens
NELIPLIYLENPR

SEQ ID NO: 297 - protein, 15 aa, Homo sapiens
HFVINLIGDFEVAEK

SEQ ID NO: 298 - protein, 20 aa, Homo sapiens
GEVLPLPEANFPSFPLPHHK

SEQ ID NO: 299 - protein, 16 aa, Homo sapiens
ATFQTPDFIVPLTDLR

SEQ ID NO: 300 - protein, 18 aa, Homo sapiens
FSVPAGIVIPSFQALTAR

SEQ ID NO: 301 - protein, 23 aa, Homo sapiens
NSLFFSAQPFEITASTNNEGNLK

SEQ ID NO: 302 - protein, 15 aa, Homo sapiens
QGIPFFAQVLLVDGK

SEQ ID NO: 303 - protein, 21 aa, Homo sapiens
QTVNLQLQPYSLVTTLNSDLK

SEQ ID NO: 304 - protein, 16 aa, Homo sapiens
TIHDLHLFIENIDFNK

SEQ ID NO: 305 - protein, 23 aa, Homo sapiens
WVQTLSEQVQEELLSSQVTQELR

SEQ ID NO: 306 - protein, 14 aa, Homo sapiens
HLIDSLIDFLNFPR

SEQ ID NO: 307 - protein, 22 aa, Homo sapiens
NFVASHIANILNSEELDIQDLK

SEQ ID NO: 308 - protein, 22 aa, Homo sapiens
AGTELVNFLSYFVELGTQPATQ

SEQ ID NO: 309 - protein, 19 aa, Homo sapiens
VSNQTLSLFFTVLQDVPVR

SEQ ID NO: 310 - protein, 21 aa, Homo sapiens
NQDVHSINLPFFETLQEYFER

SEQ ID NO: 311 - protein, 23 aa, Homo sapiens
GPEEEHLGILGPVIWAEVGDTIR

SEQ ID NO: 312 - protein, 24 aa, Homo sapiens
GIETGSEDLEILPNGLAFISSGLK

SEQ ID NO: 313 - protein, 23 aa, Homo sapiens
VYLQGLIDYYLFGNSSTVLEDSK

SEQ ID NO: 314 - protein, 21 aa, Homo sapiens
TTLTAFGFASADLIEIGLEGK

SEQ ID NO: 315 - protein, 25 aa, Homo sapiens
IDFLNNYALFLSPSAQQASWQVSAR

SEQ ID NO: 316 - protein, 20 aa, Homo sapiens
VPSYTLILPSLELPVLHVPR

SEQ ID NO: 317 - protein, 25 aa, Homo sapiens
FFSLLSGSLNSHGLELNADILGTDK

SEQ ID NO: 318 - protein, 23 aa, Homo sapiens
HYYIAAEEIIWNYAPSGIDIFTK

SEQ ID NO: 319 - protein, 20 aa, Homo sapiens
AYIFIDEAHITQALIWLSQR

SEQ ID NO: 320 - protein, 18 aa, Homo sapiens
NLQDLLQFIFQLIEDNIK

SEQ ID NO: 321 - protein, 22 aa, Homo sapiens
EFYLFPTVFDENESLLLEDNIR

SEQ ID NO: 322 - protein, 25 aa, Homo sapiens
YLSLVGQVYSTLVTYISDWWTLAAK

SEQ ID NO: 323 - protein, 21 aa, Homo sapiens
LIDLSIQNYHTFLIYITELLK

SEQ ID NO: 324 - protein, 24 aa, Homo sapiens
VHNGSEILFSYFQDLVITLPFELR

SEQ ID NO: 325 - protein, 23 aa, Homo sapiens
LEFALLFLVFDENESWYLDDNIK

(19) United States
(12) Patent Application Publication
(10) Pub. No.: US 2010/0216250 A1
(43) Pub. Date: Aug. 26, 2010

(54) Methods for Predicting Trisomy 21 in a Fetus

(76) Inventors: Mary Frances LOPEZ, Bostson, MA (US); David A. Sarracino, Andover, MA (US); Bryan E. Krastins, Somerville, MA (US); Taha Rezai, Cambridge, MA (US); Amol Prakash, Cambridge, MA (US); Michael J. Athanas, Wayland, MA (US)

Correspondence Address: THERMO FINNIGAN LLC, 355 RIVER OAKS PARKWAY, SAN JOSE, CA, 95134, US

(21) Appl. No.: 12/710,062

(22) Filed: Feb. 22, 2010

Related U.S. Application Data
(60) Provisional application No. 61/154,267, filed on Feb. 20, 2009.

(51) Int. Cl. G01N 33/50

(52) U.S. Cl. 436/86

(57) ABSTRACT

The present invention relates to biomarkers in maternal serum samples and methods for predicting whether a fetus has Trisomy 21 during the first trimester of pregnancy based on the differential expression of a combination, or subcombination, of the same.

### CROSS-REFERENCE TO RELATED APPLICATIONS

This application claims the priority benefit under 35 U.S.C. §119(e) of U.S. Provisional Patent Application No. 61/154,267 by Lopez et al., the entire disclosure of which is incorporated herein by reference.

### BACKGROUND OF THE INVENTION

Trisomy 21, or Down's syndrome, is the most frequent fetal chromosomal disorder affecting pregnant women, and is one of the most common serious congenital abnormalities found at birth (Jones, K., Down's Syndrome in Smith's recognizable patterns of human malformation, Jones, K., Editor, 1997, Philadelphia, Pa., pp. 8-13). A majority of infants born with Down's syndrome will have serious cardiac, gastrointestinal, or other abnormalities that lead to significant morbidity and mortality. In addition, Down's syndrome is one of the leading causes of mental deficiency in the United States.

Down's syndrome occurs at a rate of 1 in 600-800 pregnancies and prompts most prenatal diagnoses that involve potentially dangerous invasive procedures such as amniocentesis or chorionic villous sampling. These procedures carry a significant risk of miscarriage and therefore are typically only applied to women in high risk groups (Zournatzi V, Daniilidis A, Karidas C, Tantanasis T, Loufopoulos A, Tzafettas J. 2008 Hippocratia 1: 28-32).

In an effort to reduce the frequency of invasive screening procedures, non-invasive procedures, such as maternal blood tests that measure protein biomarkers associated with Down's syndrome in maternal serum samples, and ultrasonographic measurements of nuchal translucency and other defects (Kagan K O, Wright D, Spencer K, Molina F S, Nicolaides K H. 2008 Ultrasound Obstet. Gynecol. 5:493-502), are routinely offered to pregnant women for second trimester screening. However, the sensitivity and specificity of these tests remains limited. Moreover, the effectiveness of these procedures for diagnosing Trisomy 21 during the first trimester of pregnancy has not been sufficiently validated.

Thus, despite the availability of several screening procedures for Down's syndrome, there is a significant need for new, safe and effective biomarkers and non-invasive screening methods for diagnosing Down's syndrome, particularly during the early stages of pregnancy.

### SUMMARY OF THE INVENTION

The present invention is based, in part, on Applicants' discovery of a panel of differentially expressed proteins in maternal serum obtained during the first trimester of pregnancy, wherein the expression level of one or more of the proteins (or peptide fragments derived therefrom) is predictive of a Trisomy 21 fetus.

In one embodiment, the invention relates to a method for predicting whether a fetus has Trisomy 21. The method comprises the step of measuring the expression level of a signature peptide fragment of at least one target protein (e.g., a peptide fragment characteristic of the target protein), wherein the target protein is selected from the group consisting of apolipoprotein A-I preprotein, apolipoprotein A-II preprotein, apolipoprotein A-IV precursor, apolipoprotein B precursor, apolipoprotein C-I precursor, apolipoprotein C-II precursor, apolipoprotein C-III precursor, apolipoprotein D precursor, apolipoprotein E precursor, apolipoprotein H precursor, paraoxonase 1, pregnancy-zone protein, α-2-macroglobulin precursor, serum amyloid A4, serum amyloid P component precursor, β-globin, ceruloplasmin precursor, α-1 globin and histidine-rich glycoprotein precursor in a serum sample obtained from the mother of the fetus during the first trimester of pregnancy. According to a preferred embodiment of the invention, the expression level is measured using tandem mass spectrometry. The method also comprises the step of comparing the expression level of the signature peptide fragment(s) in the sample to a corresponding control level(s). A difference (i.e., an increase or decrease) in the expression level of the signature peptide fragment(s) in the sample relative to the corresponding control level(s) is predictive of a fetus having Trisomy 21.

In a particular embodiment, the peptide fragment in the maternal serum sample is one or more selected from the group consisting of SEQ ID NO:45, SEQ ID NO:98, SEQ ID NO:161, SEQ ID NO:175, SEQ ID NO:177, SEQ ID NO:221, SEQ ID NO:226, SEQ ID NO:233, SEQ ID NO:242, SEQ ID NO:243, SEQ ID NO:246, SEQ ID NO:249, SEQ ID NO:253, SEQ ID NO:254, SEQ ID NO:258, SEQ ID NO:265, SEQ ID NO: 267, SEQ ID NO:268, and SEQ ID NO:278.

In another embodiment, the maternal serum sample is subjected to one or more of the following steps prior to mass spectrometry: enrichment for peptide fragments bound to carrier proteins; sample reduction, alkylation and/or desalting; one dimensional SDS-polyacrylamide gel electrophoresis; trypsin proteolysis; or liquid chromatography.

As described herein, a combination or subcombination (i.e., a panel) of all or any number of these target proteins, or their signature peptide fragments, in first trimester maternal serum are useful predictors of a Trisomy 21 fetus. The present invention provides a non-invasive and safe prenatal screening method for predicting whether a fetus has Trisomy 21. The methods of the invention have the advantage of providing a diagnosis of Trisomy 21 at an earlier stage of pregnancy than many commonly practiced Trisomy 21 screening methods.

### BRIEF DESCRIPTION OF THE DRAWINGS

FIG. 1 is a workflow diagram depicting an exemplary process for identifying biomarkers of Trisomy 21 in maternal serum samples, including sample collection, preparation, and analysis steps, and verification of candidates.

FIG. 2 is a workflow diagram depicting an exemplary process for LC MS/MS analysis of biomarkers in maternal serum samples and subsequent processing of analytical data.

### DETAILED DESCRIPTION OF THE INVENTION

As described herein, Applicants have identified biomarkers that are differentially expressed in biological samples from first trimester pregnant females carrying a fetus who has Trisomy 21, relative to biological samples from first trimester pregnant females carrying a fetus who does not have Trisomy 21. Accordingly, the expression levels of such biomarkers can be used to predict whether a fetus has Trisomy 21 during the first trimester of pregnancy.

In one embodiment, the invention relates to a method for predicting whether a fetus has Trisomy 21. The method comprises the step of measuring the expression level of a peptide fragment (e.g., one or more peptide fragments) of at least one target protein selected from the group consisting of apolipoprotein A-I preprotein (NCBI Accession NP_{—}000030.1), apolipoprotein A-II preprotein (NCBI Accession NP_{—}001634.1), apolipoprotein A-IV precursor (NCBI Accession NP_{—}000473.2), apolipoprotein B precursor (NCBI Accession NP_{—}000375.2), apolipoprotein C-I precursor (NCBI Accession NP_{—}001636.1), apolipoprotein C-II precursor (NCBI Accession NP_{—}000474.2), apolipoprotein C-III precursor (NCBI Accession NP_{—}000031.1), apolipoprotein D precursor (NCBI Accession NP_{—}001638.1), apolipoprotein E precursor (NCBI Accession NP_{—}000032.1), apolipoprotein H precursor (NCBI Accession NP_{—}000033.1), paraoxonase 1 (NCBI Accession NP_{—}000437.3), pregnancy-zone protein (NCBI Accession NP_{—}002855.1), α-2-macroglobulin precursor (NCBI Accession NP_{—}000005.2), serum amyloid A4 (NCBI Accession NP_{—}006503.1), serum amyloid P component precursor (NCBI Accession NP_{—}001630.1), β-globin (NCBI Accession NP_{—}000509.1), ceruloplasmin precursor (NCBI Accession NP_{—}000087.1), α-1 globin (NCBI Accession NP_{—}000549.1) and histidine-rich glycoprotein precursor (NCBI Accession NP_{—}000403.1).

As used herein, the term “peptide fragment” generally refers to any fragment of a full-length naturally-occurring target protein (e.g., a fragment produced by proteoytic digestion). The peptide fragment can be of any size, provided that the peptide fragment is smaller (i.e., has fewer amino acids) than the full-length target protein. In a preferred embodiment, a peptide fragment has a size in the range of about 5 to about 25 amino acids. In a particular embodiment, a peptide fragment has a size in the range of about 6 to about 17 amino acids.

In one embodiment, the methods of the invention comprise measuring the expression level of a peptide fragment selected from the group consisting of SEQ ID NO:45, SEQ ID NO:98, SEQ ID NO:161, SEQ ID NO:175, SEQ ID NO:177, SEQ ID NO:221, SEQ ID NO:226, SEQ ID NO:233, SEQ ID NO:242, SEQ ID NO:243, SEQ ID NO:246, SEQ ID NO:249, SEQ ID NO:253, SEQ ID NO:254, SEQ ID NO:258, SEQ ID NO:265, SEQ ID NO: 267, SEQ ID NO:268, and SEQ ID NO:278 in a biological sample obtained from a pregnant female.

In a particular embodiment, the peptide fragment is SEQ ID NO:258 and the level of expression of the biomarker protein (paraoxanase 1) corresponding to the peptide fragment is measured by SRM mass spectrometry using an m/z transition selected from the group consisting of about 942→387, 942→516, 942→603, 942→771, 942→812, 942→868, 942→982, 942→1199, and 942→1314.

In another embodiment, the peptide fragment is SEQ ID NO:175 and the level of expression of the biomarker protein (pregnancy-zone protein) corresponding to the peptide fragment is measured by SRM mass spectrometry using an m/z transition selected from the group consisting of about 510→179, 510→244, 510→357, 510→424, 510→474, 510→520, 510→534, 510→747, and 510→847.

In a further embodiment, the peptide fragment is SEQ ID NO:254 and the level of expression of the biomarker protein (alpha-2-macroglobulin precursor) corresponding to the peptide fragment is measured by SRM mass spectrometry using an m/z transition selected from the group consisting of about 543→329, 543→343, 543→372, 543→456, 543→514, 543→543, 543→656, 543→743, and 543→891.

In an additional embodiment, the peptide fragment is SEQ ID NO:98 and the level of expression of the biomarker protein (serum amyloid A4) corresponding to the peptide fragment is measured by SRM mass spectrometry using an m/z transition selected from the group consisting of about 567→232, 567→363, 567→410, 567→467, 567→478, 567→535, 567→634, 567→691, and 567→819.

In another embodiment, the peptide fragment is SEQ ID NO:177 and the level of expression of the biomarker protein (serum amyloid P component precursor) corresponding to the peptide fragment is measured by SRM mass spectrometry using an m/z transition selected from the group consisting of about 579→232, 579→345, 579→436, 579→508, 579→621, 579→708, 579→871, 579→1001, and 579→1058.

In yet another embodiment, the peptide fragment is SEQ ID NO:242 and the level of expression of the biomarker protein (beta globin) corresponding to the peptide fragment is measured by SRM mass spectrometry using an m/z transition selected from the group consisting of about 638→303, 638→404, 638→426, 638→475, 638→525, 638→687, 638→850, 638→949, and 638→1049.

In another embodiment, the peptide fragment is SEQ ID NO:267 and the level of expression of the biomarker protein (ceruloplasmin precursor) corresponding to the peptide fragment is measured by SRM mass spectrometry using an m/z transition selected from the group consisting of about 686→331, 686→541, 686→622, 686→670, 686→783, 686→871, 686→984, and 686→1081.

In an additional embodiment, the peptide fragment is SEQ ID NO:221 and the level of expression of the biomarker protein (alpha 1 globin) corresponding to the peptide fragment is measured by SRM mass spectrometry using an m/z transition selected from the group consisting of about 612→443, 612→464, 612→475, 612→524, 612→589, 612→663, 612→712, 612→785, 612→813, 612→927, 612→1041, and 612→1189.

In a further embodiment, the peptide fragment is SEQ ID NO:278 and the level of expression of the biomarker protein (histidine-rich glycoprotein precursor) corresponding to the peptide fragment is measured by SRM mass spectrometry using an m/z transition selected from the group consisting of about 563→201, 563→288, 563→339, 563→395, 563→401, 563→477, 563→529, 563→676, and 563→789.

In another embodiment, the peptide fragment is SEQ ID NO:45 and the level of expression of the biomarker protein (apolipoprotein H precursor) corresponding to the peptide fragment is measured by SRM mass spectrometry using an m/z transition selected from the group consisting of about 512→304, 512→327, 512→361, 512→376, 512→426, 512→489, 512→652, 512→751, and 512→850.

In yet another embodiment, the peptide fragment is SEQ ID NO:246 and the level of expression of the biomarker protein (apolipoprotein B precursor) corresponding to the peptide fragment is measured by SRM mass spectrometry using an m/z transition selected from the group consisting of about 507→260, 507→328, 507→373, 507→427, 507→444, 507→654, 507→741, 507→855, and 507→912.

In a further embodiment, the peptide fragment is SEQ ID NO:253 and the level of expression of the biomarker protein (apolipoprotein E precursor) corresponding to the peptide fragment is measured by SRM mass spectrometry using an m/z transition selected from the group consisting of about 450→226, 450→288, 450→377, 450→451, 450→566, and 450→752.

In another embodiment, the peptide fragment is SEQ ID NO:268 and the level of expression of the biomarker protein (apolipoprotein A-I preprotein) corresponding to the peptide fragment is measured by SRM mass spectrometry using an m/z transition selected from the group consisting of about 807→482, 807→569, 807→670, 807→769, 807→856, 807→971, 807→1158, 807→1272, and 807→1387.

In an additional embodiment, the peptide fragment is SEQ ID NO:226 and the level of expression of the biomarker protein (apolipoprotein C-III precursor) corresponding to the peptide fragment is measured by SRM mass spectrometry using an m/z transition selected from the group consisting of about 599→260, 599→347, 599→434, 599→477, 599→581, 599→638, 599→753, 599→854, and 599→953.

In further embodiment, the peptide fragment is SEQ ID NO:233 and the level of expression of the biomarker protein (apolipoprotein C-I precursor) corresponding to the peptide fragment is measured by SRM mass spectrometry using an m/z transition selected from the group consisting of about 647→262, 647→389, 647→504, 647→591, 647→605, 647→719, 647→776, 647→923, and 647→1052.

In another embodiment, the peptide fragment is SEQ ID NO:243 and the level of expression of the biomarker protein (apolipoprotein D precursor) corresponding to the peptide fragment is measured by SRM mass spectrometry using an m/z transition selected from the group consisting of about 616→246, 616→361, 616→474, 616→502, 616→588, 616→702, 616→789, 616→890, and 616→1004.

In yet another embodiment, the peptide fragment is SEQ ID NO:249 and the level of expression of the biomarker protein (apolipoprotein A-II preprotein) corresponding to the peptide fragment is measured by SRM mass spectrometry using an m/z transition selected from the group consisting of about 579→260, 579→286, 579→373, 579→470, 579→471, 579→535, 579→684, 579→813, and 579→942.

In an additional embodiment, the peptide fragment is SEQ ID NO:265 and the level of expression of the biomarker protein (apolipoprotein A-IV precursor) corresponding to the peptide fragment is measured by SRM mass spectrometry using an m/z transition selected from the group consisting of about 603→466, 603→536, 603→546, 603→565, 603→580, 603→623, 603→701, 603→736, and 603→770.

In a further embodiment, the peptide fragment is SEQ ID NO:161 and the level of expression of the biomarker protein (apolipoprotein C-II precursor) corresponding to the peptide fragment is measured by SRM mass spectrometry using an m/z transition selected from the group consisting of about 644→305, 644→434, 644→436, 644→536, 644→620, 644→783, 644→870, 644→957, and 644→1071.

It will be recognized that the m/z values specified above are nominal (rounded integer) values and are therefore approximate, and that the preferred embodiments of the invention include the use of transitions that have precursor/product ion m/z's that are within ±0.5 of the foregoing values.

Although the methods of the invention are exemplified herein using peptide fragments of the target proteins apolipoprotein A-I preprotein, apolipoprotein A-II preprotein, apolipoprotein A-IV precursor, apolipoprotein B precursor, apolipoprotein C-I precursor, apolipoprotein C-II precursor, apolipoprotein C-III precursor, apolipoprotein D precursor, apolipoprotein E precursor, apolipoprotein H precursor, paraoxonase 1, pregnancy-zone protein, α-2-macroglobulin precursor, serum amyloid A4, serum amyloid P component precursor, β-globin, ceruloplasmin precursor, α-1 globin and histidine-rich glycoprotein precursor, the invention can also be practiced by measuring and comparing expression levels of the full-length target proteins.

As used herein, the terms “one or more” and “at least one” in the context of peptide fragments, biomarkers, and target proteins mean any one, two, three, four, etc. of the listed members within a group, in any permutation. Accordingly, the terms “one or more” and “at least one” include any two, any three, any four, etc. of the members specifically listed within a group. Thus, the invention is not limited to any single group or subset of biomarkers. It is emphasized that the terms “one or more” and “at least one” are used in the broadest sense, and are used to designate any subgroup within a group with multiple members. Similarly, the terms “at least 2,” “at least 3,” “at least 4,” etc., cover any combinations of the members within a particular group, provided that the total number of members within the combination is at least 3, at least 3, at least, 4, etc.

Target proteins suitable for use in the methods of the invention are listed in Tables 1 and 2. Preferred biomarker m/z transitions are listed in Table 2. Any combination of one or more of the biomarkers listed in Tables 1 and 2 can be used in the methods of the invention.

A combination of different biomarkers (e.g., signature peptide fragments), as described above, might significantly improve diagnostic accuracy. For example, individual biomarkers can typically detect Down's syndrome in about 30% to 80% of occurrences. Using a combination of biomarkers, a diagnostic sensitivity and specificity of anywhere between 80% and 98% may be achieved. A combination of biomarkers which act independently, through distinct biological pathways, is particularly advantageous, as such combinations are expected to significantly increase diagnostic specificity and sensitivity.

The screening methods of the invention can also be combined with existing screening techniques for the detection of Trisomy 21. Thus, the diagnostic methods described herein can be combined with an examination of one or more known biomarkers for Trisomy 21, such as, for example, one or more of serum biomarkers PAPP-A, α-fetoprotein (AFP), human chorionic gonadotropin (βhCG), unconjugated estriol (uE3), and inhibin A. In a particular embodiment, the present screening techniques can be combined with a test using PAPP-A and βhCG as independent biomarkers, or a triple-marker serum test, based on AFP, βhCG, and uE3, especially if screening is performed in the second trimester. The test might, additionally or alternatively, include inhibin-A.

The screening assays described herein can further be combined with or supplemented by other techniques used to detect Trisomy 21 including, but not limited to, ultrasonography (e.g., transabdominal ultrasonography, translucent ultrasonography), nuchal translucency (NT) measurement, and techniques for detecting chromosomal abnormalities known in the art (e.g., karyotyping).

The expression level (e.g., amount, quantity, abundance) of one or more peptide fragments of any target protein identified herein can be measured in a biological sample obtained from a pregnant female. Suitable maternal biological samples for use in the methods of the invention include, for example, a serum sample, a plasma sample, a blood sample, an amniotic fluid sample, a urine sample, a cerebrospinal fluid sample, a breast milk sample, a mucus sample, and a saliva sample. In a preferred embodiment, the biological sample is a maternal serum sample.

The biological sample can be obtained from the mother at any time during pregnancy (e.g., first trimester, second trimester, third trimester). In a preferred embodiment, the biological sample is obtained from the mother during the first trimester of pregnancy.

The level of a peptide fragment of any target protein identified herein can be determined using any suitable analytical technique known in the art for identifying protein or peptide levels in a biological sample. Such methods include, but are not limited to, mass spectrometry, immunoblot analysis, immunohistochemical methods (e.g., in situ methods based on antibody detection of a target protein or peptide in a biological sample), immunoassays (e.g., ELISA), protein microarray methods and electrophoretic methods (e.g., 2-dimensional gel electrophoresis).

In a preferred embodiment, mass spectrometry (e.g., electrospray ionization or ESI mass spectrometry) is used to determine the expression level of one or more peptides in a maternal sample. The terms “mass spectrometry” or “MS” as used herein refer to methods of filtering, detecting, and measuring ions based on their mass-to-charge ratio, or “m/z.” In general, mass spectrometry involves ionizing a sample containing one or more molecules of interest, and then m/z separating and detecting the resultant ions (or product ions derived therefrom) in a mass analyzer, such as, without limitation, a quadrupole mass filter, quadrupole ion trap, time-of-flight analyzer, FT/ICR analyzer or Orbitrap, to generate a mass spectrum representing the abundances of detected ions at different values of m/z. See, e.g., U.S. Pat. No. 6,204,500, entitled “Mass Spectrometry From Surfaces;” U.S. Pat. No. 6,107,623, entitled “Methods and Apparatus for Tandem Mass Spectrometry;” U.S. Pat. No. 6,268,144, entitled “DNA Diagnostics Based On Mass Spectrometry;” U.S. Pat. No. 6,124,137, entitled “Surface-Enhanced Photolabile Attachment And Release For Desorption And Detection Of Analytes;” Wright et al., “Protein chip surface enhanced laser desorption/ionization (SELDI) mass spectrometry: a novel protein biochip technology for detection of prostate cancer biomarkers in complex protein mixtures,” Prostate Cancer and Prostatic Diseases 2: 264-76 (1999); and Merchant and Weinberger, “Recent advancements in surface-enhanced laser desorption/ionization-time of flight-mass spectrometry,” Electrophoresis 21: 1164-67 (2000), each of which is hereby incorporated by reference in its entirety, including all tables, figures, and claims.

In a particular embodiment, tandem mass spectrometry (e.g., using a quadrapole mass spectrometer) is employed in the methods of the invention. As used herein “tandem mass spectrometry,” or “MS/MS” refers to a technique wherein a precursor ion or group of ions generated from a molecule (or molecules) of interest may be isolated or selected in an MS instrument, and these precursor ions subsequently fragmented to yield one or more fragment ions that are then analyzed in a second MS procedure. By careful selection of precursor ions, ions produced by certain analytes of interest are selectively passed to the fragmentation chamber, where collision with atoms or molecules of an inert gas occurs to produce the fragment ions. Because both the precursor and fragment ions are produced in a reproducible fashion under a given set of ionization/fragmentation conditions, the MS/MS technique can provide an extremely powerful analytical tool. For example, the combination of filtration/fragmentation can be used to eliminate interfering substances, and can be particularly useful in complex samples, such as biological samples.

Ions can be produced using a variety of methods including, but not limited to, electrospray ionization (“ESI”), and matrix-assisted laser desorption ionization (“MALDI”).

In a preferred embodiment, electrospray ionization, or ESI, mass spectrometry is used to determine the expression level of one or more peptides in a maternal sample. The term “electrospray ionization,” or “ESI,” as used herein refers to methods in which a solution is passed along a short length of capillary tube, to the end of which is applied a high positive or negative electric potential. Solution reaching the end of the tube, is vaporized (nebulized) into a jet or spray of very small droplets of solution in solvent vapor. This mist of droplets flows through an evaporation chamber which is may be heated to prevent condensation and to evaporate solvent. As the droplets get smaller the electrical surface charge density increases until such time that the natural repulsion between like charges causes ions as well as neutral molecules to be released.

The term “matrix-assisted laser desorption ionization,” or “MALDI” as used herein refers to methods in which a non-volatile sample is exposed to laser irradiation, which desorbs and ionizes analytes in the sample by various ionization pathways, including photo-ionization, protonation, deprotonation, and cluster decay. For MALDI, the sample is mixed with an energy-absorbing matrix, which facilitates desorption of analyte molecules.

The term “ionization” and “ionizing” as used herein refers to the process of generating an analyte ion having a net electrical charge equal to one or more electron units. Negative ions are those ions having a net negative charge of one or more electron units, while positive ions are those ions having a net positive charge of one or more electron units.

The term “desorption” as used herein refers to the removal of an analyte from a surface and/or the entry of an analyte into a gaseous phase.

In those embodiments, such as MS/MS, where precursor ions are isolated for further fragmentation, collision-induced dissociation (“CID”) is often used to generate the fragment ions for further detection. In CID, precursor ions undergo fragmentation induced by energetic collisions with neutral molecules or atoms. Sufficient energy must be deposited in the precursor ion so that certain bonds within the ion can be broken due to increased vibrational energy.

Briefly described, the methods encompassed by the present invention include steps of acquiring a biological sample from a pregnant female (e.g., a blood serum sample collected from a woman in her first trimester of pregnancy), optionally subjecting the sample to one or more stages of preparation to modify, purify or concentrate target proteins, measuring the amounts of one or more peptide fragments characteristic of the target protein(s) by tandem mass spectrometry, and comparing the measured amount(s) to a corresponding control value or values to predict whether the fetus has Trisomy 21.

As discussed above, in various embodiments, the biological samples used in the methods of the invention are subjected to one or more sample preparation steps prior to analysis by mass spectrometry. For example, a biological sample (e.g., a serum sample) can be enriched for target proteins or biomarkers of interest using techniques known in the art. For example, target proteins or biomarkers in a serum sample can be enriched using a chromatography-based technology that relies on the Cibachron Blue dye for enrichment of peptides and protein fragments bound to carrier proteins (such as albumin) in plasma and serum samples. Such a technique is described herein in Example 1.

Prior to MS analysis, samples can also be reduced (e.g., by treatment with dithiothreitol), alkylated (e.g., by treatment with iodoacetic acid) and/or desalted (e.g., with HyperSep™-96 C18 solid phase extraction media). Methods for carrying out such steps, which can be performed with or without a sample enrichment step, are disclosed herein in the Examples.

Samples can also be subjected to further enrichment using one or more well known electrophoretic methods such as SDS PAGE or Isoelectric focusing prior to analysis by mass spectrometry. In one embodiment, samples are subjected to 1-dimensional sodium dodecyl sulfate polyacrylamide gel electrophoresis (“1-D SDS-PAGE”).

According to preferred embodiments, the sample is digested using a suitable proteolytic enzyme, such as trypsin, before mass spectrometric analysis. The digestion step may be performed prior or subsequent to or concurrently with other sample preparation steps. Proteolytic digestion of the sample yields peptide fragments, a portion of which will be specific to corresponding target proteins, and thus may be employed as surrogates for monitoring the quantities of the corresponding target proteins by mass spectrometry. Alternative embodiments may utilize a “top-down” approach, wherein samples containing substantially intact proteins are analyzed by mass spectrometry without first being proteolytically digested.

In a preferred embodiment, samples are subjected to a liquid chromatography (LC) purification step prior to mass spectrometry. Methods of coupling liquid chromatography techniques to MS analysis are well known and widely practiced in the art. Traditional LC analysis relies on the chemical interactions between sample components and column packings, where laminar flow of the sample through the column is the basis for separation of the analyte of interest from the test sample. The skilled artisan will understand that separation in such columns is a diffusional process.

Numerous column packings are available for chromatographic separation of samples, and selection of an appropriate separation protocol is an empirical process that depends on the sample characteristics, the analyte of interest, the interfering substances present and their characteristics, etc. Various packing chemistries can be used depending on the needs (e.g., structure, polarity, and solubility of compounds being purified).

In various embodiments the columns are polar, ion exchange (both cation and anion), hydrophobic interaction, phenyl, C-2, C-8, C-18 columns, polar coating on porous polymer, or others that are commercially available. During chromatography, the separation of materials is effected by variables such as choice of eluant (also known as a “mobile phase”), choice of gradient elution and the gradient conditions, temperature, etc.

In certain embodiments, an analyte may be purified by applying a sample to a column under conditions where the analyte of interest is reversibly retained by the column packing material, while one or more other materials are not retained. In these embodiments, a first mobile phase condition can be employed where the analyte of interest is retained by the column, and a second mobile phase condition can subsequently be employed to remove retained material from the column, once the non-retained materials are washed through. Alternatively, an analyte may be purified by applying a sample to a column under mobile phase conditions where the analyte of interest elutes at a differential rate in comparison to one or more other materials. As discussed above, such procedures may enrich the amount of one or more analytes of interest relative to one or more other components of the sample.

In a preferred embodiment, one or more LC purification steps are performed “online” with subsequent MS analysis steps. The term “online” as used herein refers to steps performed without further need for operator intervention. For example, by careful selection of valves and connector plumbing, two or more chromatography columns can be connected such that material is passed from one to the next without the need for additional manual steps. The selection and operation of valves and plumbing is controlled by a computer pre-programmed to perform the necessary steps. Preferably, the chromatography system is also connected in such an on-line fashion to the detector system, e.g., an MS system. Thus, an operator may place a tray of samples in an autosampler, and the remaining operations are performed “on-line” under computer control, resulting in purification and analysis of all samples selected.

To facilitate accurate quantitation of the peptide fragments by mass spectrometry, a set of isotopically-labeled synthetic versions of the peptide fragments of interest may be added in known amounts to the sample for use as internal standards. Since the isotopically-labeled peptides have physical and chemical properties substantially identical to the corresponding surrogate peptide, they co-elute from the chromatographic column and are easily identifiable on the resultant mass spectrum. The addition of the labeled standards may occur piro or subsequent to protolytic digestion.

In a typical implementation of the invention, quantitation of peptide fragments corresponding to targeted proteins is carried out using a triple quadrupole mass spectrometer (such as the TSQ Vantage mass spectrometer, available from Thermo Fisher Scientific) operated in selective reaction monitoring (SRM) mode. As known in the art, triple quadrupole mass spectrometers include three quadrupole rod sets. A first stage of mass selection is performed in the first quadrupole rod set, and the selectively transmitted ions are fragmented in the second quadrupole rod set. The resultant fragment (product) ions are conveyed to the third quadrupole rod set, which performs a second stage of mass selection. The product ions transmitted throught the third quadrupole rod set are measured by a detector, which generates a signal representative of the numbers of selectively transmitted product ions. The RF and DC potentials applied to the first and third quadrupoles are tuned to select (respectively) precursor and product ions that have m/z's lying within narrow specified ranges. By specifying the appropriate transitions (m/z values of precursor and product ions), a peptide corresponding to a targeted protein may be measured with high degrees of sensitivity and selectivity.

Once the mass spectrometric analysis of the prepared sample has been completed, the quantities of the peptide fragments in the sample may be determined by integration of the relevant mass spectral peak areas, as known in the prior art. When isotopically-labeled internal standards are used, as described above, the quantities of the peptide fragments of interest are established via an empirically-derived or predicted relationship between peptide fragment quantity (which may be expressed as concentration) and the area ratio of the peptide fragment and internal standard peaks at specified transitions. Other implementations of the assay may utilize external standards or other expedients for peptide quantification.

The methods of embodiments of the invention comprise the step of comparing the expression level of one or more peptide fragments in a maternal biological test sample to a corresponding control level(s). In one embodiment, the corresponding control level is the level of the peptide fragment in a corresponding normal maternal biological sample. As used herein, a “corresponding normal maternal biological sample” is a sample of the same type as the maternal test sample (e.g., a serum sample), which is obtained at the same stage of pregnancy (e.g., during the first trimester) from a mother who is not carrying a Trisomy 21 fetus (e.g. a normal control). In another embodiment, the corresponding control level is a reference standard (e.g., an average or typical level of a peptide fragment biomarker in a normal maternal biological sample based on samples obtained from a plurality (i.e., two or more) of pregnant mothers having non-Trisomy 21 pregnancies).

According to embodiments of the invention, a difference (e.g., an increase or a decrease) in the expression level of a selected peptide fragment, or panel of selected peptide fragments, in the sample relative to the corresponding control level is predictive of a fetus having Trisomy 21. As used herein, “difference” refers to any statistically significant difference in the level of a selected biomarker in a test sample relative to the level of the same biomarker in a corresponding control sample. Statistical methods for determining significant differences in gene expression are well known in the art. If the level of one or more selected peptide fragments in the maternal test sample is identical to, or essentially the same as, the corresponding control level (e.g., no statistically significant difference), it is unlikely that the fetus has Trisomy 21 (i.e., a negative prediction). If the level of a peptide fragment in the maternal test sample is significantly different than the corresponding control level, as determined by an appropriate statistical test(s), it is likely that the fetus has Trisomy 21 (i.e., a positive prediction). In one embodiment, the level of the biomarker is greater (e.g., up-regulated) in the test sample relative to a corresponding control level. In another embodiment, the level of the biomarker is reduced (e.g., down-regulated) in the test sample relative to a corresponding control level. For a panel of biomarkers, selected biomarkers can be either up-regulated or down-regulated, that is, a level of expression of a selected biomarker can be increased or decreased relative to control levels.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994) provides one skilled in the art with a general guide to many of the terms used in the present application.

A detailed description of the methods utilized for discovery and validation of Trisomy 21 biomarkers is set forth in the following example.

### Example 1

### Identification of Differentially Expressed Biomarkers in First Trimester Maternal Serum Samples from Trisomy 21 and Normal Pregnancies Using Tandem Mass Spectrometry

### Materials and Methods

### Clinical Serum Samples

Maternal serum samples from Trisomy 21 and normal first trimester pregnancies were provided by the Fetal Medicine Foundation (FMF) and collected from study participants with full consent and approval. The majority of these women were found to be high risk based on the FMF risk calculation for Trisomy 21. Blood samples were collected into red top tubes (BD Vacutainer™ REF 367694 Z) which do not contain anti-coagulant. The average sample volume was 7.0 ml. Tubes were labeled and kept at 4° C. less than 8 hr prior to centrifuging. Samples were centrifuged at 1,500 rpm for 15 minutes at 4° C. At the end of the spin, the tubes were taken out carefully and placed on a rack. The separated sera were gently aspirated with a pipette, transferred to microfuge tubes and frozen. Samples were transported frozen and thawed immediately before processing. Samples from Trisomy 21 patients and healthy control patients were processed in a random order to prevent systematic errors and variations from experiment to experiment.

### Sample Processing

### Cibachron Blue Enrichment of Serum Samples

Serum samples (20 μl) were processed using ProXPRESSION™ biomarker enrichment kits (PerkinElmer) as described in Lopez M. F., et al. Clinical Chemistry 53: 1067-1074, (2007), the teachings of which are incorporated herein by reference. The final elution step was modified from the published protocol as 8M Guanidine HCL, pH 8.5 was substituted for the kit elution buffer. The Cibachron Blue dye affinity chromatography-based technology is designed to enrich the peptide and protein fragments bound to carrier proteins (such as albumin) in plasma and serum samples. After elution from the Cibachron Blue resin [PerkinElmer, Waltham, Mass.], samples were reduced and alkylated. For this procedure, 10 mM Dithiothreitol (DTT) was added and the samples were incubated at 37° C. for 1 h. The denatured samples were then alkylated with 45 mM iodoacetic acid (500 mM stock concentration in 1 M ammonium bicarbonate) in the dark for 1 h at room temperature. Residual alkylation agent was then reacted with 15 mM DTT followed by addition of trifluoroacetic acid (TFA) to a final concentration of 2% (V/V) to acidify the sample. Following reduction/alkylation, samples were desalted with HyperSep™-96 C18 solid phase extraction media (Thermo-Fisher Scientific). Briefly, the HyperSep™ C18 resin was conditioned before use with n-Propanol. Samples were then loaded on the resin and washed with 8M Guanidine HCl, 10 mM Dithiothreitol (DTT), 150 mM Tris HCL pH 8.5 diluted 4:1 with TFA to a final concentration of 0.25% (V/V) and eluted with 50% (V/V) n-Propanol in 0.1% (V/V) Formic acid.

### 1-D SDS PAGE of Enriched Samples

After desalting, samples were loaded on SDS PAGE minigels (Invitrogen) and electrophoresed according to manufacturer's instructions. After staining with Coomassie Blue (Pierce Safe Gel Stain, Thermo Fisher Scientific), the region between MW 6 kDa and 26 kDa in each sample lane was excised and subjected to proteolysis with trypsin. The extracted peptides were subsequently analyzed by LC-MS/MS.

### High Resolution LC-MS/MS

High resolution LC-MS/MS analysis was carried out on an LTQ-Orbitrap XL mass spectrometer (ThermoFisher Scientific). Samples in 5% (v/v) acetonitrile 0.1% (v/v) formic acid were injected onto a 75 μm×25 cm fused silica capillary column packed with Hypersil Gold C18AQ 5 μm media (Thermo Fisher Scientific), in a 250 μL/min gradient of 5% (v/v) acetonitrile, 0.1% (v/v) formic acid to 30% (v/v) acetonitrile, 0.1% (v/v) formic acid over the course of 180 minutes with a total run length of 240 minutes. The LTQ-Orbitrap was run in a top 5 configuration at 60K resolution for a full scan, with monoisotopic precursor selection enabled, and +1, and unassigned charge state rejected. The analysis was carried out with CID and HCD fragmentation modes. Label-free, differential analysis and protein identification were performed using the SIEVE (Thermo Fisher Scientific) algorithm and SEQUEST® (Sutton J et al., Proteomics Clin. Appl. 2008, 2, 862-881).

### Selective Reaction Monitoring (SRM) Assays

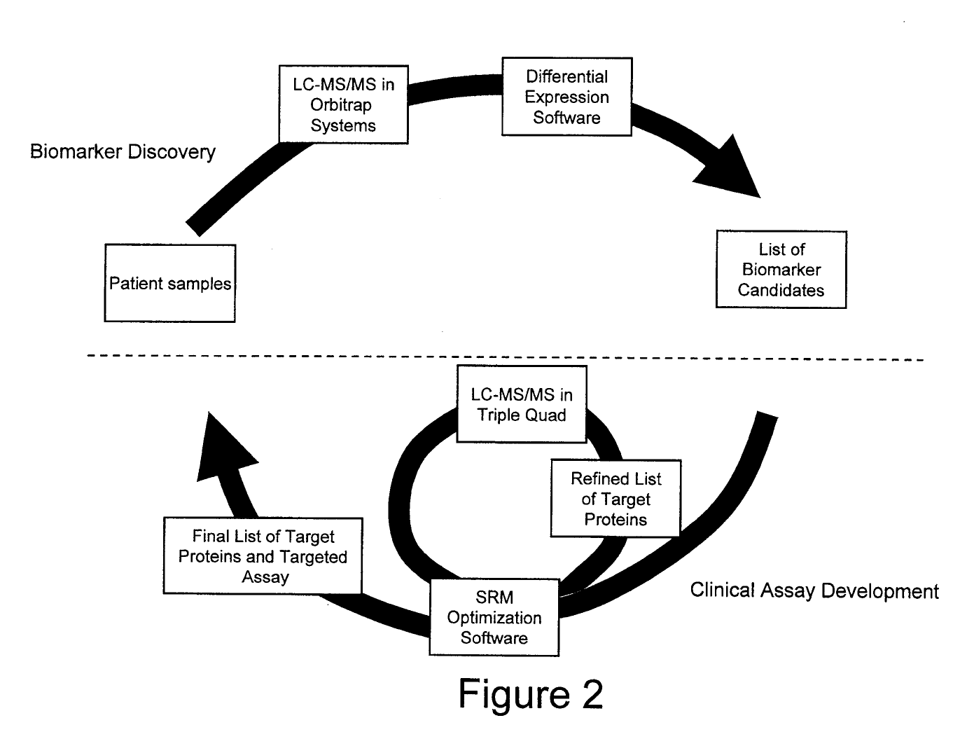

SRM assays were developed in a Quantum Ultra or Vantage triple quadrupole mass spectrometer, Surveyor MS pump, Micro Autosampler and an IonMax Source equipped with a low flow metal needle (ThermoFisher Scientific). Reverse phase separations were carried out on a 1 mm×50 mm Hypersil Gold 1.9 μm C18 particle (ThermoFisher Scientific). Solvent A was LC-MS grade water with 0.2% (v/v) formic acid, and solvent B was LC-MS grade 30% (v/v) acetonitrile with 0.2% (v/v) formic acid (Optima grade reagents, ThermoFisher Scientific). SRM workflow prototype software (available upon request from ThermoFisher Scientific) was used for targeted protein quantitation. This software algorithm can be used for predicting candidate peptides and choosing multiple fragment ions for SRM assay design, building an instrument method and a sequence file, and also for automatic peptide identity confirmation and quantitative data processing. A workflow diagram for this process is depicted in FIG. 2. Specifically, differentially expressed peptides that were identified in the LC-MS/MS discovery MS data were imported directly into SRM builder software. Transitions were chosen based on the predominant fragments observed in the discovery data (>5 transitions per peptide). Peptides were identified by the co-eluting light and AQUA-labeled heavy transitions. Moreover, the ratio of the transitions for confirmed with the discovery spectra for additional verification. Finally, time alignment and relative quantification of the transitions was performed with SRM builder.

### Results

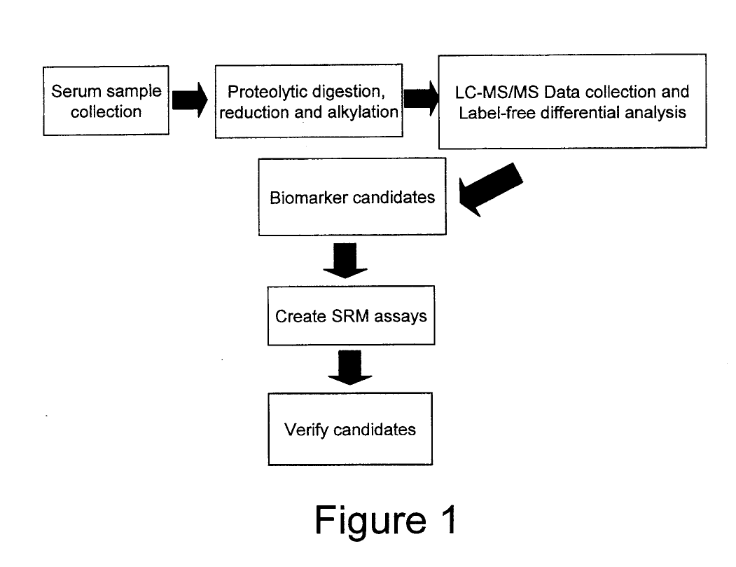

Using the techniques described above, approximately 2400 peptide fragments representing 19 target proteins were identified as being differentially expressed in first trimester maternal serum samples from Trisomy 21 pregnancies relative to normal pregnancies. The amino acid sequences, m/z transitions and parent target protein for each of the identified peptides is shown in Table 1. A workflow diagram for the entire process, including sample enrichment and subsequent analysis, is shown in FIG. 1.

| | | | | | |
|---|---|---|---|---|---|
| | | | | Parent | |
| | | | | Peptide | |
| | | | SEQ | Ion | Fragment |
| | Accession | | ID | Mass/ | Ion Mass/ |
| Protein Name | Number | Peptide Fragment | NO: | Charge | Charge |

| | | | | | |
|---|---|---|---|---|---|
| apolipoprotein B precursor | NP_000375.2 | SQAIATK | 1 | 359.7083 | 319.1971 |
| apolipoprotein B precursor | NP_000375.2 | SQAIATK | 1 | 359.7083 | 432.2811 |
| apolipoprotein B precursor | NP_000375.2 | SQAIATK | 1 | 359.7083 | 503.3182 |
| apolipoprotein B precursor | NP_000375.2 | SQAIATK | 1 | 359.7083 | 631.3768 |
| pregnancy-zone protein | NP_002855.1 | GVPIPNK | 2 | 362.7213 | 358.2079 |
| pregnancy-zone protein | NP_002855.1 | GVPIPNK | 2 | 362.7213 | 471.292 |
| pregnancy-zone protein | NP_002855.1 | GVPIPNK | 2 | 362.7213 | 568.3448 |
| pregnancy-zone protein | NP_002855.1 | GVPIPNK | 2 | 362.7213 | 667.4132 |
| alpha-2-macroglobulin precursor | NP_000005.2 | GVPIPNK | 3 | 362.7253 | 358.2079 |
| alpha-2-macroglobulin precursor | NP_000005.2 | GVPIPNK | 3 | 362.7253 | 471.292 |
| alpha-2-macroglobulin precursor | NP_000005.2 | GVPIPNK | 3 | 362.7253 | 568.3448 |
| alpha-2-macroglobulin precursor | NP_000005.2 | GVPIPNK | 3 | 362.7253 | 667.4132 |
| apolipoprotein B precursor | NP_000375.2 | INSGAHK | 4 | 363.6983 | 355.2083 |
| apolipoprotein B precursor | NP_000375.2 | INSGAHK | 4 | 363.6983 | 412.2297 |
| apolipoprotein B precursor | NP_000375.2 | INSGAHK | 4 | 363.6983 | 499.2618 |
| apolipoprotein B precursor | NP_000375.2 | INSGAHK | 4 | 363.6983 | 613.3047 |
| apolipoprotein B precursor | NP_000375.2 | LAIPEGK | 5 | 364.2211 | 333.1763 |
| apolipoprotein B precursor | NP_000375.2 | LAIPEGK | 5 | 364.2211 | 430.2291 |
| apolipoprotein B precursor | NP_000375.2 | LAIPEGK | 5 | 364.2211 | 543.3131 |
| apolipoprotein B precursor | NP_000375.2 | LAIPEGK | 5 | 364.2211 | 614.3503 |
| apolipoprotein B precursor | NP_000375.2 | VSQEGLK | 6 | 380.7136 | 317.2178 |
| apolipoprotein B precursor | NP_000375.2 | VSQEGLK | 6 | 380.7136 | 446.2604 |
| apolipoprotein B precursor | NP_000375.2 | VSQEGLK | 6 | 380.7136 | 574.319 |
| apolipoprotein B precursor | NP_000375.2 | VSQEGLK | 6 | 380.7136 | 661.351 |
| apolipoprotein C-I precursor | NP_001636.1 | QSELSAK | 7 | 381.7032 | 305.1814 |
| apolipoprotein C-I precursor | NP_001636.1 | QSELSAK | 7 | 381.7032 | 418.2655 |
| apolipoprotein C-I precursor | NP_001636.1 | QSELSAK | 7 | 381.7032 | 547.308 |
| apolipoprotein C-I precursor | NP_001636.1 | QSELSAK | 7 | 381.7032 | 634.3401 |
| apolipoprotein B precursor | NP_000375.2 | NFATSNK | 8 | 391.1956 | 348.1872 |
| apolipoprotein B precursor | NP_000375.2 | NFATSNK | 8 | 391.1956 | 449.2349 |
| apolipoprotein B precursor | NP_000375.2 | NFATSNK | 8 | 391.1956 | 520.272 |
| apolipoprotein B precursor | NP_000375.2 | NFATSNK | 8 | 391.1956 | 667.3404 |
| apolipoprotein E precursor | NP_000032.1 | DADDLQK | 9 | 402.6903 | 388.2549 |
| apolipoprotein E precursor | NP_000032.1 | DADDLQK | 9 | 402.6903 | 503.2818 |
| apolipoprotein E precursor | NP_000032.1 | DADDLQK | 9 | 402.6903 | 618.3088 |
| apolipoprotein E precursor | NP_000032.1 | DADDLQK | 9 | 402.6903 | 689.3459 |
| serum amyloid P component precursor | NP_001630.1 | AYSDLSR | 10 | 406.2009 | 375.2345 |
| serum amyloid P component precursor | NP_001630.1 | AYSDLSR | 10 | 406.2009 | 490.2614 |
| serum amyloid P component precursor | NP_001630.1 | AYSDLSR | 10 | 406.2009 | 577.2935 |
| serum amyloid P component precursor | NP_001630.1 | AYSDLSR | 10 | 406.2009 | 740.3568 |
| ceruloplasmin precursor | NP_000087.1 | GSLHANGR | 11 | 406.2121 | 346.1828 |
| ceruloplasmin precursor | NP_000087.1 | GSLHANGR | 11 | 406.2121 | 417.2199 |
| ceruloplasmin precursor | NP_000087.1 | GSLHANGR | 11 | 406.2121 | 554.2788 |
| ceruloplasmin precursor | NP_000087.1 | GSLHANGR | 11 | 406.2121 | 667.3629 |
| ceruloplasmin precursor | NP_000087.1 | GSLHANGR | 11 | 406.2121 | 754.3949 |
| apolipoprotein E precursor | NP_000032.1 | AQAWGER | 12 | 409.2012 | 361.1825 |
| apolipoprotein E precursor | NP_000032.1 | AQAWGER | 12 | 409.2012 | 547.2618 |
| apolipoprotein E precursor | NP_000032.1 | AQAWGER | 12 | 409.2012 | 618.2989 |
| apolipoprotein E precursor | NP_000032.1 | AQAWGER | 12 | 409.2012 | 746.3575 |
| alpha-2-macroglobulin precursor | NP_000005.2 | QSSEITR | 13 | 410.7116 | 389.2502 |
| alpha-2-macroglobulin precursor | NP_000005.2 | QSSEITR | 13 | 410.7116 | 518.2927 |
| alpha-2-macroglobulin precursor | NP_000005.2 | QSSEITR | 13 | 410.7116 | 605.3248 |
| alpha-2-macroglobulin precursor | NP_000005.2 | QSSEITR | 13 | 410.7116 | 692.3568 |
| apolipoprotein E precursor | NP_000032.1 | EQVAEVR | 14 | 415.722 | 403.2294 |
| apolipoprotein E precursor | NP_000032.1 | EQVAEVR | 14 | 415.722 | 474.2665 |
| apolipoprotein E precursor | NP_000032.1 | EQVAEVR | 14 | 415.722 | 573.335 |
| apolipoprotein E precursor | NP_000032.1 | EQVAEVR | 14 | 415.722 | 701.3935 |
| apolipoprotein A-IV precursor | NP_000473.2 | GNTEGLQK | 15 | 423.7194 | 388.2549 |
| apolipoprotein A-IV precursor | NP_000473.2 | GNTEGLQK | 15 | 423.7194 | 445.2764 |
| apolipoprotein A-IV precursor | NP_000473.2 | GNTEGLQK | 15 | 423.7194 | 574.319 |
| apolipoprotein A-IV precursor | NP_000473.2 | GNTEGLQK | 15 | 423.7194 | 675.3666 |
| apolipoprotein A-IV precursor | NP_000473.2 | GNTEGLQK | 15 | 423.7194 | 789.4095 |
| apolipoprotein E precursor | NP_000032.1 | ELQAAQAR | 16 | 443.7407 | 374.2141 |
| apolipoprotein E precursor | NP_000032.1 | ELQAAQAR | 16 | 443.7407 | 445.2512 |
| apolipoprotein E precursor | NP_000032.1 | ELQAAQAR | 16 | 443.7407 | 516.2883 |
| apolipoprotein E precursor | NP_000032.1 | ELQAAQAR | 16 | 443.7407 | 644.3469 |
| apolipoprotein E precursor | NP_000032.1 | ELQAAQAR | 16 | 443.7407 | 757.431 |
| apolipoprotein B precursor | NP_000375.2 | HDAHLNGK | 17 | 446.2252 | 318.1766 |
| apolipoprotein B precursor | NP_000375.2 | HDAHLNGK | 17 | 446.2252 | 431.2607 |
| apolipoprotein B precursor | NP_000375.2 | HDAHLNGK | 17 | 446.2252 | 568.3196 |
| apolipoprotein B precursor | NP_000375.2 | HDAHLNGK | 17 | 446.2252 | 639.3568 |
| apolipoprotein B precursor | NP_000375.2 | HDAHLNGK | 17 | 446.2252 | 754.3837 |
| serum amyloid A4; constitutive | NP_006503.1 | GNYDAAQR | 18 | 447.7068 | 374.2141 |
| serum amyloid A4; constitutive | NP_006503.1 | GNYDAAQR | 18 | 447.7068 | 445.2512 |
| serum amyloid A4; constitutive | NP_006503.1 | GNYDAAQR | 18 | 447.7068 | 560.2781 |
| serum amyloid A4; constitutive | NP_006503.1 | GNYDAAQR | 18 | 447.7068 | 723.3415 |
| serum amyloid A4; constitutive | NP_006503.1 | GNYDAAQR | 18 | 447.7068 | 837.3844 |
| apolipoprotein B precursor | NP_000375.2 | LNGESNLR | 19 | 451.7382 | 402.2454 |
| apolipoprotein B precursor | NP_000375.2 | LNGESNLR | 19 | 451.7382 | 489.2774 |
| apolipoprotein B precursor | NP_000375.2 | LNGESNLR | 19 | 451.7382 | 618.32 |
| apolipoprotein B precursor | NP_000375.2 | LNGESNLR | 19 | 451.7382 | 675.3415 |
| apolipoprotein B precursor | NP_000375.2 | LNGESNLR | 19 | 451.7382 | 789.3844 |
| apolipoprotein B precursor | NP_000375.2 | QELNGNTK | 20 | 452.2302 | 362.2029 |
| apolipoprotein B precursor | NP_000375.2 | QELNGNTK | 20 | 452.2302 | 419.2243 |
| apolipoprotein B precursor | NP_000375.2 | QELNGNTK | 20 | 452.2302 | 533.2673 |
| apolipoprotein B precursor | NP_000375.2 | QELNGNTK | 20 | 452.2302 | 646.3513 |
| apolipoprotein B precursor | NP_000375.2 | QELNGNTK | 20 | 452.2302 | 775.3939 |
| paraoxonase 1 | NP_000437.3 | SFNPNSPGK | 21 | 474.2327 | 301.1865 |
| paraoxonase 1 | NP_000437.3 | SFNPNSPGK | 21 | 474.2327 | 388.2185 |
| paraoxonase 1 | NP_000437.3 | SFNPNSPGK | 21 | 474.2327 | 502.2614 |
| paraoxonase 1 | NP_000437.3 | SFNPNSPGK | 21 | 474.2327 | 599.3142 |
| paraoxonase 1 | NP_000437.3 | SFNPNSPGK | 21 | 474.2327 | 713.3571 |
| paraoxonase 1 | NP_000437.3 | SFNPNSPGK | 21 | 474.2327 | 860.4255 |
| apolipoprotein A-IV precursor | NP_000473.2 | QLTPYAQR | 22 | 488.7642 | 374.2141 |
| apolipoprotein A-IV precursor | NP_000473.2 | QLTPYAQR | 22 | 488.7642 | 537.2774 |
| apolipoprotein A-IV precursor | NP_000473.2 | QLTPYAQR | 22 | 488.7642 | 634.3302 |
| apolipoprotein A-IV precursor | NP_000473.2 | QLTPYAQR | 22 | 488.7642 | 735.3779 |
| apolipoprotein A-IV precursor | NP_000473.2 | QLTPYAQR | 22 | 488.7642 | 848.4619 |
| apolipoprotein B precursor | NP_000375.2 | VNDESTEGK | 23 | 489.7224 | 333.1763 |
| apolipoprotein B precursor | NP_000375.2 | VNDESTEGK | 23 | 489.7224 | 434.224 |
| apolipoprotein B precursor | NP_000375.2 | VNDESTEGK | 23 | 489.7224 | 521.256 |
| apolipoprotein B precursor | NP_000375.2 | VNDESTEGK | 23 | 489.7224 | 650.2986 |
| apolipoprotein B precursor | NP_000375.2 | VNDESTEGK | 23 | 489.7224 | 765.3256 |
| apolipoprotein B precursor | NP_000375.2 | VNDESTEGK | 23 | 489.7224 | 879.3685 |
| apolipoprotein H precursor | NP_000033.1 | TDASDVKPC[Carboxymethyl] | 24 | 497.2133 | 405.1797 |
| apolipoprotein H precursor | NP_000033.1 | TDASDVKPC[Carboxymethyl] | 24 | 497.2133 | 504.2481 |
| apolipoprotein H precursor | NP_000033.1 | TDASDVKPC[Carboxymethyl] | 24 | 497.2133 | 619.275 |
| apolipoprotein H precursor | NP_000033.1 | TDASDVKPC[Carboxymethyl] | 24 | 497.2133 | 706.3071 |
| apolipoprotein H precursor | NP_000033.1 | TDASDVKPC[Carboxymethyl] | 24 | 497.2133 | 777.3442 |
| apolipoprotein H precursor | NP_000033.1 | TDASDVKPC[Carboxymethyl] | 24 | 497.2133 | 892.3711 |
| apolipoprotein C-I precursor | NP_001636.1 | IKQSELSAK | 25 | 502.2928 | 305.1814 |
| apolipoprotein C-I precursor | NP_001636.1 | IKQSELSAK | 25 | 502.2928 | 418.2655 |
| apolipoprotein C-I precursor | NP_001636.1 | IKQSELSAK | 25 | 502.2928 | 547.308 |
| apolipoprotein C-I precursor | NP_001636.1 | IKQSELSAK | 25 | 502.2928 | 634.3401 |
| apolipoprotein C-I precursor | NP_001636.1 | IKQSELSAK | 25 | 502.2928 | 762.3987 |
| apolipoprotein C-I precursor | NP_001636.1 | IKQSELSAK | 25 | 502.2928 | 890.4937 |
| apolipoprotein B precursor | NP_000375.2 | GAYQNNEIK | 26 | 518.7565 | 389.2389 |
| apolipoprotein B precursor | NP_000375.2 | GAYQNNEIK | 26 | 518.7565 | 503.2818 |
| apolipoprotein B precursor | NP_000375.2 | GAYQNNEIK | 26 | 518.7565 | 617.3248 |
| apolipoprotein B precursor | NP_000375.2 | GAYQNNEIK | 26 | 518.7565 | 745.3834 |
| apolipoprotein B precursor | NP_000375.2 | GAYQNNEIK | 26 | 518.7565 | 908.4467 |
| apolipoprotein B precursor | NP_000375.2 | GAYQNNEIK | 26 | 518.7565 | 979.4838 |
| apolipoprotein B precursor | NP_000375.2 | ESDEETQIK | 27 | 539.7486 | 388.2549 |
| apolipoprotein B precursor | NP_000375.2 | ESDEETQIK | 27 | 539.7486 | 489.3026 |
| apolipoprotein B precursor | NP_000375.2 | ESDEETQIK | 27 | 539.7486 | 618.3452 |
| apolipoprotein B precursor | NP_000375.2 | ESDEETQIK | 27 | 539.7486 | 747.3878 |
| apolipoprotein B precursor | NP_000375.2 | ESDEETQIK | 27 | 539.7486 | 862.4147 |
| apolipoprotein B precursor | NP_000375.2 | ESDEETQIK | 27 | 539.7486 | 949.4467 |
| histidine-rich glycoprotein precursor | NP_000403.1 | HSHESQDLR | 28 | 554.7601 | 403.2294 |
| histidine-rich glycoprotein precursor | NP_000403.1 | HSHESQDLR | 28 | 554.7601 | 531.288 |
| histidine-rich glycoprotein precursor | NP_000403.1 | HSHESQDLR | 28 | 554.7601 | 618.32 |
| histidine-rich glycoprotein precursor | NP_000403.1 | HSHESQDLR | 28 | 554.7601 | 747.3626 |
| histidine-rich glycoprotein precursor | NP_000403.1 | HSHESQDLR | 28 | 554.7601 | 884.4215 |
| histidine-rich glycoprotein precursor | NP_000403.1 | HSHESQDLR | 28 | 554.7601 | 971.4536 |
| apolipoprotein A-II preproprotein | NP_001634.1 | VKSPELQAEAK | 29 | 600.3351 | 347.192 |
| apolipoprotein A-II preproprotein | NP_001634.1 | VKSPELQAEAK | 29 | 600.3351 | 418.2291 |
| apolipoprotein A-II preproprotein | NP_001634.1 | VKSPELQAEAK | 29 | 600.3351 | 546.2877 |
| apolipoprotein A-II preproprotein | NP_001634.1 | VKSPELQAEAK | 29 | 600.3351 | 659.3717 |
| apolipoprotein A-II preproprotein | NP_001634.1 | VKSPELQAEAK | 29 | 600.3351 | 788.4143 |
| apolipoprotein A-II preproprotein | NP_001634.1 | VKSPELQAEAK | 29 | 600.3351 | 885.4671 |
| apolipoprotein A-II preproprotein | NP_001634.1 | VKSPELQAEAK | 29 | 600.3351 | 972.4991 |
| apolipoprotein A-II preproprotein | NP_001634.1 | VKSPELQAEAK | 29 | 600.3351 | 1100.594 |
| pregnancy-zone protein | NP_002855.1 | EGTHGSHVYTK | 30 | 608.2913 | 411.2233 |
| pregnancy-zone protein | NP_002855.1 | EGTHGSHVYTK | 30 | 608.2913 | 510.2917 |
| pregnancy-zone protein | NP_002855.1 | EGTHGSHVYTK | 30 | 608.2913 | 647.3506 |
| pregnancy-zone protein | NP_002855.1 | EGTHGSHVYTK | 30 | 608.2913 | 734.3826 |
| pregnancy-zone protein | NP_002855.1 | EGTHGSHVYTK | 30 | 608.2913 | 791.4041 |
| pregnancy-zone protein | NP_002855.1 | EGTHGSHVYTK | 30 | 608.2913 | 928.463 |
| pregnancy-zone protein | NP_002855.1 | EGTHGSHVYTK | 30 | 608.2913 | 1029.511 |
| pregnancy-zone protein | NP_002855.1 | EGTHGSHVYTK | 30 | 608.2913 | 1086.532 |
| ceruloplasmin precursor | NP_000087.1 | EDEDFQESNK | 31 | 620.7518 | 348.1872 |
| ceruloplasmin precursor | NP_000087.1 | EDEDFQESNK | 31 | 620.7518 | 477.2298 |
| ceruloplasmin precursor | NP_000087.1 | EDEDFQESNK | 31 | 620.7518 | 605.2884 |
| ceruloplasmin precursor | NP_000087.1 | EDEDFQESNK | 31 | 620.7518 | 752.3568 |
| ceruloplasmin precursor | NP_000087.1 | EDEDFQESNK | 31 | 620.7518 | 867.3837 |
| ceruloplasmin precursor | NP_000087.1 | EDEDFQESNK | 31 | 620.7518 | 996.4263 |
| ceruloplasmin precursor | NP_000087.1 | EDEDFQESNK | 31 | 620.7518 | 1111.453 |
| apolipoprotein E precursor | NP_000032.1 | QQTEWQSGQR | 32 | 624.2918 | 360.1985 |
| apolipoprotein E precursor | NP_000032.1 | QQTEWQSGQR | 32 | 624.2918 | 447.2305 |
| apolipoprotein E precursor | NP_000032.1 | QQTEWQSGQR | 32 | 624.2918 | 575.2891 |
| apolipoprotein E precursor | NP_000032.1 | QQTEWQSGQR | 32 | 624.2918 | 761.3683 |
| apolipoprotein E precursor | NP_000032.1 | QQTEWQSGQR | 32 | 624.2918 | 890.4109 |
| apolipoprotein E precursor | NP_000032.1 | QQTEWQSGQR | 32 | 624.2918 | 991.4586 |
| apolipoprotein E precursor | NP_000032.1 | QQTEWQSGQR | 32 | 624.2918 | 1119.517 |
| ceruloplasmin precursor | NP_000087.1 | DNEDFQESNR | 33 | 627.2551 | 376.1934 |
| ceruloplasmin precursor | NP_000087.1 | DNEDFQESNR | 33 | 627.2551 | 505.236 |
| ceruloplasmin precursor | NP_000087.1 | DNEDFQESNR | 33 | 627.2551 | 633.2946 |
| ceruloplasmin precursor | NP_000087.1 | DNEDFQESNR | 33 | 627.2551 | 780.3629 |
| ceruloplasmin precursor | NP_000087.1 | DNEDFQESNR | 33 | 627.2551 | 895.3899 |
| ceruloplasmin precursor | NP_000087.1 | DNEDFQESNR | 33 | 627.2551 | 1024.432 |
| ceruloplasmin precursor | NP_000087.1 | DNEDFQESNR | 33 | 627.2551 | 1138.475 |
| pregnancy-zone protein | NP_002855.1 | HQDGSYSTFGER | 34 | 692.2998 | 361.1825 |
| pregnancy-zone protein | NP_002855.1 | HQDGSYSTFGER | 34 | 692.2998 | 508.2509 |
| pregnancy-zone protein | NP_002855.1 | HQDGSYSTFGER | 34 | 692.2998 | 609.2985 |
| pregnancy-zone protein | NP_002855.1 | HQDGSYSTFGER | 34 | 692.2998 | 696.3306 |
| pregnancy-zone protein | NP_002855.1 | HQDGSYSTFGER | 34 | 692.2998 | 859.3939 |
| pregnancy-zone protein | NP_002855.1 | HQDGSYSTFGER | 34 | 692.2998 | 946.426 |
| pregnancy-zone protein | NP_002855.1 | HQDGSYSTFGER | 34 | 692.2998 | 1003.447 |
| pregnancy-zone protein | NP_002855.1 | HQDGSYSTFGER | 34 | 692.2998 | 1118.474 |
| pregnancy-zone protein | NP_002855.1 | HQDGSYSTFGER | 34 | 692.2998 | 1246.533 |
| apolipoprotein E precursor | NP_000032.1 | VQAAVGTSAAPVPSDNH | 35 | 810.9025 | 385.1461 |
| apolipoprotein E precursor | NP_000032.1 | VQAAVGTSAAPVPSDNH | 35 | 810.9025 | 472.1781 |
| apolipoprotein E precursor | NP_000032.1 | VQAAVGTSAAPVPSDNH | 35 | 810.9025 | 569.2309 |
| apolipoprotein E precursor | NP_000032.1 | VQAAVGTSAAPVPSDNH | 35 | 810.9025 | 668.2993 |
| apolipoprotein E precursor | NP_000032.1 | VQAAVGTSAAPVPSDNH | 35 | 810.9025 | 765.3521 |
| apolipoprotein E precursor | NP_000032.1 | VQAAVGTSAAPVPSDNH | 35 | 810.9025 | 836.3892 |
| apolipoprotein E precursor | NP_000032.1 | VQAAVGTSAAPVPSDNH | 35 | 810.9025 | 907.4263 |
| apolipoprotein E precursor | NP_000032.1 | VQAAVGTSAAPVPSDNH | 35 | 810.9025 | 994.4583 |
| apolipoprotein E precursor | NP_000032.1 | VQAAVGTSAAPVPSDNH | 35 | 810.9025 | 1095.506 |
| apolipoprotein E precursor | NP_000032.1 | VQAAVGTSAAPVPSDNH | 35 | 810.9025 | 1152.527 |
| apolipoprotein E precursor | NP_000032.1 | VQAAVGTSAAPVPSDNH | 35 | 810.9025 | 1251.596 |
| apolipoprotein E precursor | NP_000032.1 | VQAAVGTSAAPVPSDNH | 35 | 810.9025 | 1322.633 |
| apolipoprotein E precursor | NP_000032.1 | VQAAVGTSAAPVPSDNH | 35 | 810.9025 | 1393.67 |
| histidine-rich glycoprotein precursor | NP_000403.1 | HSHNNNSSDLHPHK | 36 | 812.3722 | 381.2239 |
| histidine-rich glycoprotein precursor | NP_000403.1 | HSHNNNSSDLHPHK | 36 | 812.3722 | 518.2828 |
| histidine-rich glycoprotein precursor | NP_000403.1 | HSHNNNSSDLHPHK | 36 | 812.3722 | 631.3669 |
| histidine-rich glycoprotein precursor | NP_000403.1 | HSHNNNSSDLHPHK | 36 | 812.3722 | 746.3939 |
| histidine-rich glycoprotein precursor | NP_000403.1 | HSHNNNSSDLHPHK | 36 | 812.3722 | 833.4259 |
| histidine-rich glycoprotein precursor | NP_000403.1 | HSHNNNSSDLHPHK | 36 | 812.3722 | 920.4579 |
| histidine-rich glycoprotein precursor | NP_000403.1 | HSHNNNSSDLHPHK | 36 | 812.3722 | 1034.501 |
| histidine-rich glycoprotein precursor | NP_000403.1 | HSHNNNSSDLHPHK | 36 | 812.3722 | 1148.544 |
| histidine-rich glycoprotein precursor | NP_000403.1 | HSHNNNSSDLHPHK | 36 | 812.3722 | 1262.587 |
| histidine-rich glycoprotein precursor | NP_000403.1 | HSHNNNSSDLHPHK | 36 | 812.3722 | 1399.646 |
| histidine-rich glycoprotein precursor | NP_000403.1 | HSHNNNSSDLHPHK | 36 | 812.3722 | 1486.678 |
| apolipoprotein A-II preproprotein | NP_001634.1 | SPELQAEAK | 37 | 486.7534 | 347.192 |
| apolipoprotein A-II preproprotein | NP_001634.1 | SPELQAEAK | 37 | 486.7534 | 418.2291 |
| apolipoprotein A-II preproprotein | NP_001634.1 | SPELQAEAK | 37 | 486.7534 | 546.2877 |
| apolipoprotein A-II preproprotein | NP_001634.1 | SPELQAEAK | 37 | 486.7534 | 659.3717 |
| apolipoprotein A-II preproprotein | NP_001634.1 | SPELQAEAK | 37 | 486.7534 | 788.4143 |
| apolipoprotein A-II preproprotein | NP_001634.1 | SPELQAEAK | 37 | 486.7534 | 885.4671 |
| apolipoprotein A-IV precursor | NP_000473.2 | SLAPYAQDTQEK | 38 | 675.8304 | 404.2134 |
| apolipoprotein A-IV precursor | NP_000473.2 | SLAPYAQDTQEK | 38 | 675.8304 | 505.2611 |
| apolipoprotein A-IV precursor | NP_000473.2 | SLAPYAQDTQEK | 38 | 675.8304 | 620.288 |
| apolipoprotein A-IV precursor | NP_000473.2 | SLAPYAQDTQEK | 38 | 675.8304 | 748.3466 |
| apolipoprotein A-IV precursor | NP_000473.2 | SLAPYAQDTQEK | 38 | 675.8304 | 819.3837 |
| apolipoprotein A-IV precursor | NP_000473.2 | SLAPYAQDTQEK | 38 | 675.8304 | 982.4471 |
| apolipoprotein A-IV precursor | NP_000473.2 | SLAPYAQDTQEK | 38 | 675.8304 | 1079.5 |
| apolipoprotein A-IV precursor | NP_000473.2 | SLAPYAQDTQEK | 38 | 675.8304 | 1150.537 |
| apolipoprotein A-IV precursor | NP_000473.2 | SLAPYAQDTQEK | 38 | 675.8304 | 1263.621 |
| ceruloplasmin precursor | NP_000087.1 | QYTDSTFR | 39 | 509.2354 | 423.2345 |
| ceruloplasmin precursor | NP_000087.1 | QYTDSTFR | 39 | 509.2354 | 510.2665 |
| ceruloplasmin precursor | NP_000087.1 | QYTDSTFR | 39 | 509.2354 | 625.2935 |
| ceruloplasmin precursor | NP_000087.1 | QYTDSTFR | 39 | 509.2354 | 726.3411 |
| ceruloplasmin precursor | NP_000087.1 | QYTDSTFR | 39 | 509.2354 | 889.4045 |
| apolipoprotein B precursor | NP_000375.2 | YTYNYEAESSSGVPGTADSR | 40 | 1077.467 | 377.1774 |
| apolipoprotein B precursor | NP_000375.2 | YTYNYEAESSSGVPGTADSR | 40 | 1077.467 | 448.2145 |
| apolipoprotein B precursor | NP_000375.2 | YTYNYEAESSSGVPGTADSR | 40 | 1077.467 | 549.2621 |
| apolipoprotein B precursor | NP_000375.2 | YTYNYEAESSSGVPGTADSR | 40 | 1077.467 | 606.2836 |
| apolipoprotein B precursor | NP_000375.2 | YTYNYEAESSSGVPGTADSR | 40 | 1077.467 | 703.3364 |
| apolipoprotein B precursor | NP_000375.2 | YTYNYEAESSSGVPGTADSR | 40 | 1077.467 | 802.4048 |
| apolipoprotein B precursor | NP_000375.2 | YTYNYEAESSSGVPGTADSR | 40 | 1077.467 | 859.4263 |
| apolipoprotein B precursor | NP_000375.2 | YTYNYEAESSSGVPGTADSR | 40 | 1077.467 | 946.4583 |
| apolipoprotein B precursor | NP_000375.2 | YTYNYEAESSSGVPGTADSR | 40 | 1077.467 | 1033.49 |
| apolipoprotein B precursor | NP_000375.2 | YTYNYEAESSSGVPGTADSR | 40 | 1077.467 | 1120.522 |
| apolipoprotein B precursor | NP_000375.2 | YTYNYEAESSSGVPGTADSR | 40 | 1077.467 | 1249.565 |
| apolipoprotein B precursor | NP_000375.2 | YTYNYEAESSSGVPGTADSR | 40 | 1077.467 | 1320.602 |
| apolipoprotein B precursor | NP_000375.2 | YTYNYEAESSSGVPGTADSR | 40 | 1077.467 | 1449.645 |
| apolipoprotein B precursor | NP_000375.2 | ETLEDTR | 41 | 432.2089 | 391.193 |
| apolipoprotein B precursor | NP_000375.2 | ETLEDTR | 41 | 432.2089 | 520.2356 |
| apolipoprotein B precursor | NP_000375.2 | ETLEDTR | 41 | 432.2089 | 633.3197 |
| apolipoprotein B precursor | NP_000375.2 | ETLEDTR | 41 | 432.2089 | 734.3674 |
| histidine-rich glycoprotein precursor | NP_000403.1 | QIGSVYR | 42 | 411.7271 | 437.2502 |
| histidine-rich glycoprotein precursor | NP_000403.1 | QIGSVYR | 42 | 411.7271 | 524.2822 |
| histidine-rich glycoprotein precursor | NP_000403.1 | QIGSVYR | 42 | 411.7271 | 581.3036 |
| histidine-rich glycoprotein precursor | NP_000403.1 | QIGSVYR | 42 | 411.7271 | 694.3877 |
| apolipoprotein B precursor | NP_000375.2 | AASGTTGTYQEWK | 43 | 700.3281 | 462.2342 |
| apolipoprotein B precursor | NP_000375.2 | AASGTTGTYQEWK | 43 | 700.3281 | 590.2927 |
| apolipoprotein B precursor | NP_000375.2 | AASGTTGTYQEWK | 43 | 700.3281 | 753.3561 |
| apolipoprotein B precursor | NP_000375.2 | AASGTTGTYQEWK | 43 | 700.3281 | 854.4037 |
| apolipoprotein B precursor | NP_000375.2 | AASGTTGTYQEWK | 43 | 700.3281 | 911.4252 |
| apolipoprotein B precursor | NP_000375.2 | AASGTTGTYQEWK | 43 | 700.3281 | 1012.473 |
| apolipoprotein B precursor | NP_000375.2 | AASGTTGTYQEWK | 43 | 700.3281 | 1113.521 |
| apolipoprotein B precursor | NP_000375.2 | AASGTTGTYQEWK | 43 | 700.3281 | 1170.542 |
| apolipoprotein B precursor | NP_000375.2 | AASGTTGTYQEWK | 43 | 700.3281 | 1257.574 |
| apolipoprotein B precursor | NP_000375.2 | AASGTTGTYQEWK | 43 | 700.3281 | 1328.611 |
| histidine-rich glycoprotein precursor | NP_000403.1 | IADAHLDR | 44 | 455.7407 | 403.2294 |
| histidine-rich glycoprotein precursor | NP_000403.1 | IADAHLDR | 44 | 455.7407 | 540.2883 |
| histidine-rich glycoprotein precursor | NP_000403.1 | IADAHLDR | 44 | 455.7407 | 611.3254 |
| histidine-rich glycoprotein precursor | NP_000403.1 | IADAHLDR | 44 | 455.7407 | 726.3524 |
| histidine-rich glycoprotein precursor | NP_000403.1 | IADAHLDR | 44 | 455.7407 | 797.3895 |
| apolipoprotein H precursor | NP_000033.1 | ATVVYQGER | 45 | 511.7669 | 361.1825 |
| apolipoprotein H precursor | NP_000033.1 | ATVVYQGER | 45 | 511.7669 | 489.241 |
| apolipoprotein H precursor | NP_000033.1 | ATVVYQGER | 45 | 511.7669 | 751.3728 |
| apolipoprotein H precursor | NP_000033.1 | ATVVYQGER | 45 | 511.7669 | 850.4412 |
| apolipoprotein H precursor | NP_000033.1 | ATVVYQGER | 45 | 511.7669 | 951.4889 |
| apolipoprotein A-I preproprotein | NP_000030.1 | AHVDALR | 46 | 391.2194 | 359.2396 |
| apolipoprotein A-I preproprotein | NP_000030.1 | AHVDALR | 46 | 391.2194 | 474.2665 |
| apolipoprotein A-I preproprotein | NP_000030.1 | AHVDALR | 46 | 391.2194 | 573.335 |
| apolipoprotein A-I preproprotein | NP_000030.1 | AHVDALR | 46 | 391.2194 | 710.3939 |
| pregnancy-zone protein | NP_002855.1 | LPSNVVK | 47 | 378.7343 | 345.2491 |
| pregnancy-zone protein | NP_002855.1 | LPSNVVK | 47 | 378.7343 | 459.292 |
| pregnancy-zone protein | NP_002855.1 | LPSNVVK | 47 | 378.7343 | 546.324 |
| pregnancy-zone protein | NP_002855.1 | LPSNVVK | 47 | 378.7343 | 643.3768 |
| apolipoprotein B precursor | NP_000375.2 | FFGEGTK | 48 | 393.1951 | 305.1814 |
| apolipoprotein B precursor | NP_000375.2 | FFGEGTK | 48 | 393.1951 | 434.224 |
| apolipoprotein B precursor | NP_000375.2 | FFGEGTK | 48 | 393.1951 | 491.2455 |
| apolipoprotein B precursor | NP_000375.2 | FFGEGTK | 48 | 393.1951 | 638.3138 |
| serum amyloid A4; constitutive | NP_006503.1 | FRPDGLPK | 49 | 465.2638 | 357.2491 |
| serum amyloid A4; constitutive | NP_006503.1 | FRPDGLPK | 49 | 465.2638 | 414.2705 |
| serum amyloid A4; constitutive | NP_006503.1 | FRPDGLPK | 49 | 465.2638 | 529.2975 |
| serum amyloid A4; constitutive | NP_006503.1 | FRPDGLPK | 49 | 465.2638 | 626.3503 |
| serum amyloid A4; constitutive | NP_006503.1 | FRPDGLPK | 49 | 465.2638 | 782.4514 |
| apolipoprotein E precursor | NP_000032.1 | LEEQAQQIR | 50 | 557.7962 | 416.261 |
| apolipoprotein E precursor | NP_000032.1 | LEEQAQQIR | 50 | 557.7962 | 544.3196 |
| apolipoprotein E precursor | NP_000032.1 | LEEQAQQIR | 50 | 557.7962 | 615.3568 |
| apolipoprotein E precursor | NP_000032.1 | LEEQAQQIR | 50 | 557.7962 | 743.4153 |
| apolipoprotein E precursor | NP_000032.1 | LEEQAQQIR | 50 | 557.7962 | 872.4579 |
| apolipoprotein E precursor | NP_000032.1 | LEEQAQQIR | 50 | 557.7962 | 1001.5 |
| apolipoprotein B precursor | NP_000375.2 | QAEAVLK | 51 | 379.724 | 359.2647 |
| apolipoprotein B precursor | NP_000375.2 | QAEAVLK | 51 | 379.724 | 430.3018 |
| apolipoprotein B precursor | NP_000375.2 | QAEAVLK | 51 | 379.724 | 559.3444 |
| apolipoprotein B precursor | NP_000375.2 | QAEAVLK | 51 | 379.724 | 630.3815 |
| apolipoprotein B precursor | NP_000375.2 | YYELEEK | 52 | 487.2293 | 405.1974 |
| apolipoprotein B precursor | NP_000375.2 | YYELEEK | 52 | 487.2293 | 518.2815 |
| apolipoprotein B precursor | NP_000375.2 | YYELEEK | 52 | 487.2293 | 647.3241 |
| apolipoprotein B precursor | NP_000375.2 | YYELEEK | 52 | 487.2293 | 810.3875 |
| apolipoprotein E precursor | NP_000032.1 | LAVYQAGAR | 53 | 474.7667 | 303.177 |
| apolipoprotein E precursor | NP_000032.1 | LAVYQAGAR | 53 | 474.7667 | 374.2141 |
| apolipoprotein E precursor | NP_000032.1 | LAVYQAGAR | 53 | 474.7667 | 502.2727 |
| apolipoprotein E precursor | NP_000032.1 | LAVYQAGAR | 53 | 474.7667 | 665.336 |
| apolipoprotein E precursor | NP_000032.1 | LAVYQAGAR | 53 | 474.7667 | 764.4044 |
| apolipoprotein E precursor | NP_000032.1 | LAVYQAGAR | 53 | 474.7667 | 835.4415 |
| apolipoprotein A-IV precursor | NP_000473.2 | EAVEHLQK | 54 | 477.2562 | 388.2549 |
| apolipoprotein A-IV precursor | NP_000473.2 | EAVEHLQK | 54 | 477.2562 | 525.3138 |
| apolipoprotein A-IV precursor | NP_000473.2 | EAVEHLQK | 54 | 477.2562 | 654.3564 |
| apolipoprotein A-IV precursor | NP_000473.2 | EAVEHLQK | 54 | 477.2562 | 753.4248 |
| apolipoprotein A-IV precursor | NP_000473.2 | EAVEHLQK | 54 | 477.2562 | 824.4619 |
| apolipoprotein C-II precursor | NP_000474.2 | TAAQNLYEK | 55 | 519.2667 | 439.2182 |
| apolipoprotein C-II precursor | NP_000474.2 | TAAQNLYEK | 55 | 519.2667 | 552.3022 |
| apolipoprotein C-II precursor | NP_000474.2 | TAAQNLYEK | 55 | 519.2667 | 666.3452 |
| apolipoprotein C-II precursor | NP_000474.2 | TAAQNLYEK | 55 | 519.2667 | 794.4037 |
| apolipoprotein C-II precursor | NP_000474.2 | TAAQNLYEK | 55 | 519.2667 | 865.4409 |
| apolipoprotein C-II precursor | NP_000474.2 | TAAQNLYEK | 55 | 519.2667 | 936.478 |
| apolipoprotein A-IV precursor | NP_000473.2 | LLPHANEVSQK | 56 | 618.3408 | 362.2029 |
| apolipoprotein A-IV precursor | NP_000473.2 | LLPHANEVSQK | 56 | 618.3408 | 461.2713 |
| apolipoprotein A-IV precursor | NP_000473.2 | LLPHANEVSQK | 56 | 618.3408 | 590.3138 |
| apolipoprotein A-IV precursor | NP_000473.2 | LLPHANEVSQK | 56 | 618.3408 | 704.3568 |
| apolipoprotein A-IV precursor | NP_000473.2 | LLPHANEVSQK | 56 | 618.3408 | 775.3939 |
| apolipoprotein A-IV precursor | NP_000473.2 | LLPHANEVSQK | 56 | 618.3408 | 912.4528 |
| apolipoprotein A-IV precursor | NP_000473.2 | LLPHANEVSQK | 56 | 618.3408 | 1009.506 |
| apolipoprotein A-IV precursor | NP_000473.2 | LLPHANEVSQK | 56 | 618.3408 | 1122.59 |
| apolipoprotein B precursor | NP_000375.2 | DFSAEYEEDGK | 57 | 645.262 | 319.1607 |
| apolipoprotein B precursor | NP_000375.2 | DFSAEYEEDGK | 57 | 645.262 | 448.2032 |
| apolipoprotein B precursor | NP_000375.2 | DFSAEYEEDGK | 57 | 645.262 | 577.2458 |
| apolipoprotein B precursor | NP_000375.2 | DFSAEYEEDGK | 57 | 645.262 | 740.3092 |
| apolipoprotein B precursor | NP_000375.2 | DFSAEYEEDGK | 57 | 645.262 | 869.3517 |
| apolipoprotein B precursor | NP_000375.2 | DFSAEYEEDGK | 57 | 645.262 | 940.3889 |
| apolipoprotein B precursor | NP_000375.2 | DFSAEYEEDGK | 57 | 645.262 | 1027.421 |
| apolipoprotein B precursor | NP_000375.2 | DFSAEYEEDGK | 57 | 645.262 | 1174.489 |
| apolipoprotein A-IV precursor | NP_000473.2 | TQVSTQAEQLR | 58 | 630.8307 | 416.261 |
| apolipoprotein A-IV precursor | NP_000473.2 | TQVSTQAEQLR | 58 | 630.8307 | 545.3036 |
| apolipoprotein A-IV precursor | NP_000473.2 | TQVSTQAEQLR | 58 | 630.8307 | 616.3408 |
| apolipoprotein A-IV precursor | NP_000473.2 | TQVSTQAEQLR | 58 | 630.8307 | 744.3994 |
| apolipoprotein A-IV precursor | NP_000473.2 | TQVSTQAEQLR | 58 | 630.8307 | 845.447 |
| apolipoprotein A-IV precursor | NP_000473.2 | TQVSTQAEQLR | 58 | 630.8307 | 932.4791 |
| apolipoprotein A-IV precursor | NP_000473.2 | TQVSTQAEQLR | 58 | 630.8307 | 1031.547 |
| apolipoprotein A-IV precursor | NP_000473.2 | TQVSTQAEQLR | 58 | 630.8307 | 1159.606 |
| pregnancy-zone protein | NP_002855.1 | YGAATFTR | 59 | 443.7245 | 423.2345 |
| pregnancy-zone protein | NP_002855.1 | YGAATFTR | 59 | 443.7245 | 524.2822 |
| pregnancy-zone protein | NP_002855.1 | YGAATFTR | 59 | 443.7245 | 595.3193 |
| pregnancy-zone protein | NP_002855.1 | YGAATFTR | 59 | 443.7245 | 666.3564 |
| pregnancy-zone protein | NP_002855.1 | YGAATFTR | 59 | 443.7245 | 723.3779 |
| apolipoprotein D precursor | NP_001638.1 | IPTTFENGR | 60 | 517.7709 | 346.1828 |
| apolipoprotein D precursor | NP_001638.1 | IPTTFENGR | 60 | 517.7709 | 475.2254 |
| apolipoprotein D precursor | NP_001638.1 | IPTTFENGR | 60 | 517.7709 | 622.2938 |
| apolipoprotein D precursor | NP_001638.1 | IPTTFENGR | 60 | 517.7709 | 723.3415 |
| apolipoprotein D precursor | NP_001638.1 | IPTTFENGR | 60 | 517.7709 | 824.3892 |
| apolipoprotein D precursor | NP_001638.1 | IPTTFENGR | 60 | 517.7709 | 921.4419 |
| apolipoprotein B precursor | NP_000375.2 | SNTVASLHTEK | 61 | 593.8068 | 377.2025 |
| apolipoprotein B precursor | NP_000375.2 | SNTVASLHTEK | 61 | 593.8068 | 514.2614 |
| apolipoprotein B precursor | NP_000375.2 | SNTVASLHTEK | 61 | 593.8068 | 627.3455 |
| apolipoprotein B precursor | NP_000375.2 | SNTVASLHTEK | 61 | 593.8068 | 714.3775 |
| apolipoprotein B precursor | NP_000375.2 | SNTVASLHTEK | 61 | 593.8068 | 785.4147 |
| apolipoprotein B precursor | NP_000375.2 | SNTVASLHTEK | 61 | 593.8068 | 884.483 |
| apolipoprotein B precursor | NP_000375.2 | SNTVASLHTEK | 61 | 593.8068 | 985.5308 |
| apolipoprotein B precursor | NP_000375.2 | SNTVASLHTEK | 61 | 593.8068 | 1099.574 |
| apolipoprotein A-IV precursor | NP_000473.2 | VEPYGENFNK | 62 | 598.7828 | 408.2236 |
| apolipoprotein A-IV precursor | NP_000473.2 | VEPYGENFNK | 62 | 598.7828 | 522.2665 |
| apolipoprotein A-IV precursor | NP_000473.2 | VEPYGENFNK | 62 | 598.7828 | 651.3091 |
| apolipoprotein A-IV precursor | NP_000473.2 | VEPYGENFNK | 62 | 598.7828 | 708.3306 |
| apolipoprotein A-IV precursor | NP_000473.2 | VEPYGENFNK | 62 | 598.7828 | 871.3939 |
| apolipoprotein A-IV precursor | NP_000473.2 | VEPYGENFNK | 62 | 598.7828 | 968.4467 |
| apolipoprotein A-IV precursor | NP_000473.2 | VEPYGENFNK | 62 | 598.7828 | 1097.489 |
| apolipoprotein C-II precursor | NP_000474.2 | LRDLYSK | 63 | 447.7558 | 397.2076 |
| apolipoprotein C-II precursor | NP_000474.2 | LRDLYSK | 63 | 447.7558 | 510.2917 |
| apolipoprotein C-II precursor | NP_000474.2 | LRDLYSK | 63 | 447.7558 | 625.3186 |
| apolipoprotein C-II precursor | NP_000474.2 | LRDLYSK | 63 | 447.7558 | 781.4197 |
| serum amyloid P component precursor | NP_001630.1 | IVLGQEQDSYGGK | 64 | 697.3515 | 261.1552 |
| serum amyloid P component precursor | NP_001630.1 | IVLGQEQDSYGGK | 64 | 697.3515 | 424.2185 |
| serum amyloid P component precursor | NP_001630.1 | IVLGQEQDSYGGK | 64 | 697.3515 | 511.2505 |
| serum amyloid P component precursor | NP_001630.1 | IVLGQEQDSYGGK | 64 | 697.3515 | 626.2775 |
| serum amyloid P component precursor | NP_001630.1 | IVLGQEQDSYGGK | 64 | 697.3515 | 754.3361 |
| serum amyloid P component precursor | NP_001630.1 | IVLGQEQDSYGGK | 64 | 697.3515 | 883.3787 |
| serum amyloid P component precursor | NP_001630.1 | IVLGQEQDSYGGK | 64 | 697.3515 | 1011.437 |
| serum amyloid P component precursor | NP_001630.1 | IVLGQEQDSYGGK | 64 | 697.3515 | 1068.459 |
| serum amyloid P component precursor | NP_001630.1 | IVLGQEQDSYGGK | 64 | 697.3515 | 1181.543 |
| serum amyloid P component precursor | NP_001630.1 | IVLGQEQDSYGGK | 64 | 697.3515 | 1280.611 |
| apolipoprotein B precursor | NP_000375.2 | LDNIYSSDK | 65 | 527.7562 | 349.1712 |
| apolipoprotein B precursor | NP_000375.2 | LDNIYSSDK | 65 | 527.7562 | 436.2032 |
| apolipoprotein B precursor | NP_000375.2 | LDNIYSSDK | 65 | 527.7562 | 599.2666 |
| apolipoprotein B precursor | NP_000375.2 | LDNIYSSDK | 65 | 527.7562 | 712.3506 |
| apolipoprotein B precursor | NP_000375.2 | LDNIYSSDK | 65 | 527.7562 | 826.3936 |
| apolipoprotein B precursor | NP_000375.2 | LDNIYSSDK | 65 | 527.7562 | 941.4205 |
| apolipoprotein B precursor | NP_000375.2 | VAWHYDEEK | 66 | 588.7697 | 405.1974 |
| apolipoprotein B precursor | NP_000375.2 | VAWHYDEEK | 66 | 588.7697 | 520.2244 |
| apolipoprotein B precursor | NP_000375.2 | VAWHYDEEK | 66 | 588.7697 | 683.2877 |
| apolipoprotein B precursor | NP_000375.2 | VAWHYDEEK | 66 | 588.7697 | 820.3466 |
| apolipoprotein B precursor | NP_000375.2 | VAWHYDEEK | 66 | 588.7697 | 1006.426 |
| apolipoprotein B precursor | NP_000375.2 | VAWHYDEEK | 66 | 588.7697 | 1077.463 |
| apolipoprotein B precursor | NP_000375.2 | VSSFYAK | 67 | 401.2107 | 381.2127 |
| apolipoprotein B precursor | NP_000375.2 | VSSFYAK | 67 | 401.2107 | 528.2811 |
| apolipoprotein B precursor | NP_000375.2 | VSSFYAK | 67 | 401.2107 | 615.3131 |
| apolipoprotein B precursor | NP_000375.2 | VSSFYAK | 67 | 401.2107 | 702.3452 |
| alpha-2-macroglobulin precursor | NP_000005.2 | LPPNVVEESAR | 68 | 605.825 | 333.1875 |
| alpha-2-macroglobulin precursor | NP_000005.2 | LPPNVVEESAR | 68 | 605.825 | 462.2301 |
| alpha-2-macroglobulin precursor | NP_000005.2 | LPPNVVEESAR | 68 | 605.825 | 591.2727 |
| alpha-2-macroglobulin precursor | NP_000005.2 | LPPNVVEESAR | 68 | 605.825 | 690.3411 |
| alpha-2-macroglobulin precursor | NP_000005.2 | LPPNVVEESAR | 68 | 605.825 | 789.4095 |
| alpha-2-macroglobulin precursor | NP_000005.2 | LPPNVVEESAR | 68 | 605.825 | 903.4525 |
| alpha-2-macroglobulin precursor | NP_000005.2 | LPPNVVEESAR | 68 | 605.825 | 1000.505 |
| alpha-2-macroglobulin precursor | NP_000005.2 | LPPNVVEESAR | 68 | 605.825 | 1097.558 |
| ceruloplasmin precursor | NP_000087.1 | VNKDDEEFIESNK | 69 | 783.8677 | 348.1872 |
| ceruloplasmin precursor | NP_000087.1 | VNKDDEEFIESNK | 69 | 783.8677 | 477.2298 |
| ceruloplasmin precursor | NP_000087.1 | VNKDDEEFIESNK | 69 | 783.8677 | 590.3138 |
| ceruloplasmin precursor | NP_000087.1 | VNKDDEEFIESNK | 69 | 783.8677 | 737.3823 |
| ceruloplasmin precursor | NP_000087.1 | VNKDDEEFIESNK | 69 | 783.8677 | 866.4249 |
| ceruloplasmin precursor | NP_000087.1 | VNKDDEEFIESNK | 69 | 783.8677 | 995.4675 |
| ceruloplasmin precursor | NP_000087.1 | VNKDDEEFIESNK | 69 | 783.8677 | 1110.494 |
| ceruloplasmin precursor | NP_000087.1 | VNKDDEEFIESNK | 69 | 783.8677 | 1225.521 |
| ceruloplasmin precursor | NP_000087.1 | VNKDDEEFIESNK | 69 | 783.8677 | 1353.616 |
| ceruloplasmin precursor | NP_000087.1 | VNKDDEEFIESNK | 69 | 783.8677 | 1467.659 |
| apolipoprotein B precursor | NP_000375.2 | EAQEVFK | 70 | 425.7189 | 393.2491 |
| apolipoprotein B precursor | NP_000375.2 | EAQEVFK | 70 | 425.7189 | 522.2917 |
| apolipoprotein B precursor | NP_000375.2 | EAQEVFK | 70 | 425.7189 | 650.3503 |
| apolipoprotein B precursor | NP_000375.2 | EAQEVFK | 70 | 425.7189 | 721.3874 |
| alpha 1 globin | NP_000549.1 | VGAHAGEYGAEALER | 71 | 765.3708 | 417.2451 |
| alpha 1 globin | NP_000549.1 | VGAHAGEYGAEALER | 71 | 765.3708 | 488.2822 |
| alpha 1 globin | NP_000549.1 | VGAHAGEYGAEALER | 71 | 765.3708 | 617.3248 |
| alpha 1 globin | NP_000549.1 | VGAHAGEYGAEALER | 71 | 765.3708 | 688.3619 |
| alpha 1 globin | NP_000549.1 | VGAHAGEYGAEALER | 71 | 765.3708 | 745.3834 |
| alpha 1 globin | NP_000549.1 | VGAHAGEYGAEALER | 71 | 765.3708 | 908.4467 |
| alpha 1 globin | NP_000549.1 | VGAHAGEYGAEALER | 71 | 765.3708 | 1037.489 |
| alpha 1 globin | NP_000549.1 | VGAHAGEYGAEALER | 71 | 765.3708 | 1094.511 |
| alpha 1 globin | NP_000549.1 | VGAHAGEYGAEALER | 71 | 765.3708 | 1165.548 |
| alpha 1 globin | NP_000549.1 | VGAHAGEYGAEALER | 71 | 765.3708 | 1302.607 |
| alpha 1 globin | NP_000549.1 | VGAHAGEYGAEALER | 71 | 765.3708 | 1373.644 |
| alpha 1 globin | NP_000549.1 | VGAHAGEYGAEALER | 71 | 765.3708 | 1430.665 |
| apolipoprotein E precursor | NP_000032.1 | LQAEAFQAR | 72 | 517.2749 | 374.2141 |
| apolipoprotein E precursor | NP_000032.1 | LQAEAFQAR | 72 | 517.2749 | 521.2825 |
| apolipoprotein E precursor | NP_000032.1 | LQAEAFQAR | 72 | 517.2749 | 592.3196 |
| apolipoprotein E precursor | NP_000032.1 | LQAEAFQAR | 72 | 517.2749 | 721.3622 |
| apolipoprotein E precursor | NP_000032.1 | LQAEAFQAR | 72 | 517.2749 | 792.3994 |
| apolipoprotein E precursor | NP_000032.1 | LQAEAFQAR | 72 | 517.2749 | 920.4579 |
| apolipoprotein D precursor | NP_001638.1 | VLNQELR | 73 | 436.2534 | 417.2451 |
| apolipoprotein D precursor | NP_001638.1 | VLNQELR | 73 | 436.2534 | 545.3036 |
| apolipoprotein D precursor | NP_001638.1 | VLNQELR | 73 | 436.2534 | 659.3466 |
| apolipoprotein D precursor | NP_001638.1 | VLNQELR | 73 | 436.2534 | 772.4306 |
| apolipoprotein B precursor | NP_000375.2 | AGHIAWTSSGK | 74 | 557.7856 | 291.1657 |
| apolipoprotein B precursor | NP_000375.2 | AGHIAWTSSGK | 74 | 557.7856 | 378.1978 |
| apolipoprotein B precursor | NP_000375.2 | AGHIAWTSSGK | 74 | 557.7856 | 479.2455 |
| apolipoprotein B precursor | NP_000375.2 | AGHIAWTSSGK | 74 | 557.7856 | 665.3248 |
| apolipoprotein B precursor | NP_000375.2 | AGHIAWTSSGK | 74 | 557.7856 | 736.3619 |
| apolipoprotein B precursor | NP_000375.2 | AGHIAWTSSGK | 74 | 557.7856 | 849.4459 |
| apolipoprotein B precursor | NP_000375.2 | AGHIAWTSSGK | 74 | 557.7856 | 986.5049 |
| apolipoprotein B precursor | NP_000375.2 | AGHIAWTSSGK | 74 | 557.7856 | 1043.526 |
| apolipoprotein B precursor | NP_000375.2 | AAIQALR | 75 | 371.7321 | 359.2396 |
| apolipoprotein B precursor | NP_000375.2 | AAIQALR | 75 | 371.7321 | 487.2982 |
| apolipoprotein B precursor | NP_000375.2 | AAIQALR | 75 | 371.7321 | 600.3822 |
| apolipoprotein B precursor | NP_000375.2 | AAIQALR | 75 | 371.7321 | 671.4193 |
| alpha 1 globin | NP_000549.1 | VDPVNFK | 76 | 409.724 | 408.2236 |
| alpha 1 globin | NP_000549.1 | VDPVNFK | 76 | 409.724 | 507.292 |
| alpha 1 globin | NP_000549.1 | VDPVNFK | 76 | 409.724 | 604.3448 |
| alpha 1 globin | NP_000549.1 | VDPVNFK | 76 | 409.724 | 719.3717 |
| serum amyloid A4; constitutive | NP_006503.1 | GPGGVWAAK | 77 | 421.7296 | 289.1865 |
| serum amyloid A4; constitutive | NP_006503.1 | GPGGVWAAK | 77 | 421.7296 | 475.2658 |
| serum amyloid A4; constitutive | NP_006503.1 | GPGGVWAAK | 77 | 421.7296 | 574.3342 |
| serum amyloid A4; constitutive | NP_006503.1 | GPGGVWAAK | 77 | 421.7296 | 631.3557 |
| serum amyloid A4; constitutive | NP_006503.1 | GPGGVWAAK | 77 | 421.7296 | 688.3771 |
| serum amyloid A4; constitutive | NP_006503.1 | GPGGVWAAK | 77 | 421.7296 | 785.4299 |
| apolipoprotein D precursor | NP_001638.1 | C[Carboxymethyl]PNPPVQENFDV | 78 | 829.8776 | 360.2236 |
| | | NK | | | |
| apolipoprotein D precursor | NP_001638.1 | C[Carboxymethyl]PNPPVQENFDV | 78 | 829.8776 | 475.2505 |
| | | NK | | | |
| apolipoprotein D precursor | NP_001638.1 | C[Carboxymethyl]PNPPVQENFDV | 78 | 829.8776 | 622.319 |
| | | NK | | | |
| apolipoprotein D precursor | NP_001638.1 | C[Carboxymethyl]PNPPVQENFDV | 78 | 829.8776 | 736.3619 |
| | | NK | | | |
| apolipoprotein D precursor | NP_001638.1 | C[Carboxymethyl]PNPPVQENFDV | 78 | 829.8776 | 865.4045 |
| | | NK | | | |
| apolipoprotein D precursor | NP_001638.1 | C[Carboxymethyl]PNPPVQENFDV | 78 | 829.8776 | 993.4631 |
| | | NK | | | |
| apolipoprotein D precursor | NP_001638.1 | C[Carboxymethyl]PNPPVQENFDV | 78 | 829.8776 | 1092.531 |
| | | NK | | | |
| apolipoprotein D precursor | NP_001638.1 | C[Carboxymethyl]PNPPVQENFDV | 78 | 829.8776 | 1189.584 |
| | | NK | | | |
| apolipoprotein D precursor | NP_001638.1 | C[Carboxymethyl]PNPPVQENFDV | 78 | 829.8776 | 1286.637 |
| | | NK | | | |
| apolipoprotein D precursor | NP_001638.1 | C[Carboxymethyl]PNPPVQENFDV | 78 | 829.8776 | 1400.68 |
| | | NK | | | |
| apolipoprotein D precursor | NP_001638.1 | C[Carboxymethyl]PNPPVQENFDV | 78 | 829.8776 | 1497.733 |
| | | NK | | | |
| apolipoprotein B precursor | NP_000375.2 | QIDDIDVR | 79 | 487.2511 | 389.2137 |
| apolipoprotein B precursor | NP_000375.2 | QIDDIDVR | 79 | 487.2511 | 502.2978 |
| apolipoprotein B precursor | NP_000375.2 | QIDDIDVR | 79 | 487.2511 | 617.3248 |
| apolipoprotein B precursor | NP_000375.2 | QIDDIDVR | 79 | 487.2511 | 732.3517 |
| apolipoprotein B precursor | NP_000375.2 | QIDDIDVR | 79 | 487.2511 | 845.4358 |
| histidine-rich glycoprotein precursor | NP_000403.1 | RPSEIVIGQC[Carboxymethyl]K | 80 | 644.3399 | 436.1855 |
| histidine-rich glycoprotein precursor | NP_000403.1 | RPSEIVIGQC[Carboxymethyl]K | 80 | 644.3399 | 493.207 |
| histidine-rich glycoprotein precursor | NP_000403.1 | RPSEIVIGQC[Carboxymethyl]K | 80 | 644.3399 | 606.291 |
| histidine-rich glycoprotein precursor | NP_000403.1 | RPSEIVIGQC[Carboxymethyl]K | 80 | 644.3399 | 705.3594 |
| histidine-rich glycoprotein precursor | NP_000403.1 | RPSEIVIGQC[Carboxymethyl]K | 80 | 644.3399 | 818.4435 |
| histidine-rich glycoprotein precursor | NP_000403.1 | RPSEIVIGQC[Carboxymethyl]K | 80 | 644.3399 | 947.4861 |
| histidine-rich glycoprotein precursor | NP_000403.1 | RPSEIVIGQC[Carboxymethyl]K | 80 | 644.3399 | 1034.518 |
| histidine-rich glycoprotein precursor | NP_000403.1 | RPSEIVIGQC[Carboxymethyl]K | 80 | 644.3399 | 1131.571 |
| apolipoprotein A-IV precursor | NP_000473.2 | IDQNVEELK | 81 | 544.2852 | 389.2389 |
| apolipoprotein A-IV precursor | NP_000473.2 | IDQNVEELK | 81 | 544.2852 | 518.2815 |
| apolipoprotein A-IV precursor | NP_000473.2 | IDQNVEELK | 81 | 544.2852 | 617.3499 |
| apolipoprotein A-IV precursor | NP_000473.2 | IDQNVEELK | 81 | 544.2852 | 731.3928 |
| apolipoprotein A-IV precursor | NP_000473.2 | IDQNVEELK | 81 | 544.2852 | 859.4514 |
| apolipoprotein A-IV precursor | NP_000473.2 | IDQNVEELK | 81 | 544.2852 | 974.4784 |
| apolipoprotein A-I preproprotein | NP_000030.1 | ATEHLSTLSEK | 82 | 608.3144 | 363.1869 |
| apolipoprotein A-I preproprotein | NP_000030.1 | ATEHLSTLSEK | 82 | 608.3144 | 476.2709 |
| apolipoprotein A-I preproprotein | NP_000030.1 | ATEHLSTLSEK | 82 | 608.3144 | 577.3186 |
| apolipoprotein A-I preproprotein | NP_000030.1 | ATEHLSTLSEK | 82 | 608.3144 | 664.3506 |
| apolipoprotein A-I preproprotein | NP_000030.1 | ATEHLSTLSEK | 82 | 608.3144 | 777.4347 |
| apolipoprotein A-I preproprotein | NP_000030.1 | ATEHLSTLSEK | 82 | 608.3144 | 914.4937 |
| apolipoprotein A-I preproprotein | NP_000030.1 | ATEHLSTLSEK | 82 | 608.3144 | 1043.536 |
| apolipoprotein A-I preproprotein | NP_000030.1 | ATEHLSTLSEK | 82 | 608.3144 | 1144.584 |
| pregnancy-zone protein | NP_002855.1 | YNILPEK | 83 | 438.7449 | 373.2076 |
| pregnancy-zone protein | NP_002855.1 | YNILPEK | 83 | 438.7449 | 486.2917 |
| pregnancy-zone protein | NP_002855.1 | YNILPEK | 83 | 438.7449 | 599.3757 |
| pregnancy-zone protein | NP_002855.1 | YNILPEK | 83 | 438.7449 | 713.4186 |
| alpha-2-macroglobulin precursor | NP_000005.2 | YNILPEK | 84 | 438.7469 | 373.2076 |
| alpha-2-macroglobulin precursor | NP_000005.2 | YNILPEK | 84 | 438.7469 | 486.2917 |
| alpha-2-macroglobulin precursor | NP_000005.2 | YNILPEK | 84 | 438.7469 | 599.3757 |
| alpha-2-macroglobulin precursor | NP_000005.2 | YNILPEK | 84 | 438.7469 | 713.4186 |
| apolipoprotein B precursor | NP_000375.2 | SLDEHYHIR | 85 | 585.2885 | 425.2614 |
| apolipoprotein B precursor | NP_000375.2 | SLDEHYHIR | 85 | 585.2885 | 588.3247 |
| apolipoprotein B precursor | NP_000375.2 | SLDEHYHIR | 85 | 585.2885 | 725.3836 |
| apolipoprotein B precursor | NP_000375.2 | SLDEHYHIR | 85 | 585.2885 | 854.4262 |
| apolipoprotein B precursor | NP_000375.2 | SLDEHYHIR | 85 | 585.2885 | 969.4531 |
| apolipoprotein B precursor | NP_000375.2 | SLDEHYHIR | 85 | 585.2885 | 1082.537 |
| beta globin | NP_000509.1 | LHVDPENFR | 86 | 563.7856 | 436.2297 |
| beta globin | NP_000509.1 | LHVDPENFR | 86 | 563.7856 | 565.2723 |
| beta globin | NP_000509.1 | LHVDPENFR | 86 | 563.7856 | 662.3251 |
| beta globin | NP_000509.1 | LHVDPENFR | 86 | 563.7856 | 777.3521 |
| beta globin | NP_000509.1 | LHVDPENFR | 86 | 563.7856 | 876.4205 |
| beta globin | NP_000509.1 | LHVDPENFR | 86 | 563.7856 | 1013.479 |
| pregnancy-zone protein | NP_002855.1 | EEGTDLEVTANR | 87 | 667.3151 | 360.1985 |
| pregnancy-zone protein | NP_002855.1 | EEGTDLEVTANR | 87 | 667.3151 | 461.2461 |
| pregnancy-zone protein | NP_002855.1 | EEGTDLEVTANR | 87 | 667.3151 | 560.3145 |
| pregnancy-zone protein | NP_002855.1 | EEGTDLEVTANR | 87 | 667.3151 | 689.3571 |
| pregnancy-zone protein | NP_002855.1 | EEGTDLEVTANR | 87 | 667.3151 | 802.4412 |
| pregnancy-zone protein | NP_002855.1 | EEGTDLEVTANR | 87 | 667.3151 | 917.4681 |
| pregnancy-zone protein | NP_002855.1 | EEGTDLEVTANR | 87 | 667.3151 | 1018.516 |
| pregnancy-zone protein | NP_002855.1 | EEGTDLEVTANR | 87 | 667.3151 | 1075.537 |
| pregnancy-zone protein | NP_002855.1 | EEGTDLEVTANR | 87 | 667.3151 | 1204.58 |
| apolipoprotein E precursor | NP_000032.1 | LGPLVEQGR | 88 | 484.7798 | 360.1985 |
| apolipoprotein E precursor | NP_000032.1 | LGPLVEQGR | 88 | 484.7798 | 489.241 |
| apolipoprotein E precursor | NP_000032.1 | LGPLVEQGR | 88 | 484.7798 | 588.3094 |
| apolipoprotein E precursor | NP_000032.1 | LGPLVEQGR | 88 | 484.7798 | 701.3935 |
| apolipoprotein E precursor | NP_000032.1 | LGPLVEQGR | 88 | 484.7798 | 798.4463 |
| apolipoprotein E precursor | NP_000032.1 | LGPLVEQGR | 88 | 484.7798 | 855.4677 |
| serum amyloid P component precursor | NP_001630.1 | QGYFVEAQPK | 89 | 583.7957 | 372.2236 |
| serum amyloid P component precursor | NP_001630.1 | QGYFVEAQPK | 89 | 583.7957 | 443.2607 |
| serum amyloid P component precursor | NP_001630.1 | QGYFVEAQPK | 89 | 583.7957 | 572.3033 |
| serum amyloid P component precursor | NP_001630.1 | QGYFVEAQPK | 89 | 583.7957 | 671.3717 |
| serum amyloid P component precursor | NP_001630.1 | QGYFVEAQPK | 89 | 583.7957 | 818.4401 |
| serum amyloid P component precursor | NP_001630.1 | QGYFVEAQPK | 89 | 583.7957 | 981.5035 |
| serum amyloid P component precursor | NP_001630.1 | QGYFVEAQPK | 89 | 583.7957 | 1038.525 |
| apolipoprotein C-I precursor | NP_001636.1 | EFGNTLEDK | 90 | 526.7484 | 391.1818 |
| apolipoprotein C-I precursor | NP_001636.1 | EFGNTLEDK | 90 | 526.7484 | 504.2659 |
| apolipoprotein C-I precursor | NP_001636.1 | EFGNTLEDK | 90 | 526.7484 | 605.3135 |
| apolipoprotein C-I precursor | NP_001636.1 | EFGNTLEDK | 90 | 526.7484 | 719.3564 |
| apolipoprotein C-I precursor | NP_001636.1 | EFGNTLEDK | 90 | 526.7484 | 776.3779 |
| apolipoprotein C-I precursor | NP_001636.1 | EFGNTLEDK | 90 | 526.7484 | 923.4464 |
| apolipoprotein A-I preproprotein | NP_000030.1 | AKPALEDLR | 91 | 506.7929 | 403.2294 |
| apolipoprotein A-I preproprotein | NP_000030.1 | AKPALEDLR | 91 | 506.7929 | 532.272 |
| apolipoprotein A-I preproprotein | NP_000030.1 | AKPALEDLR | 91 | 506.7929 | 645.3561 |
| apolipoprotein A-I preproprotein | NP_000030.1 | AKPALEDLR | 91 | 506.7929 | 716.3932 |
| apolipoprotein A-I preproprotein | NP_000030.1 | AKPALEDLR | 91 | 506.7929 | 813.4459 |
| apolipoprotein A-I preproprotein | NP_000030.1 | AKPALEDLR | 91 | 506.7929 | 941.5409 |
| apolipoprotein C-III precursor | NP_000031.1 | DALSSVQESQVAQQAR | 92 | 858.9292 | 374.2141 |
| apolipoprotein C-III precursor | NP_000031.1 | DALSSVQESQVAQQAR | 92 | 858.9292 | 502.2727 |
| apolipoprotein C-III precursor | NP_000031.1 | DALSSVQESQVAQQAR | 92 | 858.9292 | 573.3098 |
| apolipoprotein C-III precursor | NP_000031.1 | DALSSVQESQVAQQAR | 92 | 858.9292 | 672.3782 |
| apolipoprotein C-III precursor | NP_000031.1 | DALSSVQESQVAQQAR | 92 | 858.9292 | 800.4368 |
| apolipoprotein C-III precursor | NP_000031.1 | DALSSVQESQVAQQAR | 92 | 858.9292 | 887.4688 |
| apolipoprotein C-III precursor | NP_000031.1 | DALSSVQESQVAQQAR | 92 | 858.9292 | 1016.511 |
| apolipoprotein C-III precursor | NP_000031.1 | DALSSVQESQVAQQAR | 92 | 858.9292 | 1144.57 |
| apolipoprotein C-III precursor | NP_000031.1 | DALSSVQESQVAQQAR | 92 | 858.9292 | 1243.638 |
| apolipoprotein C-III precursor | NP_000031.1 | DALSSVQESQVAQQAR | 92 | 858.9292 | 1330.67 |
| apolipoprotein C-III precursor | NP_000031.1 | DALSSVQESQVAQQAR | 92 | 858.9292 | 1417.702 |
| apolipoprotein B precursor | NP_000375.2 | LDVTTSIGR | 93 | 481.2693 | 345.2239 |
| apolipoprotein B precursor | NP_000375.2 | LDVTTSIGR | 93 | 481.2693 | 432.256 |
| apolipoprotein B precursor | NP_000375.2 | LDVTTSIGR | 93 | 481.2693 | 533.3036 |
| apolipoprotein B precursor | NP_000375.2 | LDVTTSIGR | 93 | 481.2693 | 634.3513 |
| apolipoprotein B precursor | NP_000375.2 | LDVTTSIGR | 93 | 481.2693 | 733.4197 |
| apolipoprotein B precursor | NP_000375.2 | LDVTTSIGR | 93 | 481.2693 | 848.4467 |
| alpha-2-macroglobulin precursor | NP_000005.2 | AAQVTIQSSGTFSSK | 94 | 756.3887 | 321.1763 |
| alpha-2-macroglobulin precursor | NP_000005.2 | AAQVTIQSSGTFSSK | 94 | 756.3887 | 468.2447 |
| alpha-2-macroglobulin precursor | NP_000005.2 | AAQVTIQSSGTFSSK | 94 | 756.3887 | 569.2924 |
| alpha-2-macroglobulin precursor | NP_000005.2 | AAQVTIQSSGTFSSK | 94 | 756.3887 | 626.3138 |
| alpha-2-macroglobulin precursor | NP_000005.2 | AAQVTIQSSGTFSSK | 94 | 756.3887 | 713.3459 |
| alpha-2-macroglobulin precursor | NP_000005.2 | AAQVTIQSSGTFSSK | 94 | 756.3887 | 800.3779 |
| alpha-2-macroglobulin precursor | NP_000005.2 | AAQVTIQSSGTFSSK | 94 | 756.3887 | 928.4365 |
| alpha-2-macroglobulin precursor | NP_000005.2 | AAQVTIQSSGTFSSK | 94 | 756.3887 | 1041.521 |
| alpha-2-macroglobulin precursor | NP_000005.2 | AAQVTIQSSGTFSSK | 94 | 756.3887 | 1142.568 |
| alpha-2-macroglobulin precursor | NP_000005.2 | AAQVTIQSSGTFSSK | 94 | 756.3887 | 1241.637 |
| alpha-2-macroglobulin precursor | NP_000005.2 | AAQVTIQSSGTFSSK | 94 | 756.3887 | 1369.695 |
| alpha-2-macroglobulin precursor | NP_000005.2 | AAQVTIQSSGTFSSK | 94 | 756.3887 | 1440.732 |
| apolipoprotein B precursor | NP_000375.2 | VHELIER | 95 | 448.2534 | 417.2451 |
| apolipoprotein B precursor | NP_000375.2 | VHELIER | 95 | 448.2534 | 530.3291 |
| apolipoprotein B precursor | NP_000375.2 | VHELIER | 95 | 448.2534 | 659.3717 |
| apolipoprotein B precursor | NP_000375.2 | VHELIER | 95 | 448.2534 | 796.4306 |
| apolipoprotein B precursor | NP_000375.2 | FLDSNIK | 96 | 418.7292 | 374.2393 |
| apolipoprotein B precursor | NP_000375.2 | FLDSNIK | 96 | 418.7292 | 461.2713 |
| apolipoprotein B precursor | NP_000375.2 | FLDSNIK | 96 | 418.7292 | 576.2982 |
| apolipoprotein B precursor | NP_000375.2 | FLDSNIK | 96 | 418.7292 | 689.3823 |
| apolipoprotein B precursor | NP_000375.2 | GVISIPR | 97 | 371.2345 | 385.2552 |
| apolipoprotein B precursor | NP_000375.2 | GVISIPR | 97 | 371.2345 | 472.2873 |
| apolipoprotein B precursor | NP_000375.2 | GVISIPR | 97 | 371.2345 | 585.3713 |
| apolipoprotein B precursor | NP_000375.2 | GVISIPR | 97 | 371.2345 | 684.4398 |
| serum amyloid A4; constitutive | NP_006503.1 | EALQGVGDMGR | 98 | 566.7744 | 363.1804 |
| serum amyloid A4; constitutive | NP_006503.1 | EALQGVGDMGR | 98 | 566.7744 | 478.2073 |
| serum amyloid A4; constitutive | NP_006503.1 | EALQGVGDMGR | 98 | 566.7744 | 535.2288 |
| serum amyloid A4; constitutive | NP_006503.1 | EALQGVGDMGR | 98 | 566.7744 | 691.3187 |
| serum amyloid A4; constitutive | NP_006503.1 | EALQGVGDMGR | 98 | 566.7744 | 819.3772 |
| serum amyloid A4; constitutive | NP_006503.1 | EALQGVGDMGR | 98 | 566.7744 | 932.4613 |
| serum amyloid A4; constitutive | NP_006503.1 | EALQGVGDMGR | 98 | 566.7744 | 1003.498 |
| serum amyloid A4; constitutive | NP_006503.1 | EALQGVGDMGR | 98 | 574.7719 | 379.1753 |
| serum amyloid A4; constitutive | NP_006503.1 | EALQGVGDMGR | 98 | 574.7719 | 494.2022 |
| serum amyloid A4; constitutive | NP_006503.1 | EALQGVGDMGR | 98 | 574.7719 | 551.2237 |
| serum amyloid A4; constitutive | NP_006503.1 | EALQGVGDMGR | 98 | 574.7719 | 650.2921 |
| serum amyloid A4; constitutive | NP_006503.1 | EALQGVGDMGR | 98 | 574.7719 | 707.3135 |
| serum amyloid A4; constitutive | NP_006503.1 | EALQGVGDMGR | 98 | 574.7719 | 835.3721 |
| serum amyloid A4; constitutive | NP_006503.1 | EALQGVGDMGR | 98 | 574.7719 | 948.4562 |
| serum amyloid A4; constitutive | NP_006503.1 | EALQGVGDMGR | 98 | 574.7719 | 1019.493 |
| beta globin | NP_000509.1 | VNVDEVGGEALGR | 99 | 657.8361 | 345.2239 |
| beta globin | NP_000509.1 | VNVDEVGGEALGR | 99 | 657.8361 | 416.261 |
| beta globin | NP_000509.1 | VNVDEVGGEALGR | 99 | 657.8361 | 545.3036 |
| beta globin | NP_000509.1 | VNVDEVGGEALGR | 99 | 657.8361 | 602.3251 |
| beta globin | NP_000509.1 | VNVDEVGGEALGR | 99 | 657.8361 | 659.3466 |
| beta globin | NP_000509.1 | VNVDEVGGEALGR | 99 | 657.8361 | 758.415 |
| beta globin | NP_000509.1 | VNVDEVGGEALGR | 99 | 657.8361 | 887.4576 |
| beta globin | NP_000509.1 | VNVDEVGGEALGR | 99 | 657.8361 | 1002.484 |
| beta globin | NP_000509.1 | VNVDEVGGEALGR | 99 | 657.8361 | 1101.553 |
| beta globin | NP_000509.1 | VNVDEVGGEALGR | 99 | 657.8361 | 1215.596 |
| apolipoprotein B precursor | NP_000375.2 | FIIPSPK | 100 | 401.2471 | 331.1971 |
| apolipoprotein B precursor | NP_000375.2 | FIIPSPK | 100 | 401.2471 | 428.2498 |
| apolipoprotein B precursor | NP_000375.2 | FIIPSPK | 100 | 401.2471 | 541.3339 |
| apolipoprotein B precursor | NP_000375.2 | FIIPSPK | 100 | 401.2471 | 654.418 |
| apolipoprotein C-II precursor | NP_000474.2 | TYLPAVDEK | 101 | 518.2715 | 391.1818 |
| apolipoprotein C-II precursor | NP_000474.2 | TYLPAVDEK | 101 | 518.2715 | 490.2502 |
| apolipoprotein C-II precursor | NP_000474.2 | TYLPAVDEK | 101 | 518.2715 | 561.2873 |
| apolipoprotein C-II precursor | NP_000474.2 | TYLPAVDEK | 101 | 518.2715 | 658.3401 |
| apolipoprotein C-II precursor | NP_000474.2 | TYLPAVDEK | 101 | 518.2715 | 771.4241 |
| apolipoprotein C-II precursor | NP_000474.2 | TYLPAVDEK | 101 | 518.2715 | 934.4875 |
| apolipoprotein B precursor | NP_000375.2 | LTISEQNIQR | 102 | 601.3304 | 416.261 |
| apolipoprotein B precursor | NP_000375.2 | LTISEQNIQR | 102 | 601.3304 | 530.304 |
| apolipoprotein B precursor | NP_000375.2 | LTISEQNIQR | 102 | 601.3304 | 658.3625 |
| apolipoprotein B precursor | NP_000375.2 | LTISEQNIQR | 102 | 601.3304 | 787.4052 |
| apolipoprotein B precursor | NP_000375.2 | LTISEQNIQR | 102 | 601.3304 | 874.4372 |
| apolipoprotein B precursor | NP_000375.2 | LTISEQNIQR | 102 | 601.3304 | 987.5212 |
| apolipoprotein B precursor | NP_000375.2 | LTISEQNIQR | 102 | 601.3304 | 1088.569 |
| apolipoprotein B precursor | NP_000375.2 | ALVDTLK | 103 | 380.2342 | 361.244 |
| apolipoprotein B precursor | NP_000375.2 | ALVDTLK | 103 | 380.2342 | 476.2709 |
| apolipoprotein B precursor | NP_000375.2 | ALVDTLK | 103 | 380.2342 | 575.3394 |
| apolipoprotein B precursor | NP_000375.2 | ALVDTLK | 103 | 380.2342 | 688.4234 |
| apolipoprotein B precursor | NP_000375.2 | INNQLTLDSNTK | 104 | 680.857 | 362.2029 |
| apolipoprotein B precursor | NP_000375.2 | INNQLTLDSNTK | 104 | 680.857 | 449.2349 |
| apolipoprotein B precursor | NP_000375.2 | INNQLTLDSNTK | 104 | 680.857 | 564.2618 |
| apolipoprotein B precursor | NP_000375.2 | INNQLTLDSNTK | 104 | 680.857 | 677.3459 |
| apolipoprotein B precursor | NP_000375.2 | INNQLTLDSNTK | 104 | 680.857 | 778.3936 |
| apolipoprotein B precursor | NP_000375.2 | INNQLTLDSNTK | 104 | 680.857 | 891.4777 |
| apolipoprotein B precursor | NP_000375.2 | INNQLTLDSNTK | 104 | 680.857 | 1019.536 |
| apolipoprotein B precursor | NP_000375.2 | INNQLTLDSNTK | 104 | 680.857 | 1133.579 |
| apolipoprotein B precursor | NP_000375.2 | INNQLTLDSNTK | 104 | 680.857 | 1247.622 |
| apolipoprotein A-IV precursor | NP_000473.2 | LEPYADQLR | 105 | 552.7878 | 416.261 |
| apolipoprotein A-IV precursor | NP_000473.2 | LEPYADQLR | 105 | 552.7878 | 531.288 |
| apolipoprotein A-IV precursor | NP_000473.2 | LEPYADQLR | 105 | 552.7878 | 602.3251 |
| apolipoprotein A-IV precursor | NP_000473.2 | LEPYADQLR | 105 | 552.7878 | 765.3884 |
| apolipoprotein A-IV precursor | NP_000473.2 | LEPYADQLR | 105 | 552.7878 | 862.4412 |
| apolipoprotein A-IV precursor | NP_000473.2 | LEPYADQLR | 105 | 552.7878 | 991.4838 |
| beta globin | NP_000509.1 | VVAGVANALAHK | 106 | 575.3406 | 355.2083 |
| beta globin | NP_000509.1 | VVAGVANALAHK | 106 | 575.3406 | 468.2924 |
| beta globin | NP_000509.1 | VVAGVANALAHK | 106 | 575.3406 | 539.3295 |
| beta globin | NP_000509.1 | VVAGVANALAHK | 106 | 575.3406 | 653.3724 |
| beta globin | NP_000509.1 | VVAGVANALAHK | 106 | 575.3406 | 724.4095 |
| beta globin | NP_000509.1 | VVAGVANALAHK | 106 | 575.3406 | 823.4779 |
| beta globin | NP_000509.1 | VVAGVANALAHK | 106 | 575.3406 | 880.4994 |
| beta globin | NP_000509.1 | VVAGVANALAHK | 106 | 575.3406 | 951.5365 |
| beta globin | NP_000509.1 | VVAGVANALAHK | 106 | 575.3406 | 1050.605 |
| ceruloplasmin precursor | NP_000087.1 | EVGPTNADPVC[Carboxymethyl]L | 107 | 736.3585 | 331.2334 |
| | | AK | | | |
| ceruloplasmin precursor | NP_000087.1 | EVGPTNADPVC[Carboxymethyl]L | 107 | 736.3585 | 492.2481 |
| | | AK | | | |
| ceruloplasmin precursor | NP_000087.1 | EVGPTNADPVC[Carboxymethyl]L | 107 | 736.3585 | 591.3165 |
| | | AK | | | |
| ceruloplasmin precursor | NP_000087.1 | EVGPTNADPVC[Carboxymethyl]L | 107 | 736.3585 | 688.3693 |
| | | AK | | | |
| ceruloplasmin precursor | NP_000087.1 | EVGPTNADPVC[Carboxymethyl]L | 107 | 736.3585 | 803.3962 |
| | | AK | | | |
| ceruloplasmin precursor | NP_000087.1 | EVGPTNADPVC[Carboxymethyl]L | 107 | 736.3585 | 874.4333 |
| | | AK | | | |
| ceruloplasmin precursor | NP_000087.1 | EVGPTNADPVC[Carboxymethyl]L | 107 | 736.3585 | 988.4763 |
| | | AK | | | |
| ceruloplasmin precursor | NP_000087.1 | EVGPTNADPVC[Carboxymethyl]L | 107 | 736.3585 | 1089.524 |
| | | AK | | | |
| ceruloplasmin precursor | NP_000087.1 | EVGPTNADPVC[Carboxymethyl]L | 107 | 736.3585 | 1186.577 |
| | | AK | | | |
| ceruloplasmin precursor | NP_000087.1 | EVGPTNADPVC[Carboxymethyl]L | 107 | 736.3585 | 1243.598 |
| | | AK | | | |
| ceruloplasmin precursor | NP_000087.1 | EVGPTNADPVC[Carboxymethyl]L | 107 | 736.3585 | 1342.667 |
| | | AK | | | |
| apolipoprotein B precursor | NP_000375.2 | QHIEAIDVR | 108 | 540.7935 | 389.2137 |
| apolipoprotein B precursor | NP_000375.2 | QHIEAIDVR | 108 | 540.7935 | 502.2978 |
| apolipoprotein B precursor | NP_000375.2 | QHIEAIDVR | 108 | 540.7935 | 573.335 |
| apolipoprotein B precursor | NP_000375.2 | QHIEAIDVR | 108 | 540.7935 | 702.3775 |
| apolipoprotein B precursor | NP_000375.2 | QHIEAIDVR | 108 | 540.7935 | 815.4616 |
| apolipoprotein B precursor | NP_000375.2 | QHIEAIDVR | 108 | 540.7935 | 952.5205 |
| apolipoprotein C-III precursor | NP_000031.1 | DYWSTVK | 109 | 449.7189 | 347.2283 |
| apolipoprotein C-III precursor | NP_000031.1 | DYWSTVK | 109 | 449.7189 | 434.2604 |
| apolipoprotein C-III precursor | NP_000031.1 | DYWSTVK | 109 | 449.7189 | 620.3397 |
| apolipoprotein C-III precursor | NP_000031.1 | DYWSTVK | 109 | 449.7189 | 783.403 |
| apolipoprotein B precursor | NP_000375.2 | IHSGSFQSQVELSNDQEK | 110 | 1016.982 | 404.2134 |
| apolipoprotein B precursor | NP_000375.2 | IHSGSFQSQVELSNDQEK | 110 | 1016.982 | 519.2404 |
| apolipoprotein B precursor | NP_000375.2 | IHSGSFQSQVELSNDQEK | 110 | 1016.982 | 633.2833 |
| apolipoprotein B precursor | NP_000375.2 | IHSGSFQSQVELSNDQEK | 110 | 1016.982 | 720.3153 |
| apolipoprotein B precursor | NP_000375.2 | IHSGSFQSQVELSNDQEK | 110 | 1016.982 | 833.3994 |
| apolipoprotein B precursor | NP_000375.2 | IHSGSFQSQVELSNDQEK | 110 | 1016.982 | 962.442 |
| apolipoprotein B precursor | NP_000375.2 | IHSGSFQSQVELSNDQEK | 110 | 1016.982 | 1061.51 |
| apolipoprotein B precursor | NP_000375.2 | IHSGSFQSQVELSNDQEK | 110 | 1016.982 | 1189.569 |
| apolipoprotein B precursor | NP_000375.2 | IHSGSFQSQVELSNDQEK | 110 | 1016.982 | 1276.601 |
| apolipoprotein B precursor | NP_000375.2 | IHSGSFQSQVELSNDQEK | 110 | 1016.982 | 1404.66 |
| apolipoprotein B precursor | NP_000375.2 | LVELAHQYK | 111 | 550.8086 | 438.2342 |
| apolipoprotein B precursor | NP_000375.2 | LVELAHQYK | 111 | 550.8086 | 575.2931 |
| apolipoprotein B precursor | NP_000375.2 | LVELAHQYK | 111 | 550.8086 | 646.3302 |
| apolipoprotein B precursor | NP_000375.2 | LVELAHQYK | 111 | 550.8086 | 759.4142 |
| apolipoprotein B precursor | NP_000375.2 | LVELAHQYK | 111 | 550.8086 | 888.4568 |
| apolipoprotein B precursor | NP_000375.2 | LVELAHQYK | 111 | 550.8086 | 987.5253 |
| apolipoprotein A-IV precursor | NP_000473.2 | LAPLAEDVR | 112 | 492.2797 | 389.2137 |
| apolipoprotein A-IV precursor | NP_000473.2 | LAPLAEDVR | 112 | 492.2797 | 518.2563 |
| apolipoprotein A-IV precursor | NP_000473.2 | LAPLAEDVR | 112 | 492.2797 | 589.2935 |
| apolipoprotein A-IV precursor | NP_000473.2 | LAPLAEDVR | 112 | 492.2797 | 702.3775 |
| apolipoprotein A-IV precursor | NP_000473.2 | LAPLAEDVR | 112 | 492.2797 | 799.4303 |
| apolipoprotein A-IV precursor | NP_000473.2 | LAPLAEDVR | 112 | 492.2797 | 870.4674 |
| apolipoprotein B precursor | NP_000375.2 | TPALHFK | 113 | 407.2345 | 431.2396 |
| apolipoprotein B precursor | NP_000375.2 | TPALHFK | 113 | 407.2345 | 544.3237 |
| apolipoprotein B precursor | NP_000375.2 | TPALHFK | 113 | 407.2345 | 615.3608 |
| apolipoprotein B precursor | NP_000375.2 | TPALHFK | 113 | 407.2345 | 712.4135 |
| apolipoprotein B precursor | NP_000375.2 | FVEGSHNSTVSLTTK | 114 | 803.9072 | 349.2076 |
| apolipoprotein B precursor | NP_000375.2 | FVEGSHNSTVSLTTK | 114 | 803.9072 | 462.2917 |
| apolipoprotein B precursor | NP_000375.2 | FVEGSHNSTVSLTTK | 114 | 803.9072 | 549.3237 |
| apolipoprotein B precursor | NP_000375.2 | FVEGSHNSTVSLTTK | 114 | 803.9072 | 648.3921 |
| apolipoprotein B precursor | NP_000375.2 | FVEGSHNSTVSLTTK | 114 | 803.9072 | 749.4398 |
| apolipoprotein B precursor | NP_000375.2 | FVEGSHNSTVSLTTK | 114 | 803.9072 | 836.4718 |
| apolipoprotein B precursor | NP_000375.2 | FVEGSHNSTVSLTTK | 114 | 803.9072 | 950.5148 |
| apolipoprotein B precursor | NP_000375.2 | FVEGSHNSTVSLTTK | 114 | 803.9072 | 1087.574 |
| apolipoprotein B precursor | NP_000375.2 | FVEGSHNSTVSLTTK | 114 | 803.9072 | 1174.606 |
| apolipoprotein B precursor | NP_000375.2 | FVEGSHNSTVSLTTK | 114 | 803.9072 | 1231.627 |
| apolipoprotein B precursor | NP_000375.2 | FVEGSHNSTVSLTTK | 114 | 803.9072 | 1360.67 |
| apolipoprotein B precursor | NP_000375.2 | FVEGSHNSTVSLTTK | 114 | 803.9072 | 1459.738 |
| apolipoprotein B precursor | NP_000375.2 | TFIEDVNK | 115 | 483.2505 | 360.2236 |
| apolipoprotein B precursor | NP_000375.2 | TFIEDVNK | 115 | 483.2505 | 475.2505 |
| apolipoprotein B precursor | NP_000375.2 | TFIEDVNK | 115 | 483.2505 | 604.2932 |
| apolipoprotein B precursor | NP_000375.2 | TFIEDVNK | 115 | 483.2505 | 717.3772 |
| apolipoprotein B precursor | NP_000375.2 | TFIEDVNK | 115 | 483.2505 | 864.4456 |
| apolipoprotein B precursor | NP_000005.2 | AIGYLNTGYQR | 116 | 628.3251 | 466.2403 |
| alpha-2-macroglobulin precursor | NP_000005.2 | AIGYLNTGYQR | 116 | 628.3251 | 523.2618 |
| alpha-2-macroglobulin precursor | NP_000005.2 | AIGYLNTGYQR | 116 | 628.3251 | 624.3094 |
| alpha-2-macroglobulin precursor | NP_000005.2 | AIGYLNTGYQR | 116 | 628.3251 | 738.3524 |
| alpha-2-macroglobulin precursor | NP_000005.2 | AIGYLNTGYQR | 116 | 628.3251 | 851.4365 |
| alpha-2-macroglobulin precursor | NP_000005.2 | AIGYLNTGYQR | 116 | 628.3251 | 1014.5 |
| alpha-2-macroglobulin precursor | NP_000005.2 | AIGYLNTGYQR | 116 | 628.3251 | 1071.521 |
| alpha-2-macroglobulin precursor | NP_000005.2 | AIGYLNTGYQR | 116 | 628.3251 | 1184.605 |
| apolipoprotein A-IV precursor | NP_000473.2 | LTPYADEFK | 117 | 542.2715 | 423.2233 |
| apolipoprotein A-IV precursor | NP_000473.2 | LTPYADEFK | 117 | 542.2715 | 538.2502 |
| apolipoprotein A-IV precursor | NP_000473.2 | LTPYADEFK | 117 | 542.2715 | 609.2873 |
| apolipoprotein A-IV precursor | NP_000473.2 | LTPYADEFK | 117 | 542.2715 | 772.3506 |
| apolipoprotein A-IV precursor | NP_000473.2 | LTPYADEFK | 117 | 542.2715 | 869.4034 |
| apolipoprotein A-IV precursor | NP_000473.2 | LTPYADEFK | 117 | 542.2715 | 970.4511 |
| paraoxonase 1 | NP_000437.3 | IQNILTEEPK | 118 | 592.8297 | 373.2076 |
| paraoxonase 1 | NP_000437.3 | IQNILTEEPK | 118 | 592.8297 | 502.2502 |
| paraoxonase 1 | NP_000437.3 | IQNILTEEPK | 118 | 592.8297 | 603.2979 |
| paraoxonase 1 | NP_000437.3 | IQNILTEEPK | 118 | 592.8297 | 716.382 |
| paraoxonase 1 | NP_000437.3 | IQNILTEEPK | 118 | 592.8297 | 829.466 |
| paraoxonase 1 | NP_000437.3 | IQNILTEEPK | 118 | 592.8297 | 943.5089 |
| paraoxonase 1 | NP_000437.3 | IQNILTEEPK | 118 | 592.8297 | 1071.568 |
| beta globin | NP_000509.1 | EFTPPVQAAYQK | 119 | 689.8537 | 438.2342 |
| beta globin | NP_000509.1 | EFTPPVQAAYQK | 119 | 689.8537 | 509.2713 |
| beta globin | NP_000509.1 | EFTPPVQAAYQK | 119 | 689.8537 | 580.3084 |
| beta globin | NP_000509.1 | EFTPPVQAAYQK | 119 | 689.8537 | 708.3669 |
| beta globin | NP_000509.1 | EFTPPVQAAYQK | 119 | 689.8537 | 807.4354 |
| beta globin | NP_000509.1 | EFTPPVQAAYQK | 119 | 689.8537 | 904.4882 |
| beta globin | NP_000509.1 | EFTPPVQAAYQK | 119 | 689.8537 | 1001.541 |
| beta globin | NP_000509.1 | EFTPPVQAAYQK | 119 | 689.8537 | 1102.589 |
| beta globin | NP_000509.1 | EFTPPVQAAYQK | 119 | 689.8537 | 1249.657 |
| histidine-rich glycoprotein precursor | NP_000403.1 | ALDLINKR | 120 | 471.7902 | 417.2563 |
| histidine-rich glycoprotein precursor | NP_000403.1 | ALDLINKR | 120 | 471.7902 | 530.3403 |
| histidine-rich glycoprotein precursor | NP_000403.1 | ALDLINKR | 120 | 471.7902 | 643.4244 |
| histidine-rich glycoprotein precursor | NP_000403.1 | ALDLINKR | 120 | 471.7902 | 758.4514 |
| histidine-rich glycoprotein precursor | NP_000403.1 | ALDLINKR | 120 | 471.7902 | 871.5354 |
| apolipoprotein B precursor | NP_000375.2 | QSFDLSVK | 121 | 462.2453 | 333.2127 |
| apolipoprotein B precursor | NP_000375.2 | QSFDLSVK | 121 | 462.2453 | 446.2968 |
| apolipoprotein B precursor | NP_000375.2 | QSFDLSVK | 121 | 462.2453 | 561.3237 |
| apolipoprotein B precursor | NP_000375.2 | QSFDLSVK | 121 | 462.2453 | 708.3921 |
| apolipoprotein B precursor | NP_000375.2 | QSFDLSVK | 121 | 462.2453 | 795.4241 |
| ceruloplasmin precursor | NP_000087.1 | DDEEFIESNK | 122 | 613.2646 | 348.1872 |
| ceruloplasmin precursor | NP_000087.1 | DDEEFIESNK | 122 | 613.2646 | 477.2298 |
| ceruloplasmin precursor | NP_000087.1 | DDEEFIESNK | 122 | 613.2646 | 590.3138 |
| ceruloplasmin precursor | NP_000087.1 | DDEEFIESNK | 122 | 613.2646 | 737.3823 |
| ceruloplasmin precursor | NP_000087.1 | DDEEFIESNK | 122 | 613.2646 | 866.4249 |
| ceruloplasmin precursor | NP_000087.1 | DDEEFIESNK | 122 | 613.2646 | 995.4675 |
| ceruloplasmin precursor | NP_000087.1 | DDEEFIESNK | 122 | 613.2646 | 1110.494 |
| apolipoprotein B precursor | NP_000375.2 | LSNVLQQVK | 123 | 514.8086 | 374.2393 |
| apolipoprotein B precursor | NP_000375.2 | LSNVLQQVK | 123 | 514.8086 | 502.2978 |
| apolipoprotein B precursor | NP_000375.2 | LSNVLQQVK | 123 | 514.8086 | 615.3819 |
| apolipoprotein B precursor | NP_000375.2 | LSNVLQQVK | 123 | 514.8086 | 714.4503 |
| apolipoprotein B precursor | NP_000375.2 | LSNVLQQVK | 123 | 514.8086 | 828.4932 |
| apolipoprotein B precursor | NP_000375.2 | LSNVLQQVK | 123 | 514.8086 | 915.5253 |
| alpha-2-macroglobulin precursor | NP_000005.2 | GEAFTLK | 124 | 383.2107 | 361.244 |
| alpha-2-macroglobulin precursor | NP_000005.2 | GEAFTLK | 124 | 383.2107 | 508.3124 |
| alpha-2-macroglobulin precursor | NP_000005.2 | GEAFTLK | 124 | 383.2107 | 579.3495 |
| alpha-2-macroglobulin precursor | NP_000005.2 | GEAFTLK | 124 | 383.2107 | 708.3921 |
| apolipoprotein B precursor | NP_000375.2 | LFLEETK | 125 | 440.2448 | 377.2025 |
| apolipoprotein B precursor | NP_000375.2 | LFLEETK | 125 | 440.2448 | 506.2451 |
| apolipoprotein B precursor | NP_000375.2 | LFLEETK | 125 | 440.2448 | 619.3292 |
| apolipoprotein B precursor | NP_000375.2 | LFLEETK | 125 | 440.2448 | 766.3976 |
| apolipoprotein B precursor | NP_000375.2 | YEGLQEWEGK | 126 | 619.788 | 333.1763 |
| apolipoprotein B precursor | NP_000375.2 | YEGLQEWEGK | 126 | 619.788 | 519.2556 |
| apolipoprotein B precursor | NP_000375.2 | YEGLQEWEGK | 126 | 619.788 | 648.2982 |
| apolipoprotein B precursor | NP_000375.2 | YEGLQEWEGK | 126 | 619.788 | 776.3568 |
| apolipoprotein B precursor | NP_000375.2 | YEGLQEWEGK | 126 | 619.788 | 889.4409 |
| apolipoprotein B precursor | NP_000375.2 | YEGLQEWEGK | 126 | 619.788 | 946.4623 |
| apolipoprotein B precursor | NP_000375.2 | YEGLQEWEGK | 126 | 619.788 | 1075.505 |
| apolipoprotein B precursor | NP_000375.2 | INDVLEHVK | 127 | 533.7982 | 383.2396 |
| apolipoprotein B precursor | NP_000375.2 | INDVLEHVK | 127 | 533.7982 | 512.2822 |
| apolipoprotein B precursor | NP_000375.2 | INDVLEHVK | 127 | 533.7982 | 625.3662 |
| apolipoprotein B precursor | NP_000375.2 | INDVLEHVK | 127 | 533.7982 | 724.4346 |
| apolipoprotein B precursor | NP_000375.2 | INDVLEHVK | 127 | 533.7982 | 839.4616 |
| apolipoprotein B precursor | NP_000375.2 | INDVLEHVK | 127 | 533.7982 | 953.5045 |
| apolipoprotein B precursor | NP_000375.2 | IISDYHQQFR | 128 | 653.8306 | 450.2454 |
| apolipoprotein B precursor | NP_000375.2 | IISDYHQQFR | 128 | 653.8306 | 578.304 |
| apolipoprotein B precursor | NP_000375.2 | IISDYHQQFR | 128 | 653.8306 | 715.3629 |
| apolipoprotein B precursor | NP_000375.2 | IISDYHQQFR | 128 | 653.8306 | 878.4262 |
| apolipoprotein B precursor | NP_000375.2 | IISDYHQQFR | 128 | 653.8306 | 993.4531 |
| apolipoprotein B precursor | NP_000375.2 | IISDYHQQFR | 128 | 653.8306 | 1080.485 |
| apolipoprotein B precursor | NP_000375.2 | IISDYHQQFR | 128 | 653.8306 | 1193.569 |
| apolipoprotein A-IV precursor | NP_000473.2 | LGEVNTYAGDLQK | 129 | 704.3594 | 388.2549 |
| apolipoprotein A-IV precursor | NP_000473.2 | LGEVNTYAGDLQK | 129 | 704.3594 | 503.2818 |
| apolipoprotein A-IV precursor | NP_000473.2 | LGEVNTYAGDLQK | 129 | 704.3594 | 560.3033 |
| apolipoprotein A-IV precursor | NP_000473.2 | LGEVNTYAGDLQK | 129 | 704.3594 | 631.3404 |
| apolipoprotein A-IV precursor | NP_000473.2 | LGEVNTYAGDLQK | 129 | 704.3594 | 794.4037 |
| apolipoprotein A-IV precursor | NP_000473.2 | LGEVNTYAGDLQK | 129 | 704.3594 | 895.4514 |
| apolipoprotein A-IV precursor | NP_000473.2 | LGEVNTYAGDLQK | 129 | 704.3594 | 1009.494 |
| apolipoprotein A-IV precursor | NP_000473.2 | LGEVNTYAGDLQK | 129 | 704.3594 | 1108.563 |
| apolipoprotein A-IV precursor | NP_000473.2 | LGEVNTYAGDLQK | 129 | 704.3594 | 1237.605 |
| apolipoprotein A-IV precursor | NP_000473.2 | LGEVNTYAGDLQK | 129 | 704.3594 | 1294.627 |
| alpha 1 globin | NP_000549.1 | LRVDPVNFK | 130 | 544.3166 | 408.2236 |
| alpha 1 globin | NP_000549.1 | LRVDPVNFK | 130 | 544.3166 | 507.292 |
| alpha 1 globin | NP_000549.1 | LRVDPVNFK | 130 | 544.3166 | 604.3448 |
| alpha 1 globin | NP_000549.1 | LRVDPVNFK | 130 | 544.3166 | 719.3717 |
| alpha 1 globin | NP_000549.1 | LRVDPVNFK | 130 | 544.3166 | 818.4401 |
| alpha 1 globin | NP_000549.1 | LRVDPVNFK | 130 | 544.3166 | 974.5413 |
| pregnancy-zone protein | NP_002855.1 | DLKPAIVK | 131 | 442.2842 | 359.2647 |
| pregnancy-zone protein | NP_002855.1 | DLKPAIVK | 131 | 442.2842 | 430.3018 |
| pregnancy-zone protein | NP_002855.1 | DLKPAIVK | 131 | 442.2842 | 527.3546 |
| pregnancy-zone protein | NP_002855.1 | DLKPAIVK | 131 | 442.2842 | 655.4496 |
| pregnancy-zone protein | NP_002855.1 | DLKPAIVK | 131 | 442.2842 | 768.5336 |
| apolipoprotein E precursor | NP_000032.1 | AATVGSLAGQPLQER | 132 | 749.4046 | 432.2196 |
| apolipoprotein E precursor | NP_000032.1 | AATVGSLAGQPLQER | 132 | 749.4046 | 545.3036 |
| apolipoprotein E precursor | NP_000032.1 | AATVGSLAGQPLQER | 132 | 749.4046 | 642.3564 |
| apolipoprotein E precursor | NP_000032.1 | AATVGSLAGQPLQER | 132 | 749.4046 | 770.415 |
| apolipoprotein E precursor | NP_000032.1 | AATVGSLAGQPLQER | 132 | 749.4046 | 827.4365 |
| apolipoprotein E precursor | NP_000032.1 | AATVGSLAGQPLQER | 132 | 749.4046 | 898.4736 |
| apolipoprotein E precursor | NP_000032.1 | AATVGSLAGQPLQER | 132 | 749.4046 | 1011.558 |
| apolipoprotein E precursor | NP_000032.1 | AATVGSLAGQPLQER | 132 | 749.4046 | 1098.59 |
| apolipoprotein E precursor | NP_000032.1 | AATVGSLAGQPLQER | 132 | 749.4046 | 1155.611 |
| apolipoprotein E precursor | NP_000032.1 | AATVGSLAGQPLQER | 132 | 749.4046 | 1254.68 |
| apolipoprotein E precursor | NP_000032.1 | AATVGSLAGQPLQER | 132 | 749.4046 | 1355.727 |
| apolipoprotein E precursor | NP_000032.1 | AATVGSLAGQPLQER | 132 | 749.4046 | 1426.764 |
| histidine-rich glycoprotein precursor | NP_000403.1 | ALDLINK | 133 | 393.7396 | 374.2393 |
| histidine-rich glycoprotein precursor | NP_000403.1 | ALDLINK | 133 | 393.7396 | 487.3233 |
| histidine-rich glycoprotein precursor | NP_000403.1 | ALDLINK | 133 | 393.7396 | 602.3503 |
| histidine-rich glycoprotein precursor | NP_000403.1 | ALDLINK | 133 | 393.7396 | 715.4343 |
| alpha-2-macroglobulin precursor | NP_000005.2 | FEVQVTVPK | 134 | 523.7977 | 343.2334 |
| alpha-2-macroglobulin precursor | NP_000005.2 | FEVQVTVPK | 134 | 523.7977 | 444.2811 |
| alpha-2-macroglobulin precursor | NP_000005.2 | FEVQVTVPK | 134 | 523.7977 | 543.3495 |
| alpha-2-macroglobulin precursor | NP_000005.2 | FEVQVTVPK | 134 | 523.7977 | 671.4081 |
| alpha-2-macroglobulin precursor | NP_000005.2 | FEVQVTVPK | 134 | 523.7977 | 770.4765 |
| alpha-2-macroglobulin precursor | NP_000005.2 | FEVQVTVPK | 134 | 523.7977 | 899.5191 |
| apolipoprotein B precursor | NP_000375.2 | SGSSTASWIQNVDTK | 135 | 790.8812 | 363.1869 |
| apolipoprotein B precursor | NP_000375.2 | SGSSTASWIQNVDTK | 135 | 790.8812 | 462.2553 |
| apolipoprotein B precursor | NP_000375.2 | SGSSTASWIQNVDTK | 135 | 790.8812 | 576.2982 |
| apolipoprotein B precursor | NP_000375.2 | SGSSTASWIQNVDTK | 135 | 790.8812 | 704.3568 |
| apolipoprotein B precursor | NP_000375.2 | SGSSTASWIQNVDTK | 135 | 790.8812 | 817.4409 |
| apolipoprotein B precursor | NP_000375.2 | SGSSTASWIQNVDTK | 135 | 790.8812 | 1003.52 |
| apolipoprotein B precursor | NP_000375.2 | SGSSTASWIQNVDTK | 135 | 790.8812 | 1090.552 |
| apolipoprotein B precursor | NP_000375.2 | SGSSTASWIQNVDTK | 135 | 790.8812 | 1161.589 |
| apolipoprotein B precursor | NP_000375.2 | SGSSTASWIQNVDTK | 135 | 790.8812 | 1262.637 |
| apolipoprotein B precursor | NP_000375.2 | SGSSTASWIQNVDTK | 135 | 790.8812 | 1349.669 |
| apolipoprotein B precursor | NP_000375.2 | SGSSTASWIQNVDTK | 135 | 790.8812 | 1436.701 |
| apolipoprotein B precursor | NP_000375.2 | SGSSTASWIQNVDTK | 135 | 790.8812 | 1493.723 |
| apolipoprotein C-I precursor | NP_001636.1 | LKEFGNTLEDK | 136 | 647.3379 | 391.1818 |
| apolipoprotein C-I precursor | NP_001636.1 | LKEFGNTLEDK | 136 | 647.3379 | 504.2659 |
| apolipoprotein C-I precursor | NP_001636.1 | LKEFGNTLEDK | 136 | 647.3379 | 605.3135 |
| apolipoprotein C-I precursor | NP_001636.1 | LKEFGNTLEDK | 136 | 647.3379 | 719.3564 |
| apolipoprotein C-I precursor | NP_001636.1 | LKEFGNTLEDK | 136 | 647.3379 | 776.3779 |
| apolipoprotein C-I precursor | NP_001636.1 | LKEFGNTLEDK | 136 | 647.3379 | 923.4464 |
| apolipoprotein C-I precursor | NP_001636.1 | LKEFGNTLEDK | 136 | 647.3379 | 1180.584 |
| apolipoprotein B precursor | NP_000375.2 | QGFFPDSVNK | 137 | 569.78 | 360.2236 |
| apolipoprotein B precursor | NP_000375.2 | QGFFPDSVNK | 137 | 569.78 | 447.2556 |
| apolipoprotein B precursor | NP_000375.2 | QGFFPDSVNK | 137 | 569.78 | 562.2826 |
| apolipoprotein B precursor | NP_000375.2 | QGFFPDSVNK | 137 | 569.78 | 659.3353 |
| apolipoprotein B precursor | NP_000375.2 | QGFFPDSVNK | 137 | 569.78 | 806.4037 |
| apolipoprotein B precursor | NP_000375.2 | QGFFPDSVNK | 137 | 569.78 | 953.4722 |
| apolipoprotein B precursor | NP_000375.2 | QGFFPDSVNK | 137 | 569.78 | 1010.494 |
| ceruloplasmin precursor | NP_000087.1 | QSEDSTFYLGER | 138 | 716.323 | 361.1825 |
| ceruloplasmin precursor | NP_000087.1 | QSEDSTFYLGER | 138 | 716.323 | 474.2665 |
| ceruloplasmin precursor | NP_000087.1 | QSEDSTFYLGER | 138 | 716.323 | 637.3298 |
| ceruloplasmin precursor | NP_000087.1 | QSEDSTFYLGER | 138 | 716.323 | 784.3983 |
| ceruloplasmin precursor | NP_000087.1 | QSEDSTFYLGER | 138 | 716.323 | 885.4459 |
| ceruloplasmin precursor | NP_000087.1 | QSEDSTFYLGER | 138 | 716.323 | 972.478 |
| ceruloplasmin precursor | NP_000087.1 | QSEDSTFYLGER | 138 | 716.323 | 1087.505 |
| ceruloplasmin precursor | NP_000087.1 | QSEDSTFYLGER | 138 | 716.323 | 1216.547 |
| ceruloplasmin precursor | NP_000087.1 | QSEDSTFYLGER | 138 | 716.323 | 1303.58 |
| apolipoprotein B precursor | NP_000375.2 | INPLALK | 139 | 384.7525 | 331.2334 |
| apolipoprotein B precursor | NP_000375.2 | INPLALK | 139 | 384.7525 | 444.3175 |
| apolipoprotein B precursor | NP_000375.2 | INPLALK | 139 | 384.7525 | 541.3702 |
| apolipoprotein B precursor | NP_000375.2 | INPLALK | 139 | 384.7525 | 655.4132 |
| apolipoprotein B precursor | NP_000375.2 | LNELSFK | 140 | 425.7371 | 381.2127 |
| apolipoprotein B precursor | NP_000375.2 | LNELSFK | 140 | 425.7371 | 494.2968 |
| apolipoprotein B precursor | NP_000375.2 | LNELSFK | 140 | 425.7371 | 623.3394 |
| apolipoprotein B precursor | NP_000375.2 | LNELSFK | 140 | 425.7371 | 737.3823 |
| apolipoprotein B precursor | NP_000375.2 | TQFNNNEYSQDLDAYNTK | 141 | 1082.974 | 362.2029 |
| apolipoprotein B precursor | NP_000375.2 | TQFNNNEYSQDLDAYNTK | 141 | 1082.974 | 525.2662 |
| apolipoprotein B precursor | NP_000375.2 | TQFNNNEYSQDLDAYNTK | 141 | 1082.974 | 596.3033 |
| apolipoprotein B precursor | NP_000375.2 | TQFNNNEYSQDLDAYNTK | 141 | 1082.974 | 711.3303 |
| apolipoprotein B precursor | NP_000375.2 | TQFNNNEYSQDLDAYNTK | 141 | 1082.974 | 824.4143 |
| apolipoprotein B precursor | NP_000375.2 | TQFNNNEYSQDLDAYNTK | 141 | 1082.974 | 939.4412 |
| apolipoprotein B precursor | NP_000375.2 | TQFNNNEYSQDLDAYNTK | 141 | 1082.974 | 1067.5 |
| apolipoprotein B precursor | NP_000375.2 | TQFNNNEYSQDLDAYNTK | 141 | 1082.974 | 1154.532 |
| apolipoprotein B precursor | NP_000375.2 | TQFNNNEYSQDLDAYNTK | 141 | 1082.974 | 1317.595 |
| apolipoprotein B precursor | NP_000375.2 | TQFNNNEYSQDLDAYNTK | 141 | 1082.974 | 1446.638 |
| apolipoprotein B precursor | NP_000375.2 | LSLPDFK | 142 | 410.2342 | 409.2076 |
| apolipoprotein B precursor | NP_000375.2 | LSLPDFK | 142 | 410.2342 | 506.2604 |
| apolipoprotein B precursor | NP_000375.2 | LSLPDFK | 142 | 410.2342 | 619.3444 |
| apolipoprotein B precursor | NP_000375.2 | LSLPDFK | 142 | 410.2342 | 706.3765 |
| apolipoprotein B precursor | NP_000375.2 | VSTAFVYTK | 143 | 508.2766 | 411.2233 |
| apolipoprotein B precursor | NP_000375.2 | VSTAFVYTK | 143 | 508.2766 | 510.2917 |
| apolipoprotein B precursor | NP_000375.2 | VSTAFVYTK | 143 | 508.2766 | 657.3601 |
| apolipoprotein B precursor | NP_000375.2 | VSTAFVYTK | 143 | 508.2766 | 728.3972 |
| apolipoprotein B precursor | NP_000375.2 | VSTAFVYTK | 143 | 508.2766 | 829.4449 |
| apolipoprotein B precursor | NP_000375.2 | VSTAFVYTK | 143 | 508.2766 | 916.4769 |
| apolipoprotein B precursor | NP_000375.2 | SVSLPSLDPASAK | 144 | 636.3457 | 305.1814 |
| apolipoprotein B precursor | NP_000375.2 | SVSLPSLDPASAK | 144 | 636.3457 | 376.2185 |
| apolipoprotein B precursor | NP_000375.2 | SVSLPSLDPASAK | 144 | 636.3457 | 473.2713 |
| apolipoprotein B precursor | NP_000375.2 | SVSLPSLDPASAK | 144 | 636.3457 | 588.2982 |
| apolipoprotein B precursor | NP_000375.2 | SVSLPSLDPASAK | 144 | 636.3457 | 701.3823 |
| apolipoprotein B precursor | NP_000375.2 | SVSLPSLDPASAK | 144 | 636.3457 | 788.4143 |
| apolipoprotein B precursor | NP_000375.2 | SVSLPSLDPASAK | 144 | 636.3457 | 885.4671 |
| apolipoprotein B precursor | NP_000375.2 | SVSLPSLDPASAK | 144 | 636.3457 | 998.5511 |
| apolipoprotein B precursor | NP_000375.2 | SVSLPSLDPASAK | 144 | 636.3457 | 1085.583 |
| apolipoprotein B precursor | NP_000375.2 | SVSLPSLDPASAK | 144 | 636.3457 | 1184.652 |
| pregnancy-zone protein | NP_002855.1 | EDSPFALK | 145 | 453.732 | 331.2334 |
| pregnancy-zone protein | NP_002855.1 | EDSPFALK | 145 | 453.732 | 478.3018 |
| pregnancy-zone protein | NP_002855.1 | EDSPFALK | 145 | 453.732 | 575.3546 |
| pregnancy-zone protein | NP_002855.1 | EDSPFALK | 145 | 453.732 | 662.3867 |
| pregnancy-zone protein | NP_002855.1 | EDSPFALK | 145 | 453.732 | 777.4136 |
| apolipoprotein B precursor | NP_000375.2 | DSYDLHDLK | 146 | 553.2617 | 375.2233 |
| apolipoprotein B precursor | NP_000375.2 | DSYDLHDLK | 146 | 553.2617 | 512.2822 |
| apolipoprotein B precursor | NP_000375.2 | DSYDLHDLK | 146 | 553.2617 | 625.3662 |
| apolipoprotein B precursor | NP_000375.2 | DSYDLHDLK | 146 | 553.2617 | 740.3932 |
| apolipoprotein B precursor | NP_000375.2 | DSYDLHDLK | 146 | 553.2617 | 903.4565 |
| apolipoprotein B precursor | NP_000375.2 | DSYDLHDLK | 146 | 553.2617 | 990.4885 |
| apolipoprotein D precursor | NP_001638.1 | WYEIEK | 147 | 434.216 | 389.2389 |
| apolipoprotein D precursor | NP_001638.1 | WYEIEK | 147 | 434.216 | 518.2815 |
| apolipoprotein D precursor | NP_001638.1 | WYEIEK | 147 | 434.216 | 681.3448 |
| serum amyloid P component precursor | NP_001630.1 | AYSLFSYNTQGR | 148 | 703.8386 | 360.1985 |
| serum amyloid P component precursor | NP_001630.1 | AYSLFSYNTQGR | 148 | 703.8386 | 461.2461 |
| serum amyloid P component precursor | NP_001630.1 | AYSLFSYNTQGR | 148 | 703.8386 | 575.2891 |
| serum amyloid P component precursor | NP_001630.1 | AYSLFSYNTQGR | 148 | 703.8386 | 738.3524 |
| serum amyloid P component precursor | NP_001630.1 | AYSLFSYNTQGR | 148 | 703.8386 | 825.3844 |
| serum amyloid P component precursor | NP_001630.1 | AYSLFSYNTQGR | 148 | 703.8386 | 972.4528 |
| serum amyloid P component precursor | NP_001630.1 | AYSLFSYNTQGR | 148 | 703.8386 | 1085.537 |
| serum amyloid P component precursor | NP_001630.1 | AYSLFSYNTQGR | 148 | 703.8386 | 1172.569 |
| serum amyloid P component precursor | NP_001630.1 | AYSLFSYNTQGR | 148 | 703.8386 | 1335.632 |
| histidine-rich glycoprotein precursor | NP_000403.1 | EENDDFASFR | 149 | 615.2571 | 409.2188 |
| histidine-rich glycoprotein precursor | NP_000403.1 | EENDDFASFR | 149 | 615.2571 | 480.256 |
| histidine-rich glycoprotein precursor | NP_000403.1 | EENDDFASFR | 149 | 615.2571 | 627.3244 |
| histidine-rich glycoprotein precursor | NP_000403.1 | EENDDFASFR | 149 | 615.2571 | 742.3513 |
| histidine-rich glycoprotein precursor | NP_000403.1 | EENDDFASFR | 149 | 615.2571 | 857.3782 |
| histidine-rich glycoprotein precursor | NP_000403.1 | EENDDFASFR | 149 | 615.2571 | 971.4212 |
| histidine-rich glycoprotein precursor | NP_000403.1 | EENDDFASFR | 149 | 615.2571 | 1100.464 |
| apolipoprotein B precursor | NP_000375.2 | AQIPILR | 150 | 405.7634 | 401.2865 |
| apolipoprotein B precursor | NP_000375.2 | AQIPILR | 150 | 405.7634 | 498.3393 |
| apolipoprotein B precursor | NP_000375.2 | AQIPILR | 150 | 405.7634 | 611.4233 |
| apolipoprotein B precursor | NP_000375.2 | AQIPILR | 150 | 405.7634 | 739.4819 |
| ceruloplasmin precursor | NP_000087.1 | EHEGAIYPDNTTDFQR | 151 | 946.9241 | 450.2454 |
| ceruloplasmin precursor | NP_000087.1 | EHEGAIYPDNTTDFQR | 151 | 946.9241 | 565.2723 |
| ceruloplasmin precursor | NP_000087.1 | EHEGAIYPDNTTDFQR | 151 | 946.9241 | 666.32 |
| ceruloplasmin precursor | NP_000087.1 | EHEGAIYPDNTTDFQR | 151 | 946.9241 | 767.3677 |
| ceruloplasmin precursor | NP_000087.1 | EHEGAIYPDNTTDFQR | 151 | 946.9241 | 881.4106 |
| ceruloplasmin precursor | NP_000087.1 | EHEGAIYPDNTTDFQR | 151 | 946.9241 | 996.4376 |
| ceruloplasmin precursor | NP_000087.1 | EHEGAIYPDNTTDFQR | 151 | 946.9241 | 1093.49 |
| ceruloplasmin precursor | NP_000087.1 | EHEGAIYPDNTTDFQR | 151 | 946.9241 | 1256.554 |
| ceruloplasmin precursor | NP_000087.1 | EHEGAIYPDNTTDFQR | 151 | 946.9241 | 1369.638 |
| ceruloplasmin precursor | NP_000087.1 | EHEGAIYPDNTTDFQR | 151 | 946.9241 | 1440.675 |
| ceruloplasmin precursor | NP_000087.1 | EHEGAIYPDNTTDFQR | 151 | 946.9241 | 1497.696 |
| apolipoprotein B precursor | NP_000375.2 | FNSSYLQGTNQITGR | 152 | 843.4157 | 333.1875 |
| apolipoprotein B precursor | NP_000375.2 | FNSSYLQGTNQITGR | 152 | 843.4157 | 446.2716 |
| apolipoprotein B precursor | NP_000375.2 | FNSSYLQGTNQITGR | 152 | 843.4157 | 574.3302 |
| apolipoprotein B precursor | NP_000375.2 | FNSSYLQGTNQITGR | 152 | 843.4157 | 688.3731 |
| apolipoprotein B precursor | NP_000375.2 | FNSSYLQGTNQITGR | 152 | 843.4157 | 789.4208 |
| apolipoprotein B precursor | NP_000375.2 | FNSSYLQGTNQITGR | 152 | 843.4157 | 846.4423 |
| apolipoprotein B precursor | NP_000375.2 | FNSSYLQGTNQITGR | 152 | 843.4157 | 974.5009 |
| apolipoprotein B precursor | NP_000375.2 | FNSSYLQGTNQITGR | 152 | 843.4157 | 1087.585 |
| apolipoprotein B precursor | NP_000375.2 | FNSSYLQGTNQITGR | 152 | 843.4157 | 1250.648 |
| apolipoprotein B precursor | NP_000375.2 | FNSSYLQGTNQITGR | 152 | 843.4157 | 1337.68 |
| apolipoprotein B precursor | NP_000375.2 | FNSSYLQGTNQITGR | 152 | 843.4157 | 1424.712 |
| pregnancy-zone protein | NP_002855.1 | EILNSLDK | 153 | 466.2584 | 375.2233 |
| pregnancy-zone protein | NP_002855.1 | EILNSLDK | 153 | 466.2584 | 462.2553 |
| pregnancy-zone protein | NP_002855.1 | EILNSLDK | 153 | 466.2584 | 576.2982 |
| pregnancy-zone protein | NP_002855.1 | EILNSLDK | 153 | 466.2584 | 689.3823 |
| pregnancy-zone protein | NP_002855.1 | EILNSLDK | 153 | 466.2584 | 802.4664 |
| apolipoprotein D precursor | NP_001638.1 | NPNLPPETVDSLK | 154 | 712.375 | 347.2283 |
| apolipoprotein D precursor | NP_001638.1 | NPNLPPETVDSLK | 154 | 712.375 | 462.2553 |
| apolipoprotein D precursor | NP_001638.1 | NPNLPPETVDSLK | 154 | 712.375 | 561.3237 |
| apolipoprotein D precursor | NP_001638.1 | NPNLPPETVDSLK | 154 | 712.375 | 662.3714 |
| apolipoprotein D precursor | NP_001638.1 | NPNLPPETVDSLK | 154 | 712.375 | 791.414 |
| apolipoprotein D precursor | NP_001638.1 | NPNLPPETVDSLK | 154 | 712.375 | 888.4667 |
| apolipoprotein D precursor | NP_001638.1 | NPNLPPETVDSLK | 154 | 712.375 | 985.5195 |
| apolipoprotein D precursor | NP_001638.1 | NPNLPPETVDSLK | 154 | 712.375 | 1098.604 |
| apolipoprotein D precursor | NP_001638.1 | NPNLPPETVDSLK | 154 | 712.375 | 1212.646 |
| apolipoprotein D precursor | NP_001638.1 | NPNLPPETVDSLK | 154 | 712.375 | 1309.699 |
| apolipoprotein A-I preproprotein | NP_000030.1 | THLAPYSDELR | 155 | 651.3279 | 417.2451 |
| apolipoprotein A-I preproprotein | NP_000030.1 | THLAPYSDELR | 155 | 651.3279 | 532.272 |
| apolipoprotein A-I preproprotein | NP_000030.1 | THLAPYSDELR | 155 | 651.3279 | 619.304 |
| apolipoprotein A-I preproprotein | NP_000030.1 | THLAPYSDELR | 155 | 651.3279 | 782.3674 |
| apolipoprotein A-I preproprotein | NP_000030.1 | THLAPYSDELR | 155 | 651.3279 | 879.4201 |
| apolipoprotein A-I preproprotein | NP_000030.1 | THLAPYSDELR | 155 | 651.3279 | 950.4572 |
| apolipoprotein A-I preproprotein | NP_000030.1 | THLAPYSDELR | 155 | 651.3279 | 1063.541 |
| apolipoprotein A-I preproprotein | NP_000030.1 | THLAPYSDELR | 155 | 651.3279 | 1200.6 |
| apolipoprotein E precursor | NP_000032.1 | QWAGLVEK | 156 | 465.7558 | 375.2233 |
| apolipoprotein E precursor | NP_000032.1 | QWAGLVEK | 156 | 465.7558 | 488.3073 |
| apolipoprotein E precursor | NP_000032.1 | QWAGLVEK | 156 | 465.7558 | 545.3288 |
| apolipoprotein E precursor | NP_000032.1 | QWAGLVEK | 156 | 465.7558 | 616.3659 |
| apolipoprotein E precursor | NP_000032.1 | QWAGLVEK | 156 | 465.7558 | 802.4452 |
| paraoxonase 1 | NP_000437.3 | STVELFK | 157 | 412.2316 | 407.2647 |
| paraoxonase 1 | NP_000437.3 | STVELFK | 157 | 412.2316 | 536.3073 |
| paraoxonase 1 | NP_000437.3 | STVELFK | 157 | 412.2316 | 635.3757 |
| paraoxonase 1 | NP_000437.3 | STVELFK | 157 | 412.2316 | 736.4234 |
| apolipoprotein A-IV precursor | NP_000473.2 | ALVQQMEQLR | 158 | 608.3293 | 416.261 |
| apolipoprotein A-IV precursor | NP_000473.2 | ALVQQMEQLR | 158 | 608.3293 | 545.3036 |
| apolipoprotein A-IV precursor | NP_000473.2 | ALVQQMEQLR | 158 | 608.3293 | 676.3441 |
| apolipoprotein A-IV precursor | NP_000473.2 | ALVQQMEQLR | 158 | 608.3293 | 804.4027 |
| apolipoprotein A-IV precursor | NP_000473.2 | ALVQQMEQLR | 158 | 608.3293 | 932.4613 |
| apolipoprotein A-IV precursor | NP_000473.2 | ALVQQMEQLR | 158 | 608.3293 | 1031.53 |
| apolipoprotein A-IV precursor | NP_000473.2 | ALVQQMEQLR | 158 | 608.3293 | 1144.614 |
| apolipoprotein B precursor | NP_000375.2 | SFDYHQFVDETNDK | 159 | 872.8761 | 376.1821 |
| apolipoprotein B precursor | NP_000375.2 | SFDYHQFVDETNDK | 159 | 872.8761 | 477.2298 |
| apolipoprotein B precursor | NP_000375.2 | SFDYHQFVDETNDK | 159 | 872.8761 | 606.2724 |
| apolipoprotein B precursor | NP_000375.2 | SFDYHQFVDETNDK | 159 | 872.8761 | 721.2993 |
| apolipoprotein B precursor | NP_000375.2 | SFDYHQFVDETNDK | 159 | 872.8761 | 820.3677 |
| apolipoprotein B precursor | NP_000375.2 | SFDYHQFVDETNDK | 159 | 872.8761 | 967.4362 |
| apolipoprotein B precursor | NP_000375.2 | SFDYHQFVDETNDK | 159 | 872.8761 | 1095.495 |
| apolipoprotein B precursor | NP_000375.2 | SFDYHQFVDETNDK | 159 | 872.8761 | 1232.554 |
| apolipoprotein B precursor | NP_000375.2 | SFDYHQFVDETNDK | 159 | 872.8761 | 1395.617 |
| apolipoprotein B precursor | NP_000375.2 | QVFLYPEK | 160 | 512.2791 | 373.2076 |
| apolipoprotein B precursor | NP_000375.2 | QVFLYPEK | 160 | 512.2791 | 536.2709 |
| apolipoprotein B precursor | NP_000375.2 | QVFLYPEK | 160 | 512.2791 | 649.355 |
| apolipoprotein B precursor | NP_000375.2 | QVFLYPEK | 160 | 512.2791 | 796.4234 |
| apolipoprotein B precursor | NP_000375.2 | QVFLYPEK | 160 | 512.2791 | 895.4918 |
| apolipoprotein C-II precursor | NP_000474.2 | ESLSSYWESAK | 161 | 643.7986 | 305.1814 |
| apolipoprotein C-II precursor | NP_000474.2 | ESLSSYWESAK | 161 | 643.7986 | 434.224 |
| apolipoprotein C-II precursor | NP_000474.2 | ESLSSYWESAK | 161 | 643.7986 | 783.3666 |
| apolipoprotein C-II precursor | NP_000474.2 | ESLSSYWESAK | 161 | 643.7986 | 870.3987 |
| apolipoprotein C-II precursor | NP_000474.2 | ESLSSYWESAK | 161 | 643.7986 | 957.4307 |
| apolipoprotein C-II precursor | NP_000474.2 | ESLSSYWESAK | 161 | 643.7986 | 1070.515 |
| apolipoprotein C-II precursor | NP_000474.2 | ESLSSYWESAK | 161 | 643.7986 | 1157.547 |
| pregnancy-zone protein | NP_002855.1 | GEVFTLK | 162 | 397.2263 | 361.244 |
| pregnancy-zone protein | NP_002855.1 | GEVFTLK | 162 | 397.2263 | 508.3124 |
| pregnancy-zone protein | NP_002855.1 | GEVFTLK | 162 | 397.2263 | 607.3808 |
| pregnancy-zone protein | NP_002855.1 | GEVFTLK | 162 | 397.2263 | 736.4234 |
| alpha-2-macroglobulin precursor | NP_000005.2 | VGFYESDVM[Oxid]GR | 163 | 630.2899 | 363.1804 |
| alpha-2-macroglobulin precursor | NP_000005.2 | VGFYESDVM[Oxid]GR | 163 | 630.2899 | 462.2488 |
| alpha-2-macroglobulin precursor | NP_000005.2 | VGFYESDVM[Oxid]GR | 163 | 630.2899 | 577.2757 |
| alpha-2-macroglobulin precursor | NP_000005.2 | VGFYESDVM[Oxid]GR | 163 | 630.2899 | 664.3077 |
| alpha-2-macroglobulin precursor | NP_000005.2 | VGFYESDVM[Oxid]GR | 163 | 630.2899 | 793.3503 |
| alpha-2-macroglobulin precursor | NP_000005.2 | VGFYESDVM[Oxid]GR | 163 | 630.2899 | 956.4136 |
| alpha-2-macroglobulin precursor | NP_000005.2 | VGFYESDVM[Oxid]GR | 163 | 630.2899 | 1103.482 |
| alpha-2-macroglobulin precursor | NP_000005.2 | VGFYESDVM[Oxid]GR | 163 | 630.2899 | 1160.504 |
| apolipoprotein B precursor | NP_000375.2 | YENYELTLK | 164 | 586.7953 | 361.244 |
| apolipoprotein B precursor | NP_000375.2 | YENYELTLK | 164 | 586.7953 | 474.3281 |
| apolipoprotein B precursor | NP_000375.2 | YENYELTLK | 164 | 586.7953 | 603.3707 |
| apolipoprotein B precursor | NP_000375.2 | YENYELTLK | 164 | 586.7953 | 766.434 |
| apolipoprotein B precursor | NP_000375.2 | YENYELTLK | 164 | 586.7953 | 880.4769 |
| apolipoprotein B precursor | NP_000375.2 | YENYELTLK | 164 | 586.7953 | 1009.52 |
| pregnancy-zone protein | NP_002855.1 | SSGSLLNNAIK | 165 | 552.3064 | 331.2334 |
| pregnancy-zone protein | NP_002855.1 | SSGSLLNNAIK | 165 | 552.3064 | 445.2764 |
| pregnancy-zone protein | NP_002855.1 | SSGSLLNNAIK | 165 | 552.3064 | 559.3193 |
| pregnancy-zone protein | NP_002855.1 | SSGSLLNNAIK | 165 | 552.3064 | 672.4034 |
| pregnancy-zone protein | NP_002855.1 | SSGSLLNNAIK | 165 | 552.3064 | 785.4874 |
| pregnancy-zone protein | NP_002855.1 | SSGSLLNNAIK | 165 | 552.3064 | 872.5195 |
| pregnancy-zone protein | NP_002855.1 | SSGSLLNNAIK | 165 | 552.3064 | 929.5409 |
| pregnancy-zone protein | NP_002855.1 | SSGSLLNNAIK | 165 | 552.3064 | 1016.573 |
| apolipoprotein A-II preproprotein | NP_001634.1 | SKEQLTPLIKK | 166 | 642.8979 | 388.2913 |
| apolipoprotein A-II preproprotein | NP_001634.1 | SKEQLTPLIKK | 166 | 642.8979 | 501.3753 |
| apolipoprotein A-II preproprotein | NP_001634.1 | SKEQLTPLIKK | 166 | 642.8979 | 598.4281 |
| apolipoprotein A-II preproprotein | NP_001634.1 | SKEQLTPLIKK | 166 | 642.8979 | 699.4758 |
| apolipoprotein A-II preproprotein | NP_001634.1 | SKEQLTPLIKK | 166 | 642.8979 | 812.5599 |
| apolipoprotein A-II preproprotein | NP_001634.1 | SKEQLTPLIKK | 166 | 642.8979 | 940.6184 |
| apolipoprotein A-II preproprotein | NP_001634.1 | SKEQLTPLIKK | 166 | 642.8979 | 1069.661 |
| apolipoprotein A-II preproprotein | NP_001634.1 | SKEQLTPLIKK | 166 | 642.8979 | 1197.756 |
| pregnancy-zone protein | NP_002855.1 | QTLSWTVTPK | 167 | 580.8192 | 345.2127 |
| pregnancy-zone protein | NP_002855.1 | QTLSWTVTPK | 167 | 580.8192 | 444.2811 |
| pregnancy-zone protein | NP_002855.1 | QTLSWTVTPK | 167 | 580.8192 | 545.3288 |
| pregnancy-zone protein | NP_002855.1 | QTLSWTVTPK | 167 | 580.8192 | 731.4081 |
| pregnancy-zone protein | NP_002855.1 | QTLSWTVTPK | 167 | 580.8192 | 818.4401 |
| pregnancy-zone protein | NP_002855.1 | QTLSWTVTPK | 167 | 580.8192 | 931.5242 |
| pregnancy-zone protein | NP_002855.1 | QTLSWTVTPK | 167 | 580.8192 | 1032.572 |
| apolipoprotein B precursor | NP_000375.2 | IPSVQINFK | 168 | 523.3057 | 408.2236 |
| apolipoprotein B precursor | NP_000375.2 | IPSVQINFK | 168 | 523.3057 | 521.3077 |
| apolipoprotein B precursor | NP_000375.2 | IPSVQINFK | 168 | 523.3057 | 649.3662 |
| apolipoprotein B precursor | NP_000375.2 | IPSVQINFK | 168 | 523.3057 | 748.4346 |
| apolipoprotein B precursor | NP_000375.2 | IPSVQINFK | 168 | 523.3057 | 835.4667 |
| apolipoprotein B precursor | NP_000375.2 | IPSVQINFK | 168 | 523.3057 | 932.5195 |
| apolipoprotein B precursor | NP_000375.2 | IYSLWEHSTK | 169 | 632.322 | 335.192 |
| apolipoprotein B precursor | NP_000375.2 | IYSLWEHSTK | 169 | 632.322 | 472.2509 |
| apolipoprotein B precursor | NP_000375.2 | IYSLWEHSTK | 169 | 632.322 | 601.2935 |
| apolipoprotein B precursor | NP_000375.2 | IYSLWEHSTK | 169 | 632.322 | 787.3728 |
| apolipoprotein B precursor | NP_000375.2 | IYSLWEHSTK | 169 | 632.322 | 900.4568 |
| apolipoprotein B precursor | NP_000375.2 | IYSLWEHSTK | 169 | 632.322 | 987.4889 |
| apolipoprotein B precursor | NP_000375.2 | IYSLWEHSTK | 169 | 632.322 | 1150.552 |
| apolipoprotein D precursor | NP_001638.1 | NILTSNNIDVK | 170 | 615.8381 | 361.2076 |
| apolipoprotein D precursor | NP_001638.1 | NILTSNNIDVK | 170 | 615.8381 | 474.2917 |
| apolipoprotein D precursor | NP_001638.1 | NILTSNNIDVK | 170 | 615.8381 | 588.3346 |
| apolipoprotein D precursor | NP_001638.1 | NILTSNNIDVK | 170 | 615.8381 | 702.3775 |
| apolipoprotein D precursor | NP_001638.1 | NILTSNNIDVK | 170 | 615.8381 | 789.4095 |
| apolipoprotein D precursor | NP_001638.1 | NILTSNNIDVK | 170 | 615.8381 | 890.4572 |
| apolipoprotein D precursor | NP_001638.1 | NILTSNNIDVK | 170 | 615.8381 | 1116.625 |
| apolipoprotein H precursor | NP_000033.1 | ATFGC[Carboxymethyl]HDGYSL | 171 | 1194.486 | 409.1746 |
| | | DGPEEIEC[Carboxymethyl]TK | | | |
| apolipoprotein H precursor | NP_000033.1 | ATFGC[Carboxymethyl]HDGYSL | 171 | 1194.486 | 538.2172 |
| | | DGPEEIEC[Carboxymethyl]TK | | | |
| apolipoprotein H precursor | NP_000033.1 | ATFGC[Carboxymethyl]HDGYSL | 171 | 1194.486 | 651.3013 |
| | | DGPEEIEC[Carboxymethyl]TK | | | |
| apolipoprotein H precursor | NP_000033.1 | ATFGC[Carboxymethyl]HDGYSL | 171 | 1194.486 | 780.3439 |
| | | DGPEEIEC[Carboxymethyl]TK | | | |
| apolipoprotein H precursor | NP_000033.1 | ATFGC[Carboxymethyl]HDGYSL | 171 | 1194.486 | 909.3864 |
| | | DGPEEIEC[Carboxymethyl]TK | | | |
| apolipoprotein H precursor | NP_000033.1 | ATFGC[Carboxymethyl]HDGYSL | 171 | 1194.486 | 1006.439 |
| | | DGPEEIEC[Carboxymethyl]TK | | | |
| apolipoprotein H precursor | NP_000033.1 | ATFGC[Carboxymethyl]HDGYSL | 171 | 1194.486 | 1063.461 |
| | | DGPEEIEC[Carboxymethyl]TK | | | |
| apolipoprotein H precursor | NP_000033.1 | ATFGC[Carboxymethyl]HDGYSL | 171 | 1194.486 | 1178.488 |
| | | DGPEEIEC[Carboxymethyl]TK | | | |
| apolipoprotein H precursor | NP_000033.1 | ATFGC[Carboxymethyl]HDGYSL | 171 | 1194.486 | 1291.572 |
| | | DGPEEIEC[Carboxymethyl]TK | | | |
| apolipoprotein H precursor | NP_000033.1 | ATFGC[Carboxymethyl]HDGYSL | 171 | 1194.486 | 1378.604 |
| | | DGPEEIEC[Carboxymethyl]TK | | | |
| apolipoprotein B precursor | NP_000375.2 | FPEVDVLTK | 172 | 524.2897 | 361.244 |
| apolipoprotein B precursor | NP_000375.2 | FPEVDVLTK | 172 | 524.2897 | 460.3124 |
| apolipoprotein B precursor | NP_000375.2 | FPEVDVLTK | 172 | 524.2897 | 575.3394 |
| apolipoprotein B precursor | NP_000375.2 | FPEVDVLTK | 172 | 524.2897 | 674.4078 |
| apolipoprotein B precursor | NP_000375.2 | FPEVDVLTK | 172 | 524.2897 | 803.4504 |
| apolipoprotein B precursor | NP_000375.2 | FPEVDVLTK | 172 | 524.2897 | 900.5031 |
| apolipoprotein B precursor | NP_000375.2 | EVGTVLSQVYSK | 173 | 655.3535 | 397.2076 |
| apolipoprotein B precursor | NP_000375.2 | EVGTVLSQVYSK | 173 | 655.3535 | 496.276 |
| apolipoprotein B precursor | NP_000375.2 | EVGTVLSQVYSK | 173 | 655.3535 | 624.3346 |
| apolipoprotein B precursor | NP_000375.2 | EVGTVLSQVYSK | 173 | 655.3535 | 711.3666 |
| apolipoprotein B precursor | NP_000375.2 | EVGTVLSQVYSK | 173 | 655.3535 | 824.4507 |
| apolipoprotein B precursor | NP_000375.2 | EVGTVLSQVYSK | 173 | 655.3535 | 923.5191 |
| apolipoprotein B precursor | NP_000375.2 | EVGTVLSQVYSK | 173 | 655.3535 | 1024.567 |
| apolipoprotein B precursor | NP_000375.2 | EVGTVLSQVYSK | 173 | 655.3535 | 1081.588 |
| apolipoprotein B precursor | NP_000375.2 | EVGTVLSQVYSK | 173 | 655.3535 | 1180.657 |
| apolipoprotein B precursor | NP_000375.2 | YGMVAQVTQTLK | 174 | 669.8579 | 361.244 |
| apolipoprotein B precursor | NP_000375.2 | YGMVAQVTQTLK | 174 | 669.8579 | 489.3026 |
| apolipoprotein B precursor | NP_000375.2 | YGMVAQVTQTLK | 174 | 669.8579 | 590.3503 |
| apolipoprotein B precursor | NP_000375.2 | YGMVAQVTQTLK | 174 | 669.8579 | 689.4186 |
| apolipoprotein B precursor | NP_000375.2 | YGMVAQVTQTLK | 174 | 669.8579 | 817.4772 |
| apolipoprotein B precursor | NP_000375.2 | YGMVAQVTQTLK | 174 | 669.8579 | 888.5143 |
| apolipoprotein B precursor | NP_000375.2 | YGMVAQVTQTLK | 174 | 669.8579 | 987.5828 |
| apolipoprotein B precursor | NP_000375.2 | YGMVAQVTQTLK | 174 | 669.8579 | 1118.623 |
| apolipoprotein B precursor | NP_000375.2 | YGMVAQVTQTLK | 174 | 669.8579 | 1175.645 |
| pregnancy-zone protein | NP_002855.1 | ATVLNYLPK | 175 | 509.8002 | 357.2491 |
| pregnancy-zone protein | NP_002855.1 | ATVLNYLPK | 175 | 509.8002 | 520.3124 |
| pregnancy-zone protein | NP_002855.1 | ATVLNYLPK | 175 | 509.8002 | 634.3553 |
| pregnancy-zone protein | NP_002855.1 | ATVLNYLPK | 175 | 509.8002 | 846.5078 |
| pregnancy-zone protein | NP_002855.1 | ATVLNYLPK | 175 | 509.8002 | 947.5555 |
| apolipoprotein D precursor | NP_001638.1 | ADGTVNQIEGEATPVNLTEPAK | 176 | 1127.563 | 315.2021 |
| apolipoprotein D precursor | NP_001638.1 | ADGTVNQIEGEATPVNLTEPAK | 176 | 1127.563 | 444.2447 |
| apolipoprotein D precursor | NP_001638.1 | ADGTVNQIEGEATPVNLTEPAK | 176 | 1127.563 | 545.2924 |
| apolipoprotein D precursor | NP_001638.1 | ADGTVNQIEGEATPVNLTEPAK | 176 | 1127.563 | 658.3765 |
| apolipoprotein D precursor | NP_001638.1 | ADGTVNQIEGEATPVNLTEPAK | 176 | 1127.563 | 772.4194 |
| apolipoprotein D precursor | NP_001638.1 | ADGTVNQIEGEATPVNLTEPAK | 176 | 1127.563 | 871.4878 |
| apolipoprotein D precursor | NP_001638.1 | ADGTVNQIEGEATPVNLTEPAK | 176 | 1127.563 | 968.5406 |
| apolipoprotein D precursor | NP_001638.1 | ADGTVNQIEGEATPVNLTEPAK | 176 | 1127.563 | 1069.588 |
| apolipoprotein D precursor | NP_001638.1 | ADGTVNQIEGEATPVNLTEPAK | 176 | 1127.563 | 1140.625 |
| apolipoprotein D precursor | NP_001638.1 | ADGTVNQIEGEATPVNLTEPAK | 176 | 1127.563 | 1269.668 |
| apolipoprotein D precursor | NP_001638.1 | ADGTVNQIEGEATPVNLTEPAK | 176 | 1127.563 | 1326.689 |
| apolipoprotein D precursor | NP_001638.1 | ADGTVNQIEGEATPVNLTEPAK | 176 | 1127.563 | 1455.732 |
| serum amyloid P component precursor | NP_001630.1 | VGEYSLYIGR | 177 | 578.8035 | 345.2239 |
| serum amyloid P component precursor | NP_001630.1 | VGEYSLYIGR | 177 | 578.8035 | 508.2873 |
| serum amyloid P component precursor | NP_001630.1 | VGEYSLYIGR | 177 | 578.8035 | 621.3713 |
| serum amyloid P component precursor | NP_001630.1 | VGEYSLYIGR | 177 | 578.8035 | 871.4667 |
| serum amyloid P component precursor | NP_001630.1 | VGEYSLYIGR | 177 | 578.8035 | 1000.509 |
| serum amyloid P component precursor | NP_001630.1 | VGEYSLYIGR | 177 | 578.8035 | 1057.531 |
| apolipoprotein A-II preproprotein | NP_001634.1 | SKEQLTPLIK | 178 | 578.8505 | 373.2804 |
| apolipoprotein A-II preproprotein | NP_001634.1 | SKEQLTPLIK | 178 | 578.8505 | 470.3332 |
| apolipoprotein A-II preproprotein | NP_001634.1 | SKEQLTPLIK | 178 | 578.8505 | 571.3808 |
| apolipoprotein A-II preproprotein | NP_001634.1 | SKEQLTPLIK | 178 | 578.8505 | 684.4649 |
| apolipoprotein A-II preproprotein | NP_001634.1 | SKEQLTPLIK | 178 | 578.8505 | 812.5234 |
| apolipoprotein A-II preproprotein | NP_001634.1 | SKEQLTPLIK | 178 | 578.8505 | 1069.661 |
| apolipoprotein E precursor | NP_000032.1 | SELEEQLTPVAEETR | 179 | 865.9258 | 405.2087 |
| apolipoprotein E precursor | NP_000032.1 | SELEEQLTPVAEETR | 179 | 865.9258 | 534.2513 |
| apolipoprotein E precursor | NP_000032.1 | SELEEQLTPVAEETR | 179 | 865.9258 | 605.2884 |
| apolipoprotein E precursor | NP_000032.1 | SELEEQLTPVAEETR | 179 | 865.9258 | 704.3568 |
| apolipoprotein E precursor | NP_000032.1 | SELEEQLTPVAEETR | 179 | 865.9258 | 801.4095 |
| apolipoprotein E precursor | NP_000032.1 | SELEEQLTPVAEETR | 179 | 865.9258 | 902.4572 |
| apolipoprotein E precursor | NP_000032.1 | SELEEQLTPVAEETR | 179 | 865.9258 | 1015.541 |
| apolipoprotein E precursor | NP_000032.1 | SELEEQLTPVAEETR | 179 | 865.9258 | 1143.6 |
| apolipoprotein E precursor | NP_000032.1 | SELEEQLTPVAEETR | 179 | 865.9258 | 1272.642 |
| apolipoprotein E precursor | NP_000032.1 | SELEEQLTPVAEETR | 179 | 865.9258 | 1401.685 |
| apolipoprotein B precursor | NP_000375.2 | FIIPGLK | 180 | 394.2574 | 317.2178 |
| apolipoprotein B precursor | NP_000375.2 | FIIPGLK | 180 | 394.2574 | 414.2705 |
| apolipoprotein B precursor | NP_000375.2 | FIIPGLK | 180 | 394.2574 | 527.3546 |
| apolipoprotein B precursor | NP_000375.2 | FIIPGLK | 180 | 394.2574 | 640.4387 |
| apolipoprotein A-II preproprotein | NP_001634.1 | EQLTPLIK | 181 | 471.287 | 373.2804 |
| apolipoprotein A-II preproprotein | NP_001634.1 | EQLTPLIK | 181 | 471.287 | 470.3332 |
| apolipoprotein A-II preproprotein | NP_001634.1 | EQLTPLIK | 181 | 471.287 | 571.3808 |
| apolipoprotein A-II preproprotein | NP_001634.1 | EQLTPLIK | 181 | 471.287 | 684.4649 |
| apolipoprotein A-II preproprotein | NP_001634.1 | EQLTPLIK | 181 | 471.287 | 812.5234 |
| apolipoprotein B precursor | NP_000375.2 | HIQNIDIQHLAGK | 182 | 743.9099 | 275.1708 |
| apolipoprotein B precursor | NP_000375.2 | HIQNIDIQHLAGK | 182 | 743.9099 | 388.2549 |
| apolipoprotein B precursor | NP_000375.2 | HIQNIDIQHLAGK | 182 | 743.9099 | 525.3138 |
| apolipoprotein B precursor | NP_000375.2 | HIQNIDIQHLAGK | 182 | 743.9099 | 653.3724 |
| apolipoprotein B precursor | NP_000375.2 | HIQNIDIQHLAGK | 182 | 743.9099 | 766.4564 |
| apolipoprotein B precursor | NP_000375.2 | HIQNIDIQHLAGK | 182 | 743.9099 | 881.4834 |
| apolipoprotein B precursor | NP_000375.2 | HIQNIDIQHLAGK | 182 | 743.9099 | 994.5674 |
| apolipoprotein B precursor | NP_000375.2 | HIQNIDIQHLAGK | 182 | 743.9099 | 1108.61 |
| apolipoprotein B precursor | NP_000375.2 | HIQNIDIQHLAGK | 182 | 743.9099 | 1236.669 |
| apolipoprotein B precursor | NP_000375.2 | HIQNIDIQHLAGK | 182 | 743.9099 | 1349.753 |
| apolipoprotein B precursor | NP_000375.2 | WNFYYSPQSSPDK | 183 | 809.8622 | 359.192 |
| apolipoprotein B precursor | NP_000375.2 | WNFYYSPQSSPDK | 183 | 809.8622 | 446.224 |
| apolipoprotein B precursor | NP_000375.2 | WNFYYSPQSSPDK | 183 | 809.8622 | 533.256 |
| apolipoprotein B precursor | NP_000375.2 | WNFYYSPQSSPDK | 183 | 809.8622 | 661.3146 |
| apolipoprotein B precursor | NP_000375.2 | WNFYYSPQSSPDK | 183 | 809.8622 | 758.3674 |
| apolipoprotein B precursor | NP_000375.2 | WNFYYSPQSSPDK | 183 | 809.8622 | 845.3994 |
| apolipoprotein B precursor | NP_000375.2 | WNFYYSPQSSPDK | 183 | 809.8622 | 1008.463 |
| apolipoprotein B precursor | NP_000375.2 | WNFYYSPQSSPDK | 183 | 809.8622 | 1171.526 |
| apolipoprotein B precursor | NP_000375.2 | WNFYYSPQSSPDK | 183 | 809.8622 | 1318.594 |
| apolipoprotein B precursor | NP_000375.2 | WNFYYSPQSSPDK | 183 | 809.8622 | 1432.637 |
| apolipoprotein B precursor | NP_000375.2 | ATLYALSHAVNNYHK | 184 | 851.439 | 447.2345 |
| apolipoprotein B precursor | NP_000375.2 | ATLYALSHAVNNYHK | 184 | 851.439 | 561.2774 |
| apolipoprotein B precursor | NP_000375.2 | ATLYALSHAVNNYHK | 184 | 851.439 | 675.3204 |
| apolipoprotein B precursor | NP_000375.2 | ATLYALSHAVNNYHK | 184 | 851.439 | 774.3888 |
| apolipoprotein B precursor | NP_000375.2 | ATLYALSHAVNNYHK | 184 | 851.439 | 845.4259 |
| apolipoprotein B precursor | NP_000375.2 | ATLYALSHAVNNYHK | 184 | 851.439 | 982.4848 |
| apolipoprotein B precursor | NP_000375.2 | ATLYALSHAVNNYHK | 184 | 851.439 | 1069.517 |
| apolipoprotein B precursor | NP_000375.2 | ATLYALSHAVNNYHK | 184 | 851.439 | 1182.601 |
| apolipoprotein B precursor | NP_000375.2 | ATLYALSHAVNNYHK | 184 | 851.439 | 1253.638 |
| apolipoprotein B precursor | NP_000375.2 | ATLYALSHAVNNYHK | 184 | 851.439 | 1416.701 |
| apolipoprotein B precursor | NP_000375.2 | LQDFSDQLSDYYEK | 185 | 875.894 | 439.2182 |
| apolipoprotein B precursor | NP_000375.2 | LQDFSDQLSDYYEK | 185 | 875.894 | 602.2815 |
| apolipoprotein B precursor | NP_000375.2 | LQDFSDQLSDYYEK | 185 | 875.894 | 717.3085 |
| apolipoprotein B precursor | NP_000375.2 | LQDFSDQLSDYYEK | 185 | 875.894 | 804.3405 |
| apolipoprotein B precursor | NP_000375.2 | LQDFSDQLSDYYEK | 185 | 875.894 | 917.4246 |
| apolipoprotein B precursor | NP_000375.2 | LQDFSDQLSDYYEK | 185 | 875.894 | 1045.483 |
| apolipoprotein B precursor | NP_000375.2 | LQDFSDQLSDYYEK | 185 | 875.894 | 1160.51 |
| apolipoprotein B precursor | NP_000375.2 | LQDFSDQLSDYYEK | 185 | 875.894 | 1247.542 |
| apolipoprotein B precursor | NP_000375.2 | LQDFSDQLSDYYEK | 185 | 875.894 | 1394.61 |
| apolipoprotein B precursor | NP_000375.2 | IEFEWNTGTNVDTK | 186 | 827.3914 | 363.1869 |
| apolipoprotein B precursor | NP_000375.2 | IEFEWNTGTNVDTK | 186 | 827.3914 | 462.2553 |
| apolipoprotein B precursor | NP_000375.2 | IEFEWNTGTNVDTK | 186 | 827.3914 | 576.2982 |
| apolipoprotein B precursor | NP_000375.2 | IEFEWNTGTNVDTK | 186 | 827.3914 | 677.3459 |
| apolipoprotein B precursor | NP_000375.2 | IEFEWNTGTNVDTK | 186 | 827.3914 | 734.3674 |
| apolipoprotein B precursor | NP_000375.2 | IEFEWNTGTNVDTK | 186 | 827.3914 | 835.415 |
| apolipoprotein B precursor | NP_000375.2 | IEFEWNTGTNVDTK | 186 | 827.3914 | 949.4579 |
| apolipoprotein B precursor | NP_000375.2 | IEFEWNTGTNVDTK | 186 | 827.3914 | 1135.537 |
| apolipoprotein B precursor | NP_000375.2 | IEFEWNTGTNVDTK | 186 | 827.3914 | 1264.58 |
| apolipoprotein B precursor | NP_000375.2 | IEFEWNTGTNVDTK | 186 | 827.3914 | 1411.648 |
| apolipoprotein B precursor | NP_000375.2 | DFSLWEK | 187 | 462.7267 | 462.2342 |
| apolipoprotein B precursor | NP_000375.2 | DFSLWEK | 187 | 462.7267 | 575.3182 |
| apolipoprotein B precursor | NP_000375.2 | DFSLWEK | 187 | 462.7267 | 662.3503 |
| apolipoprotein B precursor | NP_000375.2 | DFSLWEK | 187 | 462.7267 | 809.4186 |
| serum amyloid P component precursor | NP_001630.1 | DNELLVYK | 188 | 497.2662 | 409.244 |
| serum amyloid P component precursor | NP_001630.1 | DNELLVYK | 188 | 497.2662 | 522.3281 |
| serum amyloid P component precursor | NP_001630.1 | DNELLVYK | 188 | 497.2662 | 635.4121 |
| serum amyloid P component precursor | NP_001630.1 | DNELLVYK | 188 | 497.2662 | 764.4547 |
| serum amyloid P component precursor | NP_001630.1 | DNELLVYK | 188 | 497.2662 | 878.4976 |
| apolipoprotein A-IV precursor | NP_000473.2 | LVPFATELHER | 189 | 656.3564 | 441.2199 |
| apolipoprotein A-IV precursor | NP_000473.2 | LVPFATELHER | 189 | 656.3564 | 554.304 |
| apolipoprotein A-IV precursor | NP_000473.2 | LVPFATELHER | 189 | 656.3564 | 683.3466 |
| apolipoprotein A-IV precursor | NP_000473.2 | LVPFATELHER | 189 | 656.3564 | 784.3942 |
| apolipoprotein A-IV precursor | NP_000473.2 | LVPFATELHER | 189 | 656.3564 | 855.4313 |
| apolipoprotein A-IV precursor | NP_000473.2 | LVPFATELHER | 189 | 656.3564 | 1002.5 |
| apolipoprotein A-IV precursor | NP_000473.2 | LVPFATELHER | 189 | 656.3564 | 1099.552 |
| apolipoprotein A-IV precursor | NP_000473.2 | LVPFATELHER | 189 | 656.3564 | 1198.621 |
| apolipoprotein B precursor | NP_000375.2 | NLLVALK | 190 | 385.7604 | 331.2334 |
| apolipoprotein B precursor | NP_000375.2 | NLLVALK | 190 | 385.7604 | 430.3018 |
| apolipoprotein B precursor | NP_000375.2 | NLLVALK | 190 | 385.7604 | 543.3859 |
| apolipoprotein B precursor | NP_000375.2 | NLLVALK | 190 | 385.7604 | 656.47 |
| alpha-2-macroglobulin precursor | NP_000005.2 | NEDSLVFVQTDK | 191 | 697.8436 | 363.1869 |
| alpha-2-macroglobulin precursor | NP_000005.2 | NEDSLVFVQTDK | 191 | 697.8436 | 491.2455 |
| alpha-2-macroglobulin precursor | NP_000005.2 | NEDSLVFVQTDK | 191 | 697.8436 | 590.3138 |
| alpha-2-macroglobulin precursor | NP_000005.2 | NEDSLVFVQTDK | 191 | 697.8436 | 737.3823 |
| alpha-2-macroglobulin precursor | NP_000005.2 | NEDSLVFVQTDK | 191 | 697.8436 | 836.4507 |
| alpha-2-macroglobulin precursor | NP_000005.2 | NEDSLVFVQTDK | 191 | 697.8436 | 949.5347 |
| alpha-2-macroglobulin precursor | NP_000005.2 | NEDSLVFVQTDK | 191 | 697.8436 | 1036.567 |
| alpha-2-macroglobulin precursor | NP_000005.2 | NEDSLVFVQTDK | 191 | 697.8436 | 1151.594 |
| alpha-2-macroglobulin precursor | NP_000005.2 | NEDSLVFVQTDK | 191 | 697.8436 | 1280.636 |
| apolipoprotein E precursor | NP_000032.1 | FWDYLR | 192 | 450.2242 | 451.2658 |
| apolipoprotein E precursor | NP_000032.1 | FWDYLR | 192 | 450.2242 | 752.3721 |
| paraoxonase 1 | NP_000437.3 | HANWTLTPLK | 193 | 590.8273 | 357.2491 |
| paraoxonase 1 | NP_000437.3 | HANWTLTPLK | 193 | 590.8273 | 458.2968 |
| paraoxonase 1 | NP_000437.3 | HANWTLTPLK | 193 | 590.8273 | 571.3808 |
| paraoxonase 1 | NP_000437.3 | HANWTLTPLK | 193 | 590.8273 | 672.4285 |
| paraoxonase 1 | NP_000437.3 | HANWTLTPLK | 193 | 590.8273 | 858.5078 |
| paraoxonase 1 | NP_000437.3 | HANWTLTPLK | 193 | 590.8273 | 972.5507 |
| paraoxonase 1 | NP_000437.3 | HANWTLTPLK | 193 | 590.8273 | 1043.588 |
| alpha 1 globin | NP_000549.1 | MFLSFPTTK | 194 | 536.2808 | 349.2076 |
| alpha 1 globin | NP_000549.1 | MFLSFPTTK | 194 | 536.2808 | 446.2604 |
| alpha 1 globin | NP_000549.1 | MFLSFPTTK | 194 | 536.2808 | 593.3288 |
| alpha 1 globin | NP_000549.1 | MFLSFPTTK | 194 | 536.2808 | 680.3608 |
| alpha 1 globin | NP_000549.1 | MFLSFPTTK | 194 | 536.2808 | 793.4449 |
| alpha 1 globin | NP_000549.1 | MFLSFPTTK | 194 | 536.2808 | 940.5133 |
| apolipoprotein B precursor | NP_000375.2 | DNVFDGLVR | 195 | 517.7669 | 387.2709 |
| apolipoprotein B precursor | NP_000375.2 | DNVFDGLVR | 195 | 517.7669 | 444.2924 |
| apolipoprotein B precursor | NP_000375.2 | DNVFDGLVR | 195 | 517.7669 | 559.3193 |
| apolipoprotein B precursor | NP_000375.2 | DNVFDGLVR | 195 | 517.7669 | 706.3877 |
| apolipoprotein B precursor | NP_000375.2 | DNVFDGLVR | 195 | 517.7669 | 805.4561 |
| apolipoprotein B precursor | NP_000375.2 | DNVFDGLVR | 195 | 517.7669 | 919.499 |
| apolipoprotein B precursor | NP_000375.2 | LNGEIQALELPQK | 196 | 726.9065 | 372.2236 |
| apolipoprotein B precursor | NP_000375.2 | LNGEIQALELPQK | 196 | 726.9065 | 485.3076 |
| apolipoprotein B precursor | NP_000375.2 | LNGEIQALELPQK | 196 | 726.9065 | 614.3503 |
| apolipoprotein B precursor | NP_000375.2 | LNGEIQALELPQK | 196 | 726.9065 | 727.4343 |
| apolipoprotein B precursor | NP_000375.2 | LNGEIQALELPQK | 196 | 726.9065 | 798.4714 |
| apolipoprotein B precursor | NP_000375.2 | LNGEIQALELPQK | 196 | 726.9065 | 926.53 |
| apolipoprotein B precursor | NP_000375.2 | LNGEIQALELPQK | 196 | 726.9065 | 1039.614 |
| apolipoprotein B precursor | NP_000375.2 | LNGEIQALELPQK | 196 | 726.9065 | 1168.657 |
| apolipoprotein B precursor | NP_000375.2 | LNGEIQALELPQK | 196 | 726.9065 | 1225.678 |
| apolipoprotein B precursor | NP_000375.2 | LNGEIQALELPQK | 196 | 726.9065 | 1339.721 |
| apolipoprotein B precursor | NP_000375.2 | LEIQSQVDSQHVGHSVLTAK | 197 | 1088.572 | 319.1971 |
| apolipoprotein B precursor | NP_000375.2 | LEIQSQVDSQHVGHSVLTAK | 197 | 1088.572 | 432.2811 |
| apolipoprotein B precursor | NP_000375.2 | LEIQSQVDSQHVGHSVLTAK | 197 | 1088.572 | 531.3495 |
| apolipoprotein B precursor | NP_000375.2 | LEIQSQVDSQHVGHSVLTAK | 197 | 1088.572 | 618.3815 |
| apolipoprotein B precursor | NP_000375.2 | LEIQSQVDSQHVGHSVLTAK | 197 | 1088.572 | 755.4405 |
| apolipoprotein B precursor | NP_000375.2 | LEIQSQVDSQHVGHSVLTAK | 197 | 1088.572 | 812.4619 |
| apolipoprotein B precursor | NP_000375.2 | LEIQSQVDSQHVGHSVLTAK | 197 | 1088.572 | 911.5303 |
| apolipoprotein B precursor | NP_000375.2 | LEIQSQVDSQHVGHSVLTAK | 197 | 1088.572 | 1048.589 |
| apolipoprotein B precursor | NP_000375.2 | LEIQSQVDSQHVGHSVLTAK | 197 | 1088.572 | 1176.648 |
| apolipoprotein B precursor | NP_000375.2 | LEIQSQVDSQHVGHSVLTAK | 197 | 1088.572 | 1263.68 |
| apolipoprotein B precursor | NP_000375.2 | LEIQSQVDSQHVGHSVLTAK | 197 | 1088.572 | 1378.707 |
| apolipoprotein B precursor | NP_000375.2 | LEIQSQVDSQHVGHSVLTAK | 197 | 1088.572 | 1477.775 |
| apolipoprotein H precursor | NP_000033.1 | TFYEPGEEITYSC[Carboxymethyl] | 198 | 1192.541 | 361.2188 |
| | | KPGYVSR | | | |
| apolipoprotein H precursor | NP_000033.1 | TFYEPGEEITYSC[Carboxymethyl] | 198 | 1192.541 | 524.2822 |
| | | KPGYVSR | | | |
| apolipoprotein H precursor | NP_000033.1 | TFYEPGEEITYSC[Carboxymethyl] | 198 | 1192.541 | 581.3036 |
| | | KPGYVSR | | | |
| apolipoprotein H precursor | NP_000033.1 | TFYEPGEEITYSC[Carboxymethyl] | 198 | 1192.541 | 678.3564 |
| | | KPGYVSR | | | |
| apolipoprotein H precursor | NP_000033.1 | TFYEPGEEITYSC[Carboxymethyl] | 198 | 1192.541 | 806.4514 |
| | | KPGYVSR | | | |
| apolipoprotein H precursor | NP_000033.1 | TFYEPGEEITYSC[Carboxymethyl] | 198 | 1192.541 | 967.466 |
| | | KPGYVSR | | | |
| apolipoprotein H precursor | NP_000033.1 | TFYEPGEEITYSC[Carboxymethyl] | 198 | 1192.541 | 1054.498 |
| | | KPGYVSR | | | |
| apolipoprotein H precursor | NP_000033.1 | TFYEPGEEITYSC[Carboxymethyl] | 198 | 1192.541 | 1217.561 |
| | | KPGYVSR | | | |
| apolipoprotein H precursor | NP_000033.1 | TFYEPGEEITYSC[Carboxymethyl] | 198 | 1192.541 | 1318.609 |
| | | KPGYVSR | | | |
| apolipoprotein H precursor | NP_000033.1 | TFYEPGEEITYSC[Carboxymethyl] | 198 | 1192.541 | 1431.693 |
| | | KPGYVSR | | | |
| apolipoprotein A-IV precursor | NP_000473.2 | VNSFFSTFK | 199 | 538.7742 | 395.2283 |
| apolipoprotein A-IV precursor | NP_000473.2 | VNSFFSTFK | 199 | 538.7742 | 482.2604 |
| apolipoprotein A-IV precursor | NP_000473.2 | VNSFFSTFK | 199 | 538.7742 | 629.3288 |
| apolipoprotein A-IV precursor | NP_000473.2 | VNSFFSTFK | 199 | 538.7742 | 776.3972 |
| apolipoprotein A-IV precursor | NP_000473.2 | VNSFFSTFK | 199 | 538.7742 | 863.4293 |
| apolipoprotein A-IV precursor | NP_000473.2 | VNSFFSTFK | 199 | 538.7742 | 977.4722 |
| apolipoprotein H precursor | NP_000033.1 | EHSSLAFWK | 200 | 552.7773 | 480.26 |
| apolipoprotein H precursor | NP_000033.1 | EHSSLAFWK | 200 | 552.7773 | 551.2971 |
| apolipoprotein H precursor | NP_000033.1 | EHSSLAFWK | 200 | 552.7773 | 664.3812 |
| apolipoprotein H precursor | NP_000033.1 | EHSSLAFWK | 200 | 552.7773 | 751.4132 |
| apolipoprotein H precursor | NP_000033.1 | EHSSLAFWK | 200 | 552.7773 | 838.4452 |
| apolipoprotein H precursor | NP_000033.1 | EHSSLAFWK | 200 | 552.7773 | 975.5042 |
| paraoxonase 1 | NP_000437.3 | EVQPVELPNC[Carboxymethyl]NL | 201 | 820.4216 | 359.2647 |
| | | VK | | | |
| paraoxonase 1 | NP_000437.3 | EVQPVELPNC[Carboxymethyl]NL | 201 | 820.4216 | 473.3076 |
| | | VK | | | |
| paraoxonase 1 | NP_000437.3 | EVQPVELPNC[Carboxymethyl]NL | 201 | 820.4216 | 634.3223 |
| | | VK | | | |
| paraoxonase 1 | NP_000437.3 | EVQPVELPNC[Carboxymethyl]NL | 201 | 820.4216 | 748.3652 |
| | | VK | | | |
| paraoxonase 1 | NP_000437.3 | EVQPVELPNC[Carboxymethyl]NL | 201 | 820.4216 | 845.418 |
| | | VK | | | |
| paraoxonase 1 | NP_000437.3 | EVQPVELPNC[Carboxymethyl]NL | 201 | 820.4216 | 958.5021 |
| | | VK | | | |
| paraoxonase 1 | NP_000437.3 | EVQPVELPNC[Carboxymethyl]NL | 201 | 820.4216 | 1087.545 |
| | | VK | | | |
| paraoxonase 1 | NP_000437.3 | EVQPVELPNC[Carboxymethyl]NL | 201 | 820.4216 | 1186.613 |
| | VK | | | | |
| paraoxonase 1 | NP_000437.3 | EVQPVELPNC[Carboxymethyl]NL | 201 | 820.4216 | 1283.666 |
| | | VK | | | |
| paraoxonase 1 | NP_000437.3 | EVQPVELPNC[Carboxymethyl]NL | 201 | 820.4216 | 1411.724 |
| | | VK | | | |
| apolipoprotein B precursor | NP_000375.2 | LVGFIDDAVK | 202 | 538.803 | 317.2178 |
| apolipoprotein B precursor | NP_000375.2 | LVGFIDDAVK | 202 | 538.803 | 432.2447 |
| apolipoprotein B precursor | NP_000375.2 | LVGFIDDAVK | 202 | 538.803 | 547.2717 |
| apolipoprotein B precursor | NP_000375.2 | LVGFIDDAVK | 202 | 538.803 | 660.3557 |
| apolipoprotein B precursor | NP_000375.2 | LVGFIDDAVK | 202 | 538.803 | 807.4241 |
| apolipoprotein B precursor | NP_000375.2 | LVGFIDDAVK | 202 | 538.803 | 864.4456 |
| apolipoprotein B precursor | NP_000375.2 | LVGFIDDAVK | 202 | 538.803 | 963.514 |
| apolipoprotein E precursor | NP_000032.1 | WELALGR | 203 | 422.7374 | 345.2239 |
| apolipoprotein E precursor | NP_000032.1 | WELALGR | 203 | 422.7374 | 416.261 |
| apolipoprotein E precursor | NP_000032.1 | WELALGR | 203 | 422.7374 | 529.3451 |
| apolipoprotein E precursor | NP_000032.1 | WELALGR | 203 | 422.7374 | 658.3877 |
| apolipoprotein B precursor | NP_000375.2 | ALYWVNGQVPDGVSK | 204 | 816.9227 | 333.2127 |
| apolipoprotein B precursor | NP_000375.2 | ALYWVNGQVPDGVSK | 204 | 816.9227 | 390.2342 |
| apolipoprotein B precursor | NP_000375.2 | ALYWVNGQVPDGVSK | 204 | 816.9227 | 505.2611 |
| apolipoprotein B precursor | NP_000375.2 | ALYWVNGQVPDGVSK | 204 | 816.9227 | 602.3138 |
| apolipoprotein B precursor | NP_000375.2 | ALYWVNGQVPDGVSK | 204 | 816.9227 | 701.3823 |
| apolipoprotein B precursor | NP_000375.2 | ALYWVNGQVPDGVSK | 204 | 816.9227 | 829.4409 |
| apolipoprotein B precursor | NP_000375.2 | ALYWVNGQVPDGVSK | 204 | 816.9227 | 886.4623 |
| apolipoprotein B precursor | NP_000375.2 | ALYWVNGQVPDGVSK | 204 | 816.9227 | 1000.505 |
| apolipoprotein B precursor | NP_000375.2 | ALYWVNGQVPDGVSK | 204 | 816.9227 | 1099.574 |
| apolipoprotein B precursor | NP_000375.2 | ALYWVNGQVPDGVSK | 204 | 816.9227 | 1285.653 |
| apolipoprotein B precursor | NP_000375.2 | ALYWVNGQVPDGVSK | 204 | 816.9227 | 1448.716 |
| apolipoprotein H precursor | NP_000033.1 | ATVVYQGER | 205 | 511.7669 | 652.3044 |
| apolipoprotein B precursor | NP_000375.2 | HIYAISSAALSASYK | 206 | 791.4172 | 397.2076 |
| apolipoprotein B precursor | NP_000375.2 | HIYAISSAALSASYK | 206 | 791.4172 | 468.2447 |
| apolipoprotein B precursor | NP_000375.2 | HIYAISSAALSASYK | 206 | 791.4172 | 555.2767 |
| apolipoprotein B precursor | NP_000375.2 | HIYAISSAALSASYK | 206 | 791.4172 | 668.3608 |
| apolipoprotein B precursor | NP_000375.2 | HIYAISSAALSASYK | 206 | 791.4172 | 739.3979 |
| apolipoprotein B precursor | NP_000375. | HIYAISSAALSASYK | 206 | 791.4172 | 810.4351 |
| apolipoprotein B precursor | NP_000375.2 | HIYAISSAALSASYK | 206 | 791.4172 | 897.4671 |
| apolipoprotein B precursor | NP_000375.2 | HIYAISSAALSASYK | 206 | 791.4172 | 984.4991 |
| apolipoprotein B precursor | NP_000375.2 | HIYAISSAALSASYK | 206 | 791.4172 | 1097.583 |
| apolipoprotein B precursor | NP_000375.2 | HIYAISSAALSASYK | 206 | 791.4172 | 1168.62 |
| apolipoprotein B precursor | NP_000375.2 | HIYAISSAALSASYK | 206 | 791.4172 | 1331.684 |
| apolipoprotein B precursor | NP_000375.2 | HIYAISSAALSASYK | 206 | 791.4172 | 1444.768 |
| alpha-2-macroglobulin precursor | NP_000005.2 | AHTSFQISLSVSYTGSR | 207 | 920.963 | 319.1719 |
| alpha-2-macroglobulin precursor | NP_000005.2 | AHTSFQISLSVSYTGSR | 207 | 920.963 | 420.2196 |
| alpha-2-macroglobulin precursor | NP_000005.2 | AHTSFQISLSVSYTGSR | 207 | 920.963 | 583.2829 |
| alpha-2-macroglobulin precursor | NP_000005.2 | AHTSFQISLSVSYTGSR | 207 | 920.963 | 670.3149 |
| alpha-2-macroglobulin precursor | NP_000005.2 | AHTSFQISLSVSYTGSR | 207 | 920.963 | 769.3834 |
| alpha-2-macroglobulin precursor | NP_000005.2 | AHTSFQISLSVSYTGSR | 207 | 920.963 | 856.4153 |
| alpha-2-macroglobulin precursor | NP_000005.2 | AHTSFQISLSVSYTGSR | 207 | 920.963 | 969.4995 |
| alpha-2-macroglobulin precursor | NP_000005.2 | AHTSFQISLSVSYTGSR | 207 | 920.963 | 1056.531 |
| alpha-2-macroglobulin precursor | NP_000005.2 | AHTSFQISLSVSYTGSR | 207 | 920.963 | 1169.615 |
| alpha-2-macroglobulin precursor | NP_000005.2 | AHTSFQISLSVSYTGSR | 207 | 920.963 | 1297.674 |
| alpha-2-macroglobulin precursor | NP_000005.2 | AHTSFQISLSVSYTGSR | 207 | 920.963 | 1444.743 |
| apolipoprotein B precursor | NP_000375.2 | NIILPVYDK | 208 | 537.8134 | 425.2025 |
| apolipoprotein B precursor | NP_000375.2 | NIILPVYDK | 208 | 537.8134 | 524.2709 |
| apolipoprotein B precursor | NP_000375.2 | NIILPVYDK | 208 | 537.8134 | 621.3237 |
| apolipoprotein B precursor | NP_000375.2 | NIILPVYDK | 208 | 537.8134 | 734.4078 |
| apolipoprotein B precursor | NP_000375.2 | NIILPVYDK | 208 | 537.8134 | 847.4918 |
| apolipoprotein B precursor | NP_000375.2 | NIILPVYDK | 208 | 537.8134 | 960.5759 |
| apolipoprotein B precursor | NP_000375.2 | IEIPLPFGGK | 209 | 535.8159 | 261.1552 |
| apolipoprotein B precursor | NP_000375.2 | IEIPLPFGGK | 209 | 535.8159 | 408.2236 |
| apolipoprotein B precursor | NP_000375.2 | IEIPLPFGGK | 209 | 535.8159 | 505.2764 |
| apolipoprotein B precursor | NP_000375.2 | IEIPLPFGGK | 209 | 535.8159 | 618.3604 |
| apolipoprotein B precursor | NP_000375.2 | IEIPLPFGGK | 209 | 535.8159 | 715.4132 |
| apolipoprotein B precursor | NP_000375.2 | IEIPLPFGGK | 209 | 535.8159 | 828.4973 |
| apolipoprotein B precursor | NP_000375.2 | IEIPLPFGGK | 209 | 535.8159 | 957.5399 |
| apolipoprotein C-I precursor | NP_001636.1 | MREWFSETFQK | 210 | 744.8506 | 422.2393 |
| apolipoprotein C-I precursor | NP_001636.1 | MREWFSETFQK | 210 | 744.8506 | 523.2869 |
| apolipoprotein C-I precursor | NP_001636.1 | MREWFSETFQK | 210 | 744.8506 | 652.3295 |
| apolipoprotein C-I precursor | NP_001636.1 | MREWFSETFQK | 210 | 744.8506 | 739.3616 |
| apolipoprotein C-I precursor | NP_001636.1 | MREWFSETFQK | 210 | 744.8506 | 886.4299 |
| apolipoprotein C-I precursor | NP_001636.1 | MREWFSETFQK | 210 | 744.8506 | 1072.509 |
| apolipoprotein C-I precursor | NP_001636.1 | MREWFSETFQK | 210 | 744.8506 | 1201.552 |
| apolipoprotein C-I precursor | NP_001636.1 | MREWFSETFQK | 210 | 744.8506 | 1357.653 |
| apolipoprotein C-I precursor | NP_001636.1 | MREWFSETFQK | 210 | 752.8481 | 422.2393 |
| apolipoprotein C-I precursor | NP_001636.1 | MREWFSETFQK | 210 | 752.8481 | 523.2869 |
| apolipoprotein C-I precursor | NP_001636.1 | MREWFSETFQK | 210 | 752.8481 | 652.3295 |
| apolipoprotein C-I precursor | NP_001636.1 | MREWFSETFQK | 210 | 752.8481 | 739.3616 |
| apolipoprotein C-I precursor | NP_001636.1 | MREWFSETFQK | 210 | 752.8481 | 886.4299 |
| apolipoprotein C-I precursor | NP_001636.1 | MREWFSETFQK | 210 | 752.8481 | 1072.509 |
| apolipoprotein C-I precursor | NP_001636.1 | MREWFSETFQK | 210 | 752.8481 | 1201.552 |
| apolipoprotein C-I precursor | NP_001636.1 | MREWFSETFQK | 210 | 752.8481 | 1357.653 |
| apolipoprotein B precursor | NP_000375.2 | ATVAVYLESLQDTK | 211 | 769.4091 | 363.1869 |
| apolipoprotein B precursor | NP_000375.2 | ATVAVYLESLQDTK | 211 | 769.4091 | 491.2455 |
| apolipoprotein B precursor | NP_000375.2 | ATVAVYLESLQDTK | 211 | 769.4091 | 604.3295 |
| apolipoprotein B precursor | NP_000375.2 | ATVAVYLESLQDTK | 211 | 769.4091 | 691.3616 |
| apolipoprotein B precursor | NP_000375.2 | ATVAVYLESLQDTK | 211 | 769.4091 | 820.4041 |
| apolipoprotein B precursor | NP_000375.2 | ATVAVYLESLQDTK | 211 | 769.4091 | 933.4882 |
| apolipoprotein B precursor | NP_000375.2 | ATVAVYLESLQDTK | 211 | 769.4091 | 1096.552 |
| apolipoprotein B precursor | NP_000375.2 | ATVAVYLESLQDTK | 211 | 769.4091 | 1195.62 |
| apolipoprotein B precursor | NP_000375.2 | ATVAVYLESLQDTK | 211 | 769.4091 | 1266.657 |
| apolipoprotein B precursor | NP_000375.2 | ATVAVYLESLQDTK | 211 | 769.4091 | 1365.725 |
| apolipoprotein B precursor | NP_000375.2 | ATVAVYLESLQDTK | 211 | 769.4091 | 1466.773 |
| pregnancy-zone protein | NP_002855.1 | QQNAQGGFSSTQDTVVALHALSR | 212 | 1208.104 | 375.2345 |
| pregnancy-zone protein | NP_002855.1 | QQNAQGGFSSTQDTVVALHALSR | 212 | 1208.104 | 446.2716 |
| pregnancy-zone protein | NP_002855.1 | QQNAQGGFSSTQDTVVALHALSR | 212 | 1208.104 | 583.3305 |
| pregnancy-zone protein | NP_002855.1 | QQNAQGGFSSTQDTVVALHALSR | 212 | 1208.104 | 696.4146 |
| pregnancy-zone protein | NP_002855.1 | QQNAQGGFSSTQDTVVALHALSR | 212 | 1208.104 | 767.4517 |
| pregnancy-zone protein | NP_002855.1 | QQNAQGGFSSTQDTVVALHALSR | 212 | 1208.104 | 866.5201 |
| pregnancy-zone protein | NP_002855.1 | QQNAQGGFSSTQDTVVALHALSR | 212 | 1208.104 | 965.5885 |
| pregnancy-zone protein | NP_002855.1 | QQNAQGGFSSTQDTVVALHALSR | 212 | 1208.104 | 1066.636 |
| pregnancy-zone protein | NP_002855.1 | QQNAQGGFSSTQDTVVALHALSR | 212 | 1208.104 | 1181.663 |
| pregnancy-zone protein | NP_002855.1 | QQNAQGGFSSTQDTVVALHALSR | 212 | 1208.104 | 1309.722 |
| pregnancy-zone protein | NP_002855.1 | QQNAQGGFSSTQDTVVALHALSR | 212 | 1208.104 | 1410.769 |
| pregnancy-zone protein | NP_002855.1 | QQNAQGGFSSTQDTVVALHALSR | 212 | 1208.104 | 1497.801 |
| paraoxonase 1 | NP_000437.3 | LLIGTVFHK | 213 | 514.3186 | 431.2396 |
| paraoxonase 1 | NP_000437.3 | LLIGTVFHK | 213 | 514.3186 | 530.308 |
| paraoxonase 1 | NP_000437.3 | LLIGTVFHK | 213 | 514.3186 | 631.3557 |
| paraoxonase 1 | NP_000437.3 | LLIGTVFHK | 213 | 514.3186 | 688.3771 |
| paraoxonase 1 | NP_000437.3 | LLIGTVFHK | 213 | 514.3186 | 801.4612 |
| paraoxonase 1 | NP_000437.3 | LLIGTVFHK | 213 | 514.3186 | 914.5453 |
| apolipoprotein B precursor | NP_000375.2 | VSALLTPAEQTGTWK | 214 | 801.4303 | 434.2393 |
| apolipoprotein B precursor | NP_000375.2 | VSALLTPAEQTGTWK | 214 | 801.4303 | 491.2607 |
| apolipoprotein B precursor | NP_000375.2 | VSALLTPAEQTGTWK | 214 | 801.4303 | 592.3084 |
| apolipoprotein B precursor | NP_000375.2 | VSALLTPAEQTGTWK | 214 | 801.4303 | 720.3669 |
| apolipoprotein B precursor | NP_000375.2 | VSALLTPAEQTGTWK | 214 | 801.4303 | 849.4095 |
| apolipoprotein B precursor | NP_000375.2 | VSALLTPAEQTGTWK | 214 | 801.4303 | 920.4467 |
| apolipoprotein B precursor | NP_000375.2 | VSALLTPAEQTGTWK | 214 | 801.4303 | 1017.499 |
| apolipoprotein B precursor | NP_000375.2 | VSALLTPAEQTGTWK | 214 | 801.4303 | 1118.547 |
| apolipoprotein B precursor | NP_000375.2 | VSALLTPAEQTGTWK | 214 | 801.4303 | 1231.631 |
| apolipoprotein B precursor | NP_000375.2 | VSALLTPAEQTGTWK | 214 | 801.4303 | 1344.715 |
| apolipoprotein B precursor | NP_000375.2 | VSALLTPAEQTGTWK | 214 | 801.4303 | 1415.752 |
| apolipoprotein C-I precursor | NP_001636.1 | EWFSETFQK | 215 | 601.2798 | 422.2393 |
| apolipoprotein C-I precursor | NP_001636.1 | EWFSETFQK | 215 | 601.2798 | 523.2869 |
| apolipoprotein C-I precursor | NP_001636.1 | EWFSETFQK | 215 | 601.2798 | 652.3295 |
| apolipoprotein C-I precursor | NP_001636.1 | EWFSETFQK | 215 | 601.2798 | 739.3616 |
| apolipoprotein C-I precursor | NP_001636.1 | EWFSETFQK | 215 | 601.2798 | 886.4299 |
| apolipoprotein C-I precursor | NP_001636.1 | EWFSETFQK | 215 | 601.2798 | 1072.509 |
| apolipoprotein B precursor | NP_000375.2 | VNQNLVYESGSLNFSK | 216 | 899.9521 | 381.2127 |
| apolipoprotein B precursor | NP_000375.2 | VNQNLVYESGSLNFSK | 216 | 899.9521 | 495.2556 |
| apolipoprotein B precursor | NP_000375.2 | VNQNLVYESGSLNFSK | 216 | 899.9521 | 608.3397 |
| apolipoprotein B precursor | NP_000375.2 | VNQNLVYESGSLNFSK | 216 | 899.9521 | 695.3717 |
| apolipoprotein B precursor | NP_000375.2 | VNQNLVYESGSLNFSK | 216 | 899.9521 | 752.3932 |
| apolipoprotein B precursor | NP_000375.2 | VNQNLVYESGSLNFSK | 216 | 899.9521 | 839.4252 |
| apolipoprotein B precursor | NP_000375.2 | VNQNLVYESGSLNFSK | 216 | 899.9521 | 968.4678 |
| apolipoprotein B precursor | NP_000375.2 | VNQNLVYESGSLNFSK | 216 | 899.9521 | 1131.531 |
| apolipoprotein B precursor | NP_000375.2 | VNQNLVYESGSLNFSK | 216 | 899.9521 | 1230.599 |
| apolipoprotein B precursor | NP_000375.2 | VNQNLVYESGSLNFSK | 216 | 899.9521 | 1343.684 |
| apolipoprotein B precursor | NP_000375.2 | VNQNLVYESGSLNFSK | 216 | 899.9521 | 1457.727 |
| histidine-rich glycoprotein precursor | NP_000403.1 | GGEGTGYFVDFSVR | 217 | 745.8492 | 361.2188 |
| histidine-rich glycoprotein precursor | NP_000403.1 | GGEGTGYFVDFSVR | 217 | 745.8492 | 508.2873 |
| histidine-rich glycoprotein precursor | NP_000403.1 | GGEGTGYFVDFSVR | 217 | 745.8492 | 623.3142 |
| histidine-rich glycoprotein precursor | NP_000403.1 | GGEGTGYFVDFSVR | 217 | 745.8492 | 722.3826 |
| histidine-rich glycoprotein precursor | NP_000403.1 | GGEGTGYFVDFSVR | 217 | 745.8492 | 869.451 |
| histidine-rich glycoprotein precursor | NP_000403.1 | GGEGTGYFVDFSVR | 217 | 745.8492 | 1032.514 |
| histidine-rich glycoprotein precursor | NP_000403.1 | GGEGTGYFVDFSVR | 217 | 745.8492 | 1089.536 |
| histidine-rich glycoprotein precursor | NP_000403.1 | GGEGTGYFVDFSVR | 217 | 745.8492 | 1190.583 |
| histidine-rich glycoprotein precursor | NP_000403.1 | GGEGTGYFVDFSVR | 217 | 745.8492 | 1247.605 |
| histidine-rich glycoprotein precursor | NP_000403.1 | GGEGTGYFVDFSVR | 217 | 745.8492 | 1376.648 |
| histidine-rich glycoprotein precursor | NP_000403.1 | GGEGTGYFVDFSVR | 217 | 745.8492 | 1433.669 |
| apolipoprotein B precursor | NP_000375.2 | DEPTYILNIK | 218 | 603.3243 | 374.2393 |
| apolipoprotein B precursor | NP_000375.2 | DEPTYILNIK | 218 | 603.3243 | 487.3233 |
| apolipoprotein B precursor | NP_000375.2 | DEPTYILNIK | 218 | 603.3243 | 600.4073 |
| apolipoprotein B precursor | NP_000375.2 | DEPTYILNIK | 218 | 603.3243 | 763.4707 |
| apolipoprotein B precursor | NP_000375.2 | DEPTYILNIK | 218 | 603.3243 | 864.5184 |
| apolipoprotein B precursor | NP_000375.2 | DEPTYILNIK | 218 | 603.3243 | 961.5712 |
| apolipoprotein B precursor | NP_000375.2 | DEPTYILNIK | 218 | 603.3243 | 1090.614 |
| apolipoprotein B precursor | NP_000375.2 | TEVIPPLIENR | 219 | 640.8641 | 418.2039 |
| apolipoprotein B precursor | NP_000375.2 | TEVIPPLIENR | 219 | 640.8641 | 531.288 |
| apolipoprotein B precursor | NP_000375.2 | TEVIPPLIENR | 219 | 640.8641 | 644.3721 |
| apolipoprotein B precursor | NP_000375.2 | TEVIPPLIENR | 219 | 640.8641 | 741.4248 |
| apolipoprotein B precursor | NP_000375.2 | TEVIPPLIENR | 219 | 640.8641 | 838.4776 |
| apolipoprotein B precursor | NP_000375.2 | TEVIPPLIENR | 219 | 640.8641 | 951.5616 |
| apolipoprotein B precursor | NP_000375.2 | TEVIPPLIENR | 219 | 640.8641 | 1050.63 |
| apolipoprotein B precursor | NP_000375.2 | TEVIPPLIENR | 219 | 640.8641 | 1179.673 |
| serum amyloid A4; constitutive | NP_006503.1 | EALQGVGDMGR | 220 | 566.7744 | 634.2972 |
| alpha 1 globin | NP_000549.1 | TYFPHFDLSHGSAQVK | 221 | 917.4496 | 374.2393 |
| alpha 1 globin | NP_000549.1 | TYFPHFDLSHGSAQVK | 221 | 917.4496 | 445.2764 |
| alpha 1 globin | NP_000549.1 | TYFPHFDLSHGSAQVK | 221 | 917.4496 | 532.3084 |
| alpha 1 globin | NP_000549.1 | TYFPHFDLSHGSAQVK | 221 | 917.4496 | 589.3298 |
| alpha 1 globin | NP_000549.1 | TYFPHFDLSHGSAQVK | 221 | 917.4496 | 726.3888 |
| alpha 1 globin | NP_000549.1 | TYFPHFDLSHGSAQVK | 221 | 917.4496 | 813.4208 |
| alpha 1 globin | NP_000549.1 | TYFPHFDLSHGSAQVK | 221 | 917.4496 | 926.5049 |
| alpha 1 globin | NP_000549.1 | TYFPHFDLSHGSAQVK | 221 | 917.4496 | 1041.532 |
| alpha 1 globin | NP_000549.1 | TYFPHFDLSHGSAQVK | 221 | 917.4496 | 1325.659 |
| alpha 1 globin | NP_000549.1 | TYFPHFDLSHGSAQVK | 221 | 917.4496 | 1422.712 |
| ceruloplasmin precursor | NP_000087.1 | ALYLQYTDETFR | 222 | 760.375 | 423.2345 |
| ceruloplasmin precursor | NP_000087.1 | ALYLQYTDETFR | 222 | 760.375 | 552.2771 |
| ceruloplasmin precursor | NP_000087.1 | ALYLQYTDETFR | 222 | 760.375 | 667.304 |
| ceruloplasmin precursor | NP_000087.1 | ALYLQYTDETFR | 222 | 760.375 | 768.3517 |
| ceruloplasmin precursor | NP_000087.1 | ALYLQYTDETFR | 222 | 760.375 | 931.415 |
| ceruloplasmin precursor | NP_000087.1 | ALYLQYTDETFR | 222 | 760.375 | 1059.474 |
| ceruloplasmin precursor | NP_000087.1 | ALYLQYTDETFR | 222 | 760.375 | 1172.558 |
| ceruloplasmin precursor | NP_000087.1 | ALYLQYTDETFR | 222 | 760.375 | 1335.621 |
| ceruloplasmin precursor | NP_000087.1 | ALYLQYTDETFR | 222 | 760.375 | 1448.705 |
| apolipoprotein H precursor | NP_000033.1 | VC[Carboxymethyl]PFAGILENGA | 223 | 752.3848 | 345.2239 |
| | | VR | | | |
| apolipoprotein H precursor | NP_000033.1 | VC[Carboxymethyl]PFAGILENGA | 223 | 752.3848 | 402.2454 |
| | | VR | | | |
| apolipoprotein H precursor | NP_000033.1 | VC[Carboxymethyl]PFAGILENGA | 223 | 752.3848 | 516.2883 |
| | | VR | | | |
| apolipoprotein H precursor | NP_000033.1 | VC[Carboxymethyl]PFAGILENGA | 223 | 752.3848 | 645.3309 |
| | | VR | | | |
| apolipoprotein H precursor | NP_000033.1 | VC[Carboxymethyl]PFAGILENGA | 223 | 752.3848 | 758.415 |
| | | VR | | | |
| apolipoprotein H precursor | NP_000033.1 | VC[Carboxymethyl]PFAGILENGA | 223 | 752.3848 | 871.499 |
| | | VR | | | |
| apolipoprotein H precursor | NP_000033.1 | VC[Carboxymethyl]PFAGILENGA | 223 | 752.3848 | 928.5205 |
| | | VR | | | |
| apolipoprotein H precursor | NP_000033.1 | VC[Carboxymethyl]PFAGILENGA | 223 | 752.3848 | 999.5576 |
| | | VR | | | |
| apolipoprotein H precursor | NP_000033.1 | VC[Carboxymethyl]PFAGILENGA | 223 | 752.3848 | 1146.626 |
| | | VR | | | |
| apolipoprotein H precursor | NP_000033.1 | VC[Carboxymethyl]PFAGILENGA | 223 | 752.3848 | 1243.679 |
| | | VR | | | |
| apolipoprotein H precursor | NP_000033.1 | VC[Carboxymethyl]PFAGILENGA | 223 | 752.3848 | 1404.693 |
| | | VR | | | |
| apolipoprotein A-IV precursor | NP_000473.2 | LGPHAGDVEGHLSFLEK | 224 | 903.4626 | 389.2389 |
| apolipoprotein A-IV precursor | NP_000473.2 | LGPHAGDVEGHLSFLEK | 224 | 903.4626 | 536.3073 |
| apolipoprotein A-IV precursor | NP_000473.2 | LGPHAGDVEGHLSFLEK | 224 | 903.4626 | 623.3394 |
| apolipoprotein A-IV precursor | NP_000473.2 | LGPHAGDVEGHLSFLEK | 224 | 903.4626 | 736.4234 |
| apolipoprotein A-IV precursor | NP_000473.2 | LGPHAGDVEGHLSFLEK | 224 | 903.4626 | 873.4823 |
| apolipoprotein A-IV precursor | NP_000473.2 | LGPHAGDVEGHLSFLEK | 224 | 903.4626 | 930.5038 |
| apolipoprotein A-IV precursor | NP_000473.2 | LGPHAGDVEGHLSFLEK | 224 | 903.4626 | 1059.546 |
| apolipoprotein A-IV precursor | NP_000473.2 | LGPHAGDVEGHLSFLEK | 224 | 903.4626 | 1158.615 |
| apolipoprotein A-IV precursor | NP_000473.2 | LGPHAGDVEGHLSFLEK | 224 | 903.4626 | 1273.642 |
| apolipoprotein A-IV precursor | NP_000473.2 | LGPHAGDVEGHLSFLEK | 224 | 903.4626 | 1330.663 |
| apolipoprotein A-IV precursor | NP_000473.2 | LGPHAGDVEGHLSFLEK | 224 | 903.4626 | 1401.7 |
| apolipoprotein B precursor | NP_000375.2 | ADSVVDLLSYNVQGSGETTYDHK | 225 | 1249.588 | 399.1981 |
| apolipoprotein B precursor | NP_000375.2 | ADSVVDLLSYNVQGSGETTYDHK | 225 | 1249.588 | 562.2614 |
| apolipoprotein B precursor | NP_000375.2 | ADSVVDLLSYNVQGSGETTYDHK | 225 | 1249.588 | 663.3091 |
| apolipoprotein B precursor | NP_000375.2 | ADSVVDLLSYNVQGSGETTYDHK | 225 | 1249.588 | 764.3568 |
| apolipoprotein B precursor | NP_000375.2 | ADSVVDLLSYNVQGSGETTYDHK | 225 | 1249.588 | 893.3994 |
| apolipoprotein B precursor | NP_000375.2 | ADSVVDLLSYNVQGSGETTYDHK | 225 | 1249.588 | 950.4208 |
| apolipoprotein B precursor | NP_000375.2 | ADSVVDLLSYNVQGSGETTYDHK | 225 | 1249.588 | 1037.453 |
| apolipoprotein B precursor | NP_000375.2 | ADSVVDLLSYNVQGSGETTYDHK | 225 | 1249.588 | 1094.474 |
| apolipoprotein B precursor | NP_000375.2 | ADSVVDLLSYNVQGSGETTYDHK | 225 | 1249.588 | 1222.533 |
| apolipoprotein B precursor | NP_000375.2 | ADSVVDLLSYNVQGSGETTYDHK | 225 | 1249.588 | 1321.601 |
| apolipoprotein B precursor | NP_000375.2 | ADSVVDLLSYNVQGSGETTYDHK | 225 | 1249.588 | 1435.644 |
| apolipoprotein C-III precursor | NP_000031.1 | GWVTDGFSSLK | 226 | 598.801 | 347.2283 |
| apolipoprotein C-III precursor | NP_000031.1 | GWVTDGFSSLK | 226 | 598.801 | 434.2604 |
| apolipoprotein C-III precursor | NP_000031.1 | GWVTDGFSSLK | 226 | 598.801 | 581.3288 |
| apolipoprotein C-III precursor | NP_000031.1 | GWVTDGFSSLK | 226 | 598.801 | 638.3503 |
| apolipoprotein C-III precursor | NP_000031.1 | GWVTDGFSSLK | 226 | 598.801 | 753.3772 |
| apolipoprotein C-III precursor | NP_000031.1 | GWVTDGFSSLK | 226 | 598.801 | 953.4933 |
| apolipoprotein C-III precursor | NP_000031.1 | GWVTDGFSSLK | 226 | 598.801 | 1139.573 |
| apolipoprotein B precursor | NP_000375.2 | EFQVPTFTIPK | 227 | 653.8557 | 357.2491 |
| apolipoprotein B precursor | NP_000375.2 | EFQVPTFTIPK | 227 | 653.8557 | 458.2968 |
| apolipoprotein B precursor | NP_000375.2 | EFQVPTFTIPK | 227 | 653.8557 | 605.3652 |
| apolipoprotein B precursor | NP_000375.2 | EFQVPTFTIPK | 227 | 653.8557 | 706.4128 |
| apolipoprotein B precursor | NP_000375.2 | EFQVPTFTIPK | 227 | 653.8557 | 803.4656 |
| apolipoprotein B precursor | NP_000375.2 | EFQVPTFTIPK | 227 | 653.8557 | 902.5341 |
| apolipoprotein B precursor | NP_000375.2 | EFQVPTFTIPK | 227 | 653.8557 | 1030.593 |
| apolipoprotein B precursor | NP_000375.2 | EFQVPTFTIPK | 227 | 653.8557 | 1177.661 |
| apolipoprotein B precursor | NP_000375.2 | NIQEYLSILTDPDGK | 228 | 853.4358 | 319.1607 |
| apolipoprotein B precursor | NP_000375.2 | NIQEYLSILTDPDGK | 228 | 853.4358 | 416.2134 |
| apolipoprotein B precursor | NP_000375.2 | NIQEYLSILTDPDGK | 228 | 853.4358 | 531.2404 |
| apolipoprotein B precursor | NP_000375.2 | NIQEYLSILTDPDGK | 228 | 853.4358 | 632.288 |
| apolipoprotein B precursor | NP_000375.2 | NIQEYLSILTDPDGK | 228 | 853.4358 | 745.3721 |
| apolipoprotein B precursor | NP_000375.2 | NIQEYLSILTDPDGK | 228 | 853.4358 | 858.4562 |
| apolipoprotein B precursor | NP_000375.2 | NIQEYLSILTDPDGK | 228 | 853.4358 | 945.4882 |
| apolipoprotein B precursor | NP_000375.2 | NIQEYLSILTDPDGK | 228 | 853.4358 | 1058.572 |
| apolipoprotein B precursor | NP_000375.2 | NIQEYLSILTDPDGK | 228 | 853.4358 | 1221.636 |
| apolipoprotein B precursor | NP_000375.2 | NIQEYLSILTDPDGK | 228 | 853.4358 | 1350.678 |
| apolipoprotein B precursor | NP_000375.2 | NIQEYLSILTDPDGK | 228 | 853.4358 | 1478.737 |
| ceruloplasmin precursor | NP_000087.1 | GAYPLSIEPIGVR | 229 | 686.3852 | 331.2083 |
| ceruloplasmin precursor | NP_000087.1 | GAYPLSIEPIGVR | 229 | 686.3852 | 444.2924 |
| ceruloplasmin precursor | NP_000087.1 | GAYPLSIEPIGVR | 229 | 686.3852 | 670.3877 |
| ceruloplasmin precursor | NP_000087.1 | GAYPLSIEPIGVR | 229 | 686.3852 | 783.4717 |
| ceruloplasmin precursor | NP_000087.1 | GAYPLSIEPIGVR | 229 | 686.3852 | 870.5038 |
| ceruloplasmin precursor | NP_000087.1 | GAYPLSIEPIGVR | 229 | 686.3852 | 983.5878 |
| ceruloplasmin precursor | NP_000087.1 | GAYPLSIEPIGVR | 229 | 686.3852 | 1080.641 |
| ceruloplasmin precursor | NP_000087.1 | GAYPLSIEPIGVR | 229 | 686.3852 | 1243.704 |
| ceruloplasmin precursor | NP_000087.1 | GAYPLSIEPIGVR | 229 | 686.3852 | 1314.741 |
| apolipoprotein A-IV precursor | NP_000473.2 | DKVNSFFSTFK | 230 | 660.3351 | 395.2283 |
| apolipoprotein A-IV precursor | NP_000473.2 | DKVNSFFSTFK | 230 | 660.3351 | 482.2604 |
| apolipoprotein A-IV precursor | NP_000473.2 | DKVNSFFSTFK | 230 | 660.3351 | 629.3288 |
| apolipoprotein A-IV precursor | NP_000473.2 | DKVNSFFSTFK | 230 | 660.3351 | 776.3972 |
| apolipoprotein A-IV precursor | NP_000473.2 | DKVNSFFSTFK | 230 | 660.3351 | 863.4293 |
| apolipoprotein A-IV precursor | NP_000473.2 | DKVNSFFSTFK | 230 | 660.3351 | 977.4722 |
| apolipoprotein A-IV precursor | NP_000473.2 | DKVNSFFSTFK | 230 | 660.3351 | 1076.541 |
| apolipoprotein A-IV precursor | NP_000473.2 | DKVNSFFSTFK | 230 | 660.3351 | 1204.635 |
| apolipoprotein A-I preproprotein | NP_000030.1 | LLDNWDSVTSTFSK | 231 | 806.8963 | 381.2127 |
| apolipoprotein A-I preproprotein | NP_000030.1 | LLDNWDSVTSTFSK | 231 | 806.8963 | 482.2604 |
| apolipoprotein A-I preproprotein | NP_000030.1 | LLDNWDSVTSTFSK | 231 | 806.8963 | 569.2924 |
| apolipoprotein A-I preproprotein | NP_000030.1 | LLDNWDSVTSTFSK | 231 | 806.8963 | 670.3401 |
| apolipoprotein A-I preproprotein | NP_000030.1 | LLDNWDSVTSTFSK | 231 | 806.8963 | 769.4085 |
| apolipoprotein A-I preproprotein | NP_000030.1 | LLDNWDSVTSTFSK | 231 | 806.8963 | 856.4406 |
| apolipoprotein A-I preproprotein | NP_000030.1 | LLDNWDSVTSTFSK | 231 | 806.8963 | 1157.547 |
| apolipoprotein A-I preproprotein | NP_000030.1 | LLDNWDSVTSTFSK | 231 | 806.8963 | 1271.59 |
| apolipoprotein A-I preproprotein | NP_000030.1 | LLDNWDSVTSTFSK | 231 | 806.8963 | 1386.617 |
| apolipoprotein A-I preproprotein | NP_000030.1 | LLDNWDSVTSTFSK | 231 | 806.8963 | 1499.701 |
| apolipoprotein B precursor | NP_000375.2 | LPQQANDYLNSFNWER | 232 | 997.9714 | 490.2403 |
| apolipoprotein B precursor | NP_000375.2 | LPQQANDYLNSFNWER | 232 | 997.9714 | 604.2833 |
| apolipoprotein B precursor | NP_000375.2 | LPQQANDYLNSFNWER | 232 | 997.9714 | 751.3516 |
| apolipoprotein B precursor | NP_000375.2 | LPQQANDYLNSFNWER | 232 | 997.9714 | 838.3837 |
| apolipoprotein B precursor | NP_000375.2 | LPQQANDYLNSFNWER | 232 | 997.9714 | 952.4266 |
| apolipoprotein B precursor | NP_000375.2 | LPQQANDYLNSFNWER | 232 | 997.9714 | 1065.511 |
| apolipoprotein B precursor | NP_000375.2 | LPQQANDYLNSFNWER | 232 | 997.9714 | 1228.574 |
| apolipoprotein B precursor | NP_000375.2 | LPQQANDYLNSFNWER | 232 | 997.9714 | 1343.601 |
| apolipoprotein B precursor | NP_000375. | LPQQANDYLNSFNWER | 232 | 997.9714 | 1457.644 |
| apolipoprotein C-I precursor | NP_001636.1 | LKEFGNTLEDK | 233 | 647.3379 | 1052.489 |
| apolipoprotein B precursor | NP_000375.2 | NTLELSNGVIVK | 234 | 643.8694 | 359.2647 |
| apolipoprotein B precursor | NP_000375.2 | NTLELSNGVIVK | 234 | 643.8694 | 458.3332 |
| apolipoprotein B precursor | NP_000375.2 | NTLELSNGVIVK | 234 | 643.8694 | 515.3546 |
| apolipoprotein B precursor | NP_000375.2 | NTLELSNGVIVK | 234 | 643.8694 | 629.3975 |
| apolipoprotein B precursor | NP_000375.2 | NTLELSNGVIVK | 234 | 643.8694 | 716.4296 |
| apolipoprotein B precursor | NP_000375.2 | NTLELSNGVIVK | 234 | 643.8694 | 829.5136 |
| apolipoprotein B precursor | NP_000375.2 | NTLELSNGVIVK | 234 | 643.8694 | 958.5562 |
| apolipoprotein B precursor | NP_000375.2 | NTLELSNGVIVK | 234 | 643.8694 | 1071.64 |
| apolipoprotein B precursor | NP_000375.2 | NTLELSNGVIVK | 234 | 643.8694 | 1172.688 |
| apolipoprotein B precursor | NP_000375.2 | ASGSLPYTQTLQDHLNSLK | 235 | 1037.034 | 347.2283 |
| apolipoprotein B precursor | NP_000375.2 | ASGSLPYTQTLQDHLNSLK | 235 | 1037.034 | 461.2713 |
| apolipoprotein B precursor | NP_000375.2 | ASGSLPYTQTLQDHLNSLK | 235 | 1037.034 | 574.3553 |
| apolipoprotein B precursor | NP_000375.2 | ASGSLPYTQTLQDHLNSLK | 235 | 1037.034 | 711.4142 |
| apolipoprotein B precursor | NP_000375.2 | ASGSLPYTQTLQDHLNSLK | 235 | 1037.034 | 826.4412 |
| apolipoprotein B precursor | NP_000375.2 | ASGSLPYTQTLQDHLNSLK | 235 | 1037.034 | 954.4998 |
| apolipoprotein B precursor | NP_000375.2 | ASGSLPYTQTLQDHLNSLK | 235 | 1037.034 | 1067.584 |
| apolipoprotein B precursor | NP_000375.2 | ASGSLPYTQTLQDHLNSLK | 235 | 1037.034 | 1168.631 |
| apolipoprotein B precursor | NP_000375.2 | ASGSLPYTQTLQDHLNSLK | 235 | 1037.034 | 1296.69 |
| apolipoprotein B precursor | NP_000375.2 | ASGSLPYTQTLQDHLNSLK | 235 | 1037.034 | 1397.738 |
| ceruloplasmin precursor | NP_000087.1 | ENLTAPGSDSAVFFEQGTTR | 236 | 1064.003 | 377.2137 |
| ceruloplasmin precursor | NP_000087.1 | ENLTAPGSDSAVFFEQGTTR | 236 | 1064.003 | 434.2352 |
| ceruloplasmin precursor | NP_000087.1 | ENLTAPGSDSAVFFEQGTTR | 236 | 1064.003 | 562.2938 |
| ceruloplasmin precursor | NP_000087.1 | ENLTAPGSDSAVFFEQGTTR | 236 | 1064.003 | 691.3364 |
| ceruloplasmin precursor | NP_000087.1 | ENLTAPGSDSAVFFEQGTTR | 236 | 1064.003 | 838.4048 |
| ceruloplasmin precursor | NP_000087.1 | ENLTAPGSDSAVFFEQGTTR | 236 | 1064.003 | 985.4732 |
| ceruloplasmin precursor | NP_000087.1 | ENLTAPGSDSAVFFEQGTTR | 236 | 1064.003 | 1084.542 |
| ceruloplasmin precursor | NP_000087.1 | ENLTAPGSDSAVFFEQGTTR | 236 | 1064.003 | 1155.579 |
| ceruloplasmin precursor | NP_000087.1 | ENLTAPGSDSAVFFEQGTTR | 236 | 1064.003 | 1242.611 |
| ceruloplasmin precursor | NP_000087.1 | ENLTAPGSDSAVFFEQGTTR | 236 | 1064.003 | 1357.638 |
| ceruloplasmin precursor | NP_000087.1 | ENLTAPGSDSAVFFEQGTTR | 236 | 1064.003 | 1444.67 |
| paraoxonase 1 | NP_000437.3 | VTQVYAENGTVLQGSTVASVYK | 237 | 1157.6 | 409.244 |
| paraoxonase 1 | NP_000437.3 | VTQVYAENGTVLQGSTVASVYK | 237 | 1157.6 | 496.276 |
| paraoxonase 1 | NP_000437.3 | VTQVYAENGTVLQGSTVASVYK | 237 | 1157.6 | 567.3131 |
| paraoxonase 1 | NP_000437.3 | VTQVYAENGTVLQGSTVASVYK | 237 | 1157.6 | 666.3815 |
| paraoxonase 1 | NP_000437.3 | VTQVYAENGTVLQGSTVASVYK | 237 | 1157.6 | 767.4293 |
| paraoxonase 1 | NP_000437.3 | VTQVYAENGTVLQGSTVASVYK | 237 | 1157.6 | 854.4612 |
| paraoxonase 1 | NP_000437.3 | VTQVYAENGTVLQGSTVASVYK | 237 | 1157.6 | 911.4827 |
| paraoxonase 1 | NP_000437.3 | VTQVYAENGTVLQGSTVASVYK | 237 | 1157.6 | 1039.541 |
| paraoxonase 1 | NP_000437.3 | VTQVYAENGTVLQGSTVASVYK | 237 | 1157.6 | 1152.625 |
| paraoxonase 1 | NP_000437.3 | VTQVYAENGTVLQGSTVASVYK | 237 | 1157.6 | 1251.694 |
| paraoxonase 1 | NP_000437.3 | VTQVYAENGTVLQGSTVASVYK | 237 | 1157.6 | 1352.741 |
| paraoxonase 1 | NP_000437.3 | VTQVYAENGTVLQGSTVASVYK | 237 | 1157.6 | 1409.763 |
| apolipoprotein C-II precursor | NP_000474.2 | ESLSSYWESAK | 238 | 643.7986 | 620.3033 |
| ceruloplasmin precursor | NP_000087.1 | AEEEHLGILGPQLHADVGDK | 239 | 1064.537 | 319.1607 |
| ceruloplasmin precursor | NP_000087.1 | AEEEHLGILGPQLHADVGDK | 239 | 1064.537 | 418.2291 |
| ceruloplasmin precursor | NP_000087.1 | AEEEHLGILGPQLHADVGDK | 239 | 1064.537 | 533.256 |
| ceruloplasmin precursor | NP_000087.1 | AEEEHLGILGPQLHADVGDK | 239 | 1064.537 | 604.2932 |
| ceruloplasmin precursor | NP_000087.1 | AEEEHLGILGPQLHADVGDK | 239 | 1064.537 | 741.3521 |
| ceruloplasmin precursor | NP_000087.1 | AEEEHLGILGPQLHADVGDK | 239 | 1064.537 | 854.4361 |
| ceruloplasmin precursor | NP_000087.1 | AEEEHLGILGPQLHADVGDK | 239 | 1064.537 | 982.4947 |
| ceruloplasmin precursor | NP_000087.1 | AEEEHLGILGPQLHADVGDK | 239 | 1064.537 | 1079.547 |
| ceruloplasmin precursor | NP_000087.1 | AEEEHLGILGPQLHADVGDK | 239 | 1064.537 | 1136.569 |
| ceruloplasmin precursor | NP_000087.1 | AEEEHLGILGPQLHADVGDK | 239 | 1064.537 | 1249.653 |
| ceruloplasmin precursor | NP_000087.1 | AEEEHLGILGPQLHADVGDK | 239 | 1064.537 | 1362.737 |
| ceruloplasmin precursor | NP_000087.1 | AEEEHLGILGPQLHADVGDK | 239 | 1064.537 | 1419.759 |
| pregnancy-zone protein | NP_002855.1 | MVSGFIPLKPTVK | 240 | 708.9178 | 347.2283 |
| pregnancy-zone protein | NP_002855.1 | MVSGFIPLKPTVK | 240 | 708.9178 | 444.2811 |
| pregnancy-zone protein | NP_002855.1 | MVSGFIPLKPTVK | 240 | 708.9178 | 572.3761 |
| pregnancy-zone protein | NP_002855.1 | MVSGFIPLKPTVK | 240 | 708.9178 | 685.4601 |
| pregnancy-zone protein | NP_002855.1 | MVSGFIPLKPTVK | 240 | 708.9178 | 782.5129 |
| pregnancy-zone protein | NP_002855.1 | MVSGFIPLKPTVK | 240 | 708.9178 | 895.597 |
| pregnancy-zone protein | NP_002855.1 | MVSGFIPLKPTVK | 240 | 708.9178 | 1042.665 |
| pregnancy-zone protein | NP_002855.1 | MVSGFIPLKPTVK | 240 | 708.9178 | 1099.687 |
| pregnancy-zone protein | NP_002855.1 | MVSGFIPLKPTVK | 240 | 708.9178 | 1186.719 |
| pregnancy-zone protein | NP_002855.1 | MVSGFIPLKPTVK | 240 | 708.9178 | 1285.787 |
| paraoxonase 1 | NP_000437.3 | YVYIAELLAHK | 241 | 660.3715 | 355.2083 |
| paraoxonase 1 | NP_000437.3 | YVYIAELLAHK | 241 | 660.3715 | 468.2924 |
| paraoxonase 1 | NP_000437.3 | YVYIAELLAHK | 241 | 660.3715 | 581.3764 |
| paraoxonase 1 | NP_000437.3 | YVYIAELLAHK | 241 | 660.3715 | 710.419 |
| paraoxonase 1 | NP_000437.3 | YVYIAELLAHK | 241 | 660.3715 | 781.4561 |
| paraoxonase 1 | NP_000437.3 | YVYIAELLAHK | 241 | 660.3715 | 894.5402 |
| paraoxonase 1 | NP_000437.3 | YVYIAELLAHK | 241 | 660.3715 | 1057.604 |
| paraoxonase 1 | NP_000437.3 | YVYIAELLAHK | 241 | 660.3715 | 1156.672 |
| beta globin | NP_000509.1 | LLVVYPWTQR | 242 | 637.8664 | 404.2247 |
| beta globin | NP_000509.1 | LLVVYPWTQR | 242 | 637.8664 | 590.304 |
| beta globin | NP_000509.1 | LLVVYPWTQR | 242 | 637.8664 | 850.42 |
| beta globin | NP_000509.1 | LLVVYPWTQR | 242 | 637.8664 | 949.4885 |
| beta globin | NP_000509.1 | LLVVYPWTQR | 242 | 637.8664 | 1048.557 |
| beta globin | NP_000509.1 | LLVVYPWTQR | 242 | 637.8664 | 1161.641 |
| apolipoprotein D precursor | NP_001638.1 | NILTSNNIDVK | 243 | 615.8381 | 1003.541 |
| ceruloplasmin precursor | NP_000087.1 | ELHHLQEQNVSNAFLDK | 244 | 1011.506 | 375.2233 |
| ceruloplasmin precursor | NP_000087.1 | ELHHLQEQNVSNAFLDK | 244 | 1011.506 | 522.2917 |
| ceruloplasmin precursor | NP_000087.1 | ELHHLQEQNVSNAFLDK | 244 | 1011.506 | 593.3288 |
| ceruloplasmin precursor | NP_000087.1 | ELHHLQEQNVSNAFLDK | 244 | 1011.506 | 707.3717 |
| ceruloplasmin precursor | NP_000087.1 | ELHHLQEQNVSNAFLDK | 244 | 1011.506 | 794.4037 |
| ceruloplasmin precursor | NP_000087.1 | ELHHLQEQNVSNAFLDK | 244 | 1011.506 | 893.4722 |
| ceruloplasmin precursor | NP_000087.1 | ELHHLQEQNVSNAFLDK | 244 | 1011.506 | 1007.515 |
| ceruloplasmin precursor | NP_000087.1 | ELHHLQEQNVSNAFLDK | 244 | 1011.506 | 1135.574 |
| ceruloplasmin precursor | NP_000087.1 | ELHHLQEQNVSNAFLDK | 244 | 1011.506 | 1264.616 |
| ceruloplasmin precursor | NP_000087.1 | ELHHLQEQNVSNAFLDK | 244 | 1011.506 | 1392.675 |
| apolipoprotein B precursor | NP_000375.2 | LPYTIITTPPLK | 245 | 678.9105 | 357.2491 |
| apolipoprotein B precursor | NP_000375.2 | LPYTIITTPPLK | 245 | 678.9105 | 454.3018 |
| apolipoprotein B precursor | NP_000375.2 | LPYTIITTPPLK | 245 | 678.9105 | 555.3495 |
| apolipoprotein B precursor | NP_000375.2 | LPYTIITTPPLK | 245 | 678.9105 | 656.3972 |
| apolipoprotein B precursor | NP_000375.2 | LPYTIITTPPLK | 245 | 678.9105 | 769.4813 |
| apolipoprotein B precursor | NP_000375.2 | LPYTIITTPPLK | 245 | 678.9105 | 882.5653 |
| apolipoprotein B precursor | NP_000375.2 | LPYTIITTPPLK | 245 | 678.9105 | 983.613 |
| apolipoprotein B precursor | NP_000375.2 | LPYTIITTPPLK | 245 | 678.9105 | 1146.676 |
| apolipoprotein B precursor | NP_000375.2 | LPYTIITTPPLK | 245 | 678.9105 | 1243.729 |
| apolipoprotein B precursor | NP_000375.2 | TGISPLALIK | 246 | 506.8237 | 373.2804 |
| apolipoprotein B precursor | NP_000375.2 | TGISPLALIK | 246 | 506.8237 | 444.3175 |
| apolipoprotein B precursor | NP_000375.2 | TGISPLALIK | 246 | 506.8237 | 557.4016 |
| apolipoprotein B precursor | NP_000375.2 | TGISPLALIK | 246 | 506.8237 | 654.4543 |
| apolipoprotein B precursor | NP_000375.2 | TGISPLALIK | 246 | 506.8237 | 854.5704 |
| apolipoprotein B precursor | NP_000375.2 | TGISPLALIK | 246 | 506.8237 | 911.5919 |
| pregnancy-zone protein | NP_002855.1 | ATVLNYLPK | 247 | 509.8002 | 747.4394 |
| serum amyloid P component precursor | NP_001630.1 | VGEYSLYIGR | 248 | 578.8035 | 708.4034 |
| apolipoprotein A-II preproprotein | NP_001634.1 | SKEQLTPLIK | 249 | 578.8505 | 941.566 |
| paraoxonase 1 | NP_000437.3 | VVAEGFDFANGINISPDGK | 250 | 975.4838 | 319.1607 |
| paraoxonase 1 | NP_000437.3 | VVAEGFDFANGINISPDGK | 250 | 975.4838 | 416.2134 |
| paraoxonase 1 | NP_000437.3 | VVAEGFDFANGINISPDGK | 250 | 975.4838 | 503.2455 |
| paraoxonase 1 | NP_000437.3 | VVAEGFDFANGINISPDGK | 250 | 975.4838 | 616.3295 |
| paraoxonase 1 | NP_000437.3 | VVAEGFDFANGINISPDGK | 250 | 975.4838 | 730.3724 |
| paraoxonase 1 | NP_000437.3 | VVAEGFDFANGINISPDGK | 250 | 975.4838 | 843.4565 |
| paraoxonase 1 | NP_000437.3 | VVAEGFDFANGINISPDGK | 250 | 975.4838 | 900.478 |
| paraoxonase 1 | NP_000437.3 | VVAEGFDFANGINISPDGK | 250 | 975.4838 | 1014.521 |
| paraoxonase 1 | NP_000437.3 | VVAEGFDFANGINISPDGK | 250 | 975.4838 | 1085.558 |
| paraoxonase 1 | NP_000437.3 | VVAEGFDFANGINISPDGK | 250 | 975.4838 | 1232.626 |
| paraoxonase 1 | NP_000437.3 | VVAEGFDFANGINISPDGK | 250 | 975.4838 | 1347.653 |
| paraoxonase 1 | NP_000437.3 | VVAEGFDFANGINISPDGK | 250 | 975.4838 | 1494.722 |
| apolipoprotein B precursor | NP_000375.2 | IVQILPWEQNEQVK | 251 | 862.4725 | 374.2393 |
| apolipoprotein B precursor | NP_000375.2 | IVQILPWEQNEQVK | 251 | 862.4725 | 503.2818 |
| apolipoprotein B precursor | NP_000375.2 | IVQILPWEQNEQVK | 251 | 862.4725 | 617.3248 |
| apolipoprotein B precursor | NP_000375.2 | IVQILPWEQNEQVK | 251 | 862.4725 | 745.3834 |
| apolipoprotein B precursor | NP_000375.2 | IVQILPWEQNEQVK | 251 | 862.4725 | 874.426 |
| apolipoprotein B precursor | NP_000375.2 | IVQILPWEQNEQVK | 251 | 862.4725 | 1060.505 |
| apolipoprotein B precursor | NP_000375.2 | IVQILPWEQNEQVK | 251 | 862.4725 | 1157.558 |
| apolipoprotein B precursor | NP_000375.2 | IVQILPWEQNEQVK | 251 | 862.4725 | 1270.642 |
| apolipoprotein B precursor | NP_000375.2 | IVQILPWEQNEQVK | 251 | 862.4725 | 1383.726 |
| apolipoprotein A-IV precursor | NP_000473.2 | SLAELGGHLDQQVEEFR | 252 | 964.4791 | 451.2294 |
| apolipoprotein A-IV precursor | NP_000473.2 | SLAELGGHLDQQVEEFR | 252 | 964.4791 | 580.272 |
| apolipoprotein A-IV precursor | NP_000473.2 | SLAELGGHLDQQVEEFR | 252 | 964.4791 | 679.3404 |
| apolipoprotein A-IV precursor | NP_000473.2 | SLAELGGHLDQQVEEFR | 252 | 964.4791 | 807.399 |
| apolipoprotein A-IV precursor | NP_000473.2 | SLAELGGHLDQQVEEFR | 252 | 964.4791 | 935.4576 |
| apolipoprotein A-IV precursor | NP_000473.2 | SLAELGGHLDQQVEEFR | 252 | 964.4791 | 1050.484 |
| apolipoprotein A-IV precursor | NP_000473.2 | SLAELGGHLDQQVEEFR | 252 | 964.4791 | 1163.569 |
| apolipoprotein A-IV precursor | NP_000473.2 | SLAELGGHLDQQVEEFR | 252 | 964.4791 | 1300.627 |
| apolipoprotein A-IV precursor | NP_000473.2 | SLAELGGHLDQQVEEFR | 252 | 964.4791 | 1357.649 |
| apolipoprotein A-IV precursor | NP_000473.2 | SLAELGGHLDQQVEEFR | 252 | 964.4791 | 1414.67 |
| apolipoprotein E precursor | NP_000032.1 | FWDYLR | 253 | 450.2242 | 566.2927 |
| alpha-2-macroglobulin precursor | NP_000005.2 | GHFSISIPVK | 254 | 542.8111 | 890.5341 |
| apolipoprotein B precursor | NP_000375.2 | QTIIVVLENVQR | 255 | 706.417 | 402.2454 |
| apolipoprotein B precursor | NP_000375.2 | QTIIVVLENVQR | 255 | 706.417 | 516.2883 |
| apolipoprotein B precursor | NP_000375.2 | QTIIVVLENVQR | 255 | 706.417 | 645.3309 |
| apolipoprotein B precursor | NP_000375.2 | QTIIVVLENVQR | 255 | 706.417 | 758.415 |
| apolipoprotein B precursor | NP_000375.2 | QTIIVVLENVQR | 255 | 706.417 | 857.4834 |
| apolipoprotein B precursor | NP_000375.2 | QTIIVVLENVQR | 255 | 706.417 | 956.5518 |
| apolipoprotein B precursor | NP_000375.2 | QTIIVVLENVQR | 255 | 706.417 | 1069.636 |
| apolipoprotein B precursor | NP_000375.2 | QTIIVVLENVQR | 255 | 706.417 | 1182.72 |
| apolipoprotein B precursor | NP_000375.2 | QTIIVVLENVQR | 255 | 706.417 | 1283.768 |
| apolipoprotein A-I preproprotein | NP_000030.1 | VKDLATVYVDVLK | 256 | 731.9294 | 359.2647 |
| apolipoprotein A-I preproprotein | NP_000030.1 | VKDLATVYVDVLK | 256 | 731.9294 | 474.2917 |
| apolipoprotein A-I preproprotein | NP_000030.1 | VKDLATVYVDVLK | 256 | 731.9294 | 573.3601 |
| apolipoprotein A-I preproprotein | NP_000030.1 | VKDLATVYVDVLK | 256 | 731.9294 | 736.4234 |
| apolipoprotein A-I preproprotein | NP_000030.1 | VKDLATVYVDVLK | 256 | 731.9294 | 835.4918 |
| apolipoprotein A-I preproprotein | NP_000030.1 | VKDLATVYVDVLK | 256 | 731.9294 | 936.5395 |
| apolipoprotein A-I preproprotein | NP_000030.1 | VKDLATVYVDVLK | 256 | 731.9294 | 1007.577 |
| apolipoprotein A-I preproprotein | NP_000030.1 | VKDLATVYVDVLK | 256 | 731.9294 | 1120.661 |
| apolipoprotein A-I preproprotein | NP_000030.1 | VKDLATVYVDVLK | 256 | 731.9294 | 1235.688 |
| apolipoprotein A-I preproprotein | NP_000030.1 | VKDLATVYVDVLK | 256 | 731.9294 | 1363.783 |
| apolipoprotein B precursor | NP_000375.2 | GFEPTLEALFGK | 257 | 654.8453 | 351.2021 |
| apolipoprotein B precursor | NP_000375.2 | GFEPTLEALFGK | 257 | 654.8453 | 464.2862 |
| apolipoprotein B precursor | NP_000375.2 | GFEPTLEALFGK | 257 | 654.8453 | 535.3233 |
| apolipoprotein B precursor | NP_000375.2 | GFEPTLEALFGK | 257 | 654.8453 | 664.3659 |
| apolipoprotein B precursor | NP_000375.2 | GFEPTLEALFGK | 257 | 654.8453 | 777.45 |
| apolipoprotein B precursor | NP_000375.2 | GFEPTLEALFGK | 257 | 654.8453 | 878.4976 |
| apolipoprotein B precursor | NP_000375.2 | GFEPTLEALFGK | 257 | 654.8453 | 975.5504 |
| apolipoprotein B precursor | NP_000375.2 | GFEPTLEALFGK | 257 | 654.8453 | 1104.593 |
| apolipoprotein B precursor | NP_000375.2 | GFEPTLEALFGK | 257 | 654.8453 | 1251.661 |
| paraoxonase 1 | NP_000437.3 | IFFYDSENPPASEVLR | 258 | 942.4623 | 387.2709 |
| paraoxonase 1 | NP_000437.3 | IFFYDSENPPASEVLR | 258 | 942.4623 | 516.3135 |
| paraoxonase 1 | NP_000437.3 | IFFYDSENPPASEVLR | 258 | 942.4623 | 603.3455 |
| paraoxonase 1 | NP_000437.3 | IFFYDSENPPASEVLR | 258 | 942.4623 | 674.3826 |
| paraoxonase 1 | NP_000437.3 | IFFYDSENPPASEVLR | 258 | 942.4623 | 771.4354 |
| paraoxonase 1 | NP_000437.3 | IFFYDSENPPASEVLR | 258 | 942.4623 | 982.5311 |
| paraoxonase 1 | NP_000437.3 | IFFYDSENPPASEVLR | 258 | 942.4623 | 1111.574 |
| paraoxonase 1 | NP_000437.3 | IFFYDSENPPASEVLR | 258 | 942.4623 | 1198.606 |
| paraoxonase 1 | NP_000437.3 | IFFYDSENPPASEVLR | 258 | 942.4623 | 1313.633 |
| paraoxonase 1 | NP_000437.3 | IFFYDSENPPASEVLR | 258 | 942.4623 | 1476.696 |
| apolipoprotein B precursor | NP_000375.2 | NLTDFAEQYSIQDWAK | 259 | 964.9549 | 404.2287 |
| apolipoprotein B precursor | NP_000375.2 | NLTDFAEQYSIQDWAK | 259 | 964.9549 | 519.2556 |
| apolipoprotein B precursor | NP_000375.2 | NLTDFAEQYSIQDWAK | 259 | 964.9549 | 647.3142 |
| apolipoprotein B precursor | NP_000375.2 | NLTDFAEQYSIQDWAK | 259 | 964.9549 | 760.3983 |
| apolipoprotein B precursor | NP_000375.2 | NLTDFAEQYSIQDWAK | 259 | 964.9549 | 847.4303 |
| apolipoprotein B precursor | NP_000375.2 | NLTDFAEQYSIQDWAK | 259 | 964.9549 | 1010.494 |
| apolipoprotein B precursor | NP_000375.2 | NLTDFAEQYSIQDWAK | 259 | 964.9549 | 1138.552 |
| apolipoprotein B precursor | NP_000375.2 | NLTDFAEQYSIQDWAK | 259 | 964.9549 | 1267.595 |
| apolipoprotein B precursor | NP_000375.2 | NLTDFAEQYSIQDWAK | 259 | 964.9549 | 1338.632 |
| apolipoprotein B precursor | NP_000375.2 | NLTDFAEQYSIQDWAK | 259 | 964.9549 | 1485.7 |
| apolipoprotein B precursor | NP_000375.2 | FEVDSPVYNATWSASLK | 260 | 957.4677 | 347.2283 |
| apolipoprotein B precursor | NP_000375.2 | FEVDSPVYNATWSASLK | 260 | 957.4677 | 418.2655 |
| apolipoprotein B precursor | NP_000375.2 | FEVDSPVYNATWSASLK | 260 | 957.4677 | 505.2975 |
| apolipoprotein B precursor | NP_000375.2 | FEVDSPVYNATWSASLK | 260 | 957.4677 | 691.3768 |
| apolipoprotein B precursor | NP_000375.2 | FEVDSPVYNATWSASLK | 260 | 957.4677 | 792.4245 |
| apolipoprotein B precursor | NP_000375.2 | FEVDSPVYNATWSASLK | 260 | 957.4677 | 863.4616 |
| apolipoprotein B precursor | NP_000375.2 | FEVDSPVYNATWSASLK | 260 | 957.4677 | 977.5045 |
| apolipoprotein B precursor | NP_000375.2 | FEVDSPVYNATWSASLK | 260 | 957.4677 | 1140.568 |
| apolipoprotein B precursor | NP_000375.2 | FEVDSPVYNATWSASLK | 260 | 957.4677 | 1239.636 |
| apolipoprotein B precursor | NP_000375.2 | FEVDSPVYNATWSASLK | 260 | 957.4677 | 1336.689 |
| apolipoprotein B precursor | NP_000375.2 | FEVDSPVYNATWSASLK | 260 | 957.4677 | 1423.721 |
| apolipoprotein B precursor | NP_000375.2 | GIISALLVPPETEEAK | 261 | 833.9667 | 347.192 |
| apolipoprotein B precursor | NP_000375.2 | GIISALLVPPETEEAK | 261 | 833.9667 | 476.2346 |
| apolipoprotein B precursor | NP_000375.2 | GIISALLVPPETEEAK | 261 | 833.9667 | 577.2822 |
| apolipoprotein B precursor | NP_000375.2 | GIISALLVPPETEEAK | 261 | 833.9667 | 706.3248 |
| apolipoprotein B precursor | NP_000375.2 | GIISALLVPPETEEAK | 261 | 833.9667 | 803.3776 |
| apolipoprotein B precursor | NP_000375.2 | GIISALLVPPETEEAK | 261 | 833.9667 | 900.4304 |
| apolipoprotein B precursor | NP_000375.2 | GIISALLVPPETEEAK | 261 | 833.9667 | 999.4988 |
| apolipoprotein B precursor | NP_000375.2 | GIISALLVPPETEEAK | 261 | 833.9667 | 1112.583 |
| apolipoprotein B precursor | NP_000375.2 | GIISALLVPPETEEAK | 261 | 833.9667 | 1225.667 |
| apolipoprotein B precursor | NP_000375.2 | GIISALLVPPETEEAK | 261 | 833.9667 | 1296.704 |
| apolipoprotein B precursor | NP_000375.2 | GIISALLVPPETEEAK | 261 | 833.9667 | 1383.736 |
| apolipoprotein B precursor | NP_000375.2 | GIISALLVPPETEEAK | 261 | 833.9667 | 1496.82 |
| ceruloplasmin precursor | NP_000087.1 | SVPPSASHVAPTETFTYEWTVPK | 262 | 1266.126 | 343.2334 |
| ceruloplasmin precursor | NP_000087.1 | SVPPSASHVAPTETFTYEWTVPK | 262 | 1266.126 | 444.2811 |
| ceruloplasmin precursor | NP_000087.1 | SVPPSASHVAPTETFTYEWTVPK | 262 | 1266.126 | 630.3604 |
| ceruloplasmin precursor | NP_000087.1 | SVPPSASHVAPTETFTYEWTVPK | 262 | 1266.126 | 759.403 |
| ceruloplasmin precursor | NP_000087.1 | SVPPSASHVAPTETFTYEWTVPK | 262 | 1266.126 | 922.4664 |
| ceruloplasmin precursor | NP_000087.1 | SVPPSASHVAPTETFTYEWTVPK | 262 | 1266.126 | 1023.514 |
| ceruloplasmin precursor | NP_000087.1 | SVPPSASHVAPTETFTYEWTVPK | 262 | 1266.126 | 1170.582 |
| ceruloplasmin precursor | NP_000087.1 | SVPPSASHVAPTETFTYEWTVPK | 262 | 1266.126 | 1271.63 |
| ceruloplasmin precursor | NP_000087.1 | SVPPSASHVAPTETFTYEWTVPK | 262 | 1266.126 | 1400.673 |
| apolipoprotein B precursor | NP_000375.2 | VNWEEEAASGLLTSLK | 263 | 873.949 | 347.2283 |
| apolipoprotein B precursor | NP_000375.2 | VNWEEEAASGLLTSLK | 263 | 873.949 | 448.276 |
| apolipoprotein B precursor | NP_000375.2 | VNWEEEAASGLLTSLK | 263 | 873.949 | 561.3601 |
| apolipoprotein B precursor | NP_000375.2 | VNWEEEAASGLLTSLK | 263 | 873.949 | 674.4442 |
| apolipoprotein B precursor | NP_000375.2 | VNWEEEAASGLLTSLK | 263 | 873.949 | 731.4656 |
| apolipoprotein B precursor | NP_000375.2 | VNWEEEAASGLLTSLK | 263 | 873.949 | 818.4976 |
| apolipoprotein B precursor | NP_000375.2 | VNWEEEAASGLLTSLK | 263 | 873.949 | 889.5347 |
| apolipoprotein B precursor | NP_000375.2 | VNWEEEAASGLLTSLK | 263 | 873.949 | 960.5719 |
| apolipoprotein B precursor | NP_000375.2 | VNWEEEAASGLLTSLK | 263 | 873.949 | 1089.615 |
| apolipoprotein B precursor | NP_000375.2 | VNWEEEAASGLLTSLK | 263 | 873.949 | 1218.657 |
| apolipoprotein B precursor | NP_000375.2 | VNWEEEAASGLLTSLK | 263 | 873.949 | 1347.7 |
| alpha 1 globin | NP_000549.1 | TYFPHFDLSHGSAQVK | 264 | 917.4496 | 1188.6 |
| apolipoprotein A-IV precursor | NP_000473.2 | LGPHAGDVEGHLSFLEK | 265 | 602.6442 | 579.811 |
| apolipoprotein C-III precursor | NP_000031.1 | GWVTDGFSSLK | 266 | 598.801 | 854.4249 |
| ceruloplasmin precursor | NP_000087.1 | GAYPLSIEPIGVR | 267 | 686.3852 | 541.3451 |
| apolipoprotein A-I preproprotein | NP_000030.1 | LLDNWDSVTSTFSK | 268 | 806.8963 | 971.4675 |
| ceruloplasmin precursor | NP_000087.1 | TYYIAAVEVEWDYSPQR | 269 | 1045.497 | 400.2297 |
| ceruloplasmin precursor | NP_000087.1 | TYYIAAVEVEWDYSPQR | 269 | 1045.497 | 487.2618 |
| ceruloplasmin precursor | NP_000087.1 | TYYIAAVEVEWDYSPQR | 269 | 1045.497 | 650.3251 |
| ceruloplasmin precursor | NP_000087.1 | TYYIAAVEVEWDYSPQR | 269 | 1045.497 | 765.3521 |
| ceruloplasmin precursor | NP_000087.1 | TYYIAAVEVEWDYSPQR | 269 | 1045.497 | 951.4313 |
| ceruloplasmin precursor | NP_000087.1 | TYYIAAVEVEWDYSPQR | 269 | 1045.497 | 1080.474 |
| ceruloplasmin precursor | NP_000087.1 | TYYIAAVEVEWDYSPQR | 269 | 1045.497 | 1179.542 |
| ceruloplasmin precursor | NP_000087.1 | TYYIAAVEVEWDYSPQR | 269 | 1045.497 | 1308.585 |
| ceruloplasmin precursor | NP_000087.1 | TYYIAAVEVEWDYSPQR | 269 | 1045.497 | 1407.653 |
| ceruloplasmin precursor | NP_000087.1 | TYYIAAVEVEWDYSPQR | 269 | 1045.497 | 1478.69 |
| apolipoprotein B precursor | NP_000375.2 | LAPGELTIIL | 270 | 520.3235 | 358.2695 |
| apolipoprotein B precursor | NP_000375.2 | LAPGELTIIL | 270 | 520.3235 | 459.3172 |
| apolipoprotein B precursor | NP_000375.2 | LAPGELTIIL | 270 | 520.3235 | 572.4012 |
| apolipoprotein B precursor | NP_000375.2 | LAPGELTIIL | 270 | 520.3235 | 701.4438 |
| apolipoprotein B precursor | NP_000375.2 | LAPGELTIIL | 270 | 520.3235 | 758.4653 |
| apolipoprotein B precursor | NP_000375.2 | LAPGELTIIL | 270 | 520.3235 | 855.5181 |
| apolipoprotein B precursor | NP_000375.2 | LAPGELTIIL | 270 | 520.3235 | 926.5552 |
| apolipoprotein E precursor | NP_000032.1 | SWFEPLVEDM[Oxid]QR | 271 | 776.8586 | 450.2124 |
| apolipoprotein E precursor | NP_000032.1 | SWFEPLVEDM[Oxid]QR | 271 | 776.8586 | 565.2393 |
| apolipoprotein E precursor | NP_000032.1 | SWFEPLVEDM[Oxid]QR | 271 | 776.8586 | 694.2819 |
| apolipoprotein E precursor | NP_000032.1 | SWFEPLVEDM[Oxid]QR | 271 | 776.8586 | 793.3503 |
| apolipoprotein E precursor | NP_000032.1 | SWFEPLVEDM[Oxid]QR | 271 | 776.8586 | 906.4344 |
| apolipoprotein E precursor | NP_000032.1 | SWFEPLVEDM[Oxid]QR | 271 | 776.8586 | 1003.487 |
| apolipoprotein E precursor | NP_000032.1 | SWFEPLVEDM[Oxid]QR | 271 | 776.8586 | 1132.53 |
| apolipoprotein E precursor | NP_000032.1 | SWFEPLVEDM[Oxid]QR | 271 | 776.8586 | 1279.598 |
| apolipoprotein E precursor | NP_000032.1 | SWFEPLVEDM[Oxid]QR | 271 | 776.8586 | 1465.677 |
| beta globin | NP_000509.1 | LLVVYPWTQR | 272 | 637.8664 | 687.3568 |
| apolipoprotein B precursor | NP_000375.2 | LSLESLTSYFSIESSTK | 273 | 946.4804 | 335.192 |
| apolipoprotein B precursor | NP_000375.2 | LSLESLTSYFSIESSTK | 273 | 946.4804 | 422.224 |
| apolipoprotein B precursor | NP_000375.2 | LSLESLTSYFSIESSTK | 273 | 946.4804 | 551.2666 |
| apolipoprotein B precursor | NP_000375.2 | LSLESLTSYFSIESSTK | 273 | 946.4804 | 664.3506 |
| apolipoprotein B precursor | NP_000375.2 | LSLESLTSYFSIESSTK | 273 | 946.4804 | 751.3827 |
| apolipoprotein B precursor | NP_000375.2 | LSLESLTSYFSIESSTK | 273 | 946.4804 | 898.4511 |
| apolipoprotein B precursor | NP_000375.2 | LSLESLTSYFSIESSTK | 273 | 946.4804 | 1061.514 |
| apolipoprotein B precursor | NP_000375.2 | LSLESLTSYFSIESSTK | 273 | 946.4804 | 1148.546 |
| apolipoprotein B precursor | NP_000375.2 | LSLESLTSYFSIESSTK | 273 | 946.4804 | 1249.594 |
| apolipoprotein B precursor | NP_000375.2 | LSLESLTSYFSIESSTK | 273 | 946.4804 | 1362.678 |
| apolipoprotein B precursor | NP_000375.2 | LSLESLTSYFSIESSTK | 273 | 946.4804 | 1449.71 |
| apolipoprotein B precursor | NP_000375.2 | TGISPLALIK | 274 | 506.8237 | 741.4863 |
| apolipoprotein B precursor | NP_000375.2 | YEDGTLSLTSTSDLQSGIIK | 275 | 1064.54 | 373.2804 |
| apolipoprotein B precursor | NP_000375.2 | YEDGTLSLTSTSDLQSGIIK | 275 | 1064.54 | 430.3018 |
| apolipoprotein B precursor | NP_000375.2 | YEDGTLSLTSTSDLQSGIIK | 275 | 1064.54 | 517.3339 |
| apolipoprotein B precursor | NP_000375.2 | YEDGTLSLTSTSDLQSGIIK | 275 | 1064.54 | 645.3925 |
| apolipoprotein B precursor | NP_000375.2 | YEDGTLSLTSTSDLQSGIIK | 275 | 1064.54 | 758.4765 |
| apolipoprotein B precursor | NP_000375.2 | YEDGTLSLTSTSDLQSGIIK | 275 | 1064.54 | 873.5035 |
| apolipoprotein B precursor | NP_000375.2 | YEDGTLSLTSTSDLQSGIIK | 275 | 1064.54 | 960.5355 |
| apolipoprotein B precursor | NP_000375.2 | YEDGTLSLTSTSDLQSGIIK | 275 | 1064.54 | 1061.583 |
| apolipoprotein B precursor | NP_000375.2 | YEDGTLSLTSTSDLQSGIIK | 275 | 1064.54 | 1148.615 |
| apolipoprotein B precursor | NP_000375.2 | YEDGTLSLTSTSDLQSGIIK | 275 | 1064.54 | 1249.663 |
| apolipoprotein B precursor | NP_000375.2 | YEDGTLSLTSTSDLQSGIIK | 275 | 1064.54 | 1362.747 |
| apolipoprotein B precursor | NP_000375.2 | YEDGTLSLTSTSDLQSGIIK | 275 | 1064.54 | 1449.779 |
| apolipoprotein B precursor | NP_000375.2 | DQEVLLQTFLDDASPGDK | 276 | 995.9838 | 319.1607 |
| apolipoprotein B precursor | NP_000375.2 | DQEVLLQTFLDDASPGDK | 276 | 995.9838 | 416.2134 |
| apolipoprotein B precursor | NP_000375.2 | DQEVLLQTFLDDASPGDK | 276 | 995.9838 | 503.2455 |
| apolipoprotein B precursor | NP_000375.2 | DQEVLLQTFLDDASPGDK | 276 | 995.9838 | 574.2826 |
| apolipoprotein B precursor | NP_000375.2 | DQEVLLQTFLDDASPGDK | 276 | 995.9838 | 689.3095 |
| apolipoprotein B precursor | NP_000375.2 | DQEVLLQTFLDDASPGDK | 276 | 995.9838 | 804.3364 |
| apolipoprotein B precursor | NP_000375.2 | DQEVLLQTFLDDASPGDK | 276 | 995.9838 | 917.4205 |
| apolipoprotein B precursor | NP_000375.2 | DQEVLLQTFLDDASPGDK | 276 | 995.9838 | 1064.489 |
| apolipoprotein B precursor | NP_000375.2 | DQEVLLQTFLDDASPGDK | 276 | 995.9838 | 1165.537 |
| apolipoprotein B precursor | NP_000375.2 | DQEVLLQTFLDDASPGDK | 276 | 995.9838 | 1293.595 |
| apolipoprotein B precursor | NP_000375.2 | DQEVLLQTFLDDASPGDK | 276 | 995.9838 | 1406.679 |
| apolipoprotein B precursor | NP_000375.2 | NHLQLEGLFFTNGEHTSK | 277 | 1036.513 | 335.192 |
| apolipoprotein B precursor | NP_000375.2 | NHLQLEGLFFTNGEHTSK | 277 | 1036.513 | 472.2509 |
| apolipoprotein B precursor | NP_000375.2 | NHLQLEGLFFTNGEHTSK | 277 | 1036.513 | 601.2935 |
| apolipoprotein B precursor | NP_000375.2 | NHLQLEGLFFTNGEHTSK | 277 | 1036.513 | 658.3149 |
| apolipoprotein B precursor | NP_000375.2 | NHLQLEGLFFTNGEHTSK | 277 | 1036.513 | 772.3578 |
| apolipoprotein B precursor | NP_000375.2 | NHLQLEGLFFTNGEHTSK | 277 | 1036.513 | 873.4055 |
| apolipoprotein B precursor | NP_000375.2 | NHLQLEGLFFTNGEHTSK | 277 | 1036.513 | 1020.474 |
| apolipoprotein B precursor | NP_000375.2 | NHLQLEGLFFTNGEHTSK | 277 | 1036.513 | 1167.542 |
| apolipoprotein B precursor | NP_000375.2 | NHLQLEGLFFTNGEHTSK | 277 | 1036.513 | 1280.626 |
| apolipoprotein B precursor | NP_000375.2 | NHLQLEGLFFTNGEHTSK | 277 | 1036.513 | 1337.648 |
| apolipoprotein B precursor | NP_000375.2 | NHLQLEGLFFTNGEHTSK | 277 | 1036.513 | 1466.69 |
| histidine-rich glycoprotein precursor | NP_000403.1 | DGYLFQLLR | 278 | 562.8086 | 401.2865 |
| histidine-rich glycoprotein precursor | NP_000403.1 | DGYLFQLLR | 278 | 562.8086 | 529.3451 |
| histidine-rich glycoprotein precursor | NP_000403.1 | DGYLFQLLR | 278 | 562.8086 | 789.4976 |
| histidine-rich glycoprotein precursor | NP_000403.1 | DGYLFQLLR | 278 | 562.8086 | 952.5609 |
| histidine-rich glycoprotein precursor | NP_000403.1 | DGYLFQLLR | 278 | 562.8086 | 1009.582 |
| apolipoprotein B precursor | NP_000375.2 | EQHLFLPFSYK | 279 | 704.8666 | 397.2076 |
| apolipoprotein B precursor | NP_000375.2 | EQHLFLPFSYK | 279 | 704.8666 | 544.276 |
| apolipoprotein B precursor | NP_000375.2 | EQHLFLPFSYK | 279 | 704.8666 | 641.3288 |
| apolipoprotein B precursor | NP_000375.2 | EQHLFLPFSYK | 279 | 704.8666 | 754.4128 |
| apolipoprotein B precursor | NP_000375.2 | EQHLFLPFSYK | 279 | 704.8666 | 901.4813 |
| apolipoprotein B precursor | NP_000375.2 | EQHLFLPFSYK | 279 | 704.8666 | 1014.565 |
| apolipoprotein B precursor | NP_000375.2 | EQHLFLPFSYK | 279 | 704.8666 | 1151.624 |
| apolipoprotein B precursor | NP_000375.2 | EQHLFLPFSYK | 279 | 704.8666 | 1279.683 |
| beta globin | NP_000509.1 | VLGAFSDGLAHLDNLK | 280 | 835.449 | 374.2393 |
| beta globin | NP_000509.1 | VLGAFSDGLAHLDNLK | 280 | 835.449 | 489.2662 |
| beta globin | NP_000509.1 | VLGAFSDGLAHLDNLK | 280 | 835.449 | 602.3503 |
| beta globin | NP_000509.1 | VLGAFSDGLAHLDNLK | 280 | 835.449 | 739.4092 |
| beta globin | NP_000509.1 | VLGAFSDGLAHLDNLK | 280 | 835.449 | 810.4463 |
| beta globin | NP_000509.1 | VLGAFSDGLAHLDNLK | 280 | 835.449 | 923.5303 |
| beta globin | NP_000509.1 | VLGAFSDGLAHLDNLK | 280 | 835.449 | 980.5518 |
| beta globin | NP_000509.1 | VLGAFSDGLAHLDNLK | 280 | 835.449 | 1095.579 |
| beta globin | NP_000509.1 | VLGAFSDGLAHLDNLK | 280 | 835.449 | 1182.611 |
| beta globin | NP_000509.1 | VLGAFSDGLAHLDNLK | 280 | 835.449 | 1329.679 |
| beta globin | NP_000509.1 | VLGAFSDGLAHLDNLK | 280 | 835.449 | 1400.716 |
| beta globin | NP_000509.1 | VLGAFSDGLAHLDNLK | 280 | 835.449 | 1457.738 |
| pregnancy-zone protein | NP_002855.1 | ISASSEVAFLSIQIK | 281 | 796.9484 | 388.2549 |
| pregnancy-zone protein | NP_002855.1 | ISASSEVAFLSIQIK | 281 | 796.9484 | 501.339 |
| pregnancy-zone protein | NP_002855.1 | ISASSEVAFLSIQIK | 281 | 796.9484 | 588.371 |
| pregnancy-zone protein | NP_002855.1 | ISASSEVAFLSIQIK | 281 | 796.9484 | 701.4551 |
| pregnancy-zone protein | NP_002855.1 | ISASSEVAFLSIQIK | 281 | 796.9484 | 848.5234 |
| pregnancy-zone protein | NP_002855.1 | ISASSEVAFLSIQIK | 281 | 796.9484 | 919.5606 |
| pregnancy-zone protein | NP_002855.1 | ISASSEVAFLSIQIK | 281 | 796.9484 | 1018.629 |
| pregnancy-zone protein | NP_002855.1 | ISASSEVAFLSIQIK | 281 | 796.9484 | 1147.672 |
| pregnancy-zone protein | NP_002855.1 | ISASSEVAFLSIQIK | 281 | 796.9484 | 1234.704 |
| pregnancy-zone protein | NP_002855.1 | ISASSEVAFLSIQIK | 281 | 796.9484 | 1321.736 |
| pregnancy-zone protein | NP_002855.1 | ISASSEVAFLSIQIK | 281 | 796.9484 | 1392.773 |
| pregnancy-zone protein | NP_002855.1 | ISASSEVAFLSIQIK | 281 | 796.9484 | 1479.805 |
| paraoxonase 1 | NP_000437.3 | IFFYDSENPPASEVLR | 282 | 942.4623 | 868.4882 |
| apolipoprotein B precursor | NP_000375.2 | IAIANIIDEIIEK | 283 | 727.9269 | 389.2389 |
| apolipoprotein B precursor | NP_000375.2 | IAIANIIDEIIEK | 283 | 727.9269 | 502.323 |
| apolipoprotein B precursor | NP_000375.2 | IAIANIIDEIIEK | 283 | 727.9269 | 631.3655 |
| apolipoprotein B precursor | NP_000375.2 | IAIANIIDEIIEK | 283 | 727.9269 | 746.3925 |
| apolipoprotein B precursor | NP_000375.2 | IAIANIIDEIIEK | 283 | 727.9269 | 859.4766 |
| apolipoprotein B precursor | NP_000375.2 | IAIANIIDEIIEK | 283 | 727.9269 | 972.5607 |
| apolipoprotein B precursor | NP_000375.2 | IAIANIIDEIIEK | 283 | 727.9269 | 1086.604 |
| apolipoprotein B precursor | NP_000375.2 | IAIANIIDEIIEK | 283 | 727.9269 | 1157.641 |
| apolipoprotein B precursor | NP_000375.2 | IAIANIIDEIIEK | 283 | 727.9269 | 1270.725 |
| apolipoprotein B precursor | NP_000375.2 | IAIANIIDEIIEK | 283 | 727.9269 | 1341.762 |
| apolipoprotein B precursor | NP_000375.2 | VLLDQLGTTISFER | 284 | 796.4381 | 451.2294 |
| apolipoprotein B precursor | NP_000375.2 | VLLDQLGTTISFER | 284 | 796.4381 | 538.2614 |
| apolipoprotein B precursor | NP_000375.2 | VLLDQLGTTISFER | 284 | 796.4381 | 651.3455 |
| apolipoprotein B precursor | NP_000375.2 | VLLDQLGTTISFER | 284 | 796.4381 | 752.3932 |
| apolipoprotein B precursor | NP_000375.2 | VLLDQLGTTISFER | 284 | 796.4381 | 853.4409 |
| apolipoprotein B precursor | NP_000375.2 | VLLDQLGTTISFER | 284 | 796.4381 | 910.4623 |
| apolipoprotein B precursor | NP_000375.2 | VLLDQLGTTISFER | 284 | 796.4381 | 1023.546 |
| apolipoprotein B precursor | NP_000375.2 | VLLDQLGTTISFER | 284 | 796.4381 | 1151.605 |
| apolipoprotein B precursor | NP_000375.2 | VLLDQLGTTISFER | 284 | 796.4381 | 1266.632 |
| apolipoprotein B precursor | NP_000375.2 | VLLDQLGTTISFER | 284 | 796.4381 | 1379.716 |
| apolipoprotein B precursor | NP_000375.2 | VLLDQLGTTISFER | 284 | 796.4381 | 1492.8 |
| apolipoprotein B precursor | NP_000375.2 | AQNLYQELLTQEGQASFQGLK | 285 | 1183.603 | 317.2178 |
| apolipoprotein B precursor | NP_000375.2 | AQNLYQELLTQEGQASFQGLK | 285 | 1183.603 | 445.2764 |
| apolipoprotein B precursor | NP_000375.2 | AQNLYQELLTQEGQASFQGLK | 285 | 1183.603 | 592.3448 |
| apolipoprotein B precursor | NP_000375.2 | AQNLYQELLTQEGQASFQGLK | 285 | 1183.603 | 679.3768 |
| apolipoprotein B precursor | NP_000375.2 | AQNLYQELLTQEGQASFQGLK | 285 | 1183.603 | 750.4139 |
| apolipoprotein B precursor | NP_000375.2 | AQNLYQELLTQEGQASFQGLK | 285 | 1183.603 | 878.4725 |
| apolipoprotein B precursor | NP_000375.2 | AQNLYQELLTQEGQASFQGLK | 285 | 1183.603 | 935.494 |
| apolipoprotein B precursor | NP_000375.2 | AQNLYQELLTQEGQASFQGLK | 285 | 1183.603 | 1064.536 |
| apolipoprotein B precursor | NP_000375.2 | AQNLYQELLTQEGQASFQGLK | 285 | 1183.603 | 1192.595 |
| apolipoprotein B precursor | NP_000375.2 | AQNLYQELLTQEGQASFQGLK | 285 | 1183.603 | 1293.643 |
| apolipoprotein B precursor | NP_000375.2 | AQNLYQELLTQEGQASFQGLK | 285 | 1183.603 | 1406.727 |
| apolipoprotein B precursor | NP_000375.2 | EEYFDPSIVGWTVK | 286 | 835.4091 | 347.2283 |
| apolipoprotein B precursor | NP_000375.2 | EEYFDPSIVGWTVK | 286 | 835.4091 | 533.3077 |
| apolipoprotein B precursor | NP_000375.2 | EEYFDPSIVGWTVK | 286 | 835.4091 | 590.3291 |
| apolipoprotein B precursor | NP_000375.2 | EEYFDPSIVGWTVK | 286 | 835.4091 | 689.3975 |
| apolipoprotein B precursor | NP_000375.2 | EEYFDPSIVGWTVK | 286 | 835.4091 | 802.4816 |
| apolipoprotein B precursor | NP_000375.2 | EEYFDPSIVGWTVK | 286 | 835.4091 | 889.5136 |
| apolipoprotein B precursor | NP_000375.2 | EEYFDPSIVGWTVK | 286 | 835.4091 | 986.5664 |
| apolipoprotein B precursor | NP_000375.2 | EEYFDPSIVGWTVK | 286 | 835.4091 | 1101.593 |
| apolipoprotein B precursor | NP_000375.2 | EEYFDPSIVGWTVK | 286 | 835.4091 | 1248.662 |
| apolipoprotein B precursor | NP_000375.2 | EEYFDPSIVGWTVK | 286 | 835.4091 | 1411.725 |
| apolipoprotein B precursor | NP_000375.2 | TLADLTLLDSPIK | 287 | 700.4058 | 357.2491 |
| apolipoprotein B precursor | NP_000375.2 | TLADLTLLDSPIK | 287 | 700.4058 | 444.2811 |
| apolipoprotein B precursor | NP_000375.2 | TLADLTLLDSPIK | 287 | 700.4058 | 559.308 |
| apolipoprotein B precursor | NP_000375.2 | TLADLTLLDSPIK | 287 | 700.4058 | 672.3921 |
| apolipoprotein B precursor | NP_000375.2 | TLADLTLLDSPIK | 287 | 700.4058 | 785.4762 |
| apolipoprotein B precursor | NP_000375.2 | TLADLTLLDSPIK | 287 | 700.4058 | 886.5239 |
| apolipoprotein B precursor | NP_000375.2 | TLADLTLLDSPIK | 287 | 700.4058 | 999.6079 |
| apolipoprotein B precursor | NP_000375.2 | TLADLTLLDSPIK | 287 | 700.4058 | 1114.635 |
| apolipoprotein B precursor | NP_000375.2 | TLADLTLLDSPIK | 287 | 700.4058 | 1185.672 |
| apolipoprotein B precursor | NP_000375.2 | TLADLTLLDSPIK | 287 | 700.4058 | 1298.756 |
| apolipoprotein B precursor | NP_000375.2 | TSSFALNLPTLPEVK | 288 | 808.9484 | 375.2233 |
| apolipoprotein B precursor | NP_000375.2 | TSSFALNLPTLPEVK | 288 | 808.9484 | 472.276 |
| apolipoprotein B precursor | NP_000375.2 | TSSFALNLPTLPEVK | 288 | 808.9484 | 585.3601 |
| apolipoprotein B precursor | NP_000375.2 | TSSFALNLPTLPEVK | 288 | 808.9484 | 686.4078 |
| apolipoprotein B precursor | NP_000375.2 | TSSFALNLPTLPEVK | 288 | 808.9484 | 783.4605 |
| apolipoprotein B precursor | NP_000375.2 | TSSFALNLPTLPEVK | 288 | 808.9484 | 896.5446 |
| apolipoprotein B precursor | NP_000375.2 | TSSFALNLPTLPEVK | 288 | 808.9484 | 1010.588 |
| apolipoprotein B precursor | NP_000375.2 | TSSFALNLPTLPEVK | 288 | 808.9484 | 1123.672 |
| apolipoprotein B precursor | NP_000375.2 | TSSFALNLPTLPEVK | 288 | 808.9484 | 1194.709 |
| apolipoprotein B precursor | NP_000375.2 | TSSFALNLPTLPEVK | 288 | 808.9484 | 1341.777 |
| apolipoprotein B precursor | NP_000375.2 | TSSFALNLPTLPEVK | 288 | 808.9484 | 1428.809 |
| apolipoprotein B precursor | NP_000375.2 | LEVLNFDFQANAQLSNPK | 289 | 1024.526 | 358.2079 |
| apolipoprotein B precursor | NP_000375.2 | LEVLNFDFQANAQLSNPK | 289 | 1024.526 | 445.24 |
| apolipoprotein B precursor | NP_000375.2 | LEVLNFDFQANAQLSNPK | 289 | 1024.526 | 558.324 |
| apolipoprotein B precursor | NP_000375.2 | LEVLNFDFQANAQLSNPK | 289 | 1024.526 | 686.3826 |
| apolipoprotein B precursor | NP_000375.2 | LEVLNFDFQANAQLSNPK | 289 | 1024.526 | 757.4197 |
| apolipoprotein B precursor | NP_000375.2 | LEVLNFDFQANAQLSNPK | 289 | 1024.526 | 871.4626 |
| apolipoprotein B precursor | NP_000375.2 | LEVLNFDFQANAQLSNPK | 289 | 1024.526 | 942.4998 |
| apolipoprotein B precursor | NP_000375.2 | LEVLNFDFQANAQLSNPK | 289 | 1024.526 | 1070.558 |
| apolipoprotein B precursor | NP_000375.2 | LEVLNFDFQANAQLSNPK | 289 | 1024.526 | 1217.627 |
| apolipoprotein B precursor | NP_000375.2 | LEVLNFDFQANAQLSNPK | 289 | 1024.526 | 1332.654 |
| apolipoprotein B precursor | NP_000375.2 | LEVLNFDFQANAQLSNPK | 289 | 1024.526 | 1479.722 |
| histidine-rich glycoprotein precursor | NP_000403.1 | DGYLFQLLR | 290 | 562.8086 | 676.4135 |
| histidine-rich glycoprotein precursor | NP_000403.1 | DSPVLIDFFEDTER | 291 | 841.899 | 405.2087 |
| histidine-rich glycoprotein precursor | NP_000403.1 | DSPVLIDFFEDTER | 291 | 841.899 | 520.2356 |
| histidine-rich glycoprotein precursor | NP_000403.1 | DSPVLIDFFEDTER | 291 | 841.899 | 649.2782 |
| histidine-rich glycoprotein precursor | NP_000403.1 | DSPVLIDFFEDTER | 291 | 841.899 | 796.3466 |
| histidine-rich glycoprotein precursor | NP_000403.1 | DSPVLIDFFEDTER | 291 | 841.899 | 943.415 |
| histidine-rich glycoprotein precursor | NP_000403.1 | DSPVLIDFFEDTER | 291 | 841.899 | 1058.442 |
| histidine-rich glycoprotein precursor | NP_000403.1 | DSPVLIDFFEDTER | 291 | 841.899 | 1171.526 |
| histidine-rich glycoprotein precursor | NP_000403.1 | DSPVLIDFFEDTER | 291 | 841.899 | 1284.61 |
| histidine-rich glycoprotein precursor | NP_000403.1 | DSPVLIDFFEDTER | 291 | 841.899 | 1383.678 |
| histidine-rich glycoprotein precursor | NP_000403.1 | DSPVLIDFFEDTER | 291 | 841.899 | 1480.731 |
| histidine-rich glycoprotein precursor | NP_000403.1 | ADLFYDVEALDLESPK | 292 | 912.9487 | 331.1971 |
| histidine-rich glycoprotein precursor | NP_000403.1 | ADLFYDVEALDLESPK | 292 | 912.9487 | 460.2397 |
| histidine-rich glycoprotein precursor | NP_000403.1 | ADLFYDVEALDLESPK | 292 | 912.9487 | 573.3237 |
| histidine-rich glycoprotein precursor | NP_000403.1 | ADLFYDVEALDLESPK | 292 | 912.9487 | 688.3506 |
| histidine-rich glycoprotein precursor | NP_000403.1 | ADLFYDVEALDLESPK | 292 | 912.9487 | 801.4347 |
| histidine-rich glycoprotein precursor | NP_000403.1 | ADLFYDVEALDLESPK | 292 | 912.9487 | 872.4718 |
| histidine-rich glycoprotein precursor | NP_000403.1 | ADLFYDVEALDLESPK | 292 | 912.9487 | 1001.514 |
| histidine-rich glycoprotein precursor | NP_000403.1 | ADLFYDVEALDLESPK | 292 | 912.9487 | 1100.583 |
| histidine-rich glycoprotein precursor | NP_000403.1 | ADLFYDVEALDLESPK | 292 | 912.9487 | 1215.61 |
| histidine-rich glycoprotein precursor | NP_000403.1 | ADLFYDVEALDLESPK | 292 | 912.9487 | 1378.673 |
| apolipoprotein B precursor | NP_000375.2 | FDHTNSLNIAGLSLDFSSK | 293 | 1033.513 | 321.1763 |
| apolipoprotein B precursor | NP_000375.2 | FDHTNSLNIAGLSLDFSSK | 293 | 1033.513 | 468.2447 |
| apolipoprotein B precursor | NP_000375.2 | FDHTNSLNIAGLSLDFSSK | 293 | 1033.513 | 583.2717 |
| apolipoprotein B precursor | NP_000375.2 | FDHTNSLNIAGLSLDFSSK | 293 | 1033.513 | 696.3557 |
| apolipoprotein B precursor | NP_000375.2 | FDHTNSLNIAGLSLDFSSK | 293 | 1033.513 | 783.3878 |
| apolipoprotein B precursor | NP_000375.2 | FDHTNSLNIAGLSLDFSSK | 293 | 1033.513 | 896.4718 |
| apolipoprotein B precursor | NP_000375.2 | FDHTNSLNIAGLSLDFSSK | 293 | 1033.513 | 953.4933 |
| apolipoprotein B precursor | NP_000375.2 | FDHTNSLNIAGLSLDFSSK | 293 | 1033.513 | 1024.53 |
| apolipoprotein B precursor | NP_000375.2 | FDHTNSLNIAGLSLDFSSK | 293 | 1033.513 | 1137.615 |
| apolipoprotein B precursor | NP_000375.2 | FDHTNSLNIAGLSLDFSSK | 293 | 1033.513 | 1251.657 |
| apolipoprotein B precursor | NP_000375.2 | FDHTNSLNIAGLSLDFSSK | 293 | 1033.513 | 1364.741 |
| apolipoprotein B precursor | NP_000375.2 | FDHTNSLNIAGLSLDFSSK | 293 | 1033.513 | 1451.773 |
| pregnancy-zone protein | NP_002855.1 | ALLAYAFSLLGK | 294 | 633.8765 | 317.2178 |
| pregnancy-zone protein | NP_002855.1 | ALLAYAFSLLGK | 294 | 633.8765 | 430.3018 |
| pregnancy-zone protein | NP_002855.1 | ALLAYAFSLLGK | 294 | 633.8765 | 517.3339 |
| pregnancy-zone protein | NP_002855.1 | ALLAYAFSLLGK | 294 | 633.8765 | 664.4023 |
| pregnancy-zone protein | NP_002855.1 | ALLAYAFSLLGK | 294 | 633.8765 | 735.4394 |
| pregnancy-zone protein | NP_002855.1 | ALLAYAFSLLGK | 294 | 633.8765 | 898.5027 |
| pregnancy-zone protein | NP_002855.1 | ALLAYAFSLLGK | 294 | 633.8765 | 969.5399 |
| pregnancy-zone protein | NP_002855.1 | ALLAYAFSLLGK | 294 | 633.8765 | 1082.624 |
| pregnancy-zone protein | NP_002855.1 | ALLAYAFSLLGK | 294 | 633.8765 | 1195.708 |
| pregnancy-zone protein | NP_002855.1 | LEAGINQLSFPLSSEPIQGSYR | 295 | 1203.619 | 425.2137 |
| pregnancy-zone protein | NP_002855.1 | LEAGINQLSFPLSSEPIQGSYR | 295 | 1203.619 | 482.2352 |
| pregnancy-zone protein | NP_002855.1 | LEAGINQLSFPLSSEPIQGSYR | 295 | 1203.619 | 610.2938 |
| pregnancy-zone protein | NP_002855.1 | LEAGINQLSFPLSSEPIQGSYR | 295 | 1203.619 | 723.3779 |
| pregnancy-zone protein | NP_002855.1 | LEAGINQLSFPLSSEPIQGSYR | 295 | 1203.619 | 820.4306 |
| pregnancy-zone protein | NP_002855.1 | LEAGINQLSFPLSSEPIQGSYR | 295 | 1203.619 | 949.4732 |
| pregnancy-zone protein | NP_002855.1 | LEAGINQLSFPLSSEPIQGSYR | 295 | 1203.619 | 1036.505 |
| pregnancy-zone protein | NP_002855.1 | LEAGINQLSFPLSSEPIQGSYR | 295 | 1203.619 | 1123.537 |
| pregnancy-zone protein | NP_002855.1 | LEAGINQLSFPLSSEPIQGSYR | 295 | 1203.619 | 1236.621 |
| pregnancy-zone protein | NP_002855.1 | LEAGINQLSFPLSSEPIQGSYR | 295 | 1203.619 | 1333.674 |
| pregnancy-zone protein | NP_002855.1 | LEAGINQLSFPLSSEPIQGSYR | 295 | 1203.619 | 1480.743 |
| pregnancy-zone protein | NP_002855.1 | NELIPLIYLENPR | 296 | 792.4432 | 386.2141 |
| pregnancy-zone protein | NP_002855.1 | NELIPLIYLENPR | 296 | 792.4432 | 515.2567 |
| pregnancy-zone protein | NP_002855.1 | NELIPLIYLENPR | 296 | 792.4432 | 628.3408 |
| pregnancy-zone protein | NP_002855.1 | NELIPLIYLENPR | 296 | 792.4432 | 791.4041 |
| pregnancy-zone protein | NP_002855.1 | NELIPLIYLENPR | 296 | 792.4432 | 904.4882 |
| pregnancy-zone protein | NP_002855.1 | NELIPLIYLENPR | 296 | 792.4432 | 1017.572 |
| pregnancy-zone protein | NP_002855.1 | NELIPLIYLENPR | 296 | 792.4432 | 1114.625 |
| pregnancy-zone protein | NP_002855.1 | NELIPLIYLENPR | 296 | 792.4432 | 1227.709 |
| pregnancy-zone protein | NP_002855.1 | NELIPLIYLENPR | 296 | 792.4432 | 1340.793 |
| pregnancy-zone protein | NP_002855.1 | NELIPLIYLENPR | 296 | 792.4432 | 1469.836 |
| apolipoprotein B precursor | NP_000375.2 | HFVINLIGDFEVAEK | 297 | 865.9592 | 347.192 |
| apolipoprotein B precursor | NP_000375.2 | HFVINLIGDFEVAEK | 297 | 865.9592 | 446.2604 |
| apolipoprotein B precursor | NP_000375.2 | HFVINLIGDFEVAEK | 297 | 865.9592 | 575.303 |
| apolipoprotein B precursor | NP_000375.2 | HFVINLIGDFEVAEK | 297 | 865.9592 | 722.3714 |
| apolipoprotein B precursor | NP_000375.2 | HFVINLIGDFEVAEK | 297 | 865.9592 | 837.3983 |
| apolipoprotein B precursor | NP_000375.2 | HFVINLIGDFEVAEK | 297 | 865.9592 | 894.4198 |
| apolipoprotein B precursor | NP_000375.2 | HFVINLIGDFEVAEK | 297 | 865.9592 | 1007.504 |
| apolipoprotein B precursor | NP_000375.2 | HFVINLIGDFEVAEK | 297 | 865.9592 | 1120.588 |
| apolipoprotein B precursor | NP_000375.2 | HFVINLIGDFEVAEK | 297 | 865.9592 | 1234.631 |
| apolipoprotein B precursor | NP_000375.2 | HFVINLIGDFEVAEK | 297 | 865.9592 | 1347.715 |
| apolipoprotein B precursor | NP_000375.2 | HFVINLIGDFEVAEK | 297 | 865.9592 | 1446.783 |
| histidine-rich glycoprotein precursor | NP_000403.1 | GEVLPLPEANFPSFPLPHHK | 298 | 1113.589 | 421.2301 |
| histidine-rich glycoprotein precursor | NP_000403.1 | GEVLPLPEANFPSFPLPHHK | 298 | 1113.589 | 518.2828 |
| histidine-rich glycoprotein precursor | NP_000403.1 | GEVLPLPEANFPSFPLPHHK | 298 | 1113.589 | 631.3669 |
| histidine-rich glycoprotein precursor | NP_000403.1 | GEVLPLPEANFPSFPLPHHK | 298 | 1113.589 | 728.4197 |
| histidine-rich glycoprotein precursor | NP_000403.1 | GEVLPLPEANFPSFPLPHHK | 298 | 1113.589 | 875.4881 |
| histidine-rich glycoprotein precursor | NP_000403.1 | GEVLPLPEANFPSFPLPHHK | 298 | 1113.589 | 962.5201 |
| histidine-rich glycoprotein precursor | NP_000403.1 | GEVLPLPEANFPSFPLPHHK | 298 | 1113.589 | 1059.573 |
| histidine-rich glycoprotein precursor | NP_000403.1 | GEVLPLPEANFPSFPLPHHK | 298 | 1113.589 | 1206.641 |
| histidine-rich glycoprotein precursor | NP_000403.1 | GEVLPLPEANFPSFPLPHHK | 298 | 1113.589 | 1320.684 |
| histidine-rich glycoprotein precursor | NP_000403.1 | GEVLPLPEANFPSFPLPHHK | 298 | 1113.589 | 1391.721 |
| apolipoprotein B precursor | NP_000375.2 | ATFQTPDFIVPLTDLR | 299 | 917.4909 | 403.2294 |
| apolipoprotein B precursor | NP_000375.2 | ATFQTPDFIVPLTDLR | 299 | 917.4909 | 504.2771 |
| apolipoprotein B precursor | NP_000375.2 | ATFQTPDFIVPLTDLR | 299 | 917.4909 | 617.3611 |
| apolipoprotein B precursor | NP_000375.2 | ATFQTPDFIVPLTDLR | 299 | 917.4909 | 714.4139 |
| apolipoprotein B precursor | NP_000375.2 | ATFQTPDFIVPLTDLR | 299 | 917.4909 | 813.4823 |
| apolipoprotein B precursor | NP_000375.2 | ATFQTPDFIVPLTDLR | 299 | 917.4909 | 926.5664 |
| apolipoprotein B precursor | NP_000375.2 | ATFQTPDFIVPLTDLR | 299 | 917.4909 | 1073.635 |
| apolipoprotein B precursor | NP_000375.2 | ATFQTPDFIVPLTDLR | 299 | 917.4909 | 1188.662 |
| apolipoprotein B precursor | NP_000375.2 | ATFQTPDFIVPLTDLR | 299 | 917.4909 | 1285.714 |
| apolipoprotein B precursor | NP_000375.2 | ATFQTPDFIVPLTDLR | 299 | 917.4909 | 1386.762 |
| apolipoprotein B precursor | NP_000375.2 | FSVPAGIVIPSFQALTAR | 300 | 937.5303 | 347.2032 |
| apolipoprotein B precursor | NP_000375.2 | FSVPAGIVIPSFQALTAR | 300 | 937.5303 | 460.2873 |
| apolipoprotein B precursor | NP_000375.2 | FSVPAGIVIPSFQALTAR | 300 | 937.5303 | 531.3244 |
| apolipoprotein B precursor | NP_000375.2 | FSVPAGIVIPSFQALTAR | 300 | 937.5303 | 659.3829 |
| apolipoprotein B precursor | NP_000375.2 | FSVPAGIVIPSFQALTAR | 300 | 937.5303 | 806.4514 |
| apolipoprotein B precursor | NP_000375.2 | FSVPAGIVIPSFQALTAR | 300 | 937.5303 | 893.4834 |
| apolipoprotein B precursor | NP_000375.2 | FSVPAGIVIPSFQALTAR | 300 | 937.5303 | 990.5361 |
| apolipoprotein B precursor | NP_000375.2 | FSVPAGIVIPSFQALTAR | 300 | 937.5303 | 1103.62 |
| apolipoprotein B precursor | NP_000375.2 | FSVPAGIVIPSFQALTAR | 300 | 937.5303 | 1202.689 |
| apolipoprotein B precursor | NP_000375.2 | FSVPAGIVIPSFQALTAR | 300 | 937.5303 | 1315.773 |
| apolipoprotein B precursor | NP_000375.2 | FSVPAGIVIPSFQALTAR | 300 | 937.5303 | 1372.794 |
| apolipoprotein B precursor | NP_000375.2 | FSVPAGIVIPSFQALTAR | 300 | 937.5303 | 1443.831 |
| apolipoprotein B precursor | NP_000375.2 | NSLFFSAQPFEITASTNNEGNLK | 301 | 1265.116 | 374.2393 |
| apolipoprotein B precursor | NP_000375.2 | NSLFFSAQPFEITASTNNEGNLK | 301 | 1265.116 | 431.2607 |
| apolipoprotein B precursor | NP_000375.2 | NSLFFSAQPFEITASTNNEGNLK | 301 | 1265.116 | 560.3033 |
| apolipoprotein B precursor | NP_000375.2 | NSLFFSAQPFEITASTNNEGNLK | 301 | 1265.116 | 674.3463 |
| apolipoprotein B precursor | NP_000375.2 | NSLFFSAQPFEITASTNNEGNLK | 301 | 1265.116 | 788.3892 |
| apolipoprotein B precursor | NP_000375.2 | NSLFFSAQPFEITASTNNEGNLK | 301 | 1265.116 | 889.4368 |
| apolipoprotein B precursor | NP_000375.2 | NSLFFSAQPFEITASTNNEGNLK | 301 | 1265.116 | 976.4689 |
| apolipoprotein B precursor | NP_000375.2 | NSLFFSAQPFEITASTNNEGNLK | 301 | 1265.116 | 1047.506 |
| apolipoprotein B precursor | NP_000375.2 | NSLFFSAQPFEITASTNNEGNLK | 301 | 1265.116 | 1148.554 |
| apolipoprotein B precursor | NP_000375.2 | NSLFFSAQPFEITASTNNEGNLK | 301 | 1265.116 | 1261.638 |
| apolipoprotein B precursor | NP_000375.2 | NSLFFSAQPFEITASTNNEGNLK | 301 | 1265.116 | 1390.68 |
| pregnancy-zone protein | NP_002855.1 | QGIPFFAQVLLVDGK | 302 | 816.4614 | 319.1607 |
| pregnancy-zone protein | NP_002855.1 | QGIPFFAQVLLVDGK | 302 | 816.4614 | 418.2291 |
| pregnancy-zone protein | NP_002855.1 | QGIPFFAQVLLVDGK | 302 | 816.4614 | 531.3131 |
| pregnancy-zone protein | NP_002855.1 | QGIPFFAQVLLVDGK | 302 | 816.4614 | 644.3972 |
| pregnancy-zone protein | NP_002855.1 | QGIPFFAQVLLVDGK | 302 | 816.4614 | 743.4656 |
| pregnancy-zone protein | NP_002855.1 | QGIPFFAQVLLVDGK | 302 | 816.4614 | 871.5242 |
| pregnancy-zone protein | NP_002855.1 | QGIPFFAQVLLVDGK | 302 | 816.4614 | 942.5613 |
| pregnancy-zone protein | NP_002855.1 | QGIPFFAQVLLVDGK | 302 | 816.4614 | 1089.63 |
| pregnancy-zone protein | NP_002855.1 | QGIPFFAQVLLVDGK | 302 | 816.4614 | 1236.698 |
| pregnancy-zone protein | NP_002855.1 | QGIPFFAQVLLVDGK | 302 | 816.4614 | 1333.751 |
| pregnancy-zone protein | NP_002855.1 | QGIPFFAQVLLVDGK | 302 | 816.4614 | 1446.835 |
| apolipoprotein B precursor | NP_000375.2 | QTVNLQLQPYSLVTTLNSDLK | 303 | 1188.145 | 375.2233 |
| apolipoprotein B precursor | NP_000375.2 | QTVNLQLQPYSLVTTLNSDLK | 303 | 1188.145 | 462.2553 |
| apolipoprotein B precursor | NP_000375.2 | QTVNLQLQPYSLVTTLNSDLK | 303 | 1188.145 | 576.2982 |
| apolipoprotein B precursor | NP_000375.2 | QTVNLQLQPYSLVTTLNSDLK | 303 | 1188.145 | 689.3823 |
| apolipoprotein B precursor | NP_000375.2 | QTVNLQLQPYSLVTTLNSDLK | 303 | 1188.145 | 790.4299 |
| apolipoprotein B precursor | NP_000375.2 | QTVNLQLQPYSLVTTLNSDLK | 303 | 1188.145 | 891.4777 |
| apolipoprotein B precursor | NP_000375.2 | QTVNLQLQPYSLVTTLNSDLK | 303 | 1188.145 | 990.5461 |
| apolipoprotein B precursor | NP_000375.2 | QTVNLQLQPYSLVTTLNSDLK | 303 | 1188.145 | 1103.63 |
| apolipoprotein B precursor | NP_000375.2 | QTVNLQLQPYSLVTTLNSDLK | 303 | 1188.145 | 1190.662 |
| apolipoprotein B precursor | NP_000375.2 | QTVNLQLQPYSLVTTLNSDLK | 303 | 1188.145 | 1353.725 |
| apolipoprotein B precursor | NP_000375.2 | QTVNLQLQPYSLVTTLNSDLK | 303 | 1188.145 | 1450.778 |
| apolipoprotein B precursor | NP_000375.2 | TIHDLHLFIENIDFNK | 304 | 985.0125 | 408.2236 |
| apolipoprotein B precursor | NP_000375.2 | TIHDLHLFIENIDFNK | 304 | 985.0125 | 523.2505 |
| apolipoprotein B precursor | NP_000375.2 | TIHDLHLFIENIDFNK | 304 | 985.0125 | 636.3346 |
| apolipoprotein B precursor | NP_000375.2 | TIHDLHLFIENIDFNK | 304 | 985.0125 | 750.3775 |
| apolipoprotein B precursor | NP_000375.2 | TIHDLHLFIENIDFNK | 304 | 985.0125 | 879.4201 |
| apolipoprotein B precursor | NP_000375.2 | TIHDLHLFIENIDFNK | 304 | 985.0125 | 992.5042 |
| apolipoprotein B precursor | NP_000375.2 | TIHDLHLFIENIDFNK | 304 | 985.0125 | 1139.573 |
| apolipoprotein B precursor | NP_000375.2 | TIHDLHLFIENIDFNK | 304 | 985.0125 | 1252.657 |
| apolipoprotein B precursor | NP_000375.2 | TIHDLHLFIENIDFNK | 304 | 985.0125 | 1389.716 |
| apolipoprotein E precursor | NP_000032.1 | WVQTLSEQVQEELLSSQVTQELR | 305 | 1365.701 | 417.2451 |
| apolipoprotein E precursor | NP_000032.1 | WVQTLSEQVQEELLSSQVTQELR | 305 | 1365.701 | 545.3036 |
| apolipoprotein E precursor | NP_000032.1 | WVQTLSEQVQEELLSSQVTQELR | 305 | 1365.701 | 646.3513 |
| apolipoprotein E precursor | NP_000032.1 | WVQTLSEQVQEELLSSQVTQELR | 305 | 1365.701 | 745.4197 |
| apolipoprotein E precursor | NP_000032.1 | WVQTLSEQVQEELLSSQVTQELR | 305 | 1365.701 | 873.4783 |
| apolipoprotein E precursor | NP_000032.1 | WVQTLSEQVQEELLSSQVTQELR | 305 | 1365.701 | 960.5103 |
| apolipoprotein E precursor | NP_000032.1 | WVQTLSEQVQEELLSSQVTQELR | 305 | 1365.701 | 1047.542 |
| apolipoprotein E precursor | NP_000032.1 | WVQTLSEQVQEELLSSQVTQELR | 305 | 1365.701 | 1160.626 |
| apolipoprotein E precursor | NP_000032.1 | WVQTLSEQVQEELLSSQVTQELR | 305 | 1365.701 | 1273.71 |
| apolipoprotein E precursor | NP_000032.1 | WVQTLSEQVQEELLSSQVTQELR | 305 | 1365.701 | 1402.753 |
| apolipoprotein B precursor | NP_000375.2 | HLIDSLIDFLNFPR | 306 | 850.462 | 419.2396 |
| apolipoprotein B precursor | NP_000375.2 | HLIDSLIDFLNFPR | 306 | 850.462 | 533.2825 |
| apolipoprotein B precursor | NP_000375.2 | HLIDSLIDFLNFPR | 306 | 850.462 | 646.3666 |
| apolipoprotein B precursor | NP_000375.2 | HLIDSLIDFLNFPR | 306 | 850.462 | 793.435 |
| apolipoprotein B precursor | NP_000375.2 | HLIDSLIDFLNFPR | 306 | 850.462 | 908.4619 |
| apolipoprotein B precursor | NP_000375.2 | HLIDSLIDFLNFPR | 306 | 850.462 | 1021.546 |
| apolipoprotein B precursor | NP_000375.2 | HLIDSLIDFLNFPR | 306 | 850.462 | 1134.63 |
| apolipoprotein B precursor | NP_000375.2 | HLIDSLIDFLNFPR | 306 | 850.462 | 1221.662 |
| apolipoprotein B precursor | NP_000375.2 | HLIDSLIDFLNFPR | 306 | 850.462 | 1336.689 |
| apolipoprotein B precursor | NP_000375.2 | HLIDSLIDFLNFPR | 306 | 850.462 | 1449.773 |
| apolipoprotein B precursor | NP_000375.2 | NFVASHIANILNSEELDIQDLK | 307 | 1242.142 | 375.2233 |
| apolipoprotein B precursor | NP_000375.2 | NFVASHIANILNSEELDIQDLK | 307 | 1242.142 | 503.2818 |
| apolipoprotein B precursor | NP_000375.2 | NFVASHIANILNSEELDIQDLK | 307 | 1242.142 | 616.3659 |
| apolipoprotein B precursor | NP_000375.2 | NFVASHIANILNSEELDIQDLK | 307 | 1242.142 | 731.3928 |
| apolipoprotein B precursor | NP_000375.2 | NFVASHIANILNSEELDIQDLK | 307 | 1242.142 | 844.4769 |
| apolipoprotein B precursor | NP_000375.2 | NFVASHIANILNSEELDIQDLK | 307 | 1242.142 | 973.5195 |
| apolipoprotein B precursor | NP_000375.2 | NFVASHIANILNSEELDIQDLK | 307 | 1242.142 | 1102.562 |
| apolipoprotein B precursor | NP_000375.2 | NFVASHIANILNSEELDIQDLK | 307 | 1242.142 | 1189.594 |
| apolipoprotein B precursor | NP_000375.2 | NFVASHIANILNSEELDIQDLK | 307 | 1242.142 | 1303.637 |
| apolipoprotein B precursor | NP_000375.2 | NFVASHIANILNSEELDIQDLK | 307 | 1242.142 | 1416.721 |
| apolipoprotein A-II preproprotein | NP_001634.1 | AGTELVNFLSYFVELGTQPATQ | 308 | 1193.102 | 319.1607 |
| apolipoprotein A-II preproprotein | NP_001634.1 | AGTELVNFLSYFVELGTQPATQ | 308 | 1193.102 | 416.2134 |
| apolipoprotein A-II preproprotein | NP_001634.1 | AGTELVNFLSYFVELGTQPATQ | 308 | 1193.102 | 544.272 |
| apolipoprotein A-II preproprotein | NP_001634.1 | AGTELVNFLSYFVELGTQPATQ | 308 | 1193.102 | 645.3197 |
| apolipoprotein A-II preproprotein | NP_001634.1 | AGTELVNFLSYFVELGTQPATQ | 308 | 1193.102 | 702.3411 |
| apolipoprotein A-II preproprotein | NP_001634.1 | AGTELVNFLSYFVELGTQPATQ | 308 | 1193.102 | 815.4252 |
| apolipoprotein A-II preproprotein | NP_001634.1 | AGTELVNFLSYFVELGTQPATQ | 308 | 1193.102 | 944.4678 |
| apolipoprotein A-II preproprotein | NP_001634.1 | AGTELVNFLSYFVELGTQPATQ | 308 | 1193.102 | 1043.536 |
| apolipoprotein A-II preproprotein | NP_001634.1 | AGTELVNFLSYFVELGTQPATQ | 308 | 1193.102 | 1190.605 |
| apolipoprotein A-II preproprotein | NP_001634.1 | AGTELVNFLSYFVELGTQPATQ | 308 | 1193.102 | 1353.668 |
| apolipoprotein A-II preproprotein | NP_001634.1 | AGTELVNFLSYFVELGTQPATQ | 308 | 1193.102 | 1440.7 |
| alpha-2-macroglobulin precursor | NP_000005.2 | VSNQTLSLFFTVLQDVPVR | 309 | 1082.094 | 371.2396 |
| alpha-2-macroglobulin precursor | NP_000005.2 | VSNQTLSLFFTVLQDVPVR | 309 | 1082.094 | 470.308 |
| alpha-2-macroglobulin precursor | NP_000005.2 | VSNQTLSLFFTVLQDVPVR | 309 | 1082.094 | 585.335 |
| alpha-2-macroglobulin precursor | NP_000005.2 | VSNQTLSLFFTVLQDVPVR | 309 | 1082.094 | 713.3935 |
| alpha-2-macroglobulin precursor | NP_000005.2 | VSNQTLSLFFTVLQDVPVR | 309 | 1082.094 | 826.4776 |
| alpha-2-macroglobulin precursor | NP_000005.2 | VSNQTLSLFFTVLQDVPVR | 309 | 1082.094 | 925.546 |
| alpha-2-macroglobulin precursor | NP_000005.2 | VSNQTLSLFFTVLQDVPVR | 309 | 1082.094 | 1026.594 |
| alpha-2-macroglobulin precursor | NP_000005.2 | VSNQTLSLFFTVLQDVPVR | 309 | 1082.094 | 1173.662 |
| alpha-2-macroglobulin precursor | NP_000005.2 | VSNQTLSLFFTVLQDVPVR | 309 | 1082.094 | 1320.73 |
| alpha-2-macroglobulin precursor | NP_000005.2 | VSNQTLSLFFTVLQDVPVR | 309 | 1082.094 | 1433.815 |
| apolipoprotein B precursor | NP_000375.2 | NQDVHSINLPFFETLQEYFER | 310 | 1313.632 | 451.2294 |
| apolipoprotein B precursor | NP_000375.2 | NQDVHSINLPFFETLQEYFER | 310 | 1313.632 | 614.2927 |
| apolipoprotein B precursor | NP_000375.2 | NQDVHSINLPFFETLQEYFER | 310 | 1313.632 | 743.3353 |
| apolipoprotein B precursor | NP_000375.2 | NQDVHSINLPFFETLQEYFER | 310 | 1313.632 | 871.3939 |
| apolipoprotein B precursor | NP_000375.2 | NQDVHSINLPFFETLQEYFER | 310 | 1313.632 | 984.478 |
| apolipoprotein B precursor | NP_000375.2 | NQDVHSINLPFFETLQEYFER | 310 | 1313.632 | 1085.526 |
| apolipoprotein B precursor | NP_000375.2 | NQDVHSINLPFFETLQEYFER | 310 | 1313.632 | 1214.568 |
| apolipoprotein B precursor | NP_000375.2 | NQDVHSINLPFFETLQEYFER | 310 | 1313.632 | 1361.637 |
| ceruloplasmin precursor | NP_000087.1 | GPEEEHLGILGPVIWAEVGDTIR | 311 | 1244.148 | 389.2502 |
| ceruloplasmin precursor | NP_000087.1 | GPEEEHLGILGPVIWAEVGDTIR | 311 | 1244.148 | 504.2771 |
| ceruloplasmin precursor | NP_000087.1 | GPEEEHLGILGPVIWAEVGDTIR | 311 | 1244.148 | 561.2985 |
| ceruloplasmin precursor | NP_000087.1 | GPEEEHLGILGPVIWAEVGDTIR | 311 | 1244.148 | 660.3669 |
| ceruloplasmin precursor | NP_000087.1 | GPEEEHLGILGPVIWAEVGDTIR | 311 | 1244.148 | 789.4095 |
| ceruloplasmin precursor | NP_000087.1 | GPEEEHLGILGPVIWAEVGDTIR | 311 | 1244.148 | 860.4467 |
| ceruloplasmin precursor | NP_000087.1 | GPEEEHLGILGPVIWAEVGDTIR | 311 | 1244.148 | 1046.526 |
| ceruloplasmin precursor | NP_000087.1 | GPEEEHLGILGPVIWAEVGDTIR | 311 | 1244.148 | 1159.61 |
| ceruloplasmin precursor | NP_000087.1 | GPEEEHLGILGPVIWAEVGDTIR | 311 | 1244.148 | 1258.678 |
| ceruloplasmin precursor | NP_000087.1 | GPEEEHLGILGPVIWAEVGDTIR | 311 | 1244.148 | 1355.731 |
| ceruloplasmin precursor | NP_000087.1 | GPEEEHLGILGPVIWAEVGDTIR | 311 | 1244.148 | 1412.753 |
| paraoxonase 1 | NP_000437.3 | GIETGSEDLEILPNGLAFISSGLK | 312 | 1230.647 | 317.2178 |
| paraoxonase 1 | NP_000437.3 | GIETGSEDLEILPNGLAFISSGLK | 312 | 1230.647 | 404.2498 |
| paraoxonase 1 | NP_000437.3 | GIETGSEDLEILPNGLAFISSGLK | 312 | 1230.647 | 491.2818 |
| paraoxonase 1 | NP_000437.3 | GIETGSEDLEILPNGLAFISSGLK | 312 | 1230.647 | 604.3659 |
| paraoxonase 1 | NP_000437.3 | GIETGSEDLEILPNGLAFISSGLK | 312 | 1230.647 | 751.4343 |
| paraoxonase 1 | NP_000437.3 | GIETGSEDLEILPNGLAFISSGLK | 312 | 1230.647 | 822.4714 |
| paraoxonase 1 | NP_000437.3 | GIETGSEDLEILPNGLAFISSGLK | 312 | 1230.647 | 935.5555 |
| paraoxonase 1 | NP_000437.3 | GIETGSEDLEILPNGLAFISSGLK | 312 | 1230.647 | 992.577 |
| paraoxonase 1 | NP_000437.3 | GIETGSEDLEILPNGLAFISSGLK | 312 | 1230.647 | 1106.62 |
| paraoxonase 1 | NP_000437.3 | GIETGSEDLEILPNGLAFISSGLK | 312 | 1230.647 | 1203.673 |
| paraoxonase 1 | NP_000437.3 | GIETGSEDLEILPNGLAFISSGLK | 312 | 1230.647 | 1316.757 |
| paraoxonase 1 | NP_000437.3 | GIETGSEDLEILPNGLAFISSGLK | 312 | 1230.647 | 1429.841 |
| serum amyloid A4; constitutive | NP_006503.1 | VYLQGLIDYYLFGNSSTVLEDSK | 313 | 1312.66 | 349.1712 |
| serum amyloid A4; constitutive | NP_006503.1 | VYLQGLIDYYLFGNSSTVLEDSK | 313 | 1312.66 | 478.2138 |
| serum amyloid A4; constitutive | NP_006503.1 | VYLQGLIDYYLFGNSSTVLEDSK | 313 | 1312.66 | 591.2979 |
| serum amyloid A4; constitutive | NP_006503.1 | VYLQGLIDYYLFGNSSTVLEDSK | 313 | 1312.66 | 690.3663 |
| serum amyloid A4; constitutive | NP_006503.1 | VYLQGLIDYYLFGNSSTVLEDSK | 313 | 1312.66 | 791.414 |
| serum amyloid A4; constitutive | NP_006503.1 | VYLQGLIDYYLFGNSSTVLEDSK | 313 | 1312.66 | 878.446 |
| serum amyloid A4; constitutive | NP_006503.1 | VYLQGLIDYYLFGNSSTVLEDSK | 313 | 1312.66 | 965.478 |
| serum amyloid A4; constitutive | NP_006503.1 | VYLQGLIDYYLFGNSSTVLEDSK | 313 | 1312.66 | 1079.521 |
| serum amyloid A4; constitutive | NP_006503.1 | VYLQGLIDYYLFGNSSTVLEDSK | 313 | 1312.66 | 1136.542 |
| serum amyloid A4; constitutive | NP_006503.1 | VYLQGLIDYYLFGNSSTVLEDSK | 313 | 1312.66 | 1283.611 |
| serum amyloid A4; constitutive | NP_006503.1 | VYLQGLIDYYLFGNSSTVLEDSK | 313 | 1312.66 | 1396.695 |
| apolipoprotein B precursor | NP_000375.2 | TTLTAFGFASADLIEIGLEGK | 314 | 1077.57 | 333.1763 |
| apolipoprotein B precursor | NP_000375.2 | TTLTAFGFASADLIEIGLEGK | 314 | 1077.57 | 446.2604 |
| apolipoprotein B precursor | NP_000375.2 | TTLTAFGFASADLIEIGLEGK | 314 | 1077.57 | 503.2818 |
| apolipoprotein B precursor | NP_000375.2 | TTLTAFGFASADLIEIGLEGK | 314 | 1077.57 | 616.3659 |
| apolipoprotein B precursor | NP_000375.2 | TTLTAFGFASADLIEIGLEGK | 314 | 1077.57 | 745.4085 |
| apolipoprotein B precursor | NP_000375.2 | TTLTAFGFASADLIEIGLEGK | 314 | 1077.57 | 858.4926 |
| apolipoprotein B precursor | NP_000375.2 | TTLTAFGFASADLIEIGLEGK | 314 | 1077.57 | 971.5766 |
| apolipoprotein B precursor | NP_000375.2 | TTLTAFGFASADLIEIGLEGK | 314 | 1077.57 | 1086.604 |
| apolipoprotein B precursor | NP_000375.2 | TTLTAFGFASADLIEIGLEGK | 314 | 1077.57 | 1157.641 |
| apolipoprotein B precursor | NP_000375.2 | TTLTAFGFASADLIEIGLEGK | 314 | 1077.57 | 1244.673 |
| apolipoprotein B precursor | NP_000375.2 | TTLTAFGFASADLIEIGLEGK | 314 | 1077.57 | 1315.71 |
| apolipoprotein B precursor | NP_000375.2 | TTLTAFGFASADLIEIGLEGK | 314 | 1077.57 | 1462.778 |
| apolipoprotein B precursor | NP_000375.2 | IDFLNNYALFLSPSAQQASWQV | 315 | 1413.714 | 333.1875 |
| | | SAR | | | |
| apolipoprotein B precursor | NP_000375.2 | IDFLNNYALFLSPSAQQASWQV | 315 | 1413.714 | 432.256 |
| | | SAR | | | |
| apolipoprotein B precursor | NP_000375.2 | IDFLNNYALFLSPSAQQASWQV | 315 | 1413.714 | 560.3145 |
| | | SAR | | | |
| apolipoprotein B precursor | NP_000375.2 | IDFLNNYALFLSPSAQQASWQV | 315 | 1413.714 | 746.3939 |
| | | SAR | | | |
| apolipoprotein B precursor | NP_000375.2 | IDFLNNYALFLSPSAQQASWQV | 315 | 1413.714 | 833.4259 |
| | | SAR | | | |
| apolipoprotein B precursor | NP_000375.2 | IDFLNNYALFLSPSAQQASWQV | 315 | 1413.714 | 904.463 |
| | | SAR | | | |
| apolipoprotein B precursor | NP_000375.2 | IDFLNNYALFLSPSAQQASWQV | 315 | 1413.714 | 1032.522 |
| | | SAR | | | |
| apolipoprotein B precursor | NP_000375.2 | IDFLNNYALFLSPSAQQASWQV | 315 | 1413.714 | 1160.58 |
| | | SAR | | | |
| apolipoprotein B precursor | NP_000375.2 | IDFLNNYALFLSPSAQQASWQV | 315 | 1413.714 | 1231.617 |
| | | SAR | | | |
| apolipoprotein B precursor | NP_000375.2 | IDFLNNYALFLSPSAQQASWQV | 315 | 1413.714 | 1318.649 |
| | | SAR | | | |
| apolipoprotein B precursor | NP_000375.2 | IDFLNNYALFLSPSAQQASWQV | 315 | 1413.714 | 1415.702 |
| | | SAR | | | |
| apolipoprotein B precursor | NP_000375.2 | IADFELPTIIVPEQTIEIPSIK | 316 | 1233.691 | 347.2283 |
| apolipoprotein B precursor | NP_000375.2 | IADFELPTIIVPEQTIEIPSIK | 316 | 1233.691 | 444.2811 |
| apolipoprotein B precursor | NP_000375.2 | IADFELPTIIVPEQTIEIPSIK | 316 | 1233.691 | 557.3652 |
| apolipoprotein B precursor | NP_000375.2 | IADFELPTIIVPEQTIEIPSIK | 316 | 1233.691 | 686.4078 |
| apolipoprotein B precursor | NP_000375.2 | IADFELPTIIVPEQTIEIPSIK | 316 | 1233.691 | 799.4918 |
| apolipoprotein B precursor | NP_000375.2 | IADFELPTIIVPEQTIEIPSIK | 316 | 1233.691 | 900.5395 |
| apolipoprotein B precursor | NP_000375.2 | IADFELPTIIVPEQTIEIPSIK | 316 | 1233.691 | 1028.598 |
| apolipoprotein B precursor | NP_000375.2 | IADFELPTIIVPEQTIEIPSIK | 316 | 1233.691 | 1157.641 |
| apolipoprotein B precursor | NP_000375.2 | IADFELPTIIVPEQTIEIPSIK | 316 | 1233.691 | 1254.693 |
| apolipoprotein B precursor | NP_000375.2 | IADFELPTIIVPEQTIEIPSIK | 316 | 1233.691 | 1353.762 |
| apolipoprotein B precursor | NP_000375.2 | IADFELPTIIVPEQTIEIPSIK | 316 | 1233.691 | 1466.846 |
| apolipoprotein B precursor | NP_000375.2 | VPSYTLILPSLELPVLHVPR | 316 | 1122.162 | 371.2396 |
| apolipoprotein B precursor | NP_000375.2 | VPSYTLILPSLELPVLHVPR | 316 | 1122.162 | 508.2985 |
| apolipoprotein B precursor | NP_000375.2 | VPSYTLILPSLELPVLHVPR | 316 | 1122.162 | 621.3826 |
| apolipoprotein B precursor | NP_000375.2 | VPSYTLILPSLELPVLHVPR | 316 | 1122.162 | 720.451 |
| apolipoprotein B precursor | NP_000375.2 | VPSYTLILPSLELPVLHVPR | 316 | 1122.162 | 817.5037 |
| apolipoprotein B precursor | NP_000375.2 | VPSYTLILPSLELPVLHVPR | 316 | 1122.162 | 930.5878 |
| apolipoprotein B precursor | NP_000375.2 | VPSYTLILPSLELPVLHVPR | 316 | 1122.162 | 1059.63 |
| apolipoprotein B precursor | NP_000375.2 | VPSYTLILPSLELPVLHVPR | 316 | 1122.162 | 1172.714 |
| apolipoprotein B precursor | NP_000375.2 | VPSYTLILPSLELPVLHVPR | 316 | 1122.162 | 1259.746 |
| apolipoprotein B precursor | NP_000375.2 | VPSYTLILPSLELPVLHVPR | 316 | 1122.162 | 1356.799 |
| apolipoprotein B precursor | NP_000375.2 | VPSYTLILPSLELPVLHVPR | 316 | 1122.162 | 1469.883 |
| apolipoprotein B precursor | NP_000375.2 | FFSLLSGSLNSHGLELNADILGT | 317 | 1324.682 | 363.1869 |
| | | DK | | | |
| apolipoprotein B precursor | NP_000375.2 | FFSLLSGSLNSHGLELNADILGT | 317 | 1324.682 | 420.2083 |
| | | DK | | | |
| apolipoprotein B precursor | NP_000375.2 | FFSLLSGSLNSHGLELNADILGT | 317 | 1324.682 | 533.2924 |
| | | DK | | | |
| apolipoprotein B precursor | NP_000375.2 | FFSLLSGSLNSHGLELNADILGT | 317 | 1324.682 | 646.3765 |
| | | DK | | | |
| apolipoprotein B precursor | NP_000375.2 | FFSLLSGSLNSHGLELNADILGT | 317 | 1324.682 | 761.4034 |
| | | DK | | | |
| apolipoprotein B precursor | NP_000375.2 | FFSLLSGSLNSHGLELNADILGT | 317 | 1324.682 | 832.4406 |
| | | DK | | | |
| apolipoprotein B precursor | NP_000375.2 | FFSLLSGSLNSHGLELNADILGT | 317 | 1324.682 | 946.4835 |
| | | DK | | | |
| apolipoprotein B precursor | NP_000375.2 | FFSLLSGSLNSHGLELNADILGT | 317 | 1324.682 | 1059.568 |
| | | DK | | | |
| apolipoprotein B precursor | NP_000375.2 | FFSLLSGSLNSHGLELNADILGT | 317 | 1324.682 | 1188.61 |
| | | DK | | | |
| apolipoprotein B precursor | NP_000375.2 | FFSLLSGSLNSHGLELNADILGT | 317 | 1324.682 | 1301.694 |
| | | DK | | | |
| apolipoprotein B precursor | NP_000375.2 | FFSLLSGSLNSHGLELNADILGT | 317 | 1324.682 | 1358.716 |
| | | DK | | | |
| apolipoprotein B precursor | NP_000375.2 | FFSLLSGSLNSHGLELNADILGT | 317 | 1324.682 | 1495.775 |
| | | DK | | | |
| ceruloplasmin precursor | NP_000087.1 | HYYIAAEEIIWNYAPSGIDIFTK | 318 | 1357.679 | 395.2283 |
| ceruloplasmin precursor | NP_000087.1 | HYYIAAEEIIWNYAPSGIDIFTK | 318 | 1357.679 | 508.3124 |
| ceruloplasmin precursor | NP_000087.1 | HYYIAAEEIIWNYAPSGIDIFTK | 318 | 1357.679 | 623.3394 |
| ceruloplasmin precursor | NP_000087.1 | HYYIAAEEIIWNYAPSGIDIFTK | 318 | 1357.679 | 736.4234 |
| ceruloplasmin precursor | NP_000087.1 | HYYIAAEEIIWNYAPSGIDIFTK | 318 | 1357.679 | 793.4449 |
| ceruloplasmin precursor | NP_000087.1 | HYYIAAEEIIWNYAPSGIDIFTK | 318 | 1357.679 | 880.4769 |
| ceruloplasmin precursor | NP_000087.1 | HYYIAAEEIIWNYAPSGIDIFTK | 318 | 1357.679 | 977.5297 |
| ceruloplasmin precursor | NP_000087.1 | HYYIAAEEIIWNYAPSGIDIFTK | 318 | 1357.679 | 1048.567 |
| ceruloplasmin precursor | NP_000087.1 | HYYIAAEEIIWNYAPSGIDIFTK | 318 | 1357.679 | 1211.63 |
| ceruloplasmin precursor | NP_000087.1 | HYYIAAEEIIWNYAPSGIDIFTK | 318 | 1357.679 | 1325.673 |
| alpha-2-macroglobulin precursor | NP_000005.2 | AYIFIDEAHITQALIWLSQR | 319 | 1194.639 | 390.209 |
| alpha-2-macroglobulin precursor | NP_000005.2 | AYIFIDEAHITQALIWLSQR | 319 | 1194.639 | 503.2931 |
| alpha-2-macroglobulin precursor | NP_000005.2 | AYIFIDEAHITQALIWLSQR | 319 | 1194.639 | 689.3724 |
| alpha-2-macroglobulin precursor | NP_000005.2 | AYIFIDEAHITQALIWLSQR | 319 | 1194.639 | 802.4564 |
| alpha-2-macroglobulin precursor | NP_000005.2 | AYIFIDEAHITQALIWLSQR | 319 | 1194.639 | 915.5405 |
| alpha-2-macroglobulin precursor | NP_000005.2 | AYIFIDEAHITQALIWLSQR | 319 | 1194.639 | 986.5776 |
| alpha-2-macroglobulin precursor | NP_000005.2 | AYIFIDEAHITQALIWLSQR | 319 | 1194.639 | 1114.636 |
| alpha-2-macroglobulin precursor | NP_000005.2 | AYIFIDEAHITQALIWLSQR | 319 | 1194.639 | 1215.684 |
| alpha-2-macroglobulin precursor | NP_000005.2 | AYIFIDEAHITQALIWLSQR | 319 | 1194.639 | 1328.768 |
| alpha-2-macroglobulin precursor | NP_000005.2 | AYIFIDEAHITQALIWLSQR | 319 | 1194.639 | 1465.827 |
| apolipoprotein B precursor | NP_000375.2 | NLQDLLQFIFQLIEDNIK | 320 | 1102.602 | 374.2393 |
| apolipoprotein B precursor | NP_000375.2 | NLQDLLQFIFQLIEDNIK | 320 | 1102.602 | 489.2662 |
| apolipoprotein B precursor | NP_000375.2 | NLQDLLQFIFQLIEDNIK | 320 | 1102.602 | 618.3088 |
| apolipoprotein B precursor | NP_000375.2 | NLQDLLQFIFQLIEDNIK | 320 | 1102.602 | 731.3928 |
| apolipoprotein B precursor | NP_000375.2 | NLQDLLQFIFQLIEDNIK | 320 | 1102.602 | 844.4769 |
| apolipoprotein B precursor | NP_000375.2 | NLQDLLQFIFQLIEDNIK | 320 | 1102.602 | 972.5355 |
| apolipoprotein B precursor | NP_000375.2 | NLQDLLQFIFQLIEDNIK | 320 | 1102.602 | 1119.604 |
| apolipoprotein B precursor | NP_000375.2 | NLQDLLQFIFQLIEDNIK | 320 | 1102.602 | 1232.688 |
| apolipoprotein B precursor | NP_000375.2 | NLQDLLQFIFQLIEDNIK | 320 | 1102.602 | 1379.756 |
| ceruloplasmin precursor | NP_000087.1 | EFYLFPTVFDENESLLLEDNIR | 321 | 1352.163 | 402.2454 |
| ceruloplasmin precursor | NP_000087.1 | EFYLFPTVFDENESLLLEDNIR | 321 | 1352.163 | 517.2723 |
| ceruloplasmin precursor | NP_000087.1 | EFYLFPTVFDENESLLLEDNIR | 321 | 1352.163 | 646.3149 |
| ceruloplasmin precursor | NP_000087.1 | EFYLFPTVFDENESLLLEDNIR | 321 | 1352.163 | 759.399 |
| ceruloplasmin precursor | NP_000087.1 | EFYLFPTVFDENESLLLEDNIR | 321 | 1352.163 | 872.483 |
| ceruloplasmin precursor | NP_000087.1 | EFYLFPTVFDENESLLLEDNIR | 321 | 1352.163 | 985.5671 |
| ceruloplasmin precursor | NP_000087.1 | EFYLFPTVFDENESLLLEDNIR | 321 | 1352.163 | 1072.599 |
| ceruloplasmin precursor | NP_000087.1 | EFYLFPTVFDENESLLLEDNIR | 321 | 1352.163 | 1201.642 |
| ceruloplasmin precursor | NP_000087.1 | EFYLFPTVFDENESLLLEDNIR | 321 | 1352.163 | 1315.685 |
| ceruloplasmin precursor | NP_000087.1 | EFYLFPTVFDENESLLLEDNIR | 321 | 1352.163 | 1444.727 |
| apolipoprotein B precursor | NP_000375.2 | YLSLVGQVYSTLVTYISDWWTL | 322 | 1439.257 | 289.1865 |
| | | AAK | | | |
| apolipoprotein B precursor | NP_000375.2 | YLSLVGQVYSTLVTYISDWWTL | 322 | 1439.257 | 402.2705 |
| | | AAK | | | |
| apolipoprotein B precursor | NP_000375.2 | YLSLVGQVYSTLVTYISDWWTL | 322 | 1439.257 | 503.3182 |
| | | AAK | | | |
| apolipoprotein B precursor | NP_000375.2 | YLSLVGQVYSTLVTYISDWWTL | 322 | 1439.257 | 689.3975 |
| | | AAK | | | |
| apolipoprotein B precursor | NP_000375.2 | YLSLVGQVYSTLVTYISDWWTL | 322 | 1439.257 | 875.4769 |
| | | AAK | | | |
| apolipoprotein B precursor | NP_000375.2 | YLSLVGQVYSTLVTYISDWWTL | 322 | 1439.257 | 990.5038 |
| | | AAK | | | |
| apolipoprotein B precursor | NP_000375.2 | YLSLVGQVYSTLVTYISDWWTL | 322 | 1439.257 | 1077.536 |
| | | AAK | | | |
| apolipoprotein B precursor | NP_000375.2 | YLSLVGQVYSTLVTYISDWWTL | 322 | 1439.257 | 1190.62 |
| | | AAK | | | |
| apolipoprotein B precursor | NP_000375.2 | YLSLVGQVYSTLVTYISDWWTL | 322 | 1439.257 | 1353.683 |
| | | AAK | | | |
| apolipoprotein B precursor | NP_000375.2 | YLSLVGQVYSTLVTYISDWWTL | 322 | 1439.257 | 1454.731 |
| | | AAK | | | |
| apolipoprotein B precursor | NP_000375.2 | LIDLSIQNYHTFLIYITELLK | 323 | 1275.714 | 373.2804 |
| apolipoprotein B precursor | NP_000375.2 | LIDLSIQNYHTFLIYITELLK | 323 | 1275.714 | 502.323 |
| apolipoprotein B precursor | NP_000375.2 | LIDLSIQNYHTFLIYITELLK | 323 | 1275.714 | 603.3707 |
| apolipoprotein B precursor | NP_000375.2 | LIDLSIQNYHTFLIYITELLK | 323 | 1275.714 | 716.4547 |
| apolipoprotein B precursor | NP_000375.2 | LIDLSIQNYHTFLIYITELLK | 323 | 1275.714 | 879.5181 |
| apolipoprotein B precursor | NP_000375.2 | LIDLSIQNYHTFLIYITELLK | 323 | 1275.714 | 992.6021 |
| apolipoprotein B precursor | NP_000375.2 | LIDLSIQNYHTFLIYITELLK | 323 | 1275.714 | 1105.686 |
| apolipoprotein B precursor | NP_000375.2 | LIDLSIQNYHTFLIYITELLK | 323 | 1275.714 | 1252.755 |
| apolipoprotein B precursor | NP_000375.2 | LIDLSIQNYHTFLIYITELLK | 323 | 1275.714 | 1353.802 |
| apolipoprotein B precursor | NP_000375.2 | LIDLSIQNYHTFLIYITELLK | 323 | 1275.714 | 1490.861 |
| apolipoprotein B precursor | NP_000375.2 | VHNGSEILFSYFQDLVITLPFELR | 324 | 1419.247 | 417.2451 |
| apolipoprotein B precursor | NP_000375.2 | VHNGSEILFSYFQDLVITLPFELR | 324 | 1419.247 | 564.3135 |
| apolipoprotein B precursor | NP_000375.2 | VHNGSEILFSYFQDLVITLPFELR | 324 | 1419.247 | 661.3662 |
| apolipoprotein B precursor | NP_000375.2 | VHNGSEILFSYFQDLVITLPFELR | 324 | 1419.247 | 774.4503 |
| apolipoprotein B precursor | NP_000375.2 | VHNGSEILFSYFQDLVITLPFELR | 324 | 1419.247 | 875.498 |
| apolipoprotein B precursor | NP_000375.2 | VHNGSEILFSYFQDLVITLPFELR | 324 | 1419.247 | 988.582 |
| apolipoprotein B precursor | NP_000375.2 | VHNGSEILFSYFQDLVITLPFELR | 324 | 1419.247 | 1087.651 |
| apolipoprotein B precursor | NP_000375.2 | VHNGSEILFSYFQDLVITLPFELR | 324 | 1419.247 | 1200.734 |
| apolipoprotein B precursor | NP_000375.2 | VHNGSEILFSYFQDLVITLPFELR | 324 | 1419.247 | 1315.761 |
| apolipoprotein B precursor | NP_000375.2 | VHNGSEILFSYFQDLVITLPFELR | 324 | 1419.247 | 1443.82 |
| ceruloplasmin precursor | NP_000087.1 | LEFALLFLVFDENESWYLDDNIK | 325 | 1417.202 | 374.2393 |
| ceruloplasmin precursor | NP_000087.1 | LEFALLFLVFDENESWYLDDNIK | 325 | 1417.202 | 489.2662 |
| ceruloplasmin precursor | NP_000087.1 | LEFALLFLVFDENESWYLDDNIK | 325 | 1417.202 | 604.2932 |
| ceruloplasmin precursor | NP_000087.1 | LEFALLFLVFDENESWYLDDNIK | 325 | 1417.202 | 717.3772 |
| ceruloplasmin precursor | NP_000087.1 | LEFALLFLVFDENESWYLDDNIK | 325 | 1417.202 | 880.4406 |
| ceruloplasmin precursor | NP_000087.1 | LEFALLFLVFDENESWYLDDNIK | 325 | 1417.202 | 1066.52 |
| ceruloplasmin precursor | NP_000087.1 | LEFALLFLVFDENESWYLDDNIK | 325 | 1417.202 | 1153.552 |
| ceruloplasmin precursor | NP_000087.1 | LEFALLFLVFDENESWYLDDNIK | 325 | 1417.202 | 1282.594 |
| ceruloplasmin precursor | NP_000087.1 | LEFALLFLVFDENESWYLDDNIK | 325 | 1417.202 | 1396.637 |

### Example 2

### Mass Spectrometry Methods for Determining Expression Levels of Pre-Selected Biomarkers that are Predictive of Trisomy 21 Pregnancies During the First Trimester

### Materials and Methods

### Clinical Serum Samples

The clinical samples used in this Example are as described herein in Example 1.

Serum Processing without Enrichment

Serum samples without enrichment were prepared by diluting serum 1:4 v/v with 8M GuHC1/150 mM Tris/10 mM DTT pH 8.5. Samples were incubated at 37° C. for 60 minutes and then cooled to room temperature. Next, 500 mM iodoacetic acid/1M Tris pH 8.5 was added to each sample to a final concentration of 40 mM. Samples were alkylated in the dark at room temperature for 60 minutes. The reaction was quenched with the addition of 2 M DTT to a final concentration of 5 mM. Post quench, samples were diluted to 1 ml with the addition of 50 mM Tris/5 mM CaCl₂, pH 8.0 and 10 μg of sequencing grade trypsin (Promega) were added to each sample prior to incubation at 37° C. for 24 hours. The digestion reaction was quenched with the addition of TFA to 1%. Subsequent to digestion, the samples were processed by solid phase extraction for desalting and clean-up using Hypersep™ C18 50 mg 96 well plates (ThermoFisher). Plates were equilibrated once with 100% ACN and then rinsed twice with 0.25% TFA. Samples were then loaded and wells were rinsed 5 times with 0.25% TFA. The peptides were eluted with 400 μl of 75% ACN/0.1% formic acid at 300 rcf in a centrifuge equipped with a swinging bucket rotor. After elution, the samples were lyophilized. Just before loading on the mass spectrometer, peptides were resuspended in 97% H₂O/3% ACN/0.25% formic acid.

### High Resolution LC-MS/MS

High resolution LC-MS/MS analysis was carried out as described herein in Example 1.

### Selective Reaction Monitoring (SRM) Assays

SRM assays were performed as described herein in Example 1.

### Results

Using the techniques described above, the expression levels of 171 peptide fragments representing 19 target proteins were shown to be differentially expressed in first trimester maternal serum samples from Trisomy 21 pregnancies relative to normal pregnancies. The amino acid sequences, m/z transitions and parent target protein for each of the identified peptides are shown in Table 2.

| | NCBI | | SEQ | | | |
|---|---|---|---|---|---|---|
| | Accession | Amino Acid Sequence | ID | Parent Peptide | Fragment | Fragmentation |
| Protein Name | No. | of Peptide Fragment | NO: | m/z | Ion Ion m/z | Energy |
| paraoxonase 1 | NP_000437.3 | IFFYDSENPPASEVLR | 258 | 942.4623413 | 868.4882 | 35.35772 |
| paraoxonase 1 | NP_000437.3 | IFFYDSENPPASEVLR | 258 | 942.4623413 | 982.5311 | 35.35772 |
| paraoxonase 1 | NP_000437.3 | IFFYDSENPPASEVLR | 258 | 942.4623413 | 603.3455 | 35.35772 |
| paraoxonase 1 | NP_000437.3 | IFFYDSENPPASEVLR | 258 | 942.4623413 | 771.4354 | 35.35772 |
| paraoxonase 1 | NP_000437.3 | IFFYDSENPPASEVLR | 258 | 942.4623413 | 1198.606 | 35.35772 |
| paraoxonase 1 | NP_000437.3 | IFFYDSENPPASEVLR | 258 | 942.4623413 | 1313.633 | 35.35772 |
| paraoxonase 1 | NP_000437.3 | IFFYDSENPPASEVLR | 258 | 942.4623413 | 812.3858 | 35.35772 |
| paraoxonase 1 | NP_000437.3 | IFFYDSENPPASEVLR | 258 | 942.4623413 | 387.2709 | 35.35772 |
| paraoxonase 1 | NP_000437.3 | IFFYDSENPPASEVLR | 258 | 942.4623413 | 516.3135 | 35.35772 |
| pregnancy-zone protein | NP_002855.1 | ATVLNYLPK | 175 | 509.8002319 | 747.4394 | 20.64721 |
| pregnancy-zone protein | NP_002855.1 | ATVLNYLPK | 175 | 509.8002319 | 846.5078 | 20.64721 |
| pregnancy-zone protein | NP_002855.1 | ATVLNYLPK | 175 | 509.8002319 | 244.165 | 20.64721 |
| pregnancy-zone protein | NP_002855.1 | ATVLNYLPK | 175 | 509.8002319 | 634.3553 | 20.64721 |
| pregnancy-zone protein | NP_002855.1 | ATVLNYLPK | 175 | 509.8002319 | 423.7575 | 20.64721 |
| pregnancy-zone protein | NP_002855.1 | ATVLNYLPK | 175 | 509.8002319 | 357.2491 | 20.64721 |
| pregnancy-zone protein | NP_002855.1 | ATVLNYLPK | 175 | 509.8002319 | 520.3124 | 20.64721 |
| pregnancy-zone protein | NP_002855.1 | ATVLNYLPK | 175 | 509.8002319 | 474.2814 | 20.64721 |
| pregnancy-zone protein | NP_002855.1 | ATVLNYLPK | 175 | 509.8002319 | 179.1282 | 20.64721 |
| alpha-2-macroglobulin | NP_000005.2 | GHFSISIPVK | 254 | 542.8110962 | 890.5341 | 21.76958 |
| precursor | | | | | | |
| alpha-2-macroglubulin | NP_000005.2 | GHFSISIPVK | 254 | 542.8110962 | 343.2334 | 21.76958 |
| precursor | | | | | | |
| alpha-2-macroglubulin | NP_000005.2 | GHFSISIPVK | 254 | 542.8110962 | 743.4656 | 21.76958 |
| precursor | | | | | | |
| alpha-2-macroglubulin | NP_000005.2 | GHFSISIPVK | 254 | 542.8110962 | 656.4336 | 21.76958 |
| precursor | | | | | | |
| alpha-2-macroglubulin | NP_000005.2 | GHFSISIPVK | 254 | 542.8110962 | 456.3175 | 21.76958 |
| precursor | | | | | | |
| alpha-2-macroglubulin | NP_000005.2 | GHFSISIPVK | 254 | 542.8110962 | 514.3001 | 21.76958 |
| precursor | | | | | | |
| alpha-2-macroglubulin | NP_000005.2 | GHFSISIPVK | 254 | 542.8110962 | 543.3495 | 21.76958 |
| precursor | | | | | | |
| alpha-2-macroglubulin | NP_000005.2 | GHFSISIPVK | 254 | 542.8110962 | 372.2365 | 21.76958 |
| precursor | | | | | | |
| alpha-2-macroglubulin | NP_000005.2 | GHFSISIPVK | 254 | 542.8110962 | 328.7204 | 21.76958 |
| precursor | | | | | | |
| serum amyloid A4; | NP_006503.1 | EALQGVGDMGR | 98 | 566.7744141 | 634.2972 | 22.58433 |
| constitutive | | | | | | |
| serum amyloid A4; | NP_006503.1 | EALQGVGDMGR | 98 | 566.7744141 | 535.2288 | 22.58433 |
| constitutive | | | | | | |
| serum amyloid A4; | NP-006503.1 | EALQGVGDMGR | 98 | 566.7744141 | 691.3187 | 22.58433 |
| constitutive | | | | | | |
| serum amyloid A4; | NP_006503.1 | EALQGVGDMGR | 98 | 566.7744141 | 466.7343 | 22.58433 |
| constitutive | | | | | | |
| serum amyloid A4; | NP_006503.1 | EALQGVGDMGR | 98 | 566.7744141 | 363.1804 | 22.58433 |
| constitutive | | | | | | |
| serum amyloid A4; | NP_006503.1 | EALQGVGDMGR | 98 | 566.7744141 | 819.3772 | 22.58433 |
| constitutive | | | | | | |
| serum amyloid A4; | NP_006503.1 | EALQGVGDMGR | 98 | 566.7744141 | 478.2073 | 22.58433 |
| constitutive | | | | | | |
| serum amyloid A4; | NP_006503.1 | EALQGVGDMGR | 98 | 566.7744141 | 410.1922 | 22.58433 |
| constitutive | | | | | | |
| serum amyloid A4; | NP_006503.1 | EALQGVGDMGR | 98 | 566.7744141 | 232.1399 | 22.58433 |
| constitutive | | | | | | |
| serum amyloid P component | NP_001630.1 | VGEYSLYIGR | 177 | 578.8034668 | 708.4034 | 22.99332 |
| precursor | | | | | | |
| serum amyloid P component | NP_001630.1 | VGEYSLYIGR | 177 | 578.8034668 | 508.2873 | 22.99332 |
| precursor | | | | | | |
| serum amyloid P component | NP_001630.1 | VGEYSLYIGR | 177 | 578.8034668 | 871.4667 | 22.99332 |
| precursor | | | | | | |
| serum amyloid P component | NP_001630.1 | VGEYSLYIGR | 177 | 578.8034668 | 621.3713 | 22.99332 |
| precursor | | | | | | |
| serum amyloid P component | NP_001630.1 | VGEYSLYIGR | 177 | 578.8034668 | 345.2239 | 22.99332 |
| precursor | | | | | | |
| serum amyloid P component | NP_001630.1 | VGEYSLYIGR | 177 | 578.8034668 | 232.1399 | 22.99332 |
| precursor | | | | | | |
| serum amyloid P component | NP_001630.1 | VGEYSLYIGR | 177 | 578.8034668 | 1057.531 | 22.99332 |
| precursor | | | | | | |
| serum amyloid P component | NP_001630.1 | VGEYSLYIGR | 177 | 578.8034668 | 1000.509 | 22.99332 |
| precursor | | | | | | |
| serum amyloid P component | NP_001630.1 | VGEYSLYIGR | 177 | 578.8034668 | 436.237 | 22.99332 |
| precursor | | | | | | |
| beta globin | NP_000509.1 | LLVVYPWTQR | 242 | 637.866394 | 687.3568 | 25.00146 |
| beta globin | NP_000509.1 | LLVVYPWTQR | 242 | 637.866394 | 850.42 | 25.00146 |
| beta globin | NP_000509.1 | LLVVYPWTQR | 242 | 637.866394 | 949.4885 | 25.00146 |
| beta globin | NP_000509.1 | LLVVYPWTQR | 242 | 637.866394 | 425.7137 | 25.00146 |
| beta globin | NP_000509.1 | LLVVYPWTQR | 242 | 637.866394 | 1048.557 | 25.00146 |
| beta globin | NP_000509.1 | LLVVYPWTQR | 242 | 637.866394 | 524.7821 | 25.00146 |
| beta globin | NP_000509.1 | LLVVYPWTQR | 242 | 637.866394 | 475.2479 | 25.00146 |
| beta globin | NP_000509.1 | LLVVYPWTQR | 242 | 637.866394 | 303.177 | 25.00146 |
| beta globin | NP_000509.1 | LLVVYPWTQR | 242 | 637.866394 | 404.2247 | 25.00146 |
| ceruloplasmin precursor | NP_000087.1 | GAYPLSIEPIGVR | 267 | 686.3851929 | 540.824 | 26.6511 |
| ceruloplasmin precursor | NP_000087.1 | GAYPLSIEPIGVR | 267 | 686.3851929 | 670.3877 | 26.6511 |
| ceruloplasmin precursor | NP_000087.1 | GAYPLSIEPIGVR | 267 | 686.3851929 | 870.5038 | 26.6511 |
| ceruloplasmin precursor | NP_000087.1 | GAYPLSIEPIGVR | 267 | 686.3851929 | 622.3556 | 26.6511 |
| ceruloplasmin precursor | NP_000087.1 | GAYPLSIEPIGVR | 267 | 686.3851929 | 1080.641 | 26.6511 |
| ceruloplasmin precursor | NP_000087.1 | GAYPLSIEPIGVR | 267 | 686.3851929 | 783.4717 | 26.6511 |
| ceruloplasmin precursor | NP_000087.1 | GAYPLSIEPIGVR | 267 | 686.3851929 | 983.5878 | 26.6511 |
| ceruloplasmin precursor | NP_000087.1 | GAYPLSIEPIGVR | 267 | 686.3851929 | 331.2083 | 26.6511 |
| ceruloplasmin precursor | NP_000087.1 | GAYPLSIEPIGVR | 267 | 686.3851929 | 541.3451 | 26.6511 |
| alpha 1 globin | NP_000549.1 | TYFPHFDLSHGSAQVK | 221 | 611.968811 | 711.8596 | 30.24063 |
| alpha 1 globin | NP_000549.1 | TYFPHFDLSHGSAQVK | 221 | 611.968811 | 785.3938 | 30.24063 |
| alpha 1 globin | NP_000549.1 | TYFPHFDLSHGSAQVK | 221 | 611.968811 | 442.5579 | 30.24063 |
| alpha 1 globin | NP_000549.1 | TYFPHFDLSHGSAQVK | 221 | 611.968811 | 523.9316 | 30.24063 |
| alpha 1 globin | NP_000549.1 | TYFPHFDLSHGSAQVK | 221 | 611.968811 | 663.3332 | 30.24063 |
| alpha 1 globin | NP_000549.1 | TYFPHFDLSHGSAQVK | 221 | 611.968811 | 474.9088 | 30.24063 |
| alpha 1 globin | NP_000549.1 | TYFPHFDLSHGSAQVK | 221 | 611.968811 | 589.3298 | 30.24063 |
| alpha 1 globin | NP_000549.1 | TYFPHFDLSHGSAQVK | 221 | 611.968811 | 463.7561 | 30.24063 |
| alpha 1 globin | NP_000549.1 | TYFPHFDLSHGSAQVK | 221 | 917.449585 | 1041.532 | 34.50729 |
| alpha 1 globin | NP_000549.1 | TYFPHFDLSHGSAQVK | 221 | 917.449585 | 926.5049 | 34.50729 |
| alpha 1 globin | NP_000549.1 | TYFPHFDLSHGSAQVK | 221 | 917.449585 | 813.4208 | 34.50729 |
| alpha 1 globin | NP_000549.1 | TYFPHFDLSHGSAQVK | 221 | 917.449585 | 1188.6 | 34.50729 |
| histidine-rich glycoprotein | NP_000403.1 | DGYLFQLLR | 278 | 562.8085938 | 676.4135 | 22.44949 |
| precursor | | | | | | |
| histidine-rich glycoprotein | NP_000403.1 | DGYLFQLLR | 278 | 562.8085938 | 789.4976 | 22.44949 |
| precursor | | | | | | |
| histidine-rich glycoprotein | NP_000403.1 | DGYLFQLLR | 278 | 562.8085938 | 529.3451 | 22.44949 |
| precursor | | | | | | |
| histidine-rich glycoprotein | NP_000403.1 | DGYLFQLLR | 278 | 562.8085938 | 288.2025 | 22.44949 |
| precursor | | | | | | |
| histidine-rich glycoprotein | NP_000403.1 | DGYLFQLLR | 278 | 562.8085938 | 401.2865 | 22.44949 |
| precursor | | | | | | |
| histidine-rich glycoprotein | NP_000403.1 | DGYLFQLLR | 278 | 562.8085938 | 201.1469 | 22.44949 |
| precursor | | | | | | |
| histidine-rich glycoprotein | NP_000403.1 | DGYLFQLLR | 278 | 562.8085938 | 476.7841 | 22.44949 |
| precursor | | | | | | |
| histidine-rich glycoprotein | NP_000403.1 | DGYLFQLLR | 278 | 562.8085938 | 395.2524 | 22.44949 |
| precursor | | | | | | |
| histidine-rich glycoprotein | NP_000403.1 | DGYLFQLLR | 278 | 562.8085938 | 338.7104 | 22.44949 |
| precursor 3 | | | | | | |
| apolipoprotein H precursor 3 | NP_000033.1 | ATVVYQGER | 45 | 511.7669067 | 652.3044 | 20.71407 |
| apolipoprotein H precursor | NP_000033.1 | ATVVYQGER | 45 | 511.7669067 | 751.3728 | 20.71407 |
| apolipoprotein H precursor | NP_000033.1 | ATVVYQGER | 45 | 511.7669067 | 425.7242 | 20.71407 |
| apolipoprotein H precursor | NP_000033.1 | ATVVYQGER | 45 | 511.7669067 | 361.1825 | 20.71407 |
| apolipoprotein H precursor | NP_000033.1 | ATVVYQGER | 45 | 511.7669067 | 850.4412 | 20.71407 |
| apolipoprotein H precursor | NP_000033.1 | ATVVYQGER | 45 | 511.7669067 | 489.241 | 20.71407 |
| apolipoprotein H precursor | NP_000033.1 | ATVVYQGER | 45 | 511.7669067 | 304.161 | 20.71407 |
| apolipoprotein H precursor | NP_000033.1 | ATVVYQGER | 45 | 511.7669067 | 326.6558 | 20.71407 |
| apolipoprotein H precursor | NP_000033.1 | ATVVYQGER | 45 | 511.7669067 | 376.19 | 20.71407 |
| apolipoprotein B precursor | NP_000375.2 | IGISPLALIK | 246 | 506.8237 | 741.4863 | 20.54601 |
| apolipoprotein B precursor | NP_000375.2 | IGISPLALIK | 246 | 506.8237 | 654.4543 | 20.54601 |
| apolipoprotein B precursor | NP_000375.2 | IGISPLALIK | 246 | 506.8237 | 854.5704 | 20.54601 |
| apolipoprotein B precursor | NP_000375.2 | IGISPLALIK | 246 | 506.8237 | 427.7888 | 20.54601 |
| apolipoprotein B precursor | NP_000375.2 | IGISPLALIK | 246 | 506.8237 | 260.1963 | 20.54601 |
| apolipoprotein B precursor | NP_000375.2 | IGISPLALIK | 246 | 506.8237 | 327.7308 | 20.54601 |
| apolipoprotein B precursor | NP_000375.2 | IGISPLALIK | 246 | 506.8237 | 373.2804 | 20.54601 |
| apolipoprotein B precursor | NP_000375.2 | IGISPLALIK | 246 | 506.8237 | 444.3175 | 20.54601 |
| apolipoprotein B precursor | NP_000375.2 | IGISPLALIK | 246 | 506.8237 | 911.5919 | 20.54601 |
| apolipoprotein E precursor | NP_000032.1 | FWDYLR | 253 | 450.2241516 | 566.2927 | 18.62162 |
| apolipoprotein E precursor | NP_000032.1 | FWDYLR | 253 | 450.2241516 | 752.3721 | 18.62162 |
| apolipoprotein E precursor | NP_000032.1 | FWDYLR | 253 | 450.2241516 | 288.2025 | 18.62162 |
| apolipoprotein E precursor | NP_000032.1 | FWDYLR | 253 | 450.2241516 | 376.6897 | 18.62162 |
| apolipoprotein E precursor | NP_000032.1 | FWDYLR | 253 | 450.2241516 | 451.2658 | 18.62162 |
| apolipoprotein E precursor | NP_000032.1 | FWDYLR | 253 | 450.2241516 | 226.1365 | 18.62162 |
| apolipoprotein A-I | NP_000030.1 | LLDNWDSVTSTFSK | 268 | 806.8963013 | 971.4675 | 30.74847 |
| preproprotein | | | | | | |
| apolipoprotein A-I | NP_000030.1 | LLDNWDSVTSTFSK | 268 | 806.8963013 | 670.3401 | 30.74847 |
| preproprotein | | | | | | |
| apolipoprotein A-I | NP_000030.1 | LLDNWDSVTSTFSK | 268 | 806.8963013 | 856.4406 | 30.74847 |
| preproprotein | | | | | | |
| apolipoprotein A-I | NP_000030.1 | LLDNWDSVTSTFSK | 268 | 806.8963013 | 569.2924 | 30.74847 |
| preproprotein | | | | | | |
| apolipoprotein A-I | NP_000030.1 | LLDNWDSVTSTFSK | 268 | 806.8963013 | 1386.617 | 30.74847 |
| preproprotein | | | | | | |
| apolipoprotein A-I | NP_000030.1 | LLDNWDSVTSTFSK | 268 | 806.8963013 | 1157.547 | 30.74847 |
| preproprotein | | | | | | |
| apolipoprotein A-I | NP_000030.1 | LLDNWDSVTSTFSK | 268 | 806.8963013 | 769.4085 | 30.74847 |
| preproprotein | | | | | | |
| apolipoprotein A-I | NP_000030.1 | LLDNWDSVTSTFSK | 268 | 806.8963013 | 1271.59 | 30.74847 |
| preproprotein | | | | | | |
| apolipoprotein A-I | NP_000030.1 | LLDNWDSVTSTFSK | 268 | 806.8963013 | 482.2604 | 30.74847 |
| preproprotein | | | | | | |
| apolipoprotein C-III | NP_000031.1 | GWVTDGFSSLK | 226 | 598.8009644 | 854.4249 | 23.67323 |
| precursor | | | | | | |
| apolipoprotein C-III | NP_000031.1 | GWVTDGFSSLK | 226 | 598.8009644 | 953.4933 | 23.67323 |
| precursor | | | | | | |
| apolipoprotein C-III | NP_000031.1 | GWVTDGFSSLK | 226 | 598.8009644 | 753.3772 | 23.67323 |
| precursor | | | | | | |
| apolipoprotein C-III | NP_000031.1 | GWVTDGFSSLK | 226 | 598.8009644 | 638.3503 | 23.67323 |
| precursor | | | | | | |
| apolipoprotein C-III | NP_000031.1 | GWVTDGFSSLK | 226 | 598.8009644 | 434.2604 | 23.67323 |
| precursor | | | | | | |
| apolipoprotein C-III | NP_000031.1 | GWVTDGFSSLK | 226 | 598.8009644 | 581.3288 | 23.67323 |
| precursor | | | | | | |
| apolipoprotein C-III | NP_000031.1 | GWVTDGFSSLK | 226 | 598.8009644 | 347.2283 | 23.67323 |
| precursor | | | | | | |
| apolipoprotein C-III | NP_000031.1 | GWVTDGFSSLK | 226 | 598.8009644 | 477.2503 | 23.67323 |
| precursor | | | | | | |
| apolipoprotein C-III | NP_000031.1 | GWVTDGFSSLK | 226 | 598.8009644 | 260.1963 | 23.67323 |
| precursor | | | | | | |
| apolipoprotein C-I precursor | NP_001636.1 | LKEFGNTLEDK | 233 | 647.3378906 | 1052.489 | 25.32349 |
| apolipoprotein C-I precursor | NP_001636.1 | LKEFGNTLEDK | 233 | 647.3378906 | 923.4464 | 25.32349 |
| apolipoprotein C-I precursor | NP_001636.1 | LKEFGNTLEDK | 233 | 647.3378906 | 776.3779 | 25.32349 |
| apolipoprotein C-I precursor | NP_001636.1 | LKEFGNTLEDK | 233 | 647.3378906 | 590.7956 | 25.32349 |
| apolipoprotein C-I precursor | NP_001636.1 | LKEFGNTLEDK | 233 | 647.3378906 | 262.1392 | 25.32349 |
| apolipoprotein C-I precursor | NP_001636.1 | LKEFGNTLEDK | 233 | 647.3378906 | 719.3564 | 25.32349 |
| apolipoprotein C-I precursor | NP_001636.1 | LKEFGNTLEDK | 233 | 647.3378906 | 504.2659 | 25.32349 |
| apolipoprotein C-I precursor | NP_001636.1 | LKEFGNTLEDK | 233 | 647.3378906 | 388.6926 | 25.32349 |
| apolipoprotein C-I precursor | NP_001636.1 | LKEFGNTLEDK | 233 | 647.3378906 | 605.3135 | 25.32349 |
| apolipoprotein D precursor | NP_001638.1 | NILTSNNIDVK | 243 | 615.8380737 | 1003.541 | 24.25249 |
| apolipoprotein D precursor | NP_001638.1 | NILTSNNIDVK | 243 | 615.8380737 | 890.4572 | 24.25249 |
| apolipoprotein D precursor | NP_001638.1 | NILTSNNIDVK | 243 | 615.8380737 | 789.4095 | 24.25249 |
| apolipoprotein D precursor | NP_001638.1 | NILTSNNIDVK | 243 | 615.8380737 | 361.2076 | 24.25249 |
| apolipoprotein D precursor | NP_001638.1 | NILTSNNIDVK | 243 | 615.8380737 | 246.1807 | 24.25249 |
| apolipoprotein D precursor | NP_001638.1 | NILTSNNIDVK | 243 | 615.8380737 | 502.2743 | 24.25249 |
| apolipoprotein D precursor | NP_001638.1 | NILTSNNIDVK | 243 | 615.8380737 | 588.3346 | 24.25249 |
| apolipoprotein D precursor | NP_001638.1 | NILTSNNIDVK | 243 | 615.8380737 | 702.3775 | 24.25249 |
| apolipoprotein D precursor | NP_001638.1 | NILTSNNIDVK | 243 | 615.8380737 | 474.2917 | 24.25249 |
| apolipoprotein A-II | NP_001634.1 | SKEQLTPLIK | 249 | 578.8504639 | 941.566 | 22.99492 |
| preproprotein | | | | | | |
| apolipoprotein A-II | NP_001634.1 | SKEQLTPLIK | 249 | 578.8504639 | 470.3332 | 22.99492 |
| preproprotein | | | | | | |
| apolipoprotein A-II | NP_001634.1 | SKEQLTPLIK | 249 | 578.8504639 | 812.5234 | 22.99492 |
| preproprotein | | | | | | |
| apolipoprotein A-II | NP_001634.1 | SKEQLTPLTK | 249 | 578.8504639 | 684.4649 | 22.99492 |
| preproprotein | | | | | | |
| apolipoprotein A-II | NP_001634.1 | SKEQLTPLIK | 249 | 578.8504639 | 471.2867 | 22.99492 |
| preproprotein | | | | | | |
| apolipoprotein A-II | NP_001634.1 | SKEQLTPLIK | 249 | 578.8504639 | 260.1963 | 22.99492 |
| preproprotein | | | | | | |
| apolipoprotein A-II | NP_001634.1 | SKEQLTPLIK | 249 | 578.8504639 | 535.3342 | 22.99492 |
| preproprotein | | | | | | |
| apolipoprotein A-II | NP_001634.1 | SKEQLTPLIK | 249 | 578.8504639 | 373.2804 | 22.99492 |
| preproprotein | | | | | | |
| apolipoprotein A-II | NP_001634.1 | SKEQLTPLIK | 249 | 578.8504639 | 286.1941 | 22.99492 |
| preproprotein | | | | | | |
| apolipoprotein A-IV | NP_000473.2 | LGPHAGDVEGHLSFLEK | 265 | 602.6442261 | 579.811 | 29.83035 |
| precursor | | | | | | |
| apolipoprotein A-IV | NP_000473.2 | LGPHAGDVEGHLSFLEK | 265 | 602.6442261 | 536.3073 | 29.83035 |
| precursor | | | | | | |
| apolipoprotein A-IV | NP_000473.2 | LGPHAGDVEGHLSFLEK | 265 | 602.6442261 | 545.9422 | 29.83035 |
| precursor | | | | | | |
| apolipoprotein A-IV | NP_000473.2 | LGPHAGDVEGHLSFLEK | 265 | 602.6442261 | 736.4234 | 29.83035 |
| precursor | | | | | | |
| apolipoprotein A-IV | NP_000473.2 | LGPHAGDVEGHLSFLEK | 265 | 602.6442261 | 701.3538 | 29.83035 |
| precursor | | | | | | |
| apolipoprotein A-IV | NP_000473.2 | LGPHAGDVEGHLSFLEK | 265 | 602.6442261 | 564.9493 | 29.83035 |
| precursor | | | | | | |
| apolipoprotein A-IV | NP_000473.2 | LGPHAGDVEGHLSFLEK | 265 | 602.6442261 | 465.7555 | 29.83035 |
| precursor | | | | | | |
| apolipoprotein A-IV | NP_000473.2 | LGPHAGDVEGHLSFLEK | 265 | 602.6442261 | 623.3394 | 29.83035 |
| precursor | | | | | | |
| apolipoprotein A-IV | NP_000473.2 | LGPHAGDVEGHLSFLEK | 265 | 602.6442261 | 769.8832 | 29.83035 |
| precursor | | | | | | |
| apolipoprotein C-II precursor | NP_000474.2 | ESLSSYWESAK | 161 | 643.798584 | 620.3033 | 25.20315 |
| apolipoprotein C-II precursor | NP_000474.2 | ESLSSYWESAK | 161 | 643.798584 | 870.3987 | 25.20315 |
| apolipoprotein C-II precursor | NP_000474.2 | ESLSSYWESAK | 161 | 643.798584 | 783.3666 | 25.20315 |
| apolipoprotein C-II precursor | NP_000474.2 | ESLSSYWESAK | 161 | 643.798584 | 434.224 | 25.20315 |
| apolipoprotein C-II precursor | NP_000474.2 | ESLSSYWESAK | 161 | 643.798584 | 957.4307 | 25.20315 |
| apolipoprotein C-II precursor | NP_000474.2 | ESLSSYWESAK | 161 | 643.798584 | 305.1814 | 25.20315 |
| apolipoprotein C-II precursor | NP_000474.2 | ESLSSYWESAK | 161 | 643.798584 | 1070.515 | 25.20315 |
| apolipoprotein C-II precursor | NP_000474.2 | ESLSSYWESAK | 161 | 643.798584 | 535.761 | 25.20315 |
| apolipoprotein C-II precursor | NP_000474.2 | ESLSSYWESAKI | 161 | 643.7985844 | 35.703 | 25.20315 |

The relevant teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method for predicting whether a fetus has Trisomy 21, comprising the steps of:
(a) measuring the expression level of a peptide fragment of at least one target protein selected from the group consisting of apolipoprotein A-I preprotein, apolipoprotein A-II preprotein, apolipoprotein A-IV precursor, apolipoprotein B precursor, apolipoprotein C-I precursor, apolipoprotein C-II precursor, apolipoprotein C-III precursor, apolipoprotein D precursor, apolipoprotein E precursor, apolipoprotein H precursor, paraoxonase 1, pregnancy-zone protein, α-2-macroglobulin precursor, serum amyloid A4, serum amyloid P component precursor, β-globin, ceruloplasmin precursor, α-1 globin and histidine-rich glycoprotein precursor in a serum sample obtained from the mother of the fetus during the first trimester of pregnancy, wherein the expression level is measured using tandem mass spectrometry; and
(b) comparing the expression level of the peptide fragment in the sample to a corresponding control level, wherein a difference in the expression level of the peptide fragment in the sample relative to the corresponding control level is predictive of a fetus having Trisomy 21.

2. The method of claim 1, wherein the peptide fragment is selected from the group consisting of SEQ ID NO:45, SEQ ID NO:98, SEQ ID NO:161, SEQ ID NO:175, SEQ ID NO:177, SEQ ID NO:221, SEQ ID NO:226, SEQ ID NO:233, SEQ ID NO:242, SEQ ID NO:243, SEQ ID NO:246, SEQ ID NO:249, SEQ ID NO:253, SEQ ID NO:254, SEQ ID NO:258, SEQ ID NO:265, SEQ ID NO: 267, SEQ ID NO:268, and SEQ ID NO:278.

3. The method of claim 2, wherein the peptide fragment is SEQ ID NO:258 and wherein the expression level of the peptide fragment is measured by selective reaction monitoring (SRM) tandem mass spectrometry using an m/z transition selected from the group consisting of about 942→387, 942→516, 942→603, 942→771, 942→812, 942→868, 942→982, 942→1199, and 942→1314.

4. The method of claim 2, wherein the peptide fragment is SEQ ID NO:175 and wherein the expression level of the peptide fragment is measured by selective reaction monitoring (SRM) tandem mass spectrometry using an m/z transition selected from the group consisting of about 510→179, 510→244, 510→357, 510→424, 510→474, 510→520, 510→534, 510→747, and 510→847.

5. The method of claim 2, wherein the peptide fragment is SEQ ID NO:254 and wherein the expression level of the peptide fragment is measured by selective reaction monitoring (SRM) tandem mass spectrometry using an m/z transition selected from the group consisting of about 543→329, 543→343, 543→372, 543→456, 543→514, 543→543, 543→656, 543→743, and 543→891.

6. The method of claim 2, wherein the peptide fragment is SEQ ID NO:98 and wherein the expression level of the peptide fragment is measured by selective reaction monitoring (SRM) tandem mass spectrometry using an m/z transition selected from the group consisting of about 567→232, 567→363, 567→410, 567→467, 567→478, 567→535, 567→634, 567→691, and 567→819.

7. The method of claim 2, wherein the peptide fragment is SEQ ID NO:177 and wherein the expression level of the peptide fragment is measured by selective reaction monitoring (SRM) tandem mass spectrometry using an m/z transition selected from the group consisting of about 579→232, 579→345, 579→436, 579→508, 579→621, 579→708, 579→871, 579→1001, and 579→1058.

8. The method of claim 2, wherein the peptide fragment is SEQ ID NO:242 and wherein the expression level of the peptide fragment is measured by selective reaction monitoring (SRM) tandem mass spectrometry using an m/z transition selected from the group consisting of about 638→303, 638→404, 638→426, 638→475, 638→525, 638→687, 638→850, 638→949, and 638→1049.

9. The method of claim 2, wherein the peptide fragment is SEQ ID NO:267 and wherein the expression level of the peptide fragment is measured by selective reaction monitoring (SRM) tandem mass spectrometry using an m/z transition selected from the group consisting of about 686→331, 686→541, 686→622, 686→670, 686→783, 686→871, 686→984, and 686→1081.

10. The method of claim 2, wherein the peptide fragment is SEQ ID NO:221 and wherein the expression level of the peptide fragment is measured by selective reaction monitoring (SRM) tandem mass spectrometry using an m/z transition selected from the group consisting of about 612→443, 612→464, 612→475, 612→524, 612→589, 612→663, 612→712, 612→785, 612→813, 612→927, 612→1041, and 612→1189.

11. The method of claim 2, wherein the peptide fragment is SEQ ID NO:278 and wherein the expression level of the peptide fragment is measured by selective reaction monitoring (SRM) tandem mass spectrometry using an m/z transition selected from the group consisting of about 563→201, 563→288, 563→339, 563→395, 563→401, 563→477, 563→529, 563→676, and 563→789.

12. The method of claim 2, wherein the peptide fragment is SEQ ID NO:45 and wherein the expression level of the peptide fragment is measured by selective reaction monitoring (SRM) tandem mass spectrometry using an m/z transition selected from the group consisting of about 512→304, 512→327, 512→361, 512→376, 512→426, 512→489, 512→652, 512→751, and 512→850.

13. The method of claim 2, wherein the peptide fragment is SEQ ID NO:246 and wherein the expression level of the peptide fragment is measured by selective reaction monitoring (SRM) tandem mass spectrometry using an m/z transition selected from the group consisting of about 507→260, 507→328, 507→373, 507→427, 507→444, 507→654, 507→741, 507→855, and 507→912.

14. The method of claim 2, wherein the peptide fragment is SEQ ID NO:253 and wherein the expression level of the peptide fragment is measured by selective reaction monitoring (SRM) tandem mass spectrometry using an m/z transition selected from the group consisting of about 450→226, 450→288, 450→377, 450→451, 450→566, and 450→752.

15. The method of claim 2, wherein the peptide fragment is SEQ ID NO:268 and wherein the expression level of the peptide fragment is measured by selective reaction monitoring (SRM) tandem mass spectrometry using an m/z transition selected from the group consisting of about 807→482, 807→569, 807→670, 807→769, 807→856, 807→971, 807→1158, 807→1272, and 807→1387.

16. The method of claim 2, wherein the peptide fragment is SEQ ID NO:226 and wherein the expression level of the peptide fragment is measured by selective reaction monitoring (SRM) tandem mass spectrometry using an m/z transition selected from the group consisting of about 599→260, 599→347, 599→434, 599→477, 599→581, 599→638, 599→753, 599→854, and 599→953.

17. The method of claim 2, wherein the peptide fragment is SEQ ID NO:233 and wherein the expression level of the peptide fragment is measured by selective reaction monitoring (SRM) tandem mass spectrometry using an m/z transition selected from the group consisting of about 647→262, 647→389, 647→504, 647→591, 647→605, 647→719, 647→776, 647→923, and 647→1052.

18. The method of claim 2, wherein the peptide fragment is SEQ ID NO:243 and wherein the expression level of the peptide fragment is measured by selective reaction monitoring (SRM) tandem mass spectrometry using an m/z transition selected from the group consisting of about 616→246, 616→361, 616→474, 616→502, 616→588, 616→702, 616→789, 616→890, and 616→1004.

19. The method of claim 2, wherein the peptide fragment is SEQ ID NO:249 and wherein the expression level of the peptide fragment is measured by selective reaction monitoring (SRM) tandem mass spectrometry using an m/z transition selected from the group consisting of about 579→260, 579→286, 579→373, 579→470, 579→471, 579→535, 579→684, 579→813, and 579→942.

20. The method of claim 2, wherein the peptide fragment is SEQ ID NO:265 and wherein the expression level of the peptide fragment is measured by selective reaction monitoring (SRM) tandem mass spectrometry using an m/z transition selected from the group consisting of about 603→466, 603→536, 603→546, 603→565, 603→580, 603→623, 603→701, 603→736, and 603→770.

21. The method of claim 2, wherein the peptide fragment is SEQ ID NO:161 and wherein the expression level of the peptide fragment is measured by selective reaction monitoring (SRM) tandem mass spectrometry using an m/z transition selected from the group consisting of about 644→305, 644→434, 644→436, 644→536, 644→620, 644→783, 644→870, 644→957, and 644→1071.

22. The method of claim 1, wherein the sample is enriched for peptide fragments bound to carrier proteins prior to mass spectrometry.

23. The method of claim 1, wherein the sample is reduced, alkylated and desalted prior to mass spectrometry.

24. The method of claim 1, wherein the sample is subjected to one dimensional SDS-polyacrylamide gel electrophoresis prior to mass spectrometry.

25. The method of claim 1, wherein the sample is subjected to isoelectric focusing prior to mass spectrometry.

26. The method of claim 1, wherein proteins from the sample are subjected to trypsin proteolysis prior to mass spectrometry.

27. The method of claim 1, wherein proteins from the sample are subjected to liquid chromatography prior to mass spectrometry.

28. The method of claim 1, wherein the tandem mass spectrometry is electrospray ionization tandem mass spectrometry.

29. The method of claim 28, wherein the electrospray ionization tandem mass spectrometry is performed using a quadrapole mass spectrometer.
